(19) United States
(12) Patent Application Publication
(10) Pub. No.: US 2025/0049895 A1
(43) Pub. Date: Feb. 13, 2025

(54) TREATING & PREVENTING E COLI INFECTIONS

(71) Applicant: SNIPR Biome ApS, Copenhagen Ø (DK)

(72) Inventors: Jakob Krause HAABER, Copenhagen Ø (DK); Szabolcs SEMSEY, Copenhagen Ø (DK); Mette GROVE, Copenhagen Ø (DK); Birgitte DAMHOLT, Copenhagen Ø (DK); Dziuginta JASINSKYTE, Copenhagen Ø (DK); Yilmaz Emre GENÇAY, Copenhagen Ø (DK)

(73) Assignee: SNIPR Biome ApS, Copenhagen Ø (DK)

(21) Appl. No.: 18/778,686

(22) Filed: Jul. 19, 2024

Related U.S. Application Data
(62) Division of application No. 17/812,359, filed on Jul. 13, 2022, now Pat. No. 12,076,375.

(30) Foreign Application Priority Data
Jun. 29, 2022 (GB) 2209518.6

(51) Int. Cl. A61K 38/46; A61K 35/76; A61P 31/04; C12N 15/113

(52) U.S. Cl. CPC A61K 38/465; A61K 35/76; A61P 31/04; C12N 15/113; C12N 2310/20

(57) ABSTRACT

The invention relates to methods and compositions for treating or preventing an infection by E coli cells in human or animal subjects. The method comprises administering to the subject a plurality of transduction particles that encode a nuclease for targeting the genomes of B2 phylogroup E coli cells.

### CROSS REFERENCE TO RELATED APPLICATIONS

This application is a divisional of U.S. application Ser. No. 17/812,359, filed on Jul. 13, 2022, which claims benefit of Great Britain Application No.: GB2209518.6, filed on Jun. 29, 2022, the disclosures of each of which are herein incorporated by reference in their entirety.

### REFERENCE TO AN ELECTRONIC SEQUENCE LISTING

The content of the electronic sequence listing (786212001410seqlist.xml; Size: 181,625 bytes; and Date of Creation: Jul. 15, 2024) is herein incorporated by reference in its entirety.

### TECHNICAL FIELD

The technology described herein relates to methods and compositions for treating or preventing an infection by E coli cells in human or animal subjects. The method comprises administering to the subject a plurality of transduction particles that encode a nuclease for targeting the genomes of B2 phylogroup E coli cells.

### BACKGROUND

E coli infection has been identified as harmful or life-threatening in various settings, such as UTI infections, transplant patients, cancer patients and other patients that are immunocompromised or on immunosuppressant.

Nuclease targeting of E coli, such as by means of CRISPR/Cas systems, has been proposed with delivery using transduction particles that can target the nuclease to E coli cells for chromosomal or episomal cutting, thereby killing cells or reducing their growth or proliferation. Suitable transduction particles are phage or engineered particles (such as non-self-replicative transduction particles) comprising capsids that contain nucleic acid encoding at least crRNAs or gRNAs (or additionally a Cas nuclease) for targeting. Advantageously, selective targeting can be achieved which is not possible using conventional antibiotics, such as broad-spectrum antibiotics. Such selective targeting can avoid killing of beneficial species and strains in treated patients. Indeed, disturbances of the microbiome with broad-spectrum antibiotics is a risk-factor in the prophylactic management of cancer patients at risk of febrile neutropenia.

Bacteriophage (phage) therapy has been used prior to the broad availability of antibiotics, but has now re-gained interest due to the rise in bacterial antimicrobial resistance (AMR) combined with several successful individual case reports.

Bacteriophages (phages) are a phylum of viruses that infect bacteria, and are distinct from the animal and plant viruses. Phages can have either a “lytic” life cycle, a “lysogenic” life cycle that can potentially become lytic, or a “non-lytic” life cycle. Phages replicating through the lytic cycle cause lysis of the host bacterial cell as a normal part of their life cycles. Phages replicating through the lysogenic cycles are called temperate phages, and can either replicate by means of the lytic life cycle and cause lysis of the host bacterium, or they can incorporate their DNA into the host bacterial DNA and become noninfectious prophages.

The natural capability of phages to infect and kill bacteria, together with the specificity of the phage-bacterial interactions, is the basic phenomena on which the concept of phage therapy is built. Therefore, phages that possess lytic life cycle are suitable candidates for phage therapy.

International Patent Application No. WO 00/69269 discloses the use of certain phage strain for treating infections caused by Vancomycin-sensitive as well as resistant strains of Enterococcus faecium, and International Patent Application No. WO 01/93904 discloses the use of bacteriophage, alone or in combination with other anti-microbial means, for preventing or treating gastrointestinal diseases associated with the species of the genus Clostridium.

US Patent Application No. 2002/0001590 discloses the use of phage therapy against multi-drug resistant bacteria, specifically methicillin-resistant Staphylococcus aureus, and International Patent Application No. WO 02/07742 discloses the development of bacteriophage having multiple host range.

The use of phage therapy for the treatment of specific bacterial-infectious disease is disclosed, for example, in US Patent Application Nos. 2002/0044922; 2002/0058027 and International Patent Application No. WO 01/93904.

US20160333348 describes the use of CRISPR/Cas systems delivered to host bacterial cells using phage as vectors.

Amongst the several phylogroups of E coli, it has been observed that antibitic-resitant (eg, fluoroquinolone (FQ)-resistant) and multi-drug resistant (MDR) strains are frequently found in the B2 phylogroup. B2 strains ST131 and ST1193 have been found that are associated with antibiotic resistance. ST131 is a globally dominant multidrug resistant clone associated with high rates in rUTI. ST131 is a major contributor to hospital- and community-acquired UTI, as well as E coli bloodstream infections and infections in companion animals and poultry. Originally identified in 2008, ST131 is associated with the worldwide spread of the CTX-M-15 extended spectrum β-lactamase (ESBL) resistance gene. ST131 is now strongly associated with multidrug resistance (MDR), including resistance to fluoroquinolones. Recent reports have also identified strains that are resistant to last-line carbapenems. Sequence type 1193 has recently emerged as a new, virulent and resistant lineage among fluoroquinolone resistant E coli.

Classic antibiotics, such as FQ and broad-spectrum antibiotics are not, thus, sufficiently effective for combatting such infections. There is, therefore, a need to find alternative means to address these infections.

### SUMMARY OF THE INVENTION

The invention provides means for treating or preventing B2 phylogroup E coli infections in humans and animals by combining the use of selective killing with nucleases targeted using specific types of transduction particles. The particles of the invention target by adhesion to LPS, LamB or Tsx, which has surprisingly been found highly advantageous for killing and inhibiting growth of B2 E coli cells of many different strains (including the potentially lethal ST131 and ST1193 strains). As exemplified herein, surprisingly more than 10 different ST131 strains were killed (plaques formed) and more than 10 different ST1193 strains were killed (plaques formed).

The invention finds utility, for example, to treat or prevent potentially life-threating B2 phylogroup E coli infections in patients, such as in immunosuppressed, cancer, transplant and UTI patients, who are susceptible to infection by B2 strains (and often by multiple different B2 strains). As demonstrated in the Examples, the invention is useful for preventing E coli B2 phylogroup bacteraemia (infection of the blood stream by E coli) in a subject.

To this end, the invention provides:

### In a First Configuration

### In a First Aspect:

A composition comprising a plurality of transduction particles for use in a method of treating or preventing an infection by E coli cells in a human or animal subject, wherein the method comprises administering the particles to the subject, wherein
(a) each particle comprises a nucleic acid encoding a nuclease for targeting the genomes of E coli cells, wherein the said administered particles contact the cells and introduce therein the nucleic acid, wherein the nuclease is expressed in the cells and cuts genomic DNA of the cells, thereby killing the cells or reducing growth or proliferation of the cells in the subject;
(b) the E coli cells are cells of E coli phylogroup B2; and
(c) each particle comprises an adhesion moiety for recognising and binding to a cognate moiety selected from a LPS, LamB and Tsx displayed on the surface of the phylogroup B2 E coli cells.

### In a Second Aspect:

A composition comprising a plurality of transduction particles for use in a method of treating or preventing E coli bacteriaemia in a human or animal subject, wherein the method comprises administering the particles to the subject, wherein
(a) each particle comprises a nucleic acid encoding a nuclease for targeting the genomes of cells of said E coli, wherein the said administered particles contact the cells and introduce therein the nucleic acid, wherein the nuclease is expressed in the cells and cuts genomic DNA of the cells, thereby killing the cells or reducing growth or proliferation of the cells in the subject;
(b) the E coli cells are cells of E coli phylogroup B2; and
(c) each particle comprises an adhesion moiety for recognising and binding to a cognate moiety selected from a LPS, LamB and Tsx displayed on the surface of the phylogroup B2 E coli cells.

### In a Second Configuration

### In a First Aspect:

A method for treating or preventing an infection by E coli cells in a human or animal subject, the method comprising administering to the subject a plurality of transduction particles, wherein
(a) each particle comprises a nucleic acid encoding a nuclease for targeting the genomes of E coli cells, wherein the said administered particles contact the cells and introduce therein the nucleic acid, wherein the nuclease is expressed in the cells and cuts genomic DNA of the cells, thereby killing the cells or reducing growth or proliferation of the cells in the subject;
(b) the E coli cells are cells of E coli phylogroup B2; and
(c) each particle comprises an adhesion moiety for recognising and binding to a cognate moiety selected from a LPS, LamB and Tsx displayed on the surface of the phylogroup B2 E coli cells.

### In a Second Aspect:

A method for treating or preventing an infection by E coli cells in a human or animal subject, the method comprising administering to the subject a plurality of transduction particles, wherein
(a) each particle comprises a nucleic acid encoding a crRNA or guide RNA that is operable with a Cas nuclease for chromosomal targeting in the cells, wherein the said administered particles contact the cells and introduce therein the nucleic acid, wherein the crRNA or guide RNA is expressed and guides the Cas nuclease wherein the nuclease cuts the chromosomes of the cells, thereby killing the cells or reducing growth or proliferation of the cells in the subject;
(b) the E coli cells are cells of E coli phylogroup B2; and
(c) each particle comprises an adhesion moiety for recognising and binding to a cognate moiety selected from a LPS, LamB and Tsx displayed on the surface of the phylogroup B2 E coli cells.

### In a Third Aspect:

A method for treating or preventing E coli bacteriaemia in a human or animal subject, the method comprising administering to the subject a plurality of transduction particles, wherein
(a) each particle comprises a nucleic acid encoding a nuclease for targeting the genomes of cells of said E coli, wherein the said administered particles contact the cells and introduce therein the nucleic acid, wherein the nuclease is expressed in the cells and cuts genomic DNA of the cells, thereby killing the cells or reducing growth or proliferation of the cells in the subject;
(b) the E coli cells are cells of E coli phylogroup B2; and
(c) each particle comprises an adhesion moiety for recognising and binding to a cognate moiety selected from a LPS, LamB and Tsx displayed on the surface of the phylogroup B2 E coli cells.

### In a Fourth Aspect:

A method for treating or preventing E coli bacteriaemia in a human or animal subject, the method comprising administering to the subject a plurality of transduction particles, wherein
(a) each particle comprises a nucleic acid encoding a crRNA or guide RNA that is operable with a Cas nuclease for chromosomal targeting in cells of said E coli, wherein the said administered particles contact the cells and introduce therein the nucleic acid, wherein the crRNA or guide RNA is expressed and guides the Cas nuclease wherein the nuclease cuts the chromosomes of the cells, thereby killing the cells or reducing growth or proliferation of the cells in the subject;
(b) the E coli cells are cells of E coli phylogroup B2; and
(c) each particle comprises an adhesion moiety for recognising and binding to a cognate moiety selected from a LPS, LamB and Tsx displayed on the surface of the phylogroup B2 E coli cells.

### In a Third Configuration

### In a First Aspect:

Use of a composition comprising a plurality of transduction particles in a method for treating or preventing an infection by phylogroup B2 E coli cells in a human or animal subject, the method comprising administering to the subject the composition, wherein
(a) each particle comprises a nucleic acid encoding a nuclease for targeting the genomes of E coli cells, wherein the said administered particles contact the cells and introduce therein the nucleic acid, wherein the nuclease is expressed in the cells and cuts genomic DNA of the cells, thereby killing the cells or reducing growth or proliferation of the cells in the subject; and
(b) each particle comprises an adhesion moiety for recognising and binding to a cognate moiety selected from a LPS, LamB and Tsx displayed on the surface of the phylogroup B2 E coli cells.

### In a Second Aspect:

Use of a composition comprising a plurality of transduction particles in a method for treating or preventing an infection by phylogroup B2 E coli cells in a human or animal subject, the method comprising administering to the subject the composition, wherein
(a) each particle comprises a nucleic acid encoding a crRNA or guide RNA that is operable with a Cas nuclease for chromosomal targeting in the cells, wherein the said administered particles contact the cells and introduce therein the nucleic acid, wherein the crRNA or guide RNA is expressed and guides the Cas nuclease wherein the nuclease cuts the chromosomes of the cells, thereby killing the cells or reducing growth or proliferation of the cells in the subject;
(b) the E coli cells are cells of E coli phylogroup B2; and
(c) each particle comprises an adhesion moiety for recognising and binding to a cognate moiety selected from a LPS, LamB and Tsx displayed on the surface of the phylogroup B2 E coli cells.

### In a Third Aspect:

Use of a composition comprising a plurality of transduction particles in a method for treating or preventing phylogroup B2 E coli bacteraemia in a human or animal subject, the method comprising administering to the subject the composition, wherein
(a) each particle comprises a nucleic acid encoding a nuclease for targeting the genomes of cells of said E coli, wherein the said administered particles contact the cells and introduce therein the nucleic acid, wherein the nuclease is expressed in the cells and cuts genomic DNA of the cells, thereby killing the cells or reducing growth or proliferation of the cells in the subject; and
(b) each particle comprises an adhesion moiety for recognising and binding to a cognate moiety selected from a LPS, LamB and Tsx displayed on the surface of the phylogroup B2 E coli cells.

### In a Fourth Aspect:

Use of a composition comprising a plurality of transduction particles in a method for treating or preventing phylogroup B2 E coli bacteraemia in a human or animal subject, the method comprising administering to the subject the composition, wherein
(a) each particle comprises a nucleic acid encoding a crRNA or guide RNA that is operable with a Cas nuclease for chromosomal targeting in cells of said E coli, wherein the said administered particles contact the cells and introduce therein the nucleic acid, wherein the crRNA or guide RNA is expressed and guides the Cas nuclease wherein the nuclease cuts the chromosomes of the cells, thereby killing the cells or reducing growth or proliferation of the cells in the subject;
(b) the E coli cells are cells of E coli phylogroup B2; and
(c) each particle comprises an adhesion moiety for recognising and binding to a cognate moiety selected from a LPS, LamB and Tsx displayed on the surface of the phylogroup B2 E coli cells.

### BRIEF DESCRIPTION OF THE DRA WINGS

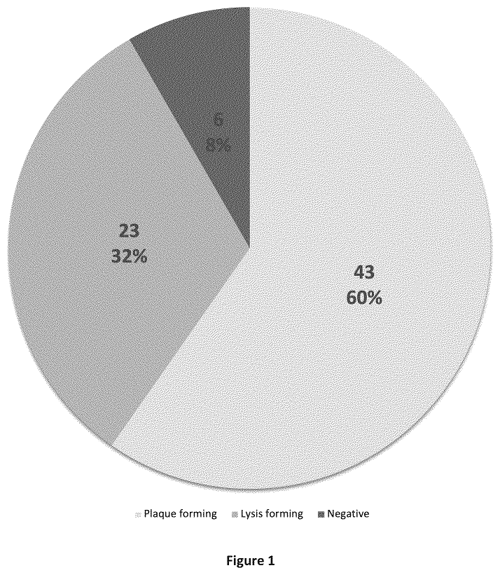

FIG. 1: Killing and growth inhibition activity of a composition of the invention when tested against E coli of several different phylogroups and different strains within each group (E coli taken from clinical samples); the composition was surprisingly highly effective at killing across many phylogroups (and particularly in the B2 phylogroup, where multiple strains-including those known to be potentially life-threatening and related with antibiotic resistance-were killed or whose growth was effectively inhibited). The 6 negatives comprised non E coli and non PQ resistant samples.

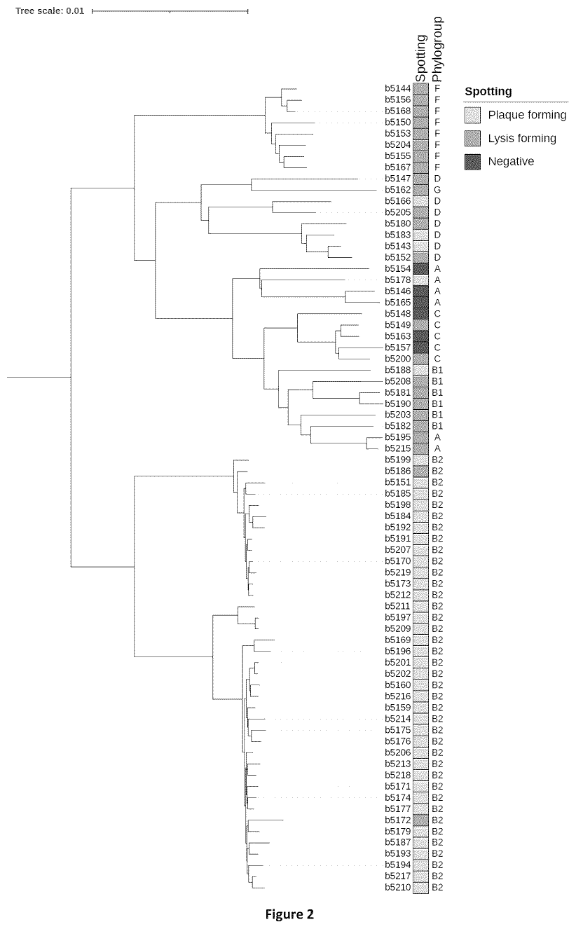

FIG. 2: Phylo-grouping of strains used in the study and showing particularly advantageous and extensive targeting of many clinical B2 phylogroup strains.

### DETAILED DESCRIPTION

The invention finds application to combat harmful or life-threatening B2 E coli infections in various settings, such as UTI infections, transplant patients, cancer patients and other patients that are immunocompromised or on immunosuppressant.

Cancer treatment continues to advance and survival rates for people with hematological malignancies are increasing. However, this population is immunocompromised and chemotherapy regimens cause bone-marrow suppression and gastrointestinal mucositis with associated increased intestinal permeability. Translocation of gut bacteria, including E coli, from the gastrointestinal tract is a frequent cause of bloodstream infections (BSIs). The mortality-related to BSIs can be up to 50%; thus, antimicrobial prophylaxis is applied in people at risk of febrile neutropenia. There are no approved therapies for the prevention of BSIs in patients with hematological cancers, yet fluoroquinolones are used off-label in the United States. This antibiotic prophylaxis practice is at odds with the emerging paradigm that maintaining a normal microbiome is important for upholding immunological tonus potentially benefiting the outcome of oncology treatments. Indeed, disturbances of the microbiome with broad-spectrum antibiotics is a risk-factor in the prophylactic management of patients at risk of febrile neutropenia. Beyond the side effects of fluoroquinolones, including safety warnings and precautions, bacterial resistance is rising and approaching 60% in USA.

In immunocompromised patients with hematological malignancies at risk of developing neutropenia, E coli is responsible for 25.1-30% of all bacteraemia cases with a 35.8% 90-day mortality rate. Moreover, up to 65% of E coli isolated as the causative pathogen from BSIs in patients with hematological cancers undergoing hematopoietic stem cell transplantation (HSCT) were resistant to fluoroquinolones. Accordingly, novel narrow-spectrum treatment and prophylactic options are needed to prevent infections in these vulnerable patients. The invention addresses this need.

To this end, the invention provides compositions, methods and uses according to the above Configurations. There is, thus, provided the following description with numbered Embodiments.

1. A composition comprising a plurality of transduction particles for use in a method of treating or preventing an infection by E coli cells in a human or animal subject, wherein the method comprises administering the particles to the subject, wherein
(a) each particle comprises a nucleic acid encoding a nuclease for targeting the genomes of E coli cells, wherein the said administered particles contact the cells and introduce therein the nucleic acid, wherein the nuclease is expressed in the cells and cuts genomic DNA of the cells, thereby killing the cells or reducing growth or proliferation of the cells in the subject;
(b) the E coli cells are cells of E coli phylogroup B2; and
(c) each particle comprises an adhesion moiety for recognising and binding to a cognate moiety selected from a LPS, LamB and Tsx displayed on the surface of phylogroup B2 E coli cells.

2. A method for treating or preventing an infection by E coli cells in a human or animal subject, the method comprising administering to the subject a plurality of transduction particles, wherein the method comprises administering the particles to the subject, wherein
(a) each particle comprises a nucleic acid encoding a nuclease for targeting the genomes of E coli cells, wherein the said administered particles contact the cells and introduce therein the nucleic acid, wherein the nuclease is expressed in the cells and cuts genomic DNA of the cells, thereby killing the cells or reducing growth or proliferation of the cells in the subject;
(b) the E coli cells are cells of E coli phylogroup B2; and
(c) each particle comprises an adhesion moiety for recognising and binding to a cognate moiety selected from a LPS, LamB and Tsx displayed on the surface of the phylogroup B2 E coli cells.

Optionally, the genomic DNA is chromosomal DNA of the cells. Additionally or alternatively, the genomic DNA is plasmid DNA of the cells.

Optionally, each particle comprises a nucleic acid encoding a nuclease for chromosomal targeting, wherein the said administered particles contact the cells and introduce therein the nucleic acid, wherein the nuclease is expressed in the cells and cuts the chromosomes of the cells, thereby killing the cells or reducing growth or proliferation of the cells in the subject.

The human may be a male or female. The human may be an adult or child. The human may be 18 years of age or older, eg, 40, 50, 60, 70 80 or older. The human may be younger than 18, eg, a teenager, eg, a baby, eg, up to 5 years of age, eg, up to 2 years of age. The animal may be be a livestock or companion animal eg, a dog or cat). The animal may be a bird (eg, a poultry bird, eg, a chicken, turkey or duck, preferably a chicken), cow, sheep, goat or pig (eg, a neonatal swine or a swine under 6 months of age).

The infection may be a bloodstream infection. The infection may be a nosocomial infection.

In an example, each adhesion moiety is a tail fibre protein. In an example, each particle comprises a phage tail fibre that comprises or is fused to a said adhesion moiety. In an example, each adhesion moiety is an antibody fragment, eg, an antibody single variable domain. In an example, each adhesion moiety is a nanobody. In an example, each adhesion moiety comprises an antibody binding site that is capable of binding to the cognate moiety. For example, each adhesion moiety comprises an antibody single variable domain (ie, a dAb), such as a nanobody. In an example, each particle comprises one or more phage tail fibres or spikes, each fibre or spike comprising a said adhesion moiety.

For example, at least 2, 3 or 4 different types of said transduction particle are administered to the subject and each type comprises one or a plurality of types of tail fibres comprising adhesion moieties, wherein the other particle types do not comprise said one or plurality of tail fibre types. In an example, each type of said plurality of tail fibre types differs from the other types by the type of adhesion moiety it comprises.

Optionally, the particles comprise adhesion moieties for binding to LPS, LamB and Tsx. Optionally, the particles comprise adhesion moieties for binding to LamB and Tsx. Optionally, the particles comprise adhesion moieties for binding to LPS and Tsx. Optionally, the particles comprise adhesion moieties for binding to LPS and LamB.

In Gram-negative bacteria, the peptidoglycan layer is relatively thin and is located inward of the outer membrane, the major component of the cell wall. These two layers are connected by Braun's lipoproteins. The outer membrane is a sophisticated structure composed of a lipid bilayer ornamented with proteins, polysaccharides and lipids; the latter two molecules form the LPS layer. LPSs are complexes that consist of three parts: lipid A, the core polysaccharide and the O-polysaccharide. Lipid A is, in general, composed of fatty acids attached to glucosamine phosphate disaccharides. The core polysaccharide is connected to the lipid A through a ketodeoxyoctonate linker. The core polysaccharide and the O-polysaccharide (O-chain or O-antigen) contain several units of sugar residues extending outward to the outer membrane. Cells that contain all three components of the LPS are denominated as smooth(S) type and those that lack the O-polysaccharide portion are distinguished as rough (R) type.

Optionally, the LPS is smooth LPS or rough LPS.

For example, the particles comprise at least one type of particle whose adhesion moiety is capable of binding to O-antigen of LPS.

E coli is a very versatile species for which diversity has been explored from various perspectives highlighting, for example, phylogenetic groupings, pathovars as well as a wide range of O serotypes. The highly variable O-antigen, the most external part of the lipopolysaccharide component of the outer membrane of E coli, is linked to the innermost lipid A through the core region of LPS of which 5 different structures, denominated K-12, R1, R2, R3 and R4, have been characterized so far. Phylogroups B2 and C strains are mainly dominated by the R1 type. Strains within phylogroup B2 may carry a K-12 core, eg, belonging to the complex STc131, one of the major clone of extra-intestinal pathogenic E coli (ExPEC) strains.

Preferably, the LPS comprises a R1 core region. In an example, the LPS comprises a R2 core region. In an example, the LPS comprises a R3 core region. In an example, the LPS comprises a R4 core region. In an example, the LPS comprises a K-12 core region.

Optionally, the LamB comprises the amino acid of SEQ ID NO: 1 or an amino acid sequence that is at least 70, 80, 90 or 95% identical to SEQ ID NO: 1. Optionally, the Tsx comprises the amino acid of SEQ ID NO: 2 or an amino acid sequence that is at least 70, 80, 90 or 95% identical to SEQ ID NO: 2. Optionally, the LamB is encoded by the nucleotide sequence of SEQ ID NO: 3 or an amino acid sequence that is at least 70, 80, 90 or 95% identical to SEQ ID NO: 3. Optionally, the Tsx is encoded by the nucleotide sequence of SEQ ID NO: 4 or an amino acid sequence that is at least 70, 80, 90 or 95% identical to SEQ ID NO: 4.

The Escherichia coli tsx gene encodes an integral outer-membrane protein (Tsx) that functions as a substrate-specific channel for deoxynucleosides and the antibiotic albicidin. In an example, the Nucleoside-specific channel-forming protein Tsx of E coli has a Uniprot Accession Number of POA927 or is a homologue thereof. In an example, the maltose outer membrane porin (maltoporin) LamB of E coli has a Uniprot Accession Number of P02943 or is a homologue thereof.

Homologue: A gene, nucleotide or protein sequence related to a second gene, nucleotide or protein sequence by descent from a common ancestral DNA or protein sequence. The term, homologue, may apply to the relationship between genes separated by the event of or to the relationship between genes separated by the event of genetic duplication.

In an embodiment, the E coli cells comprise UPEC E coli. In an embodiment, the E coli cells comprise intestinal pathogenic E coli (ExPEC) cells.

3. The composition or method of Embodiment 1 or 2 respectively, wherein the method is for treating or preventing infection of the subject by an E coli strain selected from the group ST131, ST1193, ST648, ST315, ST405, ST361, ST88 and ST453.

In a preferred example, the strain is ST1193. In another preferred example, the strain is ST131.

4. The composition or method of any preceding Embodiment, wherein the method is for treating or preventing infection of the subject by a plurality of different phylogroup B2 strains of E coli; optionally wherein the plurality comprises E coli ST131 and ST1193 cells.

E coli ST131 and/or ST1193 have been found to be virulent and associated with fluoroquinolone resistance. As shown in the Example section herein, the invention is useful for treating or preventing infection by such strains. Optionally, said plurality of strains comprises E coli ST131 and/or ST1193 strains. Optionally, said plurality of strains comprises fluoroquinolone-resistant strains. For example, said plurality of strains comprises E coli ST131 and/or ST1193 fluoroquinolone-resistant strains. E coli strains B2-ST73 (CH24-30); B2-ST73 (CH24-103); B2-ST131 (CH40-30); B2-ST141 (CH52-5); B2-ST372 (CH103-9); B2-ST404 (CH14-27); B2-ST404 (CH14-807) and B2-ST1193 (CH14-64) have been found in UTI settings. In an embodiment (eg, wherein the subject is suffering from or at risk of UTI), the B2 E coli comprise one or more strains selected from B2-ST73 (CH24-30); B2-ST73 (CH24-103); B2-ST131 (CH40-30); B2-ST141 (CH52-5); B2-ST372 (CH103-9); B2-ST404 (CH14-27); B2-ST404 (CH14-807) and B2-ST1193 (CH14-64).

5. The composition or method of any preceding Embodiment, wherein the subject is a transplant or cancer patient (optionally a haematological cancer patient), or wherein the patient is suffering from or at risk of a urinary tract infection (UTI); and optionally wherein the transplant is a solid organ or stem cell transplant (optionally a haematopoietic cell transplant) or wherein the transplant is a transplant of a medical device.

For example, the subject is a haematological cancer patient suffering from neutropenia. For example, the subject is a haematopoietic stem cell transplant patient.

A suitable medical device may be, eg, heart device (eg, ventricular assist device, such as a left ventricular assist device (LVAD)), a catheter (eg, a biliary catheter) or a prosthesis (eg, a joint) prosthesis.

In an example, the patient is suffering from a sequestered B2 phylogroup E coli infection. In an example, the E coli are sequestered B2 phylogroup E coli.

For example, the subject is suffering from or at risk of acute bacterial sinusitis, pneumonia, urinary tract infections, chronic prostatitis or gastroenteritis caused by B2 phylogroup E coli. For example, the subject is a male human prostate surgery patient.

6. The composition or method of any preceding Embodiment, wherein the method is carried out prior to the subject receiving a transplant.

For example, the transplant is a solid organ or stem cell transplant (optionally a haematopoietic cell transplant).

7. The composition or method of any preceding Embodiment, wherein the B2 E coli cells comprise a strain of E coli that causes sepsis, septicaemia or diarrhoea in humans.

Enterohemorrhagic Escherichia coli (EHEC) serotype O157: H7 is a human pathogen responsible for outbreaks of bloody diarrhoea and haemolytic uremic syndrome (HUS) worldwide. Conventional antimicrobials trigger an SOS response in EHEC that promotes the release of the potent Shiga toxin that is responsible for much of the morbidity and mortality associated with EHEC infection. Cattle are a natural reservoir of EHEC, and approximately 75% of EHEC outbreaks are linked to the consumption of contaminated bovine-derived products. EHEC causes disease in humans but is asymptomatic in adult ruminants. Characteristics of E. coli serotype O157: H7 (EHEC) infection includes abdominal cramps and bloody diarrhoea, as well as the life-threatening complication haemolytic uremic syndrome (HUS). Currently there is a need for a treatment for EHEC infections (Goldwater and Bettelheim, 2012). The use of conventional antibiotics exacerbates Shiga toxin-mediated cytotoxicity. In an epidemiology study conducted by the Centers for Disease Control and Prevention, patients treated with antibiotics for EHEC enteritis had a higher risk of developing HUS (Slutsker et al., 1998). Additional studies support the contraindication of antibiotics in EHEC infection; children on antibiotic therapy for hemorrhagic colitis associated with EHEC had an increased chance of developing HUS (Wong et al., 2000; Zimmerhackl, 2000; Safdar et al., 2002; Tarr et al., 2005). Conventional antibiotics promote Shiga toxin production by enhancing the replication and expression of stx genes that are encoded within a chromosomally integrated lambdoid prophage genome. The approach of the present invention may rely on nuclease cutting of target cell genomic DNA. Stx induction also promotes phage-mediated lysis of the EHEC cell envelope, allowing for the release and dissemination of Shiga toxin into the environment (Karch et al., 1999; Matsushiro et al., 1999; Wagner et al., 2002). Thus, advantageously, the invention provides alternative means for treating B2 phylogroup EHEC in human and animal subjects. In an example, the subject (eg, a human) is suffering from or at risk of haemolytic uremic syndrome (HUS), eg, the subject is suffering from an E coli infection, such as an EHEC E coli infection.

8. The composition or method of any preceding Embodiment for preventing haemolytic uremic syndrome (HUS), a UTI infection, sepsis, septicaemia or diarrhoea in the subject.

The composition or method may be for treating or preventing a blood stream infection by pathogenic B2 phylogroup E coli cells in the subject.

9. The composition or method of any preceding Embodiment, wherein each particle comprises a phage capsid containing the nucleic acid; optionally wherein the capsid comprises capsid proteins of a T-even (optionally T4) or lambda phage.

As is known the to the skilled addressee, transduction particles are operable to infect their cognate host cells to introduce therein nucleic acid by transduction.

10. The composition or method of any preceding Embodiment, wherein each particle is a phage (optionally a lytic phage) or packaged phagemid.

11. The composition or method of any preceding Embodiment, wherein at least 2, 3 or 4 different types of transduction particle are administered to the subject.

For example, each particle types comprises a type of adhesion moiety or collection of adhesion moiety types that differs from the other types of particles.

In an example, 2 different types of transduction particle are administered to the subject. In an example, 3 different types of transduction particle are administered to the subject. In an example, 4 different types of transduction particle are administered to the subject. In an example, 5 different types of transduction particle are administered to the subject. In an example, 6 different types of transduction particle are administered to the subject.

12. The composition or method of any preceding Embodiment, wherein a first type of transduction particle and a second type of transduction particle are administered to the subject, wherein the first type of particle comprises a first adhesion moiety that is capable of recognising and binding to a first cognate moiety selected from the group LPS, LamB and Tsx displayed on B2 E coli, and the second type of particle comprises a second adhesion moiety that is capable of recognising and binding to a second cognate moiety selected from said group, wherein the first and second adhesion moieties different from each other.

For example, the first and second adhesion moieties different from each other by their tail fibres, optionally wherein the first adhesion moiety is cognate to LPS and the second moiety is cognate to LamB; or optionally wherein the first adhesion moiety is cognate to LPS and the second moiety is cognate to Tsx; optionally wherein the first adhesion moiety is cognate to Tsx and the second moiety is cognate to LamB.

For example, each particle comprises a phage capsid containing a said nucleic acid; wherein at least 2, 3 or 4 different types of transduction particle are administered to the subject; and wherein a first type of transduction particle and a second type of transduction particle are administered to the subject, wherein the first type of particle comprises a first adhesion moiety that is capable of recognising and binding to a first cognate moiety selected from the group LPS, LamB and Tsx displayed on B2 E coli, and the second type of particle comprises a second adhesion moiety that is capable of recognising and binding to a second cognate moiety selected from said group, wherein the first and second adhesion moieties different from each other.

For example, each particle comprises a phage capsid containing a said nucleic acid; wherein at least 2, 3 or 4 different types of transduction particle are administered to the subject; and wherein a first type of transduction particle and a second type of transduction particle are administered to the subject, wherein the first type of particle comprises a first adhesion moiety that is capable of recognising and binding to LPS displayed on B2 E coli, and the second type of particle comprises a second adhesion moiety that is capable of recognising and binding to LamB displayed on B2 E coli.

For example, each particle comprises a phage capsid containing a said nucleic acid; wherein at least 2, 3 or 4 different types of transduction particle are administered to the subject; and wherein a first type of transduction particle and a second type of transduction particle are administered to the subject, wherein the first type of particle comprises a first adhesion moiety that is capable of recognising and binding to Tsx displayed on B2 E coli, and the second type of particle comprises a second adhesion moiety that is capable of recognising and binding to LamB displayed on B2 E coli.

For example, each particle comprises a phage capsid containing a said nucleic acid; wherein at least 2, 3 or 4 different types of transduction particle are administered to the subject; and wherein a first type of transduction particle and a second type of transduction particle are administered to the subject, wherein the first type of particle comprises a first adhesion moiety that is capable of recognising and binding to LPS displayed on B2 E coli, and the second type of particle comprises a second adhesion moiety that is capable of recognising and binding to Tsx displayed on B2 E coli.

For example, each particle comprises a phage capsid containing a said nucleic acid; wherein at least 3 different types of transduction particle are administered to the subject; and wherein a first type of transduction particle, a second type of transduction particle and a third type of transduction particle are administered to the subject, wherein the first type of particle comprises a first adhesion moiety that is capable of recognising and binding to LPS displayed on B2 E coli, the second type of particle comprises a second adhesion moiety that is capable of recognising and binding to LamB displayed on B2 E coli, and the third type of particle comprises a second adhesion moiety that is capable of recognising and binding to Tsx displayed on B2 E coli.

For example, each particle comprises a phage capsid containing a said nucleic acid; wherein at least 4 different types of transduction particle are administered to the subject; and wherein a first type of transduction particle, a second type of transduction particle, a third type of transduction particle and a fourth type of transduction particle are administered to the subject, wherein the first type of particle comprises a first adhesion moiety that is capable of recognising and binding to LPS displayed on B2 E coli, the second type of particle comprises a second adhesion moiety that is capable of recognising and binding to LamB displayed on B2 E coli, the third type of particle comprises a second adhesion moiety that is capable of recognising and binding to Tsx displayed on B2 E coli, and the fourth type of particle comprises a second adhesion moiety that is capable of recognising and binding to LPS displayed on B2 E coli, wherein the adhesion moieties of said particles are different from each other.

For example, each particle comprises a phage capsid containing a said nucleic acid; wherein at least 4 different types of transduction particle are administered to the subject; and wherein a first type of transduction particle, a second type of transduction particle, a third type of transduction particle and a fourth type of transduction particle are administered to the subject, wherein the first type of particle comprises a first adhesion moiety that is capable of recognising and binding to LPS displayed on B2 E coli, the second type of particle comprises a second adhesion moiety that is capable of recognising and binding to LamB displayed on B2 E coli, the third type of particle comprises a second adhesion moiety that is capable of recognising and binding to Tsx displayed on B2 E coli, and the fourth type of particle comprises a second adhesion moiety that is capable of recognising and binding to LamB displayed on B2 E coli, wherein the adhesion moieties of said particles are different from each other.

For example, each particle comprises a phage capsid containing a said nucleic acid; wherein at least 4 different types of transduction particle are administered to the subject; and wherein a first type of transduction particle, a second type of transduction particle, a third type of transduction particle and a fourth type of transduction particle are administered to the subject, wherein the first type of particle comprises a first adhesion moiety that is capable of recognising and binding to LPS displayed on B2 E coli, the second type of particle comprises a second adhesion moiety that is capable of recognising and binding to LamB displayed on B2 E coli, the third type of particle comprises a second adhesion moiety that is capable of recognising and binding to Tsx displayed on B2 E coli, and the fourth type of particle comprises a second adhesion moiety that is capable of recognising and binding to Tsx displayed on B2 E coli, wherein the adhesion moieties of said particles are different from each other.

13. The composition or method of Embodiment 12, wherein the first and second cognate moieties are different from each other.

In an alternative, the first and second cognate moieties are identical. In an alternative, the first and second cognate moieties are LPS. In an alternative, the first and second cognate moieties are Tsx. In an alternative, the first and second cognate moieties are LamB.

14. The composition or method of any preceding Embodiment, wherein the nucleic acid of each particle comprises a nucleotide sequence (N1) encoding said nuclease, wherein each particle is a synthetic T-even phage (optionally a T4 phage) comprising an insertion of N1 into the genome of the phage, wherein the region is between the pin (protease inhibitor) gene and the iPII (internal protein) gene.

Optionally, the phage is
(a) a synthetic T-even (eg, a T4) phage that comprises a deletion of DNA from, and/or an insertion of heterologous DNA into, a region of the genome of the phage corresponding to a region between coordinates
   (i) 1887 and 8983;
   (ii) 2625 and 8092;
   (iii) 1904 and 8113;
   (iv) 2668 and 7178;
   (v) 7844 and 11117;
   (vi) 8643 and 10313;
   (vii) 9231 and 13383;
   (viii) 9480 and 12224;
   (ix) 8454 and 17479; or
   (x) 9067 and 16673;
wherein coordinates are with reference to wild-type T4 phage genome (SEQ ID NO: 5);

15. The composition or method of any preceding Embodiment, wherein said nuclease is a guided nuclease, optionally a Cas, meganuclease, zinc finger nuclease or TALEN.

16. The composition or method of any preceding Embodiment, wherein the nuclease is a Type I, II, III, IV, V or VI nuclease, optionally a Cas9 or a Cas3.

17. The composition or method of any preceding Embodiment, wherein at least 1×10⁷ PFU of particles are administered to the subject.

In an example, 1×10⁸ to 1×10¹³ PFU of particles are administered to the subject. In an example, 1×10⁸ to 1×10¹² PFU of particles are administered to the subject. In an example, 1×10¹⁰ to 1×10¹² PFU of particles are administered to the subject.

18. The composition or method of any preceding Embodiment, wherein the particles are administered to the subject at an MOI (multiplicity of infection) of at least 0.01.

Optionally, the particles are administered to the subject at an MOI of no more than 1. Optionally, the particles are administered to the subject at an MOI from 0.001 to 1. Optionally, the particles are administered to the subject at an MOI from 0.01 to 1. Optionally, the particles are administered to the subject at an MOI from 0.1 to 1.

19. The composition or method of any preceding Embodiment, wherein the strain or at least one strain is an antibiotic-resistant or MDR strain; and/or wherein the strain or at least one strain is a B2-I strain.

For example, the strain or at least one strain is a strain selected from B2-I (STc131), B2-II, B2-IX, and B2-VI.

For example, at least one MDR strain is resistant to fluoroquinolone and the strain is a beta-lactamase (ESBL)-producing E coli.

20. The composition or method of Embodiment 19, wherein the antibiotic is fluoroquinolone (optionally levofloxacin), carbapenem or vancomycin; and/or wherein the E coli are beta-lactamase (ESBL)-producing E coli.

Optionally, the antibiotic is selected from ciprofloxacin (eg, Cipro™), gemifloxacin (eg, Factive™), levofloxacin (eg, Levaquin™), moxifloxacin (eg, Avelox™), and ofloxacin.

For example, the E coli produce CTX-M-15. CTX-M-15 is the most abundant enzyme in ESBL-producing E. coli causing human infections.

Preferably, the antibiotic is fluoroquinolone (FQ). For example, the FQ is levofloxacin. Levofloxacin, sold under the brand name Levaquin™ among others, is an antibiotic medication. It is used to treat a number of bacterial infections including acute bacterial sinusitis, pneumonia, urinary tract infections, chronic prostatitis, and some types of gastroenteritis. Levofloxacin prophylaxis is recommended to prevent gram-negative bloodstream infections (BSIs) in patients with prolonged chemotherapy-induced neutropenia. However, increasing fluoroquinolone resistance may decrease the effectiveness of this approach (eg. Clin Infect Dis. 2021 Oct. 5; 73 (7): 1257-1265. doi: 10.1093/cid/ciab404, “Colonization With Fluoroquinolone-Resistant Enterobacterales Decreases the Effectiveness of Fluoroquinolone Prophylaxis in Hematopoietic Cell Transplant Recipients, Michael J Satlin et al). This study found that in the patients tested, nearly one-third of hematopoietic cell transplantation (HCT) recipients with pretransplant fluoroquinolone-resistant Enterobacterales (FQRE) colonization developed gram-negative bloodstream infections (BSIs) while receiving levofloxacin prophylaxis, and infections were typically caused by their colonizing strains. In contrast, levofloxacin prophylaxis was highly effective in patients not initially colonized with FQRE. The authors found that 23% of patients admitted for HCT were colonized with FQRE and E. coli was the predominant species. Patients with hematologic malignancies who receive intensive chemotherapy, including those undergoing hematopoietic cell transplantation (HCT), frequently develop severe neutropenia and gastrointestinal mucositis, placing them at high risk of developing bloodstream infections (BSIs) from gram-negative enteric bacteria (Enterobacterales). Neutropenic patients often suffer severe consequences from BSIs caused by Enterobacterales, with mortality rates as high as 15%-20%. Moreover, many fluoroquinolone-resistant Enterobacterales (FQRE) also harbor extended-spectrum β-lactamases (ESBLs); thus, breakthrough infections that occur despite fluoroquinolone prophylaxis may be resistant to first-line antimicrobial therapies for fever and neutropenia. Finally, adverse effects of fluoroquinolones have become increasingly apparent, including Clostridioides difficile infection, aortic dissection and rupture, dysglycemia, tendinopathy, QT interval prolongation, and mental status changes. Thus, fluoroquinolones should only be administered to patients when they are likely to provide clinical benefit to justify these potential adverse effects. Thus, Although fluoroquinolones may decrease the risk of gram-negative BSI in many patients, those who are colonized with FQRE may not benefit from fluoroquinolone prophylaxis.

The high rate and absence of risk factors for FQRE colonization suggest that FQRE are prevalent in the community. Indeed, a study of urinary isolates among outpatients in the United States demonstrated that 12% of E. coli isolates from young women and 29% of E. coli isolates from elderly women were fluoroquinolone resistant. A surveillance study of 1831 urinary E. coli isolates from 2017 found that one-quarter were FQ resistant. Furthermore, 13%-16% of men undergoing transrectal prostate biopsies were found to be colonized with fluoroquinolone-resistant E. coli. Nearly one-half of fluoroquinolone-resistant E coli isolates in the study were ST131, a common sequence type that has spread throughout the world and whose isolates are frequently fluoroquinolone resistant and ESBL producers.

Bacteraemia caused by extended-spectrum β-lactamase (ESBL)-producing Enterobacteriaceae (ESBL-E), such as E coli, is associated with inadequate empirical therapy and substantial mortality in neutropenic patients (see eg, “Colonization With Levofloxacin-resistant Extended-spectrum β-Lactamase-producing Enterobacteriaceae and Risk of Bacteremia in Hematopoietic Stem Cell Transplant Recipients”, Satlin M J et al, Clin Infect Dis. 2018 Nov. 13: 67 (11): 1720-1728. doi: 10.1093/cid/ciy363). The study found that HSCT recipients who are colonized with levofloxacin-resistant ESBL-E pre-transplant and receive levofloxacin prophylaxis have high rates of bacteraemia from their colonizing strain during neutropenia. In this single-centre study of 312 HSCT recipients, it was found that 10% of patients were colonized with ESBL-E prior to their transplant. Nearly one-third of patients with pre-transplant ESBL-E colonization developed subsequent ESBL-E bacteraemia while neutropenic after their transplant, compared to <1% of patients who were not initially colonized with ESBL-E. Furthermore, the bloodstream and gastrointestinal ESBL-E had identical MLST and PFGE profiles in all cases, suggesting that these patients developed bacteraemia from their colonizing isolates.

In an example, the composition or method of the invention is for preventing the translocation of B2 phylogroup E coli from the gastrointestinal tract to the blood stream of the subject, thereby preventing or reducing bacteriaemia in the patient.

In an example, the composition or method of the invention is for preventing the translocation of B2 phylogroup E coli from the urinary tract to the blood stream of the subject, thereby preventing or reducing bacteriaemia in the patient.

In an example, the E coli are comprised by the gastrointestinal tract of the subject. Optionally, in these examples, the composition is administered orally to the subject.

In another example, the E coli are comprised by the urinary tract of the subject. For example, the infection is a kidney, bladder or urethra infection. Optionally, in these examples, the composition is administered to the urinary tract of the subject, such as by a catheter.

21. The composition or method of any preceding Embodiment, wherein
(i) the nuclease is a Cas,
(ii) each particle comprises a phage capsid containing the nucleic acid; and
(iii) wherein a first type of transduction particle and a second type of transduction particle are administered to the subject, wherein the first type of particle comprises a first adhesion moiety that is capable of recognising and binding to a first cognate moiety selected from the group LPS, LamB and Tsx displayed on B2 strain E coli, and the second type of particle comprises a second adhesion moiety that is capable of recognising and binding to a second cognate moiety selected from said group, wherein the first and second adhesion moieties different from each other.

22. A method for treating or preventing an infection by E coli cells in a human or animal subject, the method comprising administering to the subject a plurality of transduction particles, wherein
(a) each particle comprises a nucleic acid encoding a crRNA or guide RNA that is operable with a Cas nuclease for chromosomal targeting in the cells, wherein the said administered particles contact the cells and introduce therein the nucleic acid, wherein the crRNA or guide RNA is expressed and guides the Cas nuclease wherein the nuclease cuts the chromosomes of the cells, thereby killing the cells or reducing growth or proliferation of the cells in the subject;
(b) the E coli cells are cells of E coli phylogroup B2; and
(c) each particle comprises an adhesion moiety for recognising and binding to a cognate moiety selected from a LPS, LamB and Tsx displayed on the surface of the phylogroup B2 E coli cells.

Each nucleic acid preferably encodes a plurality of different cRNAs comprising spacer sequences that target E coli chromosomal genes. For example, Each nucleic acid preferably encodes a plurality of different cRNAs comprising spacer sequences that target 2, 3 or 4 E coli chromosomal genes selected from fimH, bolA, rpoH, IptA and murA.

Optionally, each crRNA or guide RNA comprises a spacer that targets an E coli gene selected from the group fimH, bolA, rpoH, IptA and murA. Optionally, each nucleic acid encodes a plurality of different cRNAs or guide RNAs, wherein the cRNAs or guide RNAs target at least 2, 3 or 4 (or targets all of) E coli genes selected from the group fimH, bolA, rpoH, IptA and murA. Optionally, each nucleic acid encodes a plurality of different cRNAs or guide RNAs, wherein the cRNAs or guide RNAs target fimH and bolA. Optionally, each nucleic acid encodes a plurality of different cRNAs or guide RNAs, wherein the cRNAs or guide RNAs target rpoH and lptA. Optionally, each nucleic acid encodes a plurality of different cRNAs or guide RNAs, wherein the cRNAs or guide RNAs target fimH and murA. Optionally, each crRNA or guide RNA comprises a spacer sequence that is complementary to an E coli gene selected from the group fimH, bolA, rpoH, IptA and murA. Optionally, each crRNA or guide RNA comprises a spacer sequence that is at least 80, 90 or 95% identical to a nucleotide sequence selected from the group SEQ ID NO: 6-10.

Optionally, each nucleic acid encodes a first crRNA, second crRNA, third crRNA, fourth crRNA and fifth cRNA, wherein the cRNAs are different from each other and each crRNA targets a B2 phylogroup E coli gene. Optionally, each nucleic acid encodes a first crRNA, second crRNA, third crRNA, fourth crRNA and fifth cRNA, wherein the cRNAs are different from each other and each crRNA is complementary to a B2 phylogroup E coli gene. Optionally, each nucleic acid encodes a first crRNA, second crRNA, third crRNA, fourth crRNA and fifth cRNA, wherein the cRNAs comprise respectively SEQ ID NO: 6, SEQ ID NO: 7, SEQ ID NO: 8, SEQ ID NO: 9 and SEQ ID NO: 10. Optionally, each nucleic acid encodes a first crRNA, second crRNA, third crRNA, fourth crRNA and fifth cRNA, wherein the cRNAs comprise respectively a nucleotide sequence that is at least 80% identical to SEQ ID NO: 6, a nucleotide sequence that is at least 80% identical to SEQ ID NO: 7, a nucleotide sequence that is at least 80% identical to SEQ ID NO: 8, a nucleotide sequence that is at least 80% identical to SEQ ID NO: 9 and a nucleotide sequence that is at least 80% identical to SEQ ID NO: 10. Optionally, each nucleic acid encodes a first crRNA, second crRNA, third crRNA, fourth crRNA and fifth cRNA, wherein the cRNAs comprise respectively a nucleotide sequence that is at least 90% identical to SEQ ID NO: 6, a nucleotide sequence that is at least 90% identical to SEQ ID NO: 7, a nucleotide sequence that is at least 90% identical to SEQ ID NO: 8, a nucleotide sequence that is at least 90% identical to SEQ ID NO: 9 and a nucleotide sequence that is at least 90% identical to SEQ ID NO: 10. Optionally, each nucleic acid encodes a first crRNA, second crRNA, third crRNA, fourth crRNA and fifth cRNA, wherein the cRNAs comprise respectively a nucleotide sequence that is at least 95, 96, 97, 98 or 99% identical to SEQ ID NO: 6, a nucleotide sequence that is at least 95, 96, 97, 98 or 99% identical to SEQ ID NO: 7, a nucleotide sequence that is at least 95, 96, 97, 98 or 99% identical to SEQ ID NO: 8, a nucleotide sequence that is at least 95, 96, 97, 98 or 99% identical to SEQ ID NO: 9 and a nucleotide sequence that is at least 95, 96, 97, 98 or 99% identical to SEQ ID NO: 10.

23. The method of Embodiment 22, wherein the method is according to any one of Embodiments 1-20, optionally except that the nuclease is an endogenous nuclease of the cells and is not encoded by the nucleic acid comprised by the particles.

24. A composition comprising a plurality of transduction particles for use in a method of treating or preventing an infection by E coli cells in a human or animal subject according to Embodiment 22 or 23, wherein
(a) each particle comprises a nucleic acid encoding a crRNA or guide RNA that is operable with a Cas nuclease for chromosomal targeting in the cells, wherein the said administered particles contact the cells and introduce therein the nucleic acid, wherein the crRNA or guide RNA is expressed and guides the Cas nuclease wherein the nuclease cuts the chromosomes of the cells, thereby killing the cells or reducing growth or proliferation of the cells in the subject;
(b) the E coli cells are cells of E coli phylogroup B2; and
(c) each particle comprises an adhesion moiety for recognising and binding to a cognate moiety selected from a LPS, LamB and Tsx displayed on the surface of the phylogroup B2 E coli cells.

25. A method of detecting the presence of a B2 phylogroup E coli in a sample, the method comprising contacting the sample comprising B2 phylogroup E coli with a composition comprising a plurality of transduction particles, wherein
(a) each particle comprises a nucleic acid encoding a nuclease for targeting the genomes of said E coli cells, wherein the said administered particles contact the cells and introduce therein the nucleic acid, wherein the nuclease is expressed in the cells and cuts genomic DNA of the cells, thereby killing the cells or reducing growth or proliferation of the cells in the subject;
(b) each particle comprises an adhesion moiety for recognising and binding to a cognate moiety selected from a LPS, LamB and Tsx displayed on the surface of the phylogroup B2 E coli cells; and
(c) the method comprises detecting that B2 phylogroup E coli cells have been killed or the growth or proliferation thereof has been reduced.

26. A method of detecting the presence of a B2 phylogroup E coli in a sample, the method comprising contacting the sample comprising B2 phylogroup E coli with a composition comprising a plurality of transduction particles, wherein
(a) each particle comprises a nucleic acid comprising or encoding a detectable label, wherein the said administered particles contact the cells and introduce therein the nucleic acid, wherein optionally the label is expressed in the cells;
(b) each particle comprises an adhesion moiety for recognising and binding to a cognate moiety selected from a LPS, LamB and Tsx displayed on the surface of the phylogroup B2 E coli cells; and
(c) the method comprises detecting B2 phylogroup E coli cells comprising the label.

27. The method of Embodiment 26 or 27, wherein the composition comprises the features of the composition of any one of Embodiments 1, 2-21 24 and 25.

28. The method of any one of Embodiments 26-28, wherein the E coli comprise one or more E coli strains selected from the group ST131, ST1193, ST648, ST315, ST405, ST361, ST88 and ST453.

29. The method of any one of Embodiments 26-29, wherein the sample is a patient sample (eg, blood, urine, stool or saliva sample), wherein the subject is a transplant or cancer patient (optionally a haematological cancer patient), or wherein the patient is suffering from or at risk of a urinary tract infection (UTI); and optionally wherein the transplant is a solid organ or stem cell transplant (optionally a haematopoietic cell transplant).

30. The method of any one of Embodiments 26-30, wherein the nucleic acid of each particle comprises a nucleotide sequence (N1) encoding said nuclease, or comprising or encoding the label wherein each particle is a synthetic T-even phage (optionally a T4 phage) comprising an insertion of N1 into the genome of the phage, wherein the region is between the pin (protease inhibitor) gene and the iPII (internal protein) gene.

31. The method of any one of Embodiments 26-31, wherein at least 1×10⁷ PFU of particles are contacted with the sample.

32. The method of any one of Embodiments 26-32, wherein the particles are contacted with the sample at an MOI (multiplicity of infection) of at least 0.01.

Labelling for detection methods is routine for the skilled addressee. The label may, for example, be a fluorescence label, eg, GFP. The sample may be a blood, spit, sputum or cell sample.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications and all US equivalent patent applications and patents are herein incorporated by reference to the same extent as if each individual publication or patent application was specifically and individually indicated to be incorporated by reference. Reference is made to the publications mentioned herein and equivalent publications by the US Patent and Trademark Office (USPTO) or WIPO, the disclosures of which are incorporated herein by reference for providing disclosure that may be used in the present invention and/or to provide one or more features (eg, of a vector) that may be included in one or more claims herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” or similar as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The present invention is described in more detail in the following non-limiting Examples.

### Examples

### Example 1: Particle Composition for Surprisingly Targeting a Plurality of Different E coli Strains, Including B2 Phylogroup

### Overview

Patient samples containing various different E coli strains were challenged with a particle composition. The composition comprised a plurality of transduction particles bearing adhesion moieties that are able to bind to LPS, LamB or Tsx present on E coli cells. The particles comprised capsids comprising phage capsid proteins, with the capsids containing nucleic acids encoding CRISPR/Cas systems for chromosomal targeting in E coli. Each nucleic acid encoded a plurality of different cRNAs comprising spacer sequences that target E coli chromosomal genes. The ability to kill or reduce growth of E coli in the samples was determined using a plaque assay as described below. Whole genome sequencing and genome assembly were used to assign susceptible E coli strains to phylogroups. It was surprisingly seen that use of such a particle composition was able to very effectively and extensively target a plurality of different E coli strains (these strains being clinically relevant from actual patient samples). Furthermore, advantageously a large number of different strains of the B2 phylogroup were targeted and killed. This is significant, because B2 E coli strains often display antibiotic resistance (such as MDR), eg, resistance to fluoroquinolone, that causes potentially life-threating infections in patients, such as in cancer, transplant and UTI patients. In addition to the B2 group, we surprisingly also were able to successfully kill or inhibit the growth of multiple strains of E coli phylo groups B1, D. F and G.

### Patient Sampling

The samples tested (n=71) were obtained from two prospective observational studies enrolling adult (≥18 years) patients who were admitted to hospital for an autologous or allogenic hematopoietic cell transplantation (HCT) and who received levofloxacin (a fluoroquinolone, FQ) prophylaxis starting the day before the transplantation (Day-1) (Satlin 2021 and Satlin 2018). Trimethoprim-sulfamethoxazole (TMP-SMX) was administered to allogenic HCT recipients from 2 to 4 days prior to the HCT. The antibiotic treatments do not eradicate all E coli in the patients; for example, fluoroquinolone-resistant E coli would persist and thus sampling obtained E coli from patients. E coli isolates were derived from either anal swabs or fecal samples obtained upon admission for transplantation. The timing of sampling varied from Day-7 (7 days before transplantation) to Day 0 (the day of transplantation).

### General Plaque Assay: Spotting for Coverage

This procedure describes the method to assess the coverage of phage particle lysates on a panel of bacterial strains.

| Section | Procedure |
|---|---|
| Step 1 | Prepare bacterial indicator strains by inoculating 5 μl |
| | of a frozen stock into 250 μl of LB broth on a 96 well plate. |
| | Incubate overnight at 37° C. and 250 rpm. This step is done |
| | the day before the assay is performed. |
| Step 2 | Pre-acclimatize the LB plates to room temperature. |
| | Melt and acclimatize the top agar to 55° C. and |
| | add CaCl₂ and MgSO₄ to a final concentration of 5 mM. |
| Step 3 | In a culture tube mix 100 ml of the overnight indicator |
| | strain with 3 ml of pre-warmed top agar. When working with |
| | many strains, the top agar can be distributed to culture |
| | tubes and kept at 60° C. before use. |
| Step 4 | Pour the mixture on top of the room temperature LB plate |
| | and distribute evenly by swirling. |
| | Let plates solidify in a biosafety cabinet or lab bench |
| | for 5-10 minutes. Keep the lids closed. While solidifying |
| | avoid stacking plates to keep the layer of the top agar |
| | even across the plate surface |
| Step 5 | In the meantime, dilute the sample(s) or the stock of |
| | phage to the required dilutions (10-fold dilutions). |
| | Use PBS for dilutions. |
| Step 6 | Spot dilutions 0 to −9, 5 μl of each on top of your |
| | overlays and let them dry for 20 minutes (or until the spots |
| | are completely absorbed by the agar) on a bench with the |
| | lids open. Incubate plates upside down at 37° C. overnight. |
| Step 7 | Evaluation of the results: |
| | 1. If visible plaques appear, they are counted, |
| | and phage concentration is calculated. The |
| | result is recorded as YES. |
| | Calculation of phage concentration: number of plaques × 200 |
| | (to make it into ml) × dilution where you counted, i.e: |
| | If counted 5 plaques on dilution-6: 5 × 200 × 1e6 = 1e9 pfu/ml |
| | 2. If there are no visible plaques, but inhibition |
| | of growth is observed the result is recorded as |
| | LZ (lysis zone) and the lowest dilution of |
| | inhibition is noted. |
| | 3. If no plaques or inhibition is observed, the result |
| | is recorded as NO |

Spotting was performed according to the above general plaque assay. Bacterial strains were prepared by inoculating 5 μl of a frozen stock into 250 μl of LB broth in a 96 well plate. The plate was incubated at 37° C. and 250 rpm overnight. Next day, in a culture tube 100 ml of the overnight strain was mixed with 3 ml of pre-warmed top agar (at 55° C.) containing 5 mM CaCl₂) and 5 mM MgSO₄. The mixture was poured on top of pre-acclimatized LB plate and distributed evenly by swirling. The plates were left on the lab bench for 5-10 minutes to solidify. In the meantime, the particle composition was diluted in PBS buffer from 100 to 109 and 5 μL of each serial dilution were spotted on top of the overlays. Plates were left on the bench with the lid open for 20 minutes or until the spots are completely absorbed by the agar, and then incubated upside down at 37° C. overnight.

### Evaluation of the Results:

If visible plaques appeared, the results were recorded as positive, plaques were counted, and phage concentration was calculated. Calculation of phage concentration: number of plaques ×200× dilution where plaques were observed. i.e: If counted 5 plaques on dilution −6:5×200×1e6=1e9 pfu/ml

If there were no visible plaques, but inhibition of growth was observed, the result was recorded as lysis zone and the lowest dilution of inhibition is noted.

If no plaques or inhibition was observed, the results were recorded as negative.

### Whole Genome Sequencing

DNA extraction was performed using Omega Bio-tek, Mag-Bind Bacterial DNA 96 Kit. The protocol was followed, and samples were eluted in 100 μL Elution Buffer.

Sequencing libraries were generated using Illumina Nextera XT, and sequencing was performed with paired ends on an Illumina MiSeq instrument with a V2 flow cell (300 cycles). The average sequencing depth for all samples was 48x (range: 31-72x)

### Genome Assembly and Phylogenetic Tree Reconstruction

Raw data was trimmed for adaptor sequences and low-quality bases using fastp 0.22.0 (Chen et al 2018). Genomes were assembled using SKESA 2.4.0 (Souvorov et al 2018). The phylogroup of each sample was determined using EzClermont 0.7.0 (github.com/nickp60/EzClermont). Genomic distances were estimated using Mash 1.1 (Ondov et al 2016) with a kmer size of 17 and 1000 sketches. A neighbour joining tree was constructed using rapidnj 2.3.2 (Simonsen et al 2008). The final tree visualization was generated in Interactive Tree Of Life (iTOL) version 6.5.3 (Letunic et al 2021). Strain phylotyping was carried out in silico using the method disclosed in Microb Genom. 2018 July; 4 (7): e000192, Published online 2018 Jun. 19. doi: 10.1099/mgen.0.000192, PMCID: PMC6113867, PMID: 29916797, “Clermon Typing: an easy-to-use and accurate in silico method for Escherichia genus strain phylotyping, Johann Beghain et al. The set of primer sequences described in Table S1 (available in the online version of this article) was used.

### Results

It was surprisingly seen that use of such a particle composition was able to very effectively and extensively target a plurality of different E coli strains (these strains being clinically relevant from actual patient samples), see FIG. 1. Furthermore (see FIG. 2), advantageously a large number of different strains of the B2 phylogroup were targeted and killed. This is significant, because B2 E coli strains often display antibiotic resistance (such as MDR), eg, resistance to fluoroquinolone, that causes potentially life-threating infections in patients, such as in cancer, transplant and UTI patients. More than 10 different ST131 strains were killed (plaques formed) and more than 10 different ST1193 strains were killed (plaques formed). Sequence type 1193 has recently emerged as a new, virulent and resistant lineage among fluoroquinolone resistant E coli. Escherichia coli ST131 is a globally dominant multidrug resistant clone associated with high rates of rUTI. Uropathogenic E coli (UPEC) are the primary cause of urinary tract infection (UTI), being responsible of ˜90% of all cases. UPEC strains largely belong to the E coli phylogenetic groups B2 or D and are often clonal, with the most common sequence types (STs) isolated worldwide being ST69, ST73, ST95 and ST1312. The recently emerged and globally disseminated ST131 clone is a major contributor to hospital- and community-acquired UTI, as well as bloodstream infections and infections in companion animals and poultry. Originally identified in 2008, ST131 is associated with the worldwide spread of the CTX-M-15 extended spectrum B-lactamase (ESBL) resistance gene. Most ST131 strains are now strongly associated with multidrug resistance (MDR), including resistance to fluoroquinolones. Recent reports have also identified strains that are resistant to last-line carbapenems.

Significantly, we included clinical samples from patients that went on to develop E coli bacteraemia (despite pre-treatment with FQ/TMP-SMX). The composition could kill or reduce growth of E coli strains in these samples (and this is indicative of the possibility of using the composition for prophylaxis of bacteraemia in subjects). This included strains of the following Multi-Locus Sequence Typing (MLST) types (see FIG. 2): ST648, ST315, ST405, ST361, ST88, ST453, ST1193 and ST131.

In addition to the B2 group, we surprisingly also were able to successfully kill or inhibit the growth of multiple strains of E coli phylo groups B1, D, F and G.

### REFERENCES

Shifu Chen, Yanqing Zhou, Yaru Chen, Jia Gu; fastp: an ultra-fast all-in-one FASTQ preprocessor, Bioinformatics, Volume 34, Issue 17, 1 Sep. 2018, Pages i884-1890, doi.org/10.1093/bioinformatics/bty560
Alexandre Souvorov, Richa Agarwala and David J. Lipman. SKESA: strategic k-mer extension for scrupulous assemblies. Genome Biology 2018 19:153. doi.org/10.1186/s13059-018-1540-z
Ondov, B. D., Treangen, T. J., Melsted, P. et al. Mash: fast genome and metagenome distance estimation using MinHash. Genome Biol 17, 132 (2016). doi.org/10.1186/s13059-016-0997-x.
Martin Simonsen, Thomas Mailund and Christian N. S. Pedersen. Rapid Neighbour Joining. Proceedings of the 8th Workshop in Algorithms in Bioinformatics (WABI), LNBI 5251, 113-122, Springer Verlag, October 2008. doi: 10.1007/978-3-540-87361-7_10.
Letunic I and Bork P (2021) Nucleic Acids Res doi: 10.1093/nar/gkab301 Interactive Tree Of Life (iTOL) v5: an online tool for phylogenetic tree display and annotation
Michael J. Satlin et al, Colonization with Fluoroquinolone-resistant Enterobacterales decreases the effectiveness of fluoroquinolone prophylaxis in hematopoietic cell transplant recipients. Clinical Infectious Diseases 2021.
Michael J. Satlin et al, Colonization with Levofloxacin-resistant extended-spectrum β-lactamase-producing Enterobacteriaceae and risk of bacteremia in hematopoietic cell transplant recipients. Clinical Infectious Diseases 2018.

### SEQUENCES

Amino acid sequences are written in N- to C-terminal direction and DNA sequences are written in 5′ to 3′ direction.

PROTEIN
>LamB
(SEQ ID NO: 1)
MMITLRKLPLAVAVAAGVMSAQAMAVDFHGYARSGIGWTGSGGEQQCFQTTGAQSKYRL
GNECETYAELKLGQEVWKEGDKSFYFDTNVAYSVAQQNDWEATDPAFREANVQGKNLIEW
LPGSTIWAGKRFYQRHDVHMIDFYYWDISGPGAGLENIDVGFGKLSLAATRSSEAGGSSSFAS
NNIYDYTNETANDVFDVRLAQMEINPGGTLELGVDYGRANLRDNYRLVDGASKDGWLFTA
EHTQSVLKGFNKFVVQYATDSMTSQGKGLSQGSGVAFDNEKFAYNINNNGHMLRILDHGAI
SMGDNWDMMYVGMYQDINWDNDNGTKWWTVGIRPMYKWTPIMSTVMEIGYDNVESQRT
GDKNNQYKITLAQQWQAGDSIWSRPAIRVFATYAKWDEKWGYDYNGDSKVNPNYGKAVP
ADFNGGSFGRGDSDEWTFGAQMEIWW
>Tsx
(SEQ ID NO: 2)
MKKTLLAAGAVLALSSSFTVNAAENDKPQYLSDWWHQSVNVVGSYHTRFGPQIRNDTYLE
YEAFAKKDWFDFYGYADAPVFFGGNSDAKGIWNHGSPLFMEIEPRFSIDKLTNTDLSFGPFK
EWYFANNYIYDMGRNKDGRQSTWYMGLGTDIDTGLPMSLSMNVYAKYQWQNYGAANEN
EWDGYRFKIKYFVPITDLWGGQLSYIGFTNFDWGSDLGDDSGNAINGIKTRTNNSIASSHILA
LNYDHWHYSVVARYWHDGGQWNDDAELNFGNGNFNVRSTGWGGYLVVGYNF
DNA
>LamB
(SEQ ID NO: 3)
ATGATGATTACTCTGCGCAAACTTCCTCTGGCGGTTGCCGTCGCAGCGGGCGTAATGTCT
GCTCAGGCAATGGCTGTTGATTTCCACGGCTATGCACGTTCCGGTATTGGCTGGACAGGT
AGCGGCGGTGAACAACAGTGTTTCCAGACTACCGGTGCTCAAAGTAAATACCGTCTTGG
CAACGAATGTGAAACTTATGCTGAATTAAAATTGGGTCAGGAAGTGTGGAAAGAGGGCG
ATAAGAGCTTCTATTTCGACACTAACGTGGCCTATTCCGTCGCGCAACAGAATGACTGGG
AAGCTACCGATCCGGCCTTCCGTGAAGCAAACGTGCAGGGTAAAAACCTGATCGAATGG
CTGCCAGGTTCCACCATCTGGGCAGGTAAGCGCTTCTACCAACGTCATGACGTTCATATG
ATCGACTTCTACTACTGGGATATTTCTGGTCCTGGTGCCGGTCTGGAAAACATCGATGTT
GGCTTCGGTAAACTCTCTCTGGCAGCAACCCGCTCCTCTGAAGCTGGTGGTTCTTCCTCTT
TTGCCAGCAACAATATTTATGACTATACCAACGAAACCGCGAACGACGTTTTCGATGTGC
GTTTAGCGCAGATGGAAATCAACCCGGGCGGCACATTAGAACTGGGTGTCGACTACGGT
CGTGCCAACCTGCGTGATAACTATCGTCTGGTTGATGGCGCATCGAAAGACGGCTGGTTA
TTCACTGCTGAACATACTCAGAGTGTCCTGAAGGGCTTTAACAAGTTTGTTGTTCAGTAC
GCTACTGACTCGATGACCTCGCAGGGTAAAGGTCTGTCGCAGGGTTCTGGCGTCGCGTTT
GATAACGAAAAATTTGCCTACAATATCAACAACAACGGTCACATGCTGCGTATCCTCGAC
CACGGTGCGATCTCCATGGGCGACAACTGGGACATGATGTACGTGGGTATGTACCAGGA
TATCAACTGGGATAACGACAACGGCACCAAGTGGTGGACCGTTGGTATTCGCCCGATGT
ACAAGTGGACGCCAATCATGAGCACCGTGATGGAAATCGGCTACGACAACGTCGAATCC
CAGCGCACCGGCGACAAGAACAATCAGTACAAAATTACCCTCGCACAACAATGGCAGGC
TGGCGACAGCATCTGGTCACGCCCGGCTATTCGTGTCTTCGCAACCTACGCCAAGTGGGA
TGAGAAATGGGGTTATGACTACAACGGCGATAGCAAGGTTAACCCGAACTACGGCAAAG
CCGTTCCTGCTGATTTCAACGGCGGCAGCTTCGGTCGTGGCGACAGCGACGAGTGGACCT
TCGGTGCCCAGATGGAAATCTGGTGGTAA
>Tsx
(SEQ ID NO: 4)
ATGAAAAAAACATTACTGGCAGCCGGTGCGGTACTGGCGCTCTCTTCGTCTTTTACTGTC
AACGCAGCTGAAAACGACAAACCGCAGTATCTTTCCGACTGGTGGCACCAGAGCGTTAA
CGTTGTCGGAAGCTATCACACCCGTTTCGGACCGCAGATCCGCAACGATACCTACCTTGA
GTACGAAGCATTCGCTAAAAAAGACTGGTTCGACTTCTATGGTTATGCGGATGCGCCGGT
ATTCTTCGGCGGTAACTCCGATGCAAAAGGTATCTGGAACCACGGTTCTCCGCTGTTTAT
GGAAATCGAACCACGTTTCTCCATCGACAAGCTGACCAATACTGACCTTAGCTTCGGTCC
GTTCAAAGAGTGGTACTTCGCGAACAACTACATTTACGACATGGGTCGTAATAAAGATG
GTCGCCAGAGCACCTGGTACATGGGTCTGGGTACCGATATCGACACTGGCCTGCCGATG
AGCCTGTCCATGAACGTCTATGCGAAATACCAGTGGCAGAACTATGGCGCAGCGAACGA
AAACGAGTGGGACGGTTACCGTTTCAAAATTAAATACTTTGTGCCGATTACCGATCTGTG
GGGCGGTCAGCTGAGCTACATCGGCTTCACCAACTTCGACTGGGGTTCCGATTTAGGGGA
TGACAGCGGTAACGCAATCAACGGTATTAAGACCCGTACTAATAACTCTATCGCTTCCAG
CCATATTCTGGCTCTGAACTACGATCACTGGCACTACTCTGTCGTAGCTCGTTACTGGCAC
GACGGTGGTCAGTGGAACGACGATGCAGAACTGAACTTCGGCAACGGCAACTTCAACGT
TCGCTCTACCGGCTGGGGTGGTTACCTGGTAGTAGGTTACAACTTCTGA
>NC_000866.4 Enterobacteria phage T4, complete genome
(SEQ ID NO: 5)
AATTTTCCTTATTAGGCCGCAAGGGCCTTCATAGTTTTAGCGATTTGGGAAACTTCATCATCACTTAAAG
AGTTGCGATAACCGATGAAGTCGGAAACAATACGGAATTTCTTGGTAAACTCAGCAACCATTTTATCACT
GTTTTTTGAAGCATTATTTGATAATACATCAAAAAGATTAGTTACTGTCCAAATGTCATGACCGATGGTA
TCTTTTCCACCATTAAAATATACACCCTGTAATGAACTAACCATATTAGCGAGTCGTGTATATTCTTCAG
AAACTTCATCTATACTGAAGTACTTCATCATAAAATCTAACTCAGGATACTTGATAATTTTATCAATATA
TCGTTTAGCTGAACTTGAATAACCTACATACTTATCATAATCTACATCATCAAAAGCATCTACATATAAA
TCACGCAAAGCTTCAAAAATACATTGGCACTGACCGAGTTCTTTTACCTTTTTCTGTAAAAGCGGACGAA
TAACATAAAATTCATTAATGCCAATAAGATTAGCCATACGAATCAAAATATTCATAGATGGATGACAAAG
AGATGTAGTACCATCCATAGAGAAAATATCAGAACGATGCATATACGCTACATAACCAGTAATTTCATCT
GCTTCTGATGTGAGGCGTAAATAATTCCTCTTTTCCCAGCGCCCGTCTTTAATTTCAAACTTAAACGCTG
TAGCAGCTTTAGGACGAGGAGCTTTACTTTTAACTACCTTTGGAATATAACTTTTTACTAAAGCTTCAAT
TTCTGACAAATAATGAATGTTAACTTCATCACTTTCAAACATCGCCATAATATCAGGAAGCAAATCAATC
TGCGATTCTACTTCTGGATTAATAAACAGAAGACGTTCGTTATGATGAATATTCAAAGTGTTATTAAATT
CACTATCATCTAACGCACGTGCTAATCCACGGACAATATTAACACGATTTTTAATATTATCAATAACGAT
ATTAATTTTTGTTGTATTAATACCAAACAGACGATAACTTGATGCAACGGCTGAAGTTTCATGACTTTGC
TTAATGCGCTTCAGTCGAGGGTCAAGATTTACTTCATACACAACTCCCGCGTTGCATAACTTACTGTCAG
GTTCAAACATGCTCTGCATCTTTTTATATGACAGATTTTTAGTCGTGAATTTGACTGAATTACTAATCAT
ATAATCTCGAGCAGAATACCCCATCTTCATCAATTCACGATATGTGTGACGAGGAGATGTAGATTCTTTA
AATCGTTTTACATCTTCATTAAATGCTTTCTCACTGAGTTCTTTAACTCGTTCAATAATATTTTTACGAG
TGCGATCATCCAGTGAAAGAGCCTCGCGAGATGGAGCAATATCAAGTGAACCCATTGGAAACTTAATGTA
ATTCACTTCATTGCGAATGCTTAGCCAGTTACGGTCTCTAATAACACCATCGATAGGATAAACAATACCA
CCGTAGATAGCATATAATCCACCACGATCAGGCCAGTATCTTTCTGGATTTACACCGTAATAGTCATCAA
AATCCGGAAAATAATCAATTTCGCGGTCAAGACCATTAATGATAGCCAAATCTTTGAACGGTCGCATGAT
ATAAGAAACTTCATAAGCAAAGTTTCTAAAGTCTTTTTCTTCAACTGGAACTACGATTTCAATACCAGTT
TTATCATCTGGACCCATTTCTTTTACGAATGTAGGTTTAATCTGTGGACCATCACCATCCATGTAAGCTA
CATAACCACGAATTTCACCTTTATGATACGAAGTAATACTAAACGTATCAGTATAACTAAACGGAGATTT
AGAACCTAAACCAAATCCGCCAATAAAGTCATTAGATTCAGCTTTAGATGAACTGAAGTATGAATTATAC
AACCCAGGAGAATTATCATCACCTTGAATATCAAAATCACTCATACCCGGACCAAAATCTCGACAAACAA
ATCGTGGGTCTAAACGTCCAGGAACTTGTATGATAAATTTTTCAGGATTTCCATTAAGTGCATGAGCATC
AATCATGTTAGTAATCAATTCACGGACTACTGCGCGAATCTTGTTTGTATACAAATCAGATGACAGAATT
TTAAATACTTTAGGAGATGCTGTGATGCTAAATGCTTTTGATTTAGAACCATTACCAAGAATTGTTTCTT
TTTCAGTGGTGATAATCATAATTTCCTCATTAATTCATATTACGCTTAATAACTTCAGCAACTTCTAGTA
GTTCATCTTTAGTTGCGGTGTCGGATTGAATTTTATCTCTAATATCTTTAAAGCGGGTTTTAAATTCTTC
GGCTTCTCCCATATCGAAAAAGCGTTGAATGATTCTATATTCTCGATGAACTGCTTTATCAAAAAGTTCT
AAATTTACTTTATATGATTTCATTTCAATATCCTCATTTGCCCAATTAATTATACCACATCCTTGTGGTA
AAGTAAACTACTGGCTCATCCATTCTTTACGAAGGTCAGCATTATCTCCCATGAGCATTTCAAAAAGCTC
TTTCCAGTTCTCAGGAAGTTTAACAACATCATATACTGGGTTTTGAATCATCTCACGATATTCAGATTTT
TCCAAAGAGCCAAGTCCCTTAATATAACGGATGCTATGTTTAGGTAGAGCATCTTTGGCACTCTCATATT
CAGCGACTGTATAAAACCATTCTTGTTTTTTACCGACCTGAGCGATGATTACAGGAGTTTTGACAAAGCG
AATTCGTCCTTGCTCAAACAATTCTGGCCAATTACTAAAAAATCCGAGCAGAGAAGGATAAATAGAACCT
AATCCAATAATCTCTTAATTATGAGGTATTTCTATAGATAGCCCGAAGGCTATCCATCGTGATCTGCGTC
TGTCATAATAGCGACATTCGCATAGTTCATTGAAGAGGATTTAATAGAACGACGAACATTGTTCTGCAAT
TTATATTTTTTCATATCAACGCTAGAAGAATCAATTTTTACAAATTTCATTATACACCTCATAGAACTTT
TCATCAGGAATCCAACCGCGTTTAAATTCATTAAATGCTCGGCCGAATAATTTTGAATTCACAGTTATAT
TATTAACTGATTTCCATTTAGCAACTCCCGTTCGTTTATAATGATCGGGGTCATATTTCGTGACGTACCA
TTCATATAAATTTGGTATTAATTTTACAGCCTCTGGATTTGTCGCTGATTTATTATACCATGGTTTCGCT
TTTTCAAGTTCAGCTGCTTTTGCAGTAGCAGCAACAGTATTTCCAACACCAACTAATTTTTCAGTTTTAA
TGCGTGGATCATTAACATGAAGACGAAAAACTTCGCCCTTTTCATTTTTAAAGCATGTCATGCCTTTAAT
TCCAGGAAGCTTAACACCTTTATTAACACCGCATCATTCCTTTGGGTTAAATGATCCTTTAATTAATAAG
GCGCATTTACCCGATTTAACTACTTCTCATTCAACAACTTTATCTTTCATAACGTTTTTTGACCATTCAG
ATACTGCTCTTTGATGGCTAAATTCTAGCAATTTCACTATAATTTGCACTAGAACGTAAACTATTTTTCA
GTGTTTCAACATTCTATTCATCGCATATGCCATTTCACGATTACGATGAATTTTATATAGTAGAAAATAG
TGCTAAAAAGTGTTCACGAAAAGTCATGTTTCACCAAATTTTCGTTATCATCAGAACCTCCCATTGATCT
TGGAAGGATATGATGAATTTCACCCTTAAATTTAGAAACGCGGGTTTTACCCCGCACTATTAAGTCATTA
TAGATTTTTTCGTAATTCACCTACTGTTATCCATTTACCATTAATCTGTACTTCATCATTTTCATTTACG
ATAATTGTATCGCCATTTAGCTCGAAAGTAAACCACTCGCCATCTTCTTTTTCTTCAAACGCTTTTTCAC
CGAGAACTAGACCAGTGATTGCGCAAATATCAAATAGTTCTTTGTTTTTAAGCATATCTGCATAAGACAT
ACCCCAACTGTTGAGAACTTTACCACGCAATGGATAACCACCGTGAAGTTCTTTATCACGAACATCAATA
AGATATCCGATAGCCGAATCACCCTCAGTCAAGAAAAGAGTAGTATCAGCATCTTTACCGCAAAGATTCG
CTTTGATATGTTTATGAACCTTAGCTTTAGAAGCCTTTTTAGCTGCTTTAGTTTCTGCTGCTTTTTCTGC
CGCCAATTTACGAGCCAAAGCAGCTTCAATAATCGGCATTAGAATTGCTTCATTATTTAGAATATCACGT
GAAATCTTTTTAGCATCAAGTTGAATATGACTACGAATTTCGCCAAATGGAGAAGTCAAACGCTCTTTAG
TTTGACGAATCAATCGCATGTTTTTCATATCACGAACAAACATAACGATAGTCAAACATTCTTTGACACG
TGCTTTAGTCACATCAATTTTGAACTTACGTTTGATTTGTGGAATAAGGTCTTCACAAATATCATCCATA
GCGCAGTCAATGTGATGGCCACCATTCTTAGTATGAATGTTATTGACGTATGTTAATTGACGAAAACCAT
CCGGTGAACGACCAACCGCAATAGAACAATTTTCTTGCTCTTGAACAATAGCATGTTCATCATACTGCCG
TGCATATTTCTTAAAATTGCCCTGAACCTTTTTACCATTAAAGGTAAATTGAATATCAGGATAAACTACA
GCAAGTGTCTGGAGACGATCCAGTGTAATGTCAAGATAAACTTGGGACAGCTCATTAGTTTCAAATGACA
TAAAATCAGGAATGAAAGTAACACGAGTTCCTTTCCATTTTCCAGGAATATCTTCCCATGATTTATTTTC
CATGCCATTTGAACAACGAACTACAATATTATTTTGACCGTCGCCAGTTTCACCGACAAACATCACAGAA
AAAATGTTTGTCAAACTAGAACCAACACCGTTCATACCGCCGGTGACGCGTTCTTTATCATCACCAAAGT
TACCACCTGCTTTTGGAATAGTCCATGCGGCAACAGGACCAGGAATTTCTTCACCGGTAGGTGTTTTAAC
CATCGCTTGTGGAATACCGCGACCGTTATCTTCAACTGTTACTTGATTGTTTTTAATAGTAACATTAATT
TTATTCGCGAATTTAAACTTAGTACGAATACCTTCATCTACTGAGTTATCGATAATTTCATCAATAAGCT
TAACAAGACCAGGTACATACTGAACACTTTCCCATTTACCAAACATAAAGCGCTCATGCGTTTCATTAGC
AGAAGAGCCAATGTACATGCCACTACGCTTTTTGATATGTTCAATATCGCTCAGAATTTTAATTTCATTC
TTAATCATCACTTATCCTCGTTTGGTTTCGGGAATATTATACTCCGGTAATCATAAAGCTAAAGGCCCGA
AGGCCTTTTATTTAAAACGAATAGTTGAATCCTTAAAGAACAGCCCAGAACATACTGTTCCTTCTACTTT
CTGCCCGGTAGGTCCAATAGCACGAAATCCAGTATGCTGGAAATCATTTTCAGAGCAACCGAACCAATTA
TATCCAGTGATTTCAATATTAGTAAAACCACTTGAAGACAAAACTTTGGTTGCATTAATCAGCATCAGTA
CTATTAATTAAAGACACTGCTAATACTAATGCTGCAATTGAACGACTAATATATTTCATAACTACCCTTT
AAGCAAGTCGTAAAATCCATTATTCCCATGCTTAGGAAGCGGAAACTAACCGAACAGCCAGCCGATGACA
ATCAGGACATACACCAGTATCTCTTCCAGAAATTTTCTTGATTTTTTCGTATTCTTTTGCACAGTCTTTG
GATTGACATTTATAATCATAAAGCGGCATAATTATTCCTTAAAGTAAGCTTTCAACATCTGATATAAAGA
CCACGCCTGATCATTATTTTCAATAGTAACTTTCATGACTGGGAATTCTGTGAAATCTTCTATTTGTTCT
TGCTCTTTCTCTTCCTGCTCTTGCTCTTCAACCGCCTGATATGGATTTTCCACTTCATCAAAGAACCCAG
CTTCGTTAGTAGAGAGCCAGATAAAGTTTTCGTCAAGGATATCACCGCCGGCACAACGTTTGAGTACACC
TATGGATGTCATAATTTTAGTAGGACGCCCAAGATAATCAGCATCTAAAATTTTAAAAGGCTCCATACCT
AAACGTCGTGCATAGATTCCGTTATCAGTATGGTCTTTAATAAAATTTTCTTGAGCTTGTTTATTTTTAA
ATTGATACCATTTATTAACTTCAAATTTAATAGCCATTAATAAATTTCCTTCCAGTAAGTTGTGCCGTCT
TCAGTAATTTCACGAAATACACCATAAATTGGCTGTTTATCACCGACTTTCTCATACACATAAACAGAAG
TCAAGTGAGTAAACTTGCTAGTATGTTCCTTTTGAACTACTACCAAATTTGGATCAAATAATACATCTTC
AAATTCATCATTAGTGCAATTCTGAACAATTTTACGTTTCATTACAATTTCCTCATTAATTGAACAGTGG
AGCGATACGTTTCAGAAGAGTATCAACACCTTTAGCGAATTTTCCATTTTATTCTCCAAGTTGTTTTCTG
TATCAGTAGTTGATATTGATATAGTACCATAATCAACTACTGATGTATATAGTTTTATGAAAAATTTAAA
CTTTATGCATAGAGAGCATTGCTATAGTGTTTAATCCAACTTTCAGGAATGACTTTGTATGTTCCTAAAA
ATACCACGTTGTACAACTTAACACCATCTTCTACCCATTGATCGGTAATGTATCCACACATAGCGCGAGT
ATAAACAACCCTTCCATCATCTTTAATAAAGTTAAATTCACAAGGAGCAATGAACTTGATAGCCTGACCG
AGTTTCCACTTAAAGTCTACACCTACATGCGAAGTATCAATCGTTTCAATTCCTTTAGCAGGAACAGCTT
TTAAAAACGCAGACTCAAGAAATTTCGCACGAACATAGCCAAACTGGGGTTTAGACTTTCCATCTTTAGG
AATGATACGCACTTTTACTTCAGAATCTTCATCTTTAACACCATGCTTAAGCTGAATGCTTACAACTTCG
ACCAATTTTCCTGCTGCTTTAGAACGGGATTTATCAGATACACGAGCTAATTCACCAATATTAATAATCA
TAGTTATCTCTCACTTGTTAAAAAGATTTTATACTCCACGGGACCATTATACTCTGGTCCCAAGAGTTTG
TAAACTATTAATTCAAAATAGCTACCACTGCACTACGAGGAACTACGGAGTACTCTCCAGCATGAACTAC
GTTCAGAAGTTCAACGCCATCTTCCAATCCATTGGTCAGTTACCCAACCACCAATCGGATTCGCAAATGG
ACGACGAATGTAAACTGCCTTACACAACAAATCAGTCGGGTCTTCAGGTTTTTCAATTTCTGTCAATAGA
TTAAAATCTTCTTCATAGATGATGAATGATGATACCCTTCCATAATAGTTTCTAATTTCCATGTACACTG
TACCAATGAGAATTCCACTATTAACACTAATGACAGTAAAAGGATATCCGCCAGTTGTACCAAGAAGTTC
CCAAAATTTGCTATCAGTCGTATTCGACATTGTCTGAAAATCAATTTCAGATGGTTTTTTCATTTGATAC
GCAGTATTAATTTTAATCATAATTTTCTCTTTAGTTTAAGGTAATAAAGCCTTTTAGTTCGGCATAGGAT
TTACGGAACATTACTTGATGCCCGCCAATGATAACTTGGTCATCTGGTACTTCGTATACAGCAAGATAAA
ATCCTTTCGAAGCTAATTCTTCACGCTCTTCTCGTGTGAACCATTTCATCATATCATATTCGCTAGCAAA
AGCAAAATGATAAAGAGCTACAAACCATCCGGGAATATGATATTCTACTCCAACATAATCTTTCTTGAAC
TTAGTATTAATTACGATATTAGCATTTTTAACTAATAGTTTGTCTTCGTGCGGCACAGGAATTCTTTTAT
TATCATTACTATGATGCATAAAATTAGGTCTGTCATAACCTACATGTAATAACCACTCTTCACTCCATGA
ATCTATTATACTTCTATACGGCGTTATTTGAACACAAAGATCTCGGCGTATTGTTATAGCGTCTTCATAA
TTAAGAATACTAAACGATGATTCAACACGATAAATTTTCATTTTATTATCCTCAGTAGCTATGGTGTTAT
AGTACCACAACTAACCGAGGAAGTAAACAACTTTTTATCGTTTTGTTGGAAGAGATAGAGGATCGCATTC
TTCCTCTGATGGAGCATCTTCAAGACCCATAGCATATCGCAAAGCATACTTCATCATCAGGATGTCTTTC
GCACAGTCATGAATAGAATCATGTGCAACGAATCCATCTAAAGTTCCCTTTGGAAGAGGACACGTTGTCA
TATCACGAACAAGCAGAAGTGCTTCAATTCTAGTACGAATATCACGCTGATTCCAAAATTTACAAGGTTC
TAACTTAAATGTATCAAGCTCATTCTCGGAAACGCCGTTAAGACGTTGAATATCGCGAATAAGATCGACT
AAAATTGGAAAATCAAACGACATTCCACGGCACCAGCCTTGAGATTTCCAAGGATCGATATTATGTGCAT
TGATGTAATCATTAAATTTTGCAATACCGTCGATAGTGCTTACATCTTCATCGGATGGTGCAATATTTTT
TCGAGCTTCAGGAGATTGATTCTTCCACCATTCGATAGTACTTTTAGTAAAAAGACGGTGTCCTTTTTGG
CTTTTTAAATCAAATTTGATTTTAATGCCACGTGAAACTAATTCATCGAATGTTTCAACTACTTCTGGAT
TAGGGTCAAAAGCAATTACAGCCAAATCAATAACCGCTGCTTTTTCACCACTTCCCATTGTTTCAAAATC
TATAATAAAATCAAACATTAAATTTTCCTCGCTAAATCACGAATTTGACCTACAGTATAGTCTTGAATAT
AAACTTTATTAATAGGCTCATCAATAAATTTTGCCATAGATTCAATATCTTTTTGTATTTCTTCAAGACT
GTATACTATCTTTGAAGCTTTTTCGCGAATAGTGATATTTTCAGGACCCGGATTTTCTTGAATGACAACT
TTAACATTTGTCATAAGAGATTTAAACTGGTACCAACTTAATTCAATCATTAATAATCGCCTCATAAAGA
TAGCTAATTTCGCCTAAAACATAATCATTGATTGTAACAGTTTTAACTTCACCGCAAAAGAATTCTAACG
CAATTAAATCTCGTTCAATTTCTTCTAATTGAAGCATCAACTTACTAGATTCAATTTTTACAGTTTCACG
ATTTTTGCTATAAGCTATTTCATAAATTTCGCTTACTTTATCTTGAAGAAGATAAAACTGATCTTTAGTT
ATTTCCACGAATAGCTTCCTCAAATTTAATCATACATAAAACACATCATAACGACCACGGGTGACACCAA
CATAAAGAAGTTGTTGAGCTAATTCAACATCTGCATAATGAATACAAGGCGTATAAATGAAAGCACGGTC
TACAGACATACCCTGCGCTTTATGGAATGTTGATGCAGGAAGTGCTTTCACTTTACTAAACTGTGATTTA
GCATCCCAAAAATCACTCCACGGAGCTTTTCCGCCTTTGTTCCAATTTTTATAAGTTTCTGCTGTTTTAG
CTAAAAATAGGTTAAACTTATACAATTCTTCGTCAGATGAAATTATTTTAATCTTTTCACGATAATATTC
ATCATCGCCATAAGTTTCTACTGTTAAATCCCAATGACGAATTAGATATTCTCCAGGAACACCACGGGCT
TTAACAAACGTTGATGTATACTCTGCTTCTATAATACGAACTAATTGTCCGTTATTAAAAATAATTTCTG
ACACAGGCTTTCCATCAATTTTATATGTTTTAAATAATGGTTCCTGCATTACAATAATTTCACCGACAAT
AAAATCTTTATCAGTTTCAAAAATCTTTTTACGAATAATGCTATTTAACTTGTCAACAGATTTATTCGTA
AATGCCATTACGCGATTTTCAAACAAATCATCTAGTGATTTGACGATTGAAAAATAATTTACCATAAAAT
CGCGTAAAGCGGTATCACCAGTAAATCCACGTACTCCATGCCCGTCAACAACTTTATCATAATTCCACTT
ACCGTTGCGAACGTCAGTAGCTACATCAATAATAGGAGCATTACTGCGTTTAACTTCAGTGAGTTCACAC
TGATAAAAATCTTTATGTGTAAAGAATGGACTGATATAAGCAGTATTTTCTCCTGGTTCAACAGGTCTGA
TTTGCTTATTATCCCCTATTCCAATTATAGTACACCAAGGTGGAATAGTTGAAAGCAGAATTTTAAATAG
CTTTCTATCATACATTGACACTTCGTCGCAGATTAATACTCTGCATTTGGCTAAATCAGGTACTTCTTTT
TGTTCAAAAAGAACATTTTCTTCATATGTTACTGGGTTAATTTTAAGAATACTATGAATAGTACTCGCTT
CTTTCCCTGATAGTTTTGAAAGAATCTTTTTAGCTGCATGTGTAGGAGCTGCTAAAATAATACCAGTTCC
ACCCGTAGATATTAAAGCTTCAATGATGAACTTAGTAAGAGTAGTCTTACCGGTACCAGCAGGTCCATTA
ATAGTTACATGATGTTTCTTTTCTTTAATAGCCTTCATAACAATGTTAAAGGCATTTTTCTGGCCTTCGG
TCAAATCATCAAATGTCATCGTAAATTCCCTGCAATTGGTATACTAACAATACGCCCAGTATCTAAAATT
CGCTGATATAATCTTTGCGTGTCTACGTCAGGCTTAACATGTTTAACTTCTATTTTATTAAACCAAAATT
TACGTGGAGTCTCAACTAATCTTGGAATTCCCTTACCTAAAGCTAATCGATACTGCTCTTTAAGAGTGGT
AAATACTTTATCAGCAATCTTCCATTCAAAAAATACAGCAGGACGATGTTCATCAAGCGGAACTGGCGCT
GTAAATCCGTCTTTGTCTCGGTAAACTATCGCATATACATAAACCATATTATCCTCGGATAAGTTTAAAA
ATTGAACAATTTAGCGGATATCCTCTTTTCAGTTTAAGTTTATCAATAAAAGACAAATTTTGATACCGCT
CTACACCTTGAATAATTTTATCACACATATCATATTGCATTTCTGCTTCTGACAACTTTTTCACAATTTT
CCAATCCGAGCCTTTAAGAAGAACGTTCAATTTAACAACTTCAGCGCCTTCTGCTATGCGAGAACCATCA
ATACGTGCTTTAAGTGCTATAATTCTCAGCTTAATGTCAGAGGTCTGTTTTGATTTAGAAAGCTGAGAAA
TGTGTTCAATTCGATTTTCACGTTTTTTCTGTATAGCTTTAATTTGATTATAAGTCTTTTTGATTTTAGC
CCATTTCTTTTCATCTAAATTTAGTTTATGAACTTTTTTCGCAGATGAACGACCAATTCGCAAAGCAAAT
AAATCACGCTTTTCAATCAACTCTTCTAAAGTATAATCAGAACGAAATGTATTATACTTTTTCTTTACTG
CAATAACATTCCCTTTAATGTATCCAACGTTATTATCAAAACGTTCTAATGATAATTTCTCTCCTTCAAT
ACGATTATCAAAAGGTTCTCCCGAGTAAGCACAAACTTTTTGATCTAAAATGTTCTTAATGTAATTGAAG
TCTAAGTTAAAATCTTTAGAACGTCTTTTTGCAGATGCCTGAGTATGCTCTAAACGACGTTTAATTTTAC
GAATTTGGTTATTAGACAGCTTCATATTTTTCTCACATCTTACGGACGGTTAACTACTTATACTATAACA
TTTTTACTTTAACTTGTAAACAACTTTATGAAAAATGCTTTAAAACTTTCATGGTATAATGAATCTAAGT
CCTTCCATTATAGATTAAATCCTTCAAAATCAAGAGTATAGATAGTGTATGTTGAACACTTTTTATACTC
ATATCTATCTGCAATTCTAAATACACTTCCAGCTGGTATCATTACTTCTTGTTCATCTGAAACTAATTCC
ATATTACGATAACGATGACTATCCGGAAACTTAAAGTTTGGATTGTATTCTTTACAGCGTAGAGCTTTTA
TAGCATACTCCTGGAAATTGAATACCATAGGAGCTTTGAATTCAAAAATAACTTGTGTGTTATACTCTAA
ACCAGAAGCAAAATGTAGAGCTATATTTTTATCATATGAAGCTGATACGACTTTATCAAATGTAATAATA
TCAATTCCTTGATTTAATACTTGTTTAGTCTCAGCTGGAACACCTCTCCAAAGAGGTTTATCGTTTGGAA
CCAAACGAGATTTGATTATTTCATTTAACCAAGAATGGTCATCTGGTTTATTAGTAATACAATGAATTAA
AAGTTCAATTTCAGATAAATTAAACCCTTCAGAAAGTAATTCTTCACGAATAGAAGCACGCACCGATGCA
TCCATTGATTTTATTTTAAAATCTTTTAGTTGCATTACTGAGTATTTCATTCAACTACCTCAATATCATA
AACTTTAAATGTTCCAAATGAATCGTGTAATTTTTCTTTTGAAATAGAAGTTATTTTATACTTTCCAATT
GGAATCATCCATTCTTGTTCACGCACAATCATCATTAAGTTATCAGTACGCTCTGAATCTAATCCATCAG
TATCTTCATACGTGTACTTAAACTCAGTATTAGGAGAAGAAAGTATAATATCGCTGATATGGTCAGAATA
ATTAAAAGCTTTATCAGTTTTTAAACGAAGTATTGTTTCAGTGAAATATTCAGCATAAGAAAAAGAACAC
GCTGTATGCAAACTAGTAGTAAATGAATCTACCCTGTTCGTTGAAAACACTTCTCCAACTTGTAAATCTT
TAATGAGTTCTTTTGTCGATTTTGATATACCACGATATAATTGATAAGGCGATTTAGTTAAATGCTTTTT
AATGATTTCATTTAAATGCTTATGAAGAGCTTCATTCTTTTTGGCTTCCATACATTGCCAAAGAACAGAC
TGCTCAAAGTCAGTAAATTTTTCACAGACCTTTTTATACATATCATATTGAAAATCAACGCTTTCAGCTT
TTATAGATAACTGTTCAACATCTGCAAGATTAATAATCATGATAGCCTCCGTATACTTCAGAAGCTATCA
TATCATCGTTAGAAAGGAAAGTAAACAACTTTTTGAATTATTTTGCCCAGGGAGCCCAAGGCGGAGGGTC
AAGATGGTATGAAGCTAGTTCTTCTAGAAGAGCATCTGGGGCTTCAATTCCATAATTCTGTAATACTATA
CGGTACTCTTTCTTATAATCACTAGAATCATTCTGGTTATTCGTAGAATGATTATCTTCTAACATCTCAA
ATAAATCCATATTAATTCCTAGCGATAAAAACCAAATTTACGATTAGTTTCAATGATCTTTCTTTCTTCT
TCGGACATTCTCCAGCGTAGTCCAACATCAAAATGAGCCCAGACCATCCTAACAAATGCATTTATATCTT
TAATATCTTCAATAATGAATGTTTTACTTTTAGAAGGTTGACTCGCTAATTTAACTTTATCGTCTTCAAA
CATGTCAAAAAGACCATATTCATGAGCTCTTTTATAGCCTTTAATAGTTAAAGCTTCAGAAGAAGAAAAT
GGTGAATCTGTATTCTGCAAAATATCTTCAGTATGCTTTATAATTAGAATAATATTTTCTGGATATTTTC
TTTCTTTTATATCTTTAATTAAAAAATCCGGATTTTCTGCTAAAGGAATAATTAATGAGCAATTTTTATC
AAGTGTATTTACTGATTTACCTTCTTTAGACATAAATTCTATTGAATATAATTTTGCTACTTCAATCATG
TGATTTCCTTTTGCCTACTAATGGACCGTCAGGAATTTTATTTTCCTGGATATATTTCTCATTTTCTTCC
ATCATTTTACTGCCAATTTTAAGAAGCAAATCCATTGCTTCATTTGCTTTTGCTTTAGCTTCTTCTAACG
TCATATCTTTGTTCATGATTTATCACCATAGATGTCTCTCATCAATTTAAGCGCTGAGCGTTCTAGTTTC
TTTTCTTTCTCAGCACTAATCATTGATTTCATCCATTCTTCTGATTCATTCTGCATTTCTTTATTTGCTT
GTTCAACCCAACCGTCATCAATATACATTGAGTTTGGTCTATTGAACCATTCAAGCATCTTCTTCAGAAC
TTTCATTCGTTTTACCTAAAACAATAGTAGGAGCATCGTCAAATTTATGAATTTTTAGCAAATTTGGATT
TAAATTATTCCATAAAGAGGTAATAAAATATGATAGCGCACTTTCGTCCGTAATTATAAATTTATTTCCT
TTATCTATTTTCCAATCATATATTGAATCATATGAATAGAAGGATAACGGTTTATTATTATAATATGCTT
CAGCAACATCAATATAGTTAGATTTAGTAAATGCTTGAATTGCCATAAATGGAGAATTTTGTACAGTTTC
AATAATTCCGATCTTTTTAAGTTTTATTTCAACATCTTCTGGAATTGGCATTGAAATAAAATCTTCTACT
AGATACATATTATTGTCATATCTTTTATCAATCATTACTGCTACAGTAATTGGAACATCCTTGACAAACG
CCATACTAATACTATTGATAGACATTTCAAACAAAATTGCTTCCATAATTTTCCTCAATCACAAGATGTA
GATGAACAACTAGAATCACAAGAACTTCCACATGAATCACCTGCCCATACATGAACAGGAACATTAGTAT
CATATGAATCAGAACTAGACTGTGTATTCTGTGTGTTAGATGATGTAGTAGGTGTTGACCAGCGCCAAGG
ATTTTTATAATATTCTTGGGCTTCTTCATAAGTCATAGTAACTGCTTCTACTGTTCCATCTCCCATATAA
ACATATTCAACTACAGTTAAAGGAAGGTAGTCATTTGAAATAGGAACTACACCTTCCCCAGGAGTTGTAG
AGAAAAAATCCGTAAAGAAACTTTTAAACCAATTAAAGATAAACATTACAAAAAGCCTCTTTTGAATTCG
ACTTGCTTCTCACCATAATCATATCGAATCTCTACATTAAATTCGACAGAACCATCTGCGTACATCATAA
ATGAATGCACAACAACTTCTGTAGACCATGGTTGTAGTTCATATTTCTTCATTACATGTCGTGAAATGAT
AATATCTAAATCTTCATTTGGTTTAATCCAACGATTTAACATAGTACTCTCCTCTATAAGATAATTCTAT
TATACCATACTCATTTTGGAAAGTAAACCATTTAAATGAAAAAAGGACTCCCGAAGGAGTCCTTGAGTTA
TTAACCAGTTACTTTCCACAAATCTTCATTTGCAGCAATCCATTCAGTACGTTGATTTTCTTCATATACT
GTAGAATATGCTGCTTTTTCTGAAGGGAATGTCTGGTAATGAGCGCCAGAAATTTTGTGAACGTCAGAAT
AAAGAGGGAAAGCTACAGAAATTTCCTTTCCTTCAATTTTCTTATTTCCATCTAATTTCTGCTCAAATGT
TTTGATATTAACATAACCGCGAGTACTAGCCATGTAATTCTCCTTTATTTAAATTACATGATTATTTATA
CATCTTCTTTTCTGAATAAGTAAATTAAATTCTTAAGAGCCGAACTTGTTACATCATATTTTCCTTTAAG
CGCCTTTACAACCGGGCCTGTTGCTGGTTTACCTAAAGAAACCCATAACTCGTGTATTTCGCTTTTAAGT
GGTTCATGCCAATGCGGTGCTTTTCTTTGCGCCCTTGAAGTTCCTTCTGAAATCTTTTTATTTCCGCCAT
TCGAATAAAACTTTTTCATTACTTCAGATTGCCGAGCTTTTCTTTCTGCCCTATTTTGGGCTATACGTTG
TGAATTCTTCATCCGAGTTTTTGTTTCAGGATTGTTTAATCTAAGTTTATGTTCTAATCGTTGTTGCTCA
GTCCATTTTCTTCCTTCTCCACCCTGACCACCAGGAGAAATATTAATGCAAGTATCTGGGTGTTTACGTT
TTAATGCAGATATTAGCTCACGTTCAACTTCATATGATTTTTCTCTAGAACCATGGCATTTTGACCATCG
TATTTTATAATTAAATCCATACTTACGATATATGTTCCATAGTATTTTACCTGAACCCGGATATTTATCA
TTATATGGATTTACAATAAATGATTCATGTTTCCCAGCGTACCAAAAGACGCCTTTCGGCGTCCTAACTT
TTACTATATATGTTCTATAAAATTTTTGTTTAATATCCATTATCTTGACGTTCAAAATTATGTTTGTTCT
TCAGATAATAAAGTTTAAAGATTTCTTCCGCATTCATTCCAAGGCCAACAAACATATTTAATACGAAATG
AAATATATCCACAAGCTCAAATTTAATTTCGAGCTGGTCTTCGGGGGACATTTCATCAATGCGTTTTTCT
TGCGCTTCAATATAACGTGCTTTCCATTTTTTCCATACAGCAGAAGCTTCTTTTTCACCACGTGACATTT
CACCAAGAGAAGTCAGAAGTTCGCGGAATTCATCATCAATACAGTCTTTTTGTTCACGCATCCAAGAAAC
AACATCACCGGCAGTTTCTAATTTATCTGGATGATAGCAGTATTCGCGGACATTAGCCAAACGAATCTGT
AAAAACCGCTGCATATCAAGCATAACTTGCAGCGGATCTTTTTCATCACCGAGAATATCCCAGTATTCAT
TTTGAGCTTTATCAACACCTTCGATCAAATGAGCACATTCATTAAAGTGAGCCATTAGTTTTCCTTTCAA
TTCATTAATAAGTTAAATAATTATATCATTTGAGTATGTAAGCAATTAATTAAAAATATATACTTCATCA
GTTCCATTCTTTTCTTTGGAATGATATATGTTAAAGACGTATTTTTTATTAAGATGCTTTACATTATATT
TTTTAGACCATTCTTTAAGAAGAGTGTTTTCCTTTCCGTGGTGTTCTAAAACATTCGACTGCCCAAATTT
TATTCCTCTATCATTTAAAGAATCTAAAAGATTTAAAAGGTCTTTTTCTTCATCTTCTGACCAAAATTTA
TTATAATCAGCAACTGTTATGAGATACGGAGGATCTACATATACAAAATCGCCGTCTAAAATTTTAACAT
CTTTAAAATGCAATGAACTAAAGATTATTTTATCACAATTTTGTTTAAAGTGATTATATTGTTTTTCACT
ATTTTTGTTTATAGTTCTTTTTCCAAACGGAGTAGTAAAATTTCCTTTATCGTTTATACGAATCATATTA
CTAAATCCGTGAAAATGAAGAACATAAAGTAAAAGAGGATCTCTAGTTTTATTATAATCTTCACGTAATT
TCAAAAACTCTTCTTTTGATGTTTTTGATAGTTTGTATTGCTTTATTACTTTTAAAACGTCATCCCATGA
TACATTAATAAGACGCTTATACATTTCAATAATTGGTTCTTGAATATCATTGGCCAATACAGGGCCATTA
ACATTCAAAGACACTGATAAACCTCCACAAAATAAATCCACGAATCTGTTATATTTTGGAAAGTGAGATT
TGAGTTCAGGTAATAATGATTGTTTATTACCTGTATACGCGATAGCTCCTAGCATTATATTCTCTCATTT
ATTGCAGCAAAAATGAATTATACAATTCTTCATCATATGTATTTGATAGTAATACTAACACGTTTTGCGA
TAAATATTAATTTAAAGGAGGATATATATGGTACAAAAATTAATGGCACTTGTTAATGCCATAAAAGGTA
ATAAGAAACGTATAGCTTTTACTATTTCTACTATGGTAGGAATTTTACTCTGGAACTTTATTTTATCACC
TGTTGCAATTGCACATGGTGTTAATATTCCAGTAGTTACTCTTGATACATTCGTAGATTTAGCATTTGCT
TTAGTTGGGTTAATTTAAATCTTAGCATATTTAGATAGCCGCATTTTAGCCATTAACCCCTGGGCAATAT
TATTTTTCATATATTCCATAATTTGTTCAGGGGTTGCACCTTCCTTTCTAATCATATCATTAACATCTTT
TGATTTCCAGGGAGATTTATCCCAAAACATAACCCTTTCTCCTGCATCAACTAATTTAGTCATTCGTTTA
ATAGTGTCAGGGTGACGAGGTTCATTATCTAAGACCCACACACGTCTATCTTTAAATGGAACAACTTCTA
GGTCTAATTGACCGCCCGTAATAGCTATACCATTTTCAATAAAAAGTGAATCTATAGGTCCTTCTAGAAC
ATATACATCACCATCTTTAACTCGTTCGACTCCATAGATTTTTGTTGCCTCAGGATAAGCTTCGATGGTG
ATATATTTTTGAGGAGCATCTTTCTTTAATGCACGTCCTTGAAAAGACTCAGCTTTTCCATTAGCATTAT
AAATTGGAATAACAAGACGAGGCTCAGAAATTTCCTTTTTGTATGTTCCCGGTGCTATGCTATTAACTAA
TTTAGGCCATTCGGTTGTAAACCAAAGATATTTCCATTTATCCTTTGGAATACAACGAGCTTTTACGTAT
TTTATAATTGGATGGTCTTCCGCCAGTTTATCTAATCTAACACATGACGGAAGAGATTTAATTATTTTCT
TCTCGGGTTGTTTAGGAAGTTCTTTAGGTTTTTCTATTGGACGACTTTTACCTTTTTCTTTTCTTATTTC
AAAGATATACTCACGATATAAATCGGGTTCAAACTCCTTTAAATATATTCCGATTGGTGCATGATAGTTA
CAGTTATAACAATGAATATTTCCTTCATTATTATCACCATAATACCATCCACGGGCTTTATTCTGGTCGG
TTTTTGAATCTCCACAAACAGGGCATCTAAACCGTAATTTAAAAGTTGAACTATTATTTACTTGTGTGAA
TTTAGGTAAATGAGCTAATGCACGGTATGCAAACTCATTATCAATCCAAGGTATTGATGACATTTTTACT
CTTCTTTTTCTTTAGATTCCTCTTTTTTCTTTTTAGGAATCTGTTCAGGACCTTTATTTACTACAGCGCC
TGATGTTGTTCCAATAGAGATATTTTCAGGATTACCACCTGAATCTCCAGCGACCATATCTTCTTTGATA
AATTCTTTAAATGTTTTCATATTAACCTCTATTCATAAAAGCATTAAAAATTTGGTCATCAATAGAAACA
TTTACTTTAGGCTGTTTTTCAGATGGCAATTCATATCCACATATTACAATTTTATGATCAATATCAAAAT
ACACAGAAGCAATATGATTAATGATATTTTCAGTAAAGTCTAAATCAACATCAATATCTTTTTGACCAAA
GCCCAAAGGATAAATAATGCGAGTAATTCGATTATCTTTAACAAAGATTCCACCGACATACTCTGTGCTG
CGTTTAAAGTCTACACGCCGACGAAATGAAAAATATTCAGGCTCTTTATGAGCTCGGCTCATAGGACACA
ACGAATAACTAGAATAAGAGATGTCAAATCCTACGCCTTCAATATGAACTAAATTGTCATGATTAAACCA
ATTATAATCATATGCCAAGTCCATTAGATTGTCATATGTGAAAAGCACCGGATTAACATCATTGGTCACA
AGCATATAATTAGCAACTGCTATAATTTCATTATTTTTAACGAATACAAACCGTGATTGATGCGAATGGC
CTGGACCTGCGTTTTCACGATTAATGATATAACACTGGGCACCTTTATATTTGTACGTGTCTTTACACTT
GTGCATTTGATAAAGCATTATTCACCTACCACTTCAGCGATGATATTTTTGTTATTAAAGTTTTTATCGC
AATACAGAACATAATTATACTGCATTACACCACCAGACTTAAGCTGTTTTTGCACTTCAGCTTTCATTTC
AGGACGATCACGCTTAACGATATTCATAATATCTGCTTCAATTTGAGTTTCAACCTCAGTCTGATCCGCA
GTCATAGACCATTCGCACAAATCTTTATCATAACCTGCCATAGCAGGCTGAGCAGCACAAGAAGCTAAAG
CAAAAATTGTAGCAAAGATGAATTTTTTCATGATAATCTCCTCAGTAGTTTATGTTTATATAGTATCTCA
ATTTCCAACAAAAGTAAACAGTTATTTTAAAATTTCTGCGTAATCACATGTTACAAACTGTTTCTCTAAC
TTGACGATTTTACGAAAGTATCTTTTGCATTGGCGAATCTGCCTCTTCGTAGGGCGAACAGCAAACTTAA
TAAATTCCACTCGACCAAATGGAGGGCTTTCTTCTGCTGGAATATCTAACACCAATTCCCACGTATCTGC
AATAAGTGCTTTGAATTGCGTATTTTTCCTGACGTTATACGGAGTAGGTTTAAATAAAACAATATGCATA
TTATCCTCGGCAATCCACTTCACATACTTTCTTGTCATCAATGAAAGCTTTAACTAATGCTTTATTAACT
TCAGCATATTGAGTAGTAGCCCATTGAACGTCATCTTTCATCATTGTGATTTCTTTAGTAAACATGCTTT
CATTCTTAAACCACCCCATAAAAACTACCTTTACCAATTCCATAACAATCTCCTCATTTAACCGACAAGA
CTACTATACCATAGTCTTGTCAGCTTGTAAACTAAAATTTTAATTCATTCGCCAAAGCATCTAACTGAGC
TCGAGTCGATTCATTTCTTTGATAGCGATTCTGCTCAGCCTGAATCTGTTGTGAACCTGCTACTTCGTTC
ACTTCAGTTGGAGTAGAATCTTGTTCAATTTCTACCCATTTTTGATTTCCTTTTTGAACACCCATCAAAA
ACTTATTCCACTTATTCTTATCACCATATCGTGATTTGATTTGCTTAATGAGTTGTTGTTCAGCAGCTGC
TAGCTCCTCGGTTTCAATGACCGCAAGCATAAAATCGGCTGTTGCTGGAAGACCGGCAGATTCTGCAATA
TCGCTCATGTTAACATCGGAAGAGTCCCAAGCTTGTTTACCAACCTGTGCTGCAGTCCAAAGAACAGTTT
CGGTTTCAACAGCCAGAGCACGTAATTCCTCTGCAATAGCTTTAACAGTTGTGTAACTATTTTCTGAATA
AACTCTAATGCGGCAAGATTTACAAATACCTAGATAGTCGACAATAATGATTGTTGGAACAAAATTCTTC
TTGAGCTTCAATTCGTTTAAAAGTGATCGAAATGTATTAGCGTCTGCTCCACCAGTAGGATACTGTTTAA
CGATTAAACGACCAAGAGTAGATTTCTCACGCCATTTTTCCATTTTTCCTTTATACTCAGCGTAAGAAAT
ATGCCCATCATCAATGTCATCAAGAGAAACATCAAGCATATTAGCGTCAATACGTTTAGCACAGACTTCT
TCTGCCATTTCCATGGAAATGTAAAGAACATTATGTCCGAGCTGTAAATAATCTGCCGCTAATGAACACA
ATCCTAATGATTTACCAACGTTAACGCCAGCCATTAAAACGTTCAGTGTTCCAGTTTCAGCTCCGCCTTT
CGTAATTTTGTTCAGAATTCTGAGTTTAAATGGAACCTTACGAGCTTTATTCATATAAGATAGCCAACGT
GCTTCGTAGTCATCCATCCAATCATGACCAACGTAACTATCAAATGAAATTGATAATGCCTGGCGCATGA
TGTCAGGAATAGCACCAACATCCGGCATTTTCTTATTTCGTTTTTCCGGAGGAAGCTCAGCATTAGTTTG
AATTTCGATTATTTTAGACGTAGCATTAAACATCGCCCTTTGCTGAACATATTTTTCTGTTTCTTTTACT
AACCAGCTGTGGTCTTCCGGAGAATCAGCCAGTTTTGAAATAAGTGTTTTTACACCAGAATATTCTGTTT
CAGTAAATGAACTATTTTCTAATGCAACATTTAACGCATTAATAGATGGAACGCTATGGTACTCATTAAC
ATGAGATTTAATTAATTTGAATGTATTTTTAGCTGGACCACTTTCAAAATATTCTGAATCCATATATGGC
CAAACTTTTGAAAAATAAGCTTGATCAAATATGAGATGAGAAAGAATAATTTCTACCACACTTACTCCTT
AAAAGAATTTAAATTTTTTCTTTGACCTTTTATTAAATGCATCTTGCAGTTGCATTGTAATACATTTTTC
TACATGAGGAGCTAACTCAGCTTTTCTTTCTTGGTCAAGAACAGCAAAGTCCATTACAACCTTTCCATCA
ACCCAATCCAGTTTAGTTACATACACTATATGCGTAGAACCATCTTCTAGTTTAATGACAATCTCCTGGA
TAACATTTTCCATAGCAGATTTAATTATCTTAAGAGACTCATTAAAAAGACGTTCTTTTCTTTCTTCTTC
CCCCTCCGAAGAGGGGGATTCATCGATAATTTCTAGATCTAAATCTAAATCATCTTTATTCATTAAATTC
TTCCATATCACTTAGCTGTTCGAGGTCAGTTTCTAAATCAGCAGCTGATTTACTTTTACTTTCTGGAGAT
TTAAATTTTTCAACCTTTGAGTTAATCAATTCATCAACTTCAGCTTCAACAATTTCATTACTATCAATAG
CACCTAACTGATAAGCACGTTTAATAGCATCTCGGAATGGTTGATGCTTAAATAAAGGACCCCAGAATGT
AGTGCAGTTGGTATCTTTTGCACGCCAAGATTTTTCTTCGCGAATCATCTCGCCAGTTTCTTCGTCAAGA
AATTCACGAGCATACCAGCCATTTTTAGGTTTTACCACGAATCCTAATTCTAGAGCCATATCTAACAATC
CAGAATAAGGATCGATACCACCGTCAAATTTAACATCAATAAAGAATTTACTTTTTTCTTTAACGGTACG
AGATTTTTCTACATTTAGAACAAATTGATACCCCTGAAGATCAGAACCATCTTTAATCTGGCGTTTACCG
ATAATGAATACGGTATCAGCCGAATACATCGGTCCAGTACCACCTCCCATAACTGTTTTACTAAACATTT
CTTGTGTTTCGTATGTATGGTTAATAGCAATACATGGAATATTTTTAGTACTAAAATAGGGAGTTACGAT
ACGAAATAAGCTTTTCATTGTTTTAGCTCTAGTCATATCACTAACAACTTTTTCATTTAAAGCATCTTCA
GTTTCTTTCTTAGAAGCTAAGTTACCAAGTGAATCGATAAAAACGACTACCTTTTCGCCGCGTTCAATTG
CATCCAATTGATTAACCATGTCAATACGTAATTGCTCAAGTGATTGAACCGGAGTATGAATTACTCGTTC
TGGATCGACTCCCATAGACCGCAAATAAGCAGGAGTAATACCAAATTCACTATCATAAAACAAACATACT
GCATCAGGATATTGACGCATGTAAGATGACACCATTGTTAATCCAAAGTTTGATTTAAATGATTTTGATG
GACCTGCCAAAATTAACAGACCAGATTGCATACCACCAGTAATTTCACCAGAAAGTGCAATATTCATCAT
AGGAATTTTTGTTCGAACTACATCTTTTTCATTAAAGAATTTAGATGCTGTTAATTCTGCAGTCAATTTA
GAAGTAGAAGCTTTAATCAAACGAGATTTTAAATCAGACATATAATACCTTATAGTAGTGTTCTTGTTCC
ACGAAATACTTCGAGCATTCTTGCATAAGATTTATTTGGAAATCCAGCTTCAATTGCTAATTTTTTAAGC
TTAATAGCACCGGATTTTCCAGAGTTTTCCCATATTTCTTTAATCTTTTCAATGTTATCCCACATTATTG
GGTCACGTTTATGTGATGGTTTATTTCTATTATAACCATATGGATTATTAAGCAAAGCTTCTTTTCGTTT
TGCTTTAGTTTCTTCTGACTGCTTTTTGCCTAGCTGGGCTTTCCGGCAAACATCACTAAATCCAAGATGT
TTTGGTTTTCCTTTTTGAGCTTTTGAAATTTTTTCTTTGGTTTCTGATGAAACTCTATAACCAATTCTTC
TACCACCCTCTCCACCAATTTTTAAATTATAAGTCATAGGATCATTTACCACATCTATTGTTACTAATTC
TCTTTCAGCATCACGGGCTGATTTAAAATCTTTATAAAACCCAAGAATGCTTAAATTAAAATTTTTCTTA
CCATACTTTTTCTTGGCTTGTGCTAATAAAGTTCCTGATCCCATATAACCATCATTTAAATCATCGGTTG
AATGAGTTCCATAGTAAATTTTATTATTAACTAAATTAGTTATAACATAAGTATAATTATATTTCTTTTC
TTTATGTCTTTTCATGTCATTAATCAACTGCTATTCGATGAACTTCTCTCTTTTCTAATCAATATAACAA
AAGAAGTCCCAAAAAGCAAACAGACCTAAACCGATAATAAGCAATAAAGGTCCTAACATTTATTCCACCG
GTTAAAGATAAATAACTTTCTAATAATAGTTCATAATTTTTATAAATCAATAGCTTTTTTGAACGCATCT
TGCCATTCGGCTTTCTTTGCACGGGTTTTATTTAAAATATCATGTTGAATAGAAAGCATCTCTTTACGCA
AAACATCACTGTGTTTTAACTCATTGACTCTATCAATGAGTTCAGCACGATTATTTACATAAAAACGAGC
ATCATTAATAATTCGATGTTTGGTATCAAATTCTTCGTCAATTAGCATCACTGCATCAGATGCCATTGTT
TCCCAGACGCGTAAGGTAATAAAGTTGTCATTATAATTCTTGTCACCAATAATTAATGCAGCAATAGCTT
GACTATTCTTTTCAGATACCATGTTCATAGGAATTTTTCCAGTGAACACCGGAGCTTTGGTCCAAGGATA
TTTAGGATTTTTAAACTGTTTTTCTCGTGCATTGCCAAAAAACTCAATATTTAAACCGGTGTCAAATAAG
AATTCTACCATCTTGGATTCGCGTTGACCGGACCGAAATGAACCGCCATAAATAACATCCAAAGTTTTCT
TGGTAGGCTTAGATAATTGAAAATCGTTCATATGAATTTTATATTGTTCAATAGGAAAATATTCAAATTC
AATAACATTATCAACTTTCTTATGCGCAGCCTTAGCAATGTCTAAATTTATACCTTGGGAAATCACTTTA
ATTGGTGATTTAATTAATAGCTCTTCTTCAGTGTACAAATATGCCCATGGTCTATTTTTAACATTTGGCC
AAGACTGCGAAAACGGCAAACGTATATCTGTAAATAAATAATAAATTTTACTTTTGTATTTTGCCATAAA
TTTTTGCGCAGATAAAATTGCTAAATTAGGTTTACCGCCAAAAAAGTTAATAGAAGAATTAACAACTATC
AAACGGTCATAATCATTAACATCTACTTCATCAAAAGATTTAGTGTAAACACCATTTTTAAGAGAAATAA
TGTCGACATTAAGACCCATTTCAGAAATAACTTTAAAAAGATAAATAGTTTCAGAAGATGGAACAGTTTT
AAAATTAATAACATTATTACCCATATTAATTATAGCAATTTTCATATTATTCCTTTTATGTTAAACGATT
AAGCGTATTTTCCTACATAATCTTTTTTCGAAATATGTGTTTCGCCAGTTTTCCACCAATGATCAACCAA
ATAAAAATGACGAGAATACACATGTAGGCTTCCAACATTCCATATAATGGAACCTGCTTTATACTGGCGA
GTTGAATCACCTGCATTCAAATCAGATACTAATTTATCTAATACGTATTTTTGCCATGCATAATCATTAC
GGAATCCGAAGACCACGTCATTTGAGCGCATGTTAACAACCGCATTGATTTTCTTGTCACGAATCAGGTA
TTGTACTGTATTCGTGCACATGAAATCTGACATACCATCTTTATTATAGTCAAACTGCATAGATGGACGA
GTATAAATCATGATACCACGTCGAGAATCAGGATTTTGACCAAGTTCAGCTAAACACATGTCATACTGAG
CATAGTTATCTTCTGACCAGATAGCCCAACCATAATTCGAGTTAATTTCACCTTTAGAAGATGCTACTTG
TTGCCAAATCTTCGGTGTTTCACCCGGAATATCTTTAACGAACAAGCTTTTAGATTTATACCATTCAAGT
TCACGCTGAATGTATTCATCATTAAGAGCGCCAAAAATAAACGGTTCATCTGCTACAAATGATGCGCCAA
TAATTTCAATAGTTTTAACACCTGTTTTATCAACTACGAAATCTTTTTCTTTTAATGCAAGCCCCAAATG
AAGACGGATTTCTTCAACTGTCATAGAGTCACTAATCATTTAAACCTCAATTGATACATTCATATTTAAC
TTGTAACAGTAATAAACTCCAACCTAAAATAATAGTTGGAATCATAAGAGGAACCGTTACACTATAGTAT
ATACTTATTATAATCATCAAGATTAAAAGCAATGCTGCTATAATTTTGCTTTTCATTCCTTCTCTCTGAT
GATAATTACCTGATTTGGCTGCGCAGACTTTTTAGTTTCACCTGCAATTGACCAAATAAATGTAATAAAC
CAACCAATAATTGACCAGTTAAACAGTAAAGATGCGAAAAAGATTCCTACTGTCGATTTTGACCCACGCA
TCAAGGCGATAAACCATGGAAGCATGTATATAATAATAGCCAACACGCCTGAAACTAAAACCATAAAAAT
TGAACCTGCTACTAAAGTTTCCATGTTTTCCTCACTTAGGTCAAATTTTTTACACATGAATTATAAGAAT
TCACTACATACTCCATCGGAGCGTTTTTACCTGTACGCCACTGGTAATTATTAGCCCAATTTGCCCAAAG
CTCAGCGCAGTAGTTTTCAATTTTTTCTTCGCGTGTAATTACATCTGAATTACGATATGCTTGAGCAGAT
TCATCTGGACGAATAGCTTCGTCAAAATTCGCCTGCATTTGTTCTACTGTCTGTTTTGGAGCTTCTTTAT
AACACTTGACATTAGGATTATAAAATTTGCTTGAACAGTTTACAATTTTTCCTACATCAGACTGATTTAC
TACCGGTCCTTGAGCTACACAACCAGCAAGACCTAATGCAATAACCAAAATAGCGATTTTCATTTCATTC
TCCAAATCCGTATCAGTAGTTGATAGTTGTATAGTACCATGGAAGAACAGTCTTGTAAACAGTTTTGTGA
AAAAATTTTTAGGGAATCCAAAGGGTCCAGAATCATCTCTTTTTCATAAGTATAGATTTATATTACTTGT
ATGAAAAAGGGACCTGGAGGTCCTAGATTTATTCTATCAGCCAAACAGGAAGTCTAACGAAGCTTTTTCT
TCATAGTCCATGCCAGCCGATTCACACATACCCGCAAGCGGTTTAACAAACGATTTTTGGAACAAAGTTG
AGTGGTCAATCCAAGATAGCACATCAGAACGAATTTCTTTTGGAAGTTCTGTACCCGATGGCCAAGCAAT
GCACTTGTCACCAAATGGATTTCCTTCACGTAATGGAAGAACCATTACTTTATTTCCATCCAAAATTGGA
GCTACACCTAAACCGCTAACAGCTCGACGATAAGTTAGCACACCACGAATATGGAACGGGCATTTAAATC
CTGGCCAACCTTTATCATCATATTTCGCTATATCGTTCGCAGTTTTTACTTCAGCAATAACTTTATAGTC
AAGTTGACGATATTCTTTCTCGAAGTTCTTGTAGTATTCTTGGACAGACTCTTCACCTTCCTGAAGAATA
CGACGAATACTTTCTTCGAGAGCTTCTTGCACTGCTTTTGGTGTTGAACTCTGCTGAGTTTCCATACCCA
TGATTTTTAGATGCGGTTCAGCAAATCGCTTATCTTCCATATCATAAACGTTCAGAGCATAACGCTTTTT
CGCTTTCCAAAATCCACCAACGCCCTTTGAACCAAGCGGAGGGCAAGAAATAGCTTCACGGTCCATATGC
ATCAGATGCTCGCGGTTATTCATATAATCACATAACTCACGATATGCAACATCAATCATAGGTTCCATCT
TTTTCTTACCGAACTGATTCATGAATTCAACCAAATCGTTCTGCTCTTTGAATCGGTCAAGACCAACTTT
TTCAATAACTTTATCTACGCAAACATATACCGAATCAGTATCACCTGCTGCAATGAAATCTTCATCATTA
GTTCCGCATACTTTATTCAGATATTCATTAATTTTACGAGCAATCCACTGAATACCGACTTGGCCGAAAA
TTGTGATAGCAGTAGCATTTCGCAAATCATAGTAACGGAAATGAATATTACCAAGAGCACCATAAAGACT
GTTAATGAGAATTTTACGGTTCAGCTGATTTGTATTAGCAAGTGTAGCTGCTTTTTCACATTCTTCAATC
AGACTATTGAGAACAGATTCGGTGTAATTCGATAGTTCATTTAAGAAATCATCACTGAACTTAACATATC
GTTCAACTTCTGGTTTAGTTGAACAAGACCCTGCGCCTTTCATAATAATCTTTTTAATAGCTTCGGCATT
CATTTCTTCAGCGAACATTTTCTTTTTCCAGTCTTTACGCTGGAAAAATACTTTAGCGATTTCCTTTGGA
ATGATACCTTCTTGATGTTTATCATACATCCATCCATTCGGAGAACAAGAATATTCATCACTCGGTTTAG
GAGCTGTTCCTGCGATATATTCATGAATTGGATGAACTTTAAACTGACCACGAATAGTTTCAGGACTAAT
GTTAACCTGGCGAATAATGCTCGGATACAGAGACGTCAAGTCAAAACTCATAATGTATCGACGTGCAATT
GGTTTAGGTTCAAACACAAATGCACCCGGAAAACTCTGTTTAACGTGCGAACCTTGTTGAGGAATAACCT
TATGTTCACCTTTCAATGAGTTAAAAATAATAGCATCCCAAGTTTTAATAGGACTCATTACACCAGAAAA
AGGCATTTTAGCGTAATAAGACATACTTAAAACTAGATCGATAAACCCACGAATTTTATCGATTGCTTGA
ACTGATTCTACGTCAATGATGTTATAACTAATGTATCGTTGATGATTAGTCTCACGAAGTTTATTAATAG
GACCGTCGTATGGTAATTTACCTTTTTTGGTTTCATGTTGAGCAACTGATTCCAAAGAGAATGACGGCAA
ATTAGTAAAAGCGAATTTCTTGTACAAATCTAAATAATCAAGAATAGATACGCCATCAATAGAATAAATT
TCTTTGCTACCGTACATATTTTGAATTAGTTTAGATTTTACCCGACCGATTGGAGAGAAACGTTTCATAC
TACGTTCACCCAGAATCATTTTAACACGATTCATGATATACGGAACGTCAAACCCCTCAATATTCCAACC
AGTAAAAATAGCAGGTCGTTTCTGTTCCCAAAGATTGATATATTCCATGAGCATATCACGCTCATTATCG
AATGGCATATAAATTACTCGGTCAAGAATTTCTTGAGGAACTTCATCACCACCTTCACAGTCAAGCTTAG
CAGCTAACTTTGCATCCCATTTTGATACTGAACCGTACATTGAATTCAAAAGGTCGAAAACATAAAAACG
ATCGTCAATTGAATCGTAATGAGTGATAGCATCAATTTCATATTCTGCTTTCATTGGGTCAGGAAATTTA
TCACCAGTAACCTCAATGTCACAGTTAGCTACACGAACAAATTTTCGGTCATAAACAATTTCTGAACCAT
ATGTATCACTTATATAAGCGAGTTTAAAATCGTTCATACCGAGAGCTTCGAGACCGATGTCTTCCATTCG
CTTCATCCAATCTCGAGCATCTTTCATTGATGGAAATTTTTGAGGAGCGCAGTTTTTACCATAGATGTCT
TTGTATTTTGACTCTTCCTTACAATGCCTAAACATAGTTGGAAGATATTCTACTTCACGGGTACGTTCCT
TTCCATTTTCATCAATATAACGTTCAACAATGTTATTTCCGACTGTTTCAATAGAGATATAAAATTCTTT
CATAGATATTCCTTAGTTTATAGCCCGAGTTATTAGGCTCTTGATATATTATACTCCAAATAAGGGGCCG
AAGCCCCTTGCTTAATTACCAATCGTATATTTAGGAACGAGCTTCCATTCATGTTTTTGTTTAAAAGAAA
TAACTCGGAAGTTATTAGTTAAATCTTTCATAAAAGTTCTTTGACCAGGAACGATTTCAATTAGTCCCCA
ATCTTCTAATAGCCATGCAATCGAATCACGACGAACTTCATCTTCTTCTGTCATTTCAACTTGACGACCA
TCCATACGAAGCATTTCTTTAAAATGAACGATATAGTATAGTCCTTTTTTCTGAAGAATATGACAGGACT
GATATAGAACTTTATCTTTATTATTAGCAATTCCCATACGAGTCAAAGTTTCTTTTACTTTCAGAAAATC
TTCAGGTTTTTTAAGAGTAATTTCAATCATTTTACCATTCCAATGCTAGTTTTTTGAGTTGTTTCTGTTC
TTTTACGTTCTTAGTCACTTCTTTCAAAAAATCATCCGTGACTAAACCTTTTAGTTCTTTTAATACTAAA
GGAAGTTTTCCATTTTTAGTAAGAATTGATTTATAGTTAATTGCATCATTTGTATTAACTTGATACCGCT
TAGCAAGTAACTTAATAATCAATACTTCGGTGGAATCTTCAACCAGTTTTGCCCATTTACCATATCTTTT
ACCACGAGGAACTGCAGCCATTAGATAATTAAAATGAGCTTCATCACTTAAGCCTGATCCAATTAAATTC
ATAGCATATACAGCTGGCATACACTCTGGAAATTGTGATAATGCATTTTCAACCATGAATTTTGAATAAT
CTTTTTGAGCAATAGAGCATTTAGTTTTATTATTAATAGCTCCAATTATTTCAAAAAATTCATTTTCTGC
TTTTTCTTTAAAAGAATCAGCAGCGGATTGGACAGCTGTCCAATCTTTTGAATACCAAGCAACTTGATGC
TCGTTTAATTGAATATCATCTTTAAATAAGCTCATATCACTTCCACTGCATTTCGCATGCTAATTGAATG
AAAAGATAAGCTAAATGCAATTCAGTATTAGCTGCAATACCATGATACTGATTATTTTCGCCGACAATTT
CGTACATACGAATAATACTTTGTGGAGTTACACGTGAATAGATTTCTTCGGCAAGTTTACCCACGAACCA
CGAATAATCAGCCGCATATTTTGGTGCTAAAGCTCTGAGTTGTTTAACATCTTTATTTTTGAGAGACTCA
AGAACATCATCAATAGCACCACGATCGTTAGTAACCAGTGATAAAATACCAGCATCCAAAACACCTTTAG
ACGAATAACTATCGAGCTCGCCAATAGTTTTACGAAAATCAGGAAAATTCTTTTTAACCAAAGCTGCTAC
AACTTTCATATCAGCTATAGCAATTCCTTCATGCTTGCAGATTTCAGTCAATCGACGAATCATCTGCTTC
ATCATTTCAATTTTATCTTCATCAGTTGGTTGACCGAATGTAATAACTCGGCAGCGTGACTGAAGCGGTT
TAATAATACCATCAATATTATTAGCAGTAATAATAATACTACAGTTTGAACTATAAGCTTCCATAAAGGA
ACGAAGATGTCGCTGAGACTCTGCTAACCCTGAACGGTCAAATTCATCAATAACGATTACTTTTTGACGA
CCATCAAATGAAGCGGCGCTGGCAAAATTAGTCAAAGGACCACGAACGAAATCAATTTTACAATCTGACC
CATTCACAAACATCATATCAGCATTTACATCATGACATAATGCTTTTGCTACAGTTGTTTTACCTGTTCC
TGGAGAAGGAGAATGAAGAATAATATGTGGAATCTTACCTTTACTTGTAATAGATTTAAAGGTTTCTTTA
TCAAAAGCGGGAAGAATACATTCATCGATAGTAGATGGACGATATTTCTGTTCAAGAATGTGTTCTTTTT
CATTTACAGTAATCATAATTTCCTCATTCAAGTTTTAGTGTAAATTATAAAGGCCGAAGCCCTCTATTAA
AAATCGTGGGTAGAATCAGCTTCAAGAGCTACCACATAATTCGCGTGTTCACCTTCAAATTTAGCAGCAC
CTTGTTTACCTTTTGCCCAAAGCAGAAGTTTATAATTTCCTGGTTGCATTTTCATATTTGCCATATTGAT
AATGAAATTAAATGTATTTTCACCATCATAATCACCAAGAGTCAAAGAATATTTAACACGGGTCAGAGCA
GAATCTTCTACTTTATTAAAACCGTTAATTACGATTTTACCTTCTTTTACCGTGATAGCAATTGTATCAA
TTTGCAGACCACGAGATACACGCAACAGCTGTTGAAGGTCTTCAGCTTTAATTTCAGTAACAGCAGATGC
TACCGGGAATGGAATTGGTTTATTAGGAGCAACTACTGTACTCGGATCGGCTGCTGGCCAAAAAATTGTT
GAGCGGGCATCAGCAATTTTAATATTTCCATCTTCTGACTGGGAAATTTCTGCATCATCATTAACTAAAG
ACAGAATACCGAGAAAACCGTTCAAATCGTAAATTGCTACATCAAAATCAATAACGTCAGAAATATTTGC
TTCCGCATAAGTTGTACCATTAACTGCGCGAGTCATAATAAATTGACCGGATTTAAGCATAATACCAGAG
TTAATAGTAGCGAAATTTTTAAGCAGAGCAGTAGTATCTTTAGACAGTTTCATGTAATTTCCTTCAATTC
AAATGAGATTTAATTTTATAACTAATTTAATAAAGCAATTAACGATTAAAATCAGCCGCAATTGTTTCCG
CAACAATTTGAGCAGCAACAATTAGACGTTCATCTGCATTACCGCAATAATCATCTTCAAGGCGTTCACC
ACATGAAGTCATAATAAATTTAGCACCGGCGTTTAGGGATTCTGTAGTATGTTTGCGCATTAGTTCAATC
CATTTATTACTTACTTCACGATCGATAGCTTCATAATACGCATGACGAGCAGGTGCAGATTTAATTTTGT
TCTGAATAACTTCCATTGCGTTATCAGAAAGAGACAAAACCCATGCTCGACGAATTTTATTTTGGTTTTG
TGGATTTGATTCAGAACGCACGTGTTTTGGCTGAATATCTTTTACATCAACAGTATAATTCACAGTAATT
TTAGTCATAATACACCTTTAGTCATAATAATCAGTAACAGTCCAAGCTTCATTTCTATTGGACATTATTT
TTGTATATTCTGCTTTAAATGCATTCCTAAGCATAGATTCAGTAACTATATGCTCTTCATTAGAAAAATT
ATTTCTCAGAATATATCGTTTTATTTCAGGAATAGTTAATAGATGCTGTCCAGTTGAATATTCCATGTTT
TTCCTCCATAGAGATTATACTCTAATAAATTAAAGCATAATCTCTTATAAATTAAACCATTACAGTAAAT
CGACCAACTTTCTTCATTTGAAGATGCTGACCATATTCTTGCGGGTCATGGTCTTTATGCGAAATTATAA
AAACGTTAGTGTTTTTCATTGAATTTATAATATTAGCTACACCTTTAATACCTTCGGCATCAAATGACCC
ATCAAACACTTCATCAAGAATTAATGTACTAATACTAACACCAGATACGATAGAAGCAATATCACGCCAA
GTAAATAAAAGAGCAATATCGATTCGTGCCTTTTCACCTTCACTAAATGAAGCATAACTAAAATCTTCAC
GACCACGGGATTTAATTGTCTCATTAAATTCTTCATCTAATGTAAACACATAATCCGCTTCCATTATTTT
AAGATAATGGTTAATCTGCTTATTAAATAATGGAATGTACTTTTTAATAATAGCACCTTTAATACCAGAA
TCTTTGAGCATATCAGTCAAAATTCCTCGGTGGTATTTTTCCATTACTAAATTAGTTTTTGTCTTAACAA
TTTTATCAAGTTCTTCTTGAAGCAGTGCTATTTCATCAGCATGGTCAATAAACTCAGAAGATGCTTTTTC
TATAGCCGCTTTAACTTTTTTAGCTTTATCTACTGCTGCGATCAGAGATTGCTTTTTATTGCGAATATCA
TTTGCCAACGACTGCTGGGTTTTAATATTATCTCGGTATTCATCAACAAGAACTTTTAAATTATCACGAT
GTGTTGAAAGCTGTTCAAACGAATGTGTGCATTCAGAAACTTTATCTTTAATTTTAGAAACAACTTTATC
ACCGGAACTCAATTGTGACAAACAGGTTGGACATAATCCACCTTCGTGATACATATTAATGACTTTATTA
TACGAGTCAATTTTTGATTTAATTAAAAATGCTTCTTGACCGATTTTATTAAATGCATCAGTCGGGTCTT
CGTCCAAAACAATATTAACTAATCTTTCGTTAGCTTCTTCTATTTCCGATTTTAGCGTTCTAGCTTCTTT
TGCCAAATCATCATACATATTTTGTAGACGAGTAAGGTTGTCACCCGTTAATTTTTTCTGGCGTTCAACA
TTATCATTATATATTTTAATTTGTTGGATAATACTATCTTTTTTAACATCAAGCACTTGGTTCTGCGAAT
TTAATTCACGTATTAGTGCTTTATTAAGCTTATCCATTTCAGCTAATGTTCCTACCTCAAGCAGGTCTTC
CACAAGCTTTCTTCGCGCAGGGGTCGACAAACCCATGAAAGGGGTATACCCTGCTGTACCAAGGACAACA
ATCTGCTTGAAACTGGCATATGACATTCCGATAAGCTGTTCAAATTCTGCTTGGAAATCTTTACTGCTGG
CAGATTCATTAAGACGTGTACCGTTAACGGTGATTTCGAAAACGTTTGGTTTTTGTCCTCTTTTGATATA
GTACTTTTTCTCATCATATTCCATCCACAGTTCAACTAAAAGTTCTTTCTTATTTGTGCTGTTTATTAAT
TGACCTTTCTTTACATCGCGAAATGGCTTACCAAAAAGCCCAAATGTGATGGCTTCTAGCATAGTAGACT
TACCACCGCCATTTCGTCCAGTAATAAGAGTTTTTTGAACCTTATCTAATTGAATGTCAATCCCATTTTG
ACCAACTGACATTATATTTTTATATTTTACTCTATTAAGTTTAAAATTCTTCACAAAAGATTCCTTTTAA
TGTATCTTTTAGACCATTCTATCATATCATCATAATCTAAAAAGTATTCATCAAATTCAGCCATGCAAAC
AACGCCTTGTGCTGTTTTTGATGTGATATAAATTATTCCAACATATCTAGAATCTTCTTCGGTATAATCA
ATATTTGCTATGAATTCATCATTAATGTCAAATGTCGAAAACTTCACAGTATGCATCCTTAATACAAGAT
ACAGCCATATCTCGTAATGATTTAGGTGTGTCATTATCTAAAATGTTGAAGTTAAAAGATACAGCCCAGT
CAGTGCAGACAGTAACCTTTTTAATACAATTATCTTCAACCTCTAACGGTTCAAACCAGTATTCAATAAT
TTCTTTATGACCAATATCATCTTTTACTTCACATTTAAAATGCTGACTCATCATAACATTTTTAAATTCA
TCAAAAGTCATTGTGTTGCCTCTACATATAGCTGATTTGCATATTGAATAAGTGCTTCACGGTCAGAATC
AGTGATGTCTGGAATTGCATTAATATACTCTTCCATTAATGTCTGAAGCGATTGAACTTCAACTTCTTCA
CTGTCATCTGACTCGACAGAGTTATCAATCTTTGACACAACTCGTAATGAATGCACAACTTTTTCTAGTT
CAGATTCGAACTTCGTCAGATTTTTGTCTACTTCAGTTACTATAACACGTACTGATAGATTTGTAAAATC
TTTATAGTCAATTTTTCCTTTAAATGGATAATGAATTCTACGATGCCAGGTAGTATTGTTTGGAATAAAT
TCCGTTCGTTCTGTTTCTGTATCAAACATCCAGAACCCACGAGGGTCATTCTCGTCACCTGCGGTTAGTG
TCCATGGTGTCCCAATATATCTGACGTTTGCAGCCTCAGAAATAGTATGGAAGTGACCAGACCACACTTC
TTTATAAGTCTTAAGGAAATCGGGTTCAAGACCATGAGATTTCATTCCTTTATAAAAATAAAATCCATTC
AGTTCCCAGTGACCAACACAAAAAGAAGCAGATGAAGTTTTGATATGCTCAAGAATTTCACCAGTATTTT
CTTCGCACATCCAAGGAATCAAATCAATCAAACACCCGTCAAAATCTACTGTAGTAGGCTTATCATACAC
TTTAACATTAGGATATTTAGCCAAAAGCTCAGTAGAAGCATTTGGATGCATTACATTTTTATAGTGGAGA
TCGTGATTTCCTACAATAGTGTGTAATGTAATTCCAGCATCATCAAGCGTTTGAACTATTTCACGGGCAA
ACTCCATAGTTTTATGTGTGATCGCTTTTCGCACATCAAAAATATCACCGTATTGAATCCAGGTAGTAAT
TCCATTTTTCTTAGAATATTCTATCGCTTGCTTAATTCCATCAATTTGAATACCGCGAATCCACTCATCA
TCAGCTTTAACGCCTAAATGCCAATCACCTAAATTTAAAATTTTCATATATCAAGAACCGTCATTGAAAT
GCAAAATAAAATTATTGAAATAAATCCATCTGGAGTGCTAAAGAACCCAATCCAACATGCTCTAGTGAAT
AGATAAAATGCAAGAAAAAGTATCACATATCCAAGAAATATCATTATATCAAACTCCGTATAAAGCTAAA
GGGCCGAAGCCCTTTATTTTGTAATAATGTCAAACTGTTCTTTAAAGCAGAAGCTTGAATCTTGATGCTG
ATACAAAAATTCATATGCTTTTTCTCGCTCACGGTCATAAAGAGCTCGGTCAGATGACAGTTCTTTAATA
CGTTCAAATGTTGATTCCATATCATTTTCATCAAACCAAATGATACCGCTATCATGCGAGGTCAAAGGAG
TATTATCAACACGGAATTTTAAATTTTCGCCAGTAGATTTCCAAAATACCGGAATTGTTCCACATGCACC
AAGCTCGAGATGAGTATATTCGAGTGAGCGTTGTAAGTATTTCTGGTTAAGTTTACTCAACTGATATCCA
AAGCCAGATTTACTCATTCGTTCAAGCATTTCACTATTAATATAACAATCTAGGATTTGTGCCGGTTGAT
TCGGCGCGAGATTCATTTTATCAATCTCACGATTACCGTAATATTCATACGGAATACCTTTTTCCTTAAT
TGCAATAAAAGCAGGGGAACGTTCCAGACCTTCCATTACAGTGGATTTACCAGCAGGTTTTAAGAATTTT
TCATGAAAATCAAACATCTGGTAAAAACCTTTCCATGTAGTCGTACGACCAATCCAACGGTTGATATTCA
TGTTAATTTCAGAAACATCTTTCCAATAAGTTGACCGAACCTTCACAATATCCATAGGAGGCTGAAAATT
ATATACTGTCGGTGCTTCTTCAATATCATCAAACAGAGAAACAGTTTCTGGATACCATTCTTTCATCAGA
ACTTTATTAAAATCACCATTATCAGAATGGCTAAAAATAACATCAGCTCGACGAACAGTTTCTTCTAATC
CCAAATTTCGACGCAAAGAAAGAACAGAATGATCATGCTGATAAACTACAACACGAATAGAAGGTTTAAT
ATTATCTAAAAGTTTTTTATAGTTATTAATCGTAGCTTCTTGAACGGAAGTAGCAGGAACAGAATTAATA
ATTAGAATATCACAATCATTTACTAGCTTAAGTGCTTTATCGTATTCTTTAGCTAAAATAACTGGAATTG
AAAATGATTTGTGGTCATGAGAACTTGTACGAGTAAATGATTTATCTTTAGCATAAACCAAAGTTACTTC
ATGACCATTTTTAATAAACCAATCACGTTGCTCGAGTGAGAATTTTGTTACACCACAACCTTCAAGACCT
CGAGCCATAAAAATGCAAATACGCATAGTTTTCCTCTTTTCATTTAATAAATCATGTAAATAATATTTTA
TTTTCTATAAAACGCTACGAATAGGCCCAAACATATCCATAAGCAATTTTGCTTTTTCCAGATATGCACT
TTTTAATATTAGACATGCATCCTTTAACGGATACAGCTGCGTCTTTAATTCTAGGAAATACTCGTAATAA
TTTTCCAGTAGTATCATATTGAAATACTGGCACATTTCTTTTCTGAGTTATCCCACGTTTAGATTTAGAC
ATTTTTTGTTTAGTTGCATTAGACATCCTAGAGGGCTTTCCTATGTTATCAGAATAATAATATTTCCATT
GAAATCCTCCAGCGGTTTTCCTTTTACCATCTACACATTGTTTAATTGAAGTTGAACAGCTATATGACAT
ATCTTCTGCAGCATCTGTAATACATCTATATTTGCGAATAAAATTTCCATTTAAATCATATTGATAAATT
GGTTTTCCAGCATTTCGTCTAGCGTTAGACATGTATTTTTTATTATCATCATCTTTCCAGAACTCTTTCA
TGCTTTCCGATATTGAAGAAGATACAGCTGTTTTAATCCATCCATACATTTTATTATTTAGTCTTGTTCC
GTCAGAACTATAACACATCATACGAATAGCTAAAGCCAATTTAGGAAGTCTATAAATTTTAAATAATAAT
AAATGCGCGGTAAAATGTTCTTCTGGTGTCAAAAGAACTAAATTAGTTTTATCATCTGTACCACCCATAC
ATCTAGGAATTATATGATGTGTTTCAGTATAGTATGTCAAAAGACTTTTATCATTGCCTCTGTTTAGTCC
TTTTTCGATCAGTAAATTATATATGTTTAAATAATTCATTTTAGTTTATTTTACCAAAAAATTTATAAAG
CAATATAGGAGCCGAAGCTCCTATCCACATAATACGCCATACAGAGGCTCGTTAGAACTTTTAAATTTTA
TGCGCTTATATGTTATAGTTCCTTCTGCTTTAGCTTTATCATGAGCCTCTTTAAAGCGTCTCATCATTTC
CTCTCTAGAGGAACGAATTTTATTATAATCTATTTCAGAAGTCGGATGTTCATCTTTCACAGTTGCCACC
ATTTTTTGACTTGATAAGAATCAACCCACACTTTCATATTAGGGTCTGCTCTTACAATAGGAGGATTAAC
TTCTTTGATTGAACTATCACGGTATTCTTTTTCAGAAATTTTCACGCAAAGATGACCATTCAAAGATTCA
GTAAATCCTGCATTAATTTTAAGACGCTTGAATTTTACGTGTGTAAGCATCAATCATATCCTCAATCTGC
GATCTAGTAGTCTTCCAAAGAATACTGATGAGTTCATCGTTATATGGCTGTTTTAGAATATCCCGACTTT
TCTTGATAGCATATTCGTATTGAGCAAAATTATTATTTTCAGCTGCGATCTGAGCGTGCTTATAAAGACG
ATTCAGTTCGCGTTTGTTTTTAGACAATAACTTATTTGCTTTTCTTTCTGCTTCAAGACGGTTCTTTTCT
TCAATAGAAGAAATAAGCTTTTCCACTTCATCATTAATTTCGGGTTTATCAGTCATATTATTTCTCTAAT
ATAAAATAAAAATCATCATCTGTTAAATGATACCGATAGTTTAATTCTACACCATTAGATTTAAAAGCGG
TATCATACGGATTTTCTGGATCAATATCAATGTCAAGAGCTAAAACTTCCCTGAGATACATTTTAAGTAA
ATAGGGAATAGCTTCAACTTCAGGTATTTCTTCCAAGAATCCGGAGAGGTTAATCGTTAGCCTCATATAA
AAAATCCAAACTAGGAGAATCATCTACAACACTTTTCTTTTCAGCCCCCGGTGTTCTATAGGTTGATTCT
TCGTAATGCGTCATTTTATCATAGATGTCTTGAATAAAAGTTTCATCTACTAACGCAACCATATCGTCGT
CACGGCTGTCATAGACATTGTGAACGAAGTAACTATATTTCTTTGCAACTTCCTTACGTTCTTTTTTAAT
ACGTTGGACGAATGCATTAAAACAAGCTTGAGTTATATACGCATGTGGGTTTTTATATTTCGTTTCATCA
AAATTGTGAAGCCCCTTAATAGAAGCTTCTATACCATCTGCAATCATTTCTTGTTTCCAAGACTGGGTGT
ATCCTGAAAAGTTGAAACGTTTAGATAAGCCTTCTGCAATAAGCATAATGGCTAATCCGATAGTATCATT
CTGACGAACTACTTTATTTGGGTCTTTATTATTTGCTAATTCTGTTTTCCAATCAATAATAGCTTGTAAA
AGCTCTTTATTGTTTACGTAATTATATTTAGGCTTAGTTTCTGACATTTTCACCTCTTAGCTCAATTCAT
AGATCTATTATATCATAATATTTGAAGACCTATCTTAAAGCATAGAGGATGAATCAATTTCAAGCACTTC
ATCAGATTAGCCGCTCCAAGAGCTGCATCTAGTGAATCAAATTGGTCAACATATTCAATTAATTCGCCGT
AATTAGCGTATAACCACCATTGGCTAAATTCATACTCAAGGATGAATCCATTTCCTTCAATTTGAGTTAA
ACCAATGCCATTTGTATTTACTTCATACCCAGCGAGACGTAAATCGTTAATAAGAGCTTCGTTCATAATT
ATACCTTAGTAATTTTCAGGTCTGCAAATTTTTTCTTGCGTTGATTTTTCATGCGACGAATAGTTTTATC
GGAAATTTCATGCTTTTGATAAGCTTTAGATTCTACACCAAAAGCTTTAACATCAAATTCTGACAAGATA
TATTGAACCAACAATTCACGGACAGTATTGCGTCCAATCTTCTGATCATTCTGTTTCATCGTTTGATGAA
GCTCTTTTTCCCATTTATCCAAAATTTGAGGAGTTACAATATCGCCTTTTTCTAGCAAAGAAACTACTTT
ATCATATGCGTAAGAATTGATGGCGTTTTTAATTTGAATAGTCATACATTATCCTCAATTACGTTAAAAT
TTTATTATCCAAAAAGGCCGAAGCCCTTAGGCTAAACTTTTTGGCACCCTTCCAGCCTTCGTACATCATT
GCGACTGACAATGACAAAGCTCCTTCACATGCTGCATACTTATTATTCCAGAACCAATTTAGAAAAACTT
CATCCTCAATACCGTTGTGTTTCATGTTCTGGAAAAATTTGGCGCGTTCTTCAGCAATTCGCTGCGATAA
TTTAGATTCAGGATTCATTTAAATTTCCCAATTGCCATTTTCATCAATAAATTTAATCCAGTCATTTACT
GACCACTTGGTCGTATCGCCTTTTGGAGTAACATTTAAAGTGTATAGCCCTTGCTTAAAAAGCATGCGTT
TGATATTCATATTTTCCTCAGCTGTAACGATAACACTCGTTTGATTTACGTTTAGCAACTCGTTGAGAAG
TATTATAATCAAAATCATCATCAATGTAAACTGATTTTTTCAACTTTCTTACTTCACCGCGTAATTGACG
ATTTAACTCATCTTTAACTTCTGAATCAATACCTTTCATTCTACGCCATTTATCTGAGCGAAAAATGTTT
TCAACCATATCTTTATGACTTACCCCATCAGGAGCTTTACGCTTTCCGAAATAGTCATAATCACGCACTT
TTAAGTCTTTACGACGATACGTTTTACCCATGGAGTTTAATTTCCTTAGCAACTGAACTAAATACAGCAC
GATCACAAATCATACGTTTATGTAACTTGAGTATAAGATAGTAAACATCAAAACCATTTACATAAGTAAC
ACGAACAACATTACCATTCACGAGATAATACTGTCCCTTTTTAATCTCTTTATCAACCACAACCATATCA
ATTCCTCAAAGGTAATTCATATGTTAATAATACCACGGTTTGAACTTGTTGTAAACAACTTTGTGAAAAA
TATTTTAGGGAATGATAAGAAAGGAACGATAGCTTAGAATGGTAATATACAGAATGTGAGAAAGAAAGGC
CCAGAGGGCCCGTCTTAATCTTCTATGATATCTCTATCATATCCAAGTGAAATGAGAGTTTCTTTGAAGT
GTTTAATGTTCTTTTGTCTAGAATCATTAATGAAAATGACTGGATAACGAATGTTAAGAGATGTGAATCC
AGCGCGTTTAGCAAGAGATACAATCAGCGGACGATCATACTCAATCTTACCATTATTTGTAAGAACTTTA
TAGAAAGTAAAAGGAGCATTGAGCTCCTTTAGAAGTTTTGTAACTGATTGACATCCAGGACAACGACCTA
CTTCATCTGGAATTCCATAGACTTCAATCTTATTTTGTTTCACAACTATTCCTTACTAAGAGCAGCATTC
AGCTTGTTAGTAATTTTATCCAGACGCTCATTGAATTCACTAGAAGATAAGCCCTTTTCTGGAGAAATCA
AACTAATCACGAAAAATATAGCAATAAAAGGAATAAGGAAAATAGCTCCAACTGCCATAAACAGAAAGAA
TGTTACAGTTGTAAGAAAATCAGCTAAACCTTTACGAAATTTATACATTTACATTTACCTTTAATTGATT
AACCAAGCATTGATAAGCACTAAACTATACTGCGAATAAAATTCTGGACCAAAATGAAAATCATATCATT
TATAGTATCCATAATGTAATTCAATTTAATCATGTTTCCACACCCCATCGGTATTTGACCAAAGTCGCTG
ATTATCTGATCCTCGCCACAGCTTTTTGGTCGGAAGATTTTTCTCATACTTCCCATCAATAATAACATCA
ACATATTTAAGCATTTCTAGTTGTTTAATATCTTCAAACTTATATCCTGTCCACAACCAGATATCTTTCT
CCGGAAATCTTGCTTTAACCCAAGAAACTAAATTTGAAATCTCTTCTCGGTTCTGTGGATAAAGTGGGTC
ACCGCCGGTTAAGGTAAGGCCTTGGATATACGATTTGCTTAAATGGGACGCAAGTTCTTTAACGGTATTC
ATAGTGAATAACTGTCCGTTACGAGCATTCCAAGTACTACGATTATAACAACCTTCGCATTTATGCAAAC
ATCCAGTGACGAAGAGAACGACCCTACATCCAGGGCCGTTAACGAAATCGCATGGATAAATTCTATCATA
ATTCATCTTTATATTTCCAAGTATATCCCTTATGAATAGATTGAATTCCTTTGCAACATTTATAAACAGC
AGAATGAAAAAATCCTGCATTTCTTATTTCAGTTGCTCCAGTTAGTTCTATTGTATTTCCGTCTGGTGAA
TAGCCAATAACCGTTTTGGTGTTAAAGCGATTTCTAGAACTATTTTTGATATGCTCTTTATGATTAGTAG
ACAAAGTTCTTCCTGTTAGCCCATCAGATATCCTTTTTCCAAAATCATCAGGAAGAGTTACTTTAGACCT
AGCTATGCTCATTAGCTTCTTAGTTTTATCGCTTCTTTTTGCGCCACGCTGATTTTCAAATTTCTTAGCT
TTTGCATACGCGTATTGTGAAGAAGTAACTTTTCTATGCTTACTATTACTCATTATCCAATATGCATCAT
ACATCTTTCCACCATATATTTTAGCCAATAAATGGTGAGCAGTATAGTGGGCCTTATAAGTTAAAAATAC
TAAGTTGTCTAAATCATCTGAACCACCCATACTTCTTGGAATTATATGATGAAGTTCATACCCGGCTTTA
GTTTGACGGGGCTTAGATGGGTGTTCAGCAGAATTAACTAGATTTGAATAGATTCTGTCATAATTCATTG
GTGCTTAACCCTATGCATGATTTCTTTATTTTTACCGAGATTAAATCCGCGTTCGTTCGGATTTCCCAAA
TAACCACATGTTCTTCTTATGGTATTCATCTTTTTAGGATCAGTTTCTCCACAAATAGAACAAACAAATC
CGTTTTCAGTAGGAGTCATTTCATGGGTACTTCCACATGTAAAACATTTATCTACTGGCATATTAACACC
AAAATAATCTAAATGCTGTGCAGCATAATCCCACACGGCCTCAAGACCTTTTAAGTTATTTTTCATATCA
GGAAGTTCAACATAAGAAATGTGACCACCTGTCGCAATGAAATGATATGGCGCTTCACGAGAAATCTTTT
CAAACGGAGTAATATTTTCTTCTACTGAAACATGGAAACTGTTAGTGTACCAGCCTTTATCGGTAACATC
TTTTACGCTTCCATATTTTTCTGTATCGAGTTTACAGAAGCGATAACACAGGTTTTCAGCAGGAGTCGAA
TATAAACTAAAAGCAAATCCGGTTCTTTCAGTCCACTGTTTAAGATGAGCATTCATTTTAGTTAAAATTT
CTCGTCCAATATCACGACCGACAAGAATATTCAATTCGTGAATACCAATGTATCCTAAAGACACTGAACT
TCTACCGTTTTTAAATAACTCAATTATGTCGTCATCAGGTTTAAGACGAACCCCGAATGCACCTTCTTGG
TAAAGAATAGGAGCAACAGTAGCTTTAACTCCTTTTAAGGAACTAATTCTACACATCAAAGCTTCAAAAC
ATAAATCCATTCGTTCATTAAATAGCTCAACAAATTTCTGTTCATTGAACTGTGTTCCAATATAAGAATC
TAACGCGATGCGAGGAAGATTCAGTGTTACAACACCAAGATTATTGCGTCCATCAAGAATTTCATTGCCA
GTCGAATCTTTCCATACGCTCAAGAAACTACGGCAACCCATCGGAGAAACAGGAACAGATGAACCAGTGA
TAGCTTTATTGTTCTTAGCTGAAATAATATCAGGATACATCCTTTTGCTTGCGCACTCTAGAGCAAGCTG
TTTAATATCATAGTTCGGATCGTCTTTATAAAGATTAACACCTTCTTCAACGAACATAACAAGCTTAGGG
AAAATAGGAGTTATCCCATCACGACCAAGACCTTTAATACGATTTTTCAGAATTGCTTTCTGAATCATTC
GTTCAGTCCAGTCAGTTCCCGTACCAAATGTAATTGTTACAAAAGGAGTCTGTCCGTTTGAACTGAATAA
CGTGTTCACTTCATCAATGTGTTCAGTAGAGTTCGCTACTTCTCTACCCGTTTATATGAAACAATGTAAT
TTGGATAATTGTCATCTAAACACAAATCTTTAATACGACTTGGGTGGAGTTTTAAATCTTTAGCAGCATC
CACAAATGATTTATAGATATTGTCATTTATTTTAACATATTCTGGGACGGGATGAATTGTTATTTTTATA
TCTATAACATTTGGATGATTACTAACCTGTTCTTCAGAGCATTTAAGAAATTTTGCAGCTGATTTAAAAC
TTCTAAATTTATTTCCGGATTTTAAAGCTATAGAAACAGTCTTTTTAGCCGTTCTACTACCAATATTATT
TTTAACATGAGCTTCACTTCTACTATTATTTTTATAATGCTCAATCAATTTCTCTCGTATCTTATCTTTA
TGTTTTAAAGATAAGATTTTACCTTTATGGGCATTACTAAGTTTTTGCTTATGTTCTTCTGAATCCGGAT
ATTTGTTAAATTTATATCCACCTATAGATTTATTAAGAATAAATTCGTTATTAAAATATTTCCTAATAAG
CATTTCTTCATGTTTAAGGGCCGATTCATAAGAATCAAAAACTTGAAGAATTATCCACTTAGCTTTATAA
TCTTTAAGCTTTTCTTTAACAAGCTTAGATGACGAATTGTATTCTTTCCAATTTGTATCTTTACCATATA
TAGTTTTGAATTTTTTAAAACCTATATAAAAAGACTTATCAGGAAATCTTACCATATAGGTAAATGCTAC
AGAATTGGCAATTTTTAAGCTTTTTCTTAATTTCCATTTCATCGTTTCACCTCGTATTCATATTTATACG
AGGATAAACAGCTGCATGTCACCATGCAGTTCAGACTATATCTTCAACTCTTAGAGTTGTCTGCCGTTTC
GGGTCGCTTGACCCTACTCCCTTACATTCATCAGGGATAGTCGTTGGGCATTTACAGCTACTGCTGATTT
AGCAACGGATTGTCTCAGTGAGAGTTTCCCGTTTTAGGCAGATTTTACATGAGCTTGACTTACGTTAACT
CATAAGCTTGGAATGCATCGTATACGTCTTTTTCTGTTTTAGATTGAGCATAATTCAACGCATCAGCGAT
TTGCCATTTTTCTGCATCCTCAATATGTTTTGCATAGGTGCGTTTAACATAAGGAGAAAGTACTTTATCT
ACATTCGCAAAAGTCGTTCCGCCGTATTGGTGAGAAGCAACTTGCGCAGTAATTTGTGCCATAATTGCAG
TAGCAACTCCAATTGATTTAGGAGTTTCAATCTGCGCATTACCAAGCTTAAATCCGTTTTCAAGCATTCC
TTTTAAATCTACTAAACAGCAATTAGTAAATGGAAGAGCAGGGGAATAATCAATATCATGCACGTGAATA
ATTCCGCTTTCATGCGCTTTCATAATAAAAGACGGGACCATATTTTTGGCAATGTGTTTAGACACAATAC
CAGCCATAAGGTCCCGTTGAGTTGGAAAAACACGAGAATCTTTATTAGCATTCTCGTTTAAAAGGTCTTT
ATTAGTTTTATGAATTAATCCTTCAATTTCTTTTTCAATTGTCATTTTAAACTCTTTCTAAGCTGCTTCT
TGAATGAAGCTATTAATTGTGTTTTGGTGTCAGATTCATTATATTCAAATCCTCTTTGAAGCATCTCGGC
CATCATTTCCTCTTTTCCTAAACGAGAAAATTCCTTTGATTTATCTCCAACAAAGTTAGGGTGAATATTA
TTTTGGGTGTAATCGGATTTTAAATAAGTAAGTAAATTTTCTAACCATTCAAGATAATCAACACCTTGTC
CCTTTAAGCCAGAACGATTAAATTTATGCTTCATTTGACCTTCTGCAGCATTGCATAGATTACAAAGCAA
TCCACGCACCTTTCCTGCTTTTGGTCCATTTAATTCATGGTCATGGTCGAGGTGATTAGCTTGAACATCA
GGATTTAGTTCTCGTTGGCAAATTAAGCATTTACCGTTTTGTGCATCATAAAATTTCTGTTTTTCTTCTT
TGTATAATTTGCCAGTCAATAACATAATAAACCCTTACCTTAAATAGATAAGGGTATTTATTATTTTCAA
GTATTGTAAAACATTTGATGCAATCGCTTATATTGCTGAATCATTCGGTCAGAAAAAGAAATTTGAGTTT
CAAGCCATTCAATGTACTCTGCGGCAGCTTGCATTAAATTTCCTTCATAGCCATCGTTATTTTCTTGTGC
AGCTAATTTAGCTAATGCGTATGAAATACGTTCACCTTGAAAATCGGCTTTAGGCTTCTGAACAACTTGA
TTAGTTCGCTCTACAACTTCTTCAATTTCGCCATTTTCTACTGATTCAGTATTCCACAAACACCAATACG
TAATTGGCTTATCGTAGATGTTAATAATCTTTCCATCAGAAAGTTCAATTTCAATAATTCCAGTGTCAGG
CTCTATATCATCTTCACATTCTTTTACAAGTTCACGAACTTTAAAGACAGTACCTGCACTAAGTTCCGGC
CAGTAATTACACAGCCCTGTATCAGCACGATTAATTCTAAACCATTTATCTACTGTAATCATGTCCCATC
TCCATATCAATTAAGTCATTTATCGTTGGTTCATTATACACCGTTTCTTCATCAGTGTAAACCGGTTCTT
CCGGCTCTGGCTCTACAGTTTCCCATCTAGCCGCCCACCAAGGTTTTATAGCGTAATCCATTCTCGTACT
GTCTGAGTTACTACACGTTCTCGAAGCTCAACCAGTTCAACAGTAGGAACTGCATAATACCAGTCAGAAT
GGTAAGAACCTGAGCGAGATTCATTTACAGCCACATGTACATTATGTTTTGGACTAAAATAAACACACTG
ACGATATTGGCATTTACCATCTTGCACCCAGTCTTCTATTTCGATAGGTTTAAGATAGTCGGAAAAATCG
AAATCATAGTTTTCCGAAAATGCATCATGGTCTTCAATGATTTCGCTCAGAACTGCATCAACATCTAATT
TATTTTCAATATTCATTTTTCACACCACCAACTGCTTACTTCAAAATCGGGTTCGCCATGTTCCCAGAAC
CAGGGGCCGATAGGAATCCACTTACCTTCATCGGTGACGTCATATTCTTCGGATTTAAGCCCAACGTATC
GCCATTCGACTTCATAAGCGGTACCACCGATTACAACTGTGTTCTCGCCATACGGGTTAGACACAACAAA
GTCTATACCTTCTGCTTTAGCTAACCAAATCATGTATTCATAGAGTTTATATTCCAGGTCGCCTTCTGTT
CCATCGCCTAGAAAAATAATTTGTTCATTTAATTCTATCATTTAAAGTATTCCCGCAATTGGTCAAATCC
ACCAATATGACTTCCATCAGGAGCAAATACCTGAGGCATTGTTAAGCCGATTTGAGTATCACGACCTAGT
TTAGTCAGAAGCTCAGCAATTTTCTCATCATCAAAAACACCTTTTTCCGGCATAATGTTGATAAATTCAA
ACGGCTGTTTCTTCACAGTCAAAAGACGTTTTGCATTATCGCAATACACACATTTATGAATGTTGCTATC
ATAACCATATACTTTAAACATATTATTCCTTAATTCCTAGTACTTGTTTAAAAGTCTCGTCGTAATCAAG
ACTTTGGCCCGTTTGTTCTTTATGCTTGTATATAATATCACTTACTTCTGATAGCATATTTTTATATGAA
CGAGTTAAAGCAGATTTAAGCACGCTGTATCTATCAGGAAATTTACCGTGTTCATTATAATAAGCTATTG
CTAATTCACGGACTGCCTTTTCAGCAGTCTCCATATATTCTTTACGCTTTGTCATTTTCTTCTCGGTCAA
ATCGGTGTTTACAATGGCGACATTTATAACGAAGATTATTAGTCTGCCAATGGACCAATTGCACCTGTTC
AGTTCCACATTCAGGGCAATTAGGAACGTTTTTAGAAGCCCTTTCGCGACGTTCAACCATGACCATTACA
GAATCCCAATTAACAGGAGGGCTATAATCATCACAACCATAAATCTTTCCACGCATTTCCAGATCGTCTT
CTTCACCAGCCATAATAATTTTAATTAGACTAGAATTACTTGAAGCTGCAATGTCTTCTAATAGACGCTT
TTTCATTTCAATACCTCAATAGCATTACGTAAACCATTTGCTTTGGCGGTTAAATCCTTAAGAACTTTAG
TATGCTCTTCAATCTGTTTTTCAACTTCAATCAAACGGGTACTGAGATATTCGCGTTCTTCTTTCATAGT
ATCATTCCCATGATTGGGCTTTGGCGTAGGTTTAAATTTATTAGGGTCCTTTAACTTAATAACAACTTTA
GTTTCAAACAGTGAAAGACCATAATTCGTATTATCATCAAAGGATTCAACTACGTCCATTTTAGACAATA
ACGTACGAAGTTTATAAGTCAAAGAACGAATCGTTTTATTATGTTTATTAGCTTCACATGGTTTCATATA
TGGAAGATTTTTATATATTTCATGCGGAGCACAAACAATAATTAACAATTCAAATGGTCCATTCTGAGAT
GATGGAATGAATTTAGCCGTAACCCAATCTTTTACATTTACATTAATAGTACGATCATCGCCATGACAAA
ACAGTCTACGAGAATGCTTAAAAAGCATATTCACATTATTATTAAATTCGTGTGAAAACTCACTGGCAAA
TTTTTCTTTACTATCTTTAGAAAACCATTCTTCTAAATAAGTTCTTTTCACGTCGTTAATAATATTAAAT
GTAACAACATTATCATTTGATACATTAGTCTCAATGTAGCTTGGATACATAAATTTTTTAACATTATTAA
CAGCTATCACAGAAGATAGTAATCGGGAACGTTTGAACCACTCGAGCAAAGAACCAAGAGAATGTGAATA
CGCATTTTGTGTATCTTTACAAATATGGTTTTCCCATCCAATATAGTCTAAAAAATCACGAAGAATATTA
TTGATTACTTCTCTATGTCCTTTCTGATAATCACGATGTTTAGTAATAAGACTGTCAAAATAATCAATAT
AATGTTTACGAGTTTTCATGTTCTTCTCACTTGGTTAATGATTTATACTCCGAGCCATCCTTGGCTTTAA
ATTTTACTTAATTAACTGCAAAGCTTGTTCTAGACGATCAAGACGATTAACGGATTCTTCCCAAATCTTT
TTAGCCTGCTCATATTCTTTCTGCGCTTTATTAGAAATTTCTAGAACTTCTTTATAAGCTTTTTCTAGTG
CAATAACTTCTGGACGAATTTCTATCGGTTCAGGTGAATCATCAAGACATTCCATTAGTTCCTCAAGGGT
AGTTTCTTCTTTAGGAGTATTCACAATTTCATCACATTTTTGTTGGTAAATTTCTTTACTAGTAGGTGAA
TAAGCACATTTCACTTCACGAAGGCTAATTACGAGTTTATATCCTTCCCAACCGCATATATTCTTAAAAG
GATATGTATGAATATTTTCTACTGTTTCCATACAAAGTAATGCGGTCTCTAATTGACGAGTTATAGTGCT
AGCAATTGAGAAAAATTTATTAATATTCTTTTGGTTAGTTTCTGGTGTAAAAAATCCATAATTCACAAAA
ATAGATACTTTATTTTTATCAAGTTCATATTCTTTTATGATAATCATATCAGAAGCCAAAGGATGAATTT
GACGATAATCGCCATAACATAATTTAGAAGCTGATTTTAGAATTTGCTTCTTGAAAAGTCTGAAATTACT
AATCCAACGACGAGTAAAAATATTCTCAGGGTCTTCTTTATTATTGAGATGATAAGAATTAACATCACCG
AACCAATTATATCCTACTACATCTTTAGTTCGTTTAATTTCTTTCCCAAAATTACCAAGAATATCCCGAT
TAACCAAATATGAAAGAAGACGAGACTCTTTAAAGGTTTTCTTGATAACATCTTTATCTACACGGCTAGA
AATTTTACGAATTTCATGAATAAATTTAGTAGAAGAAACAATACTTGCATCAACACTATTGAGCCACTGA
TTGATAATAGAAAGAACGCTATTTTGGCCAAACATCGGTATAGCTTTATCATCAATAACGCTATTGAATG
ATTTGATATATTCGTTACGAGTCATATTAATCTCCTCAGTAGAAAGTAAGAACATTATACCACATCCTTG
TGGCAAAGTAAACTAGTTCAGTGCATTTAGTGCATTGTTCAGTTTAGAACGTTGCTTTGTCAGATTTTGA
ACCCTTGACTGAGCTTGTTCTAACGCCTTTTCAGCTTCTAGCACTTCATTGGTCGCTTTAACTAATTCAG
CATCTACTGCCTTAAGAGACTTCTCAATCGCATCCGCGTGCCATTTTTCAACAGGTTTAAGACTTGGATT
TTCAACAGGAAGAAAATTCACTTTATTGAATTTCCATGCATCTTTATTACCTGAGCTATACATCCAAAAT
TTAGGATCGTTTGATAAGTAATTAGATAAATTTAAGTTATCTTGCACTTCTTGCTTTTTCTCTTGTGTGA
CTTCGTCCTTTCTCAAAAATTCATATTTAAATGAAACGATCATATCAAGTTCATATGATGTTGTATCTAA
CTTAAATCGTTCAAAACGAGGTAAAATCTTAGATTGAACAGCAGCAACAACATCCATATACTTAAATGCT
TCTGTCAACTGCGATTTAAGGCATTCGCAAATTGAAAGAGAATTTTTTGTATTAGGTTTAAAGCTAATTC
GTGCAGTTCTATTATTTTCTTTTAACGGTCTTACTTCCATCTGAAGAGTATAACCATCAAATTTCAGATT
CTTTAAGTTAATGTCAGAGCCTTTTAATCGACTAGCAGTAGACAAAATTTGCTTTAATTGTCTACGGAAC
AGAGCAATAAATCTCCATTCAATACGATAATGATTTGGATTGAAAAATCCAGATGATAAATCGACCTTAC
TTTGACCAACGCCTTGAACAAATAGAGGATTTTTATAATCAATAGTTTTACAGAAATCGGTGAGCTGTTC
ACGAGCAGTCATTTGAGTAATATACCCTGCTTTACTAAAATTACGCACCCATTCACTGCTATTCGTATAC
TTAAAGTGATGAATAACACGATTAACTTTATCAGGGTCTAAATTATTTTCACACAAAAATGTCATAACAT
CACGAGTATAGCTGGCATTACGAACCATATCTTCAATTTGAGAACGAGTTTTCATGGTGTTCCTTAAGAT
TTAAGTAAATCAACAATTTTAATTAACTTTTCACGCTCAGATTTAGCTTTACTACTCAATCCAGATAGTC
TGAAAATTTCATCATCATATTGTTGAATAGAAATATCAGCTCTTCAATTTGCTTATTAAAATAATCAATT
TGTTCAGAATGTTTTTCGTTACTACGAACTGGTACAGGTTTTGTAGGCAATTTAGTTGAACTGGATTCAT
TCGGGCGATAAATTAAAATGCAATTTGAACCAATCGGGCATTTAGCACCAGATGAATATTCTAACGTTCC
AGCTTCTTTTAAAACTTCAAAAGCTAAACAGAGATGATGCCCCATATTAACATAATCAGTAGAGCGAGCT
CTGATGTAATAATCATCTCCACGAGTGCTAAATTTAAAGCATTTAAGGTCTTCTGTATTATTAGTTTTAA
AAATCATTTGTTTATCTAGACCTTTAGCCAATCGAGCACCTAATGCTAATAATCGTCGTTGATTTTCCCA
CAAATCTGCGATCATTTGGTCAAATGATAATTTCGGCATTAGCCGACCATAAAGGTCATATCCTTTATTA
TAATTGCGAAGGATTTCACTCGCATTAATACGAGACAACGCTCCAATTTTATTAAGGGCTTTCATAATAC
GATTAGATAAACTCATTCCAGACCCGTTTGTTCGCTGTAAATGTTTTTCTAATCCAAATCCAATATCAAC
TTTAAATTTATCTAAAATTTCAGCATGAACATCTCTGTCAATAACATTCAAATCCAAAGTTGGGTTAAAT
CTATGAAAAAATTTATCTGGCTCTCCACGACGTAAAACAGTCCATTCATTCATCATTTTTTTATTAACTA
AAGATTTAATTACTGCGTTGACATTATTAATTACTACTGACATATTTTCCTCACTCAATTTTACCAATTA
CGCGGAATAAGATTGAACAGACTATATAAGTACCACATATAGATTGAACTAATGCCATTCCAAAGAACCA
AACAATATTATCAAACCATGTCTGTTTTACGTCGAAAGGACGTAAACTTACAGTAATATTATCACCTTTT
TCTATTGAAGAATACGTCTCTGGGGAAATATATTCACTAAATCTATAACCGTCTTTGAGTTCATATACAG
CAATAAACGATAAACTAGACCCCTTTCCTTGAGTTCCTGTAAGGGTATTAACTACAGTAACATCATAATC
TTTATAATGCATATAATCATTAATTGCGTAATAACCATATGCAATTACTATACATAAACAACATATCAAT
AAATTCAATCTTTTAATTATCAACTGTTTCATAATAATCTCAATTAAAAGGGCTTAGAACCATTATACCA
TCCTTGGTATAAAGCGGTTATGCGAGTACCGTATTTAACCGTTCTTCAAACTTCCGAAGAGTGTTCTGGC
GTTCAGCTCTTTGCTTTTTGTAAGTTTCAATACGCTCTGAAATGAGAGTGTATCGTTCATTTACTGATTC
TTTCATAAAATCAGGAATTTCTCGAGAAGCTTTAATCTCGTCAAATTTATCAATAACAGCTTGCTCTTCA
GCAATTAAGTTATCATACATCAAAATATCTTTCTTGATGAACTCAATATCTTCTTGAGTTACACGAGATA
ATTTAGATGCTTTATCCTTTTTGTACTGTTCGTTAGTATCACGAGACCAGTGTAATGTACGATTTTTATT
CGTATTCTTGTAAATTTCTACAATACCAATCTCATCGATAATAACGATCCAATTCCAACGGGATTTGTAA
ATTTGTCCTCCATTAACAGTGATTTCACCTCCGATTGAGATGTCATTAAAGAACTTGCTTTGTGCTTCTG
ATTTAAATTTACCATCGTTGTAATTTACCAGGTTGAAAATATCTTTAGCGTTCATTTTGTGTTCCTCCGT
AGTTGATAGTTGTATAGTACCACAGAGGAACGGTCTTGTAAACAACTAAAAGAAACTTCTTTCACAATTT
TTTCCACTGAACCAAGCGCTCACTGCTTTCTTAGTTTCAGGAGCAGTGTTATCCATAAACCATTCAAAGG
CAGCCTTTTTATGATTCTGGAGGGCTTCTCGGGCTTTAATCTGCTCACGGTCTATTAACACTAACATATG
AGCCTTTCTTGTCACCAGGGGCTTCTTATGATTTTTTGAATACTCCCAATCATTTGTCCATCGCATCGTT
GTTGCGAATTGAAATACAGCTTCTTTAATCTTAGTTTCGTAAATTTCACGAGCCTTTGAGTATAACATCA
TTACCTCCATTTACCAGTTTAATTCTAGTCATCTTTTTGATGGCAGTCCATATAATCTATTTCTGAACTG
CCTTTTTGTCTTAGAAGGCCTCTTATGAATTTATTTCAGAAGAGTAACCCGTAGCGATTTCTTCCCAACC
GTTTTTGTCGGTCATAATAAAGTCAGCAAGATAAAGTGCAGTACGCAGTGAAACATTACGTAAACGGTTA
ACATTGACTTTCATCCATGATAATGCTTTATAAGTTTCTTCATCAGAAAGACCACGCTTTTGCATCATGT
CGGTTGAAAGAATAACATCTTCAACCCTGACCATAATTTCTTCATTAGTGTGAACACCCAAATCCAAATA
AACTGAGCGGGACACTAATGCTTGTAAATGTGGAGCAAGTTTAGTACCACGGTCTAATTCGCGGTCAATG
TCAACGTTTGTGATAAAAACAATTGTTCCTTTAAATTCGAGCTCACGCTCAATGCCTTTTTCTTCTAAGT
AAGAAGATGCAGTGCTCCAGCAGACTTTACGGGTCTCTCCAGTGTCCAGAGCAGCTTTCAGAAGATTAAG
AATGTCCATATCAGAGAAAACATCCACATCATCAATCAAAAGGACAGAATTCTCTTCACGATTATTCCAA
AGCTGTTCATAAAGACCGATACCAGAGATTTTACCGTTAATGCTTTTATACTCAATGTATCCAATATCAT
TTGCTTTATTCAAAGCTTTATCTAAAGAATATGTTTTACCAATACCCGCCGCACCAGAGATAATTAATGA
ACGAATATTTCCGTTAATAATACCATTCGTCATCATTCCCATAACATTAAATCTTTTATTAATGCGGGTT
TTCATATCTTCATATGATTCTTTAACTTCTTCAACTTTTACACCATCATATGAAATGTCTGATTTGTAAA
CCCAAACACCGCGACGTTTACCGTCAATTTCAACAAAAACTTTACCATCTCCTTGTGCATCTACCGGAGC
ATTATCAGGGAACCATTCACCTAAGAGCTCAAAAGTTCCAGAGATTTCTTTACCGAAGTACATACCCTTA
TTGATAGTTACAGTTTTCATTTTATTCTCCAAATCCGTATCAGTTGATAGTTGTATAGTACCATAAAGCT
TTATGCTTGTAAACCGTTTTGTGAAAAAATTTTGAAATAAAAAAGGGAGCCCGAAGGCTCCCTATCATTT
ATAATAACTTCGATGGTTTTCAAGATAAACCCTCTCAAGGAAGTCATCCCAGAAACTCATGTCTACTTTT
TGCTGCATACCGTTCTTAGAAGCTTCAGTAGATGCTGCTTCTACTTGATCGACCACATCTTCCAAAAACT
CTTGAACGGTTTTAAATGGATGCTTACCCAACTTCACGTCGAGAATAAATGGAGCATCTTGGAGTGGATA
AACCAAGTCACCAGTTTTGTAAATTTCCAATAGTTGAAGTCCACCACGACAAGCATGGCTCAGAGCTTTC
CAGTCAATGCCTTCATTGGCTTCGGCCTTACGAGCACGTTCGCCGTATTCAGCATCTAATTTGTTCAGTG
ACTGCTTAAGCTCAATAAGAGAAAGCGTTGTCTGATATTTACGACCCAACACTGTGTAAAACGTTTGTGG
GCCTGTTTTCTCATGATTATGGAACACCCATTCACAGAATTCGTTTTCTGGAAGACGATGCTTAATATCT
TCAACTTTAGTACGACGCTGCTTAATGGAACCATCTTCTTGGTAATCAACCCATTGCTCAGGGATTTGAT
TAACTACTTTCAATACATCGCGTAATGCAGCCAAACGAGAACCCTTGACGCCGTATTTAGAAGCTTGCTT
GCGGACATATCCTAAATATGATTTCATGTTAGTCGTATAAAAACGAGAACGGTTGTCTTGAATAAACTTC
CAMACATCAGGCAAATCGGATTTAACCACTAGTTCAGGTGGAGTGTGAAGCATATCCAATGCTACAGGTT
CACCATCTGCTGCTAATTTAAAGAAATACTTAAGACTATACAATTCGTGGTCAATATTATCTTTAGTGTT
TTTAGATGATGTGTTGTTGGTATTTTTACTCATGTGCTCTTTGACGTTTCCAATAAGAATATCGCGAGCA
GGAGGAACAAAGATTTCTTTAAAATCTACATCAGATTCTGGAGTAGAAGTTCCATAAAGATGACTACCAA
AATAAGACTTAACTACAGTTTTCATTATTAGACCTTTCATAATCTTCATTAAATTGTACAATCAATCGAT
GATAATTAGATTTTGGATATCCTAATTTACTTATATAAGTTCCGAATGACCCACGTTTAGGTCTATTTAA
TTTAATCCATAACTTATAAAGTAAGTCATAGTCTTGCCAATGTTTACCAGTTCTTTGTCTAATTTTAGAT
GAATTTGATTGTTTCTTTTTAGCTTCAAAATTATTCAAAGATTTTTTGACTGCAGCAGAGTGTTTTTCTT
TAACTCCCGGTCGTTTAAAAGCAGCTTTCACCCCTTTAGATATTTTTTCTTTAACTTCCGGTTTGTTTTG
TGCTTCTAACTGGGATTCTGAAATTTTAGCCCTTATTTCAGGAAGACTTAATGTTATCGAACGTTTTTCT
CTGTATTCATTAGACTTCCACATTTCTTTCATTGTATTAGAATGTATTTTTGAAAATTTTTCTCTAACTA
ACTGATATCCTCTTGAAGTTAATTTGAGATTTCTTCCTAAAGAATCTTCTCCAAAATTATAAAATGACCA
CCATGCATAAATTAATCCAGGCGAATTGTAATGAATTTTAGCTAAAAGCCAATGGGCTATAAAATGCTCT
CTAGCTGTTAATAAAACTAGATTATCAGAATCATCATTACCACCAATACAAGATGGAATAATATGATGCT
TTTCCGTATAAAAATTTAATTTAGATTTATCTAATTTTCTGAGTTTTCCTTTCTTAATTAAATTATTATA
TACTTTAGTATAATTCATTGGTTCTTCGTCTCATTTAATTTTGCTTTGCATTTATAACACATGTCTGTTC
CACTTGAAATAAACATATTTCCTCACTTTGAAATCATAGTTGGAATAACAGAATCAAGATAAGTCTTTAG
TGCAATAGCTTCCTCTTTCTTTAATGTAATGATATGTGATCGAAAATCATCAATTTGACGAATAGATACT
ACATCTCCCTCTTCATAGCATTTTGAAACATTTAAAATAGTTTCATCATTCTGATTACAGGAGTTAGTAA
TAATAGCATTACATTTTTTAAACCATTTTAAATTATGTTTTCTTTTAGTAGAATCATAAAAATATTTAAT
GTTAGTTATAAAATTATCCCAATTATTAATTGATAAGCACATTGACTCGCTTTTAATATTAAATCCTGGG
CATGAAGAATAAAAATGAATTTTATGCTCATCATTAATGCTTACAATTTTATCAGTGTAAGCATATTCAA
TTTGGGTTAAACGAACAATTTCGCTAGGCGTAAAATATAACATGTCATCTTCTTGCGTCAAACGATACAT
GTTATTTACTTTTTCTATAGCCAATTCACCAAAAAGTGGACTAACTTCTAATACTAGTCGTTTCGCCTCG
CTCATCATTACATACTCCTCTGAATCATATTAATAATGTTATTCACCAGATTATAAGTAAACATTGGGTA
ATTATATTGAATCATCACATATATAACAAACAAAACTTTCATTCTCTTCTCCTTGGCAGTTGACAAGATT
ACTATACCATAATCTTGTCAACTTGTAAACCATTAAATGACGTTTTCGATAAAATTTTGAAGCTTTGTAT
GAGCATCAACCATGATTTTCATTTCTTCCTTGGAAAAGCTGTGCCTCTTTCCCGAGAAGAACATTCATAG
TCAGAAACTGCATTAGCATATTCTTCAATTAGCTTCATTAAAAACATCTGTTTTTCAGTTTTCATTATTC
CACCTAATCATTTCAAGATATTGAACTAACTTAGCTTTGGATTTATCCAAATCCCTTTTAGCTGCTTCTA
TACCGTCGTATGAATATCCTTCACAATGCTCAACTGCTAATTGATATGAATCTATTTCAATATCATGCGC
TAATTTAATGATTTTTTCAAACTGTTCGCGTGTTAGCATACTTAAACTCTCGTATTATGATCGATAATTT
CATCAAGAAACATATCTAACGCTTCTACAGCATTATTAACTTTAGCTTCAAATTCTTCAATACCTGATAA
GGCAAAGTTAATGCGTTCCTCATCTGCTTTACGAATTAAAGCTACCAATTCCTTAATTTTATCCGCTTGT
TCGATACTAATCATTATTCCACCACATATGAAAGAGAGAATATTGCACACGCCATGTGAGTTGCAACTTC
ATCACACATATTATAACGTTTCTTAAGAAGTTCTACAAGTTCTTCACTAGTAACTTCATCCATGTCGACG
AAAAAATCACCATTAATGATGACGTAGATATTTCCTTCTTGATTGAGTGCTTCAATTTTCATGATGTTCT
CCTCTTTATCCGATGGTTGTATAGTACCACAGCTCAAACGGAAAGTAAACCGGTAAAATGAAAAAAAGTC
TCCCGAAGGAGACTAATGTTATTCGAGGGAAAGAAGATACTTACTCTGGTAAAACATTCCAGTAATATCA
TCTATCGTGCTTTGGATGGCTGGAGGCATTTCTTTATAAATGCTGTTAGATTGGTCTAGTATGCGATCAA
TCATTTTAATTGTGTCGGTAGGAAGTTTACTGGCATCTGGAATTGAAGGCGTGTATTTTCGACCAGAATA
CCCCAAATATTGCTCACCAAATTTATCAATCAAATCTGGCAACTCGGAAAAAATAAAATCGTATGCTTTG
TGTCTAGCATAACTTTTAGTTTCAAAATGTGCAGAATGAAAATAAGCTTGTGCAGCCATTAATAAACCTA
AGTATTCATCTGCCTTTGAAGGTTTTCCACTTTGTGAAAAGTCGCTGAATTTCATTCAGTCTCCAATTTA
ATGTTCATAATTCTAGCGTATGATTGTGCCATCTCCGCGCCTCGCTCTATACATTCAAAATCAGAAGAGC
ACGGGTCATTTTTATAGGTCGTTCTCATAAAACTATAGAATTGTTCAGACGATTCTACGCTTTTATTTTC
AAAAAGCATATAAACGTGCCTAATACCAGATTCCATAAATTTATCAAAATGAGGATCGACATTCGCTTCA
ATCGATGGAGATAAAACAAATGACAATCCTAGCATGGCAAAAAGTGCTGTTGCTTTTAAGGCCATAAAGG
CCTCCTATCATTTTTGTCCTGTATTTACTTTGTGCCGATGCACGGCCTTAACTTTATCAAGGTATTTTTC
AAAATTTCGCAATCTAGTATAGTCTGCCGGAGATTGGTTGAGTGATACTTCTCGACGCAAAGCTGAAATG
ATATTTCCAACTTCCCTACGAATTTCATCTAATTGAAGAACAGTAAGATTGCGAAGTTGCTTTTCAGTTA
ATTGTAGCATATATACCCCTTTAGTTAGATAAACCTATTTATAACTTTTGCACTAACCGAGCTTTTTAGT
TAATTCATTCCAATGTTTTCTACACAAAGAAACATAAATTTCATCACCAATACAAATTTGATTACCTTCT
TTAACTGGTGTTCCATCTTCCATTAATCGAGCTGTCATAATCGCTTTTTTACCACAATGACAAACTGCTT
TTAGTTCAATAAGTTTATCTGCAATCGCTAAAAGTTCTTTAGAACCTTCAAATAATTTTCCAGCGAAATC
AGTCCTTAGCCCATAAGCCATAACAGGAACATTATATGTATCAACAATTCGGCTCAATTGATGCACCTGT
TCAGTTTTTAAAAACTGAGCTTCATCTACAAATACGCAATGAATATCTTTTTGTGCTTCAGCCCATTTAT
AGAACTCGAAAATATCCATATCATCTGTAATAATATTCGCTTCCTGCTTAATTCCAATGCGAGAAACGAC
TTCACAGACAGAATCGCGAGTATCAATAGCAGGCTTAAGAACTAATACACTCATTCCACGTTCTTTATAA
TTATGTGCAGCAATCAAAAGAGAAGCAGATTTTCCAGCATTCATTGCTGCATAAGTAAAAATTAAACTCG
CCATCTTAGTCCTTAGTTAAATTTTCTAAATATGTTTCTAAATCATTTTCAGCTTTATCGATAGATTTTA
CTAATTCGTAATATGTTTCGGCATCTCCATATTCAGAAGATATTTCAAAAGACAAATCCTTTTCTAAACT
AATAATTTCACCAACTAAAAATAATATTTCGTTCTTTTGTTCGCGAGTAATCATAAGGAATTTATATAAT
CAATGAGTTCTTGTTCTTTATTATCGAATTCTTTAGAAAGTTCTTCGTACTCGTTTGCGCTAAAAGGACC
GCATTCATTACAAACTTTTTCCAATTCACTATTTTTATCCATAACTTCGTGGATAAGAGAAAAGAGTGTG
TCTTTTTGTTCTTTGCTTAAACTCATAACCATGTCACCTTTAAGCAGTATTCTTCTACATGCTGTTTACG
ACCTTTCTTATCAATAAAGGTATATTCAACGAATGTTCCAATGTAGTCTTTATCTACATCATGTGGACTA
TTAATTGGACATTTAGTGCGACAAATGCGTTCCCATTGACGAATAATTACTGCCTTATTCTTTGGGTCAT
ATGGATGTGGATAATGTATATTCATAATAATGGTTCCCAATCAACAATCACAATTTCTAATTTAGAGGAA
TATGTATCTAAAATCCCCTCAATAATATCCCAGTTCCCTTTACCTATGCCTGCACCAATCCTAGGCATAT
AGATTGTAGGTTTAATCAGTTTATTTTCACCAAACTCATTTAATTCTAACATACAATTCATTAAAGCGGA
ATACTCAAAATTTGGCCCTGGTTGAAATTGAGTATAAAGATTGAAGCAGTAAGCTTTATGAGTCCTAAAG
TATTTTTCATAGACTGAGTAAGAACCGAGTTTAGTTACATCACCCCATTCAGTCTGTAATTTATCAGCTT
CCAAAATTTTAGGGAAAGCTTTGGTTAATTGACCCGCTACGCCTGAACCCATAGTATGAAAACAATTACA
TCCATGTGCAATATTTTTACCTTCAGCGAAAAGGGCGACAATATCGCCCTTGATATATTTTACAATCATC
TAGTACTCAATCCTCGATTATAAGAATCTACCAAACGGTCAACCATTGAATGACAAGCGGCTTTATCTTT
CTCCTCCGCAACTGAACATTCTAAGGTATTCCACTTTTTAGCATATCGTTTTAACAATGTATCGTTTTTG
TATCTGCTTGATTTATCTCTTTCTCCGTCTTTATATGCATATATTAATTTCTGTGCAAATTCAGCTTGGC
ATGCCTTATTTTTCCCACAATAATCTGCCGCAGTGCGGTTTACATATTCTCTAATTTCAGTATATGATGT
ATCTGCTGACGCAGAAGCAGAAAATGAAATTAATCCTATACATAAAACCAAAATTTTAGTCATTTACTAT
TTCCAAAAGTTTATTATTTTTAAGGTAATTAGCCTTTTCTAGGACTTCAGAAGCATATTTAGAACCTGCT
TTAACATTCCATCCCGAATTATAAGAGGATATTGCTTTTCTTATATCGCCCTTATGTATATTTAACCAAT
AAGAAAGTTCAATGTACGCCCAGGAAGCTGAATTGGATCGTTTATTCAACATTCTTTTTATTTCAGCATC
GGTCATATTATAACCAAGTTCCTTAACTCTTGCTCGCATAGTAGGCAAATAATTTTGGAACATTCCGTAG
GCGTGATGCTTTGGTTTAGATTTTAAATTAACTCCGCCAGAGCTTTCTTGCCATAAAATGGCAGCCATTA
TATGACCTAATCCGCTCTTGTGGATATTTTTGTGTGTTTTATATTTTCCATCCTTAGAAAATTGTTCCCC
GAATTGATACGCGTAACGCATGTTATCGAGTTGGACATTACTGAAAGTATGCTCGGAGCTATGTGCCATC
ATTGAAATGGCCAATAGACCAGCGAGTAGTGCTTTTCTCATGCTTACCTCATTGAGTTTTAATTACTGCT
TTAGAAGCCTTTCCTGGTAAACGACGACTGTTGATAATTGCCATCCTACATTGAAGTGACGGGTCTTTGA
ACTTCTCGTTAGGTTTACAAACTGTAAATCCAAGCCAAAGATTTCCATCTGTGATTTCTAAACGTCCAGG
ACGATATTCAACCCCATCAATAAAATCCTCGTCAATGTCAGGACGCGGAGGCATACTCAGGAATTCATTA
ACTTCTAAAACATGGTCTTTTATTTTATGGAATAATTCAAAAACGTATGTCTCATCAATCTCCCGTTGAA
TTGCGCGATCAAGAAGATGTTGAGAATATTTTAGATGAAACGATGAGACTCCTGCTGCTTTTGATGCCTC
ACGAATCTCATTGTTAATTTGACGAAACTCCGACTCAAAGTGACGACGAAGCTTATTTCGACGGATAAAA
ACTTCTGTATTGATAGTCATGTTATTCTCCTCTTAACTGATAGAAAAATTATACCACAGTCAAGAGGAAA
AGTAAACAGTTATTCTTTAAATCTAATCAATTTATTCATAGACTTTGAAACTTCGGCACGAACCTCATGT
AGATTTTTGAGCTGTTCAAGACGCTGCGTATAGTAAGCAATTTCATCTTCTTCGAGACAGTCCTGCGAAT
CTTCTTTAAGATAACGTGCATAGTCCTGGAAAGCGTTACGGACTACTTCCTGGAAGTCATCAAGACTTTG
AATTTTCTTAGGAGCAACAGATACACGACGAGGGGCAGTATAATACTCATAACCAAACCCTGCGCTTAAT
TGAGCCATTAGTATTTTTCCTCTGGTTGGAACACTGCACGACAAGCCCACATACTGGCTTCTTTGAGTTT
CGTTTTAGCAATAGTTAACTGATCGAGACTTTCAGCATAATTCTTCGCGAATTCACAGTCTTCGCAATTA
TCTAGTGCTTCCCAGAATTCATCATATAAAGCATCAAAGATAAGTCCTAAACGAACTTCAGCGTCTTTAA
TAGCATTTACTTTACCGATTTTCTCTTCAGTATGTGGTTTATAACCCTTAATATCTTCAATCATATTTGA
CTTCCTCACCAGTACATAATACGTATTCAACTAAACGAATAGGTTCATGAATGCCATAGCCTTGAACAGA
AATTTCTGTCGTAGGATAAATTCCATCAATATCACCCATATTCCACGCTTCATTAAATTGCTGTTCGCCT
GAGTTACTAAACCACTCGCGAAAGCATTTAGCACATCTTCAGAACCTTCAATAATTATCTTTGCCATTAC
AAACTTTCAGTAAAGGTACGAGCGATAACGTCGCGCTGCTGTTCCGGAGTCAGAGAGTTAAAGCGAACTG
CATAACCGGATACACGGATGGTCAGCTGCGGATATTTTTCCGGATGCTTAACTGCATCTTCCAGAGTTTC
ATGACGCAGAACGTTAACGTTCAGGTGTTGACCACCTTCAATTTTAACTGTAGGTTGTGGCTCAATTTCA
ATTTCACGGGCATGCAAACCATAGAAAATTTCTGGGTCTACAAAAGAGTCCTCTTTAAAGGTTTTAGAGA
CAATAATTCGTGCTTGAATACCATCTTCAAAATAAATAGTACCTTTATGTGTGCCTTCAAGAATTTGATA
TGCTTTCATATAAACCTCAATTAGAAAATAAATTTATCCAAGATTGTTCTTTAATTAAAAATGGCTCAGA
ATCATATGCCATTAAACTTTGCGTAATTAATCCTTTAAAAGGTCCATCAATAAATTCCATGGTAAAATAT
GGAATTTTATTCATTAGCCGTGCATTAGGAGCAGTGCACAAAACTCTGCATCCTTTGAATACGCCTTTTT
GTAATTTGTATTGCTTGGGATAAAATTCGCTCAAAATGTTATTTTTTGCCAAAATTTCAAAATGATTCAC
CAATTTATTTTTAATAGTTTTTGGCGAAAAATAAAGATATTCGAAAAGCTGAGTGTCTGTCATCATTGCA
TTCCGATTACGAAAAACTGTGGACGAGTAATACCACCAATGCAACATTTACTATTACAGCAGTAGTGTAC
GGTGTCAATATGGACACTATAAATCTTATCCATATCAGGAGATTTGACAGGCTCATCAATTATATACAAA
ATTCGCGAAAGCTTTAAACCTCTGAACTTGCTTCCTTTATTACCAATAAAACTGCGTACAGAATCAGTAA
ATAAACGAAAACGTATATCATCATTAGAATAACGCGAAAATTCCTTTTTGATGTTATTTGCAGAAATTTT
AGCATAAGCTGAAGTATTAGAAAGAACAATAACTGTTCCGCCATCATACAACCAATTAGCAGCAAAGTTA
GTCACAGCAATTGATTTACCGGATTGACGTCCACCATCTAGTCGAAGTGTACAATACTGTTTAAGTAAGT
CTTCAAATGGCGGGATATATTCGTTTTTACAAATTTCTTCTACTCTAGCATCAGAATGGTGTGTAAAAGC
ATTCATCAGGGATAGATAAGGACCAGTTAAAAATGTTCTCATTTCTTCTCTATAAGCTCTATAAGTTTGG
GCCATTCCGTGGCACATGAATTGTCCATTTCTGTATTTACCCATTACCGCGCTTGGGCTCGACCTTATTA
CAGGTTGGCGGGAATCCCTCATATAATCATGAGGTCCAGGTTGTTCCCTTATGCATAAATCGCCTTACCG
TAGTATTTGTACCAAGTAGGACGTTGTGCAATTTTTTCATCTAAACGAGCTTGTGATATAGCAATAGAAG
CTTCATGGGGAATATAATCACCACGGAATTCCTGAGGAATATCACTAATATCCTGGACTGTAGTATCCTT
GATATTAAAACCACGTTTTAAACATTCAGCTATAAGCTCAATTTGACGTTTACGTAAGAACTCGAGCTTA
TCGTAAAAGAATGTAACATGACCTGCGCCAAGGATAAAAGTAGGACTGATTTTAAAATCACGAACACGTT
TACCGTTAGCAACATGCTTACGAACTGCACCAAAAACACGCGGCAATTCACGATATTCAGCCATTAAGTG
TTGGTCAGCCAATTCAGATACTAAAGTAAGGTTGATACGAGTCATTTTAGTGTTCTCCTGTAGTTGATAG
GTCTATAGTATCATACCTACAGGAGATGTAAACTGTTATTTATCTTTAATTGCTTTAGCTGCTTCGATAG
CCGCTTGCTGAAGGTCATCCATAGACATGCCGAACTTAGAAGCAAAGTTATCAATTTTCTTTTCGACGGC
ATTCAAAGGCTTGGCCTGTTTACCTTCATTAGCGCCAGGAAGAGCGAGAATACGCTCTCTGTCAATATAA
AGGCTTACAAGTTTCTTACGGTCTTTATCGGGCAAATCATGAAATGAATGGGCCTTTTTATTTACAGCGG
CTTCTAATTTACCTGCGCCCACTCGCGCTTCGGCAATAAATTCTTGATATGTTTTCATATGTTTCCTTTA
AATGTAAATATTTTTATTATTCTATCCTAGAATTGTGATAATATATTCACAATTCTAGGAGTTGTAAACT
GCTTTTATTTAAGCGTCCCAAGTATAAGCTTTATTAAGAATTACCACGGGCTGCATTAGCAACGGCGTAA
GCGTACTGAATATTAGCGTCTTTAAACTTACCTTTAGAGGTATCTATTTCTGCCTTAAAGCCGCCTTTAG
TCATAACATCGGCAAATTCTTTACGGAAAGCCATTGCATCAAGACCTTTCCATGATGATTTATGCTTGGC
AAATTCAAGACCTGCGAAGTTGACAGCTTTAGCCAATTTATTATCAATGACCCATTTACCGGCTTTAGGC
ACAAACTTCGGGCCTTTCTGTTTAGAGAACAGTTTTAAATTCCAGCGGCGAAGGTCTGCATCAGCTTTAA
CAAATTCTGAGTCTAAGTTACTAGCAACAACATCTTCAATATGAGCAAATTTAAGTCCGTCAACTTCAAT
ATTTAAATCGGTACGCCAGCGAAGTCCTTCCCAAGCGAAGGCTTTAAAGTCGGAAGCCTTTGTAGCTAAG
TACCGTTCAATAGGAGCTGTTTTAGGGTCAAAGCCGTTTCCTGATCGGTAGGTCCACTCATCTTTGTTAA
TGCCTTTGGCCTTTACTACAGAAGCTTCGGCAATAAATTCTTGATATGTTTTCATATGTTTCCTTTAAAT
ATTTTAATTAGTAATTGTCTATTCAAGTAATTGTGAATATACTATCACAATTCCAAGAGAAAGTAAACAG
CTTTATAGATTTTTATACGCGTCCCAAGTGCCAGTTCTAAACGTTGTAATGACTCGTTTTGCGCGATTAG
GTGTTTGATTATACCATATACTTTTAGCTAAGTTAACTGCTGCTTCATCCCAGCGTTTTTGTTGAAGCAT
ACGTAAAGAGTTAGTAAATCCTGCCACACCGGTTTCTCCCATTTGGAAAACCATATTAATCAATGCACAG
CGACGAACCGCATCAAGAGAATCATAAACCGGTTTTAATTTAGCATTTCTCAGAATTCCGCGAACAGCAG
CATCAACATCCTGATTAAAGAGTTTTTCAGCCTCATCTTTTGTAATTACACCATTGCAATTACGCCCAAT
AGCTTTATCTAATTCAGATTTAGCAGCATTAAGTGATGGACTTTTTGTAAGCAAATGACCGATGCCAATA
GTGTAATAGCCTTCTGTGTCTTTATAGATTTTAAGTCTAAGACGTTCATCTATACGTAACATTTCAAATA
TATTCATAATACCTCCTAAGTATTTATAGAAGGTATTTATAAAATTAAAAGAGGTTGTTCATTATTCGGT
AAAGTGAAGGACCCATCACATATTGCCACTGAGTACGAGGAATAAGAGCAAAAGCGTCCATCTCTGGAAT
CATAACGCCATCTTTATTTTCAAAATAAGACTCGCAACGGCAATTTCTGAACATCTCATGCTCTACTGGA
ATCGTGTAATAAAATAACTGTAGGTCTTTATTACTAGAATATTTAAATACACCTAGGTCTTCTAGAAGGT
CTGGATTATAATTGCTAAAACCAGTCTCTTCTAAACATTCTCTTCGTGCTGCATCTAATGCGCTTAAATC
AGAATTTTCTACACGGCCCTTTGGAATATCCCAACGATGTGCCATCATTCCAGTCTTACGAGAACCAGTA
ACCCGACCCATAAATAAATCTTTATCTTCTGTCATAAAGATAATACCAGCTGATAATGTTTTCATTTTAA
TTTCCTGCATTCAGTGATAAAGTTATTTAAATTTTGAGCATATTTCTTTTCATCAAAAATCTTTTGCTGT
CTGCGTAACCGCCATGGCATTTCAATGAACATACGCCATATCCCTAGATAATACCGCTGCTGTAAAAATA
TTAACAAGTATAGTTAAAAGAATCCAATCGCCTATTCTGTCCATTGGATTTTTATAAAAAAGTAAAATAC
GAATGATGATATAGGAAGACTAATGATATACCACAGAAGAACCTTCTTATCTGTGAACCAATCAGCATTC
GTTAACTTAGCGCGACCATTTTGAATACACACGAATTTATCATCTGTTACAGTAAATGGCTTAGCTGCTT
GATATCCCATTCTAAACTCCCTAATTAATCGTTTCTTTGTATCTTCGGAACAACCATTCCAATCAACTCT
ATCAACTGGAATGCCATCATCCCCATCATCTAAATCATACCAGCGAGTTTTTAAAATCATTTAATTTTCC
TGCAATCAATCACAAACTCTTTCATTGATTCATTTTCAATATAAGACATGTAGCTATTATATTCTTTTAA
TTGTATTTTGTAATCCTTTTTTCTTTGCCAATTTATTTTAAAATTATCATAATGAAAATATAAAATGATA
CCAAAGAATGAAAACAATGAAATAATTTTAGTATAACAAAGCTCGCTCCAAACTTCTATTATATCTACTG
TACCACTGATTTTTAAAATAAAACAGTCAATTAATAGTCCAATAAGACTACCTGTAAGAGCTGCAGCCAA
CGCCACAGCAAAAATTAAAAATGACTCAGAAAACGAATATTTGACTTTATTTAGCTTTGGCTTTTGCATC
GTGATTCCTTAACAAATTTCATAATTTCATTAAATTCATACTCAGCAAGTTTAAGCTGGTGTTCCTTTTT
AATCTTTTTGCACTGGGCTTTCCAATCACGTACGCGTTTACGATAATGTCTTCCTTGATACCAGTATCCT
ATCCAATTTACGGGTACTAATAAAAATGGAACTACCAATGGAAGAGTTAGCATTAACATAATTATTACGC
CAGAATCAATATCAGTCATAACATCTAAAACACCTCCAATAATCAATAGAATTACAAATGATATAGCTAT
CACAGGACCTATTAATACATCAGTAGAAATTATCTGGCGCTTTAATTCATACTTCAAAGGTTTACTTGGA
AGGTATAGTGATGGCTTTGACATATTCTCTACATTCCTTAACAAATTTTTCTAGTAATAAATCACTTTCA
AAATTAGGATTTTCCACTAATTTATCAAAAAGATCATCAACAATATTCAAGATATTTCTTTTACTAAGAA
TACGTTTATTTTCATGCTTCGTTTCAGAATCAACTATAAGAGTAAAGAAATATTTCTTTCCCTGAAATTT
TACCGTAGTATCAATATAAAATAAATTTGACTTTTGTAAATTACGTTTAAACCATGCGTCACTTAAACTA
TAAACACCGAGATAATCAAAATCGTCGTTTAAATAACAAACTGACCATTCAGGAGAAATGAAATCAGTAA
ATTCAACATCAAAATCACATGTCAATGAATGAATTGATTCAATACTGTTAATAAGTATTCCAGGACGTAT
TAAAGACTTTTTACCTCTGGAAAATCTTCCAGAAAGACTTTCATCAGTTTCATATGAAGAACCCCAATAA
TAATTACGTCCTTTTGCCATATGTTTAAGAGCATTTAGTAATTGGTCTGGAACATCAACGTGTCTTTGGA
ACTCTTCAAACATTGAATTGAAATCACTTTGCATTTTCATTCCTATTTACTCCAAGTAATAGGGGCCGAA
GCCCCTTATCATTATTTCAGAGAATTAATATATTCCTGAACATCGGCAGAGGTAGTTTCAACCCCAGAAA
TATTACCATTAAAGGTTTCAACTCGAGCAAGAGTATCTTCAATATCAACCTTAGTCAGTGCTGCAATTTC
AACTACATCATCAGCAGTACTAATTCCAAGGGCATTTGCTGCACGAGTTTCACGGATATATTCCAATTTA
ACTGCAAGTTCTTGGCGAGCATCATCTAACTCAACTACTTTCTTGGCGATTTCAATTCGCATTTCAGCAT
AACCATCAGCTTTAGTAGTCAGCTGTTCAGCTGTTCGACGATATAGCAAGCCGAGTTTAGCATGCATTGT
TACATCTTGACCTTCGGAAAGAAGCTTGCGAATTTCACGCTCTTTTGATTCGGCCTGTTTATTCTTTTCA
ACAATAAGTTCACGAATACGTTTTTCTTCATTAATAGATTTAACAGAAGCAGTTTTTAGGTCTTTAATTT
TATCAAGTAGTTTTGCTGCTGCAGCAGTATACTGTTCTTCAACAGATAGATTTTTAGCCATTGCAGAACC
AAGTTTAGTGCGAATAAACTCAACAATTTTCTTCAGTGTGTTCATAGTATTTCCTTAGGTTGGTATAATT
AGATAATATAATATCACGTTTCTAATAGATTGTAAACTTATTCTTCGTCTAGCTCGTCGATAAAGGCGTT
GATGGCCTCGATAATGGCATCATTGATAGCCAATAAAATAAAATCATCGTCAGTACCTTTAGAAGATTCT
AAAGCATTGATATATGCTTGGTTGACGAGTTCCCAAGCCTTTTTAAAATAAGGAGCTTCATCATCAGGAC
AAATATCCCGCACGCCTTCAAAGATACGTTTGGCATAATCTAACACCCATTGTACAGGCATGTTTTGAGA
ACGTTCGTTAAACTCTTTAAAGTCCTTAGATTCAAAAAGCTCTTCAGGATAATTATTTCTATTACAAAAA
GCTTTACTAAAGTTACGTTTCATAATGTTTTCCTCATTTGTATAGGCTCATAATATCTCAATCATSAGCC
TATGTAAACTTATTTCATATTATTGAAATATTCTTCTGCGATTTCGTCGTTATCATGGTAAACTTTAGAA
GACAGTTTAACATAACTTTCAGCAGTGAACATGTTAATCACAACCTTTACAGTATACCACTGACCGTCTT
CATTACCCATTACTGCGTAAGTTTCAAACATCGGATGGTCAGGACCGATAACTTTAATATCATTCACCGT
ACGACCGAAATCTTCTGAAACACATTTCATAAAGAAGTTGAAAAGTTCACCGTAATTATCCATTTTATTC
TCCAAGTTATTTTCTGTATCAGTAGTTGATAGTTGTATAGTACCATGGAAGAACAAGGATGTAAACAGTT
TTGTGAAAAAATTTTTAAAAAGTTTTAGGGAATTCTAGGGCGGAGAGGGGCAATTAAAAGATAGGATAAT
ATATTATAAAGGGTATAAACTAAATGATGCCTAGAGAGGTCTGGAAAGGCTTAGATACCAAAAAGCCCCA
ACCTTTCGGTCGGGGCTAACCGTTGCGGCAACCTTGTCGGGGTTCCACCTGCCAAGGCAAGTGTTTGTAC
GAAACGCCGGGATTCGAACCCGGTTATTAAGTAGTTGACGCTACTCAATATTTTTAAAAGGCCATATCTC
AACCATATCCGAACGTTCCGTCAAAAACGCTACTCGGCTTACGGCAAAGATATTTCCTCGAATCGATAAT
TTGGTGCGCCGTTTCTGCTGTGATGTAAGAGGGCATCAATAAACGCAAAGATTATTAACGCAATTCCTTA
CTCAGGGAACCATCAGTCCGACGACTTACCGGTAGCGACCCGGTTTCTCATTTGGTATCCCGCCCTGGGA
TCGAACCAGGACCGCAAACTTAGAAGGATCGTATGCTATCCATTACACCAGCGGGACGTAATTTAAAATT
TCATTTTTCGACCTTTAAACCATCCTTCTGGAATTGGGTCAGTTTTCTTAATACGTTTAGAAACTTTTTC
ATCTAATGAATGAATCCACATCATACCGAATTGGGAATTCTTTTCACCTTTCTGGTGATTATTTTTGGCG
TGAGATTCTTTCATTTTATTAATAGTTTCAGGAGTATGATGCTTATTTAGAAATCTGCTATTATTTAAAA
ATTTTTCCCTGTATTCAGGAGTTGACCACAAACGTTTAAATACATTTGAACCAATTTTACGATATTTTTC
TTGAAGTAAAATATCATTTTCAAAACGTGACTTAAACGATTTAGCTCCTTTTAAGCTAGCATCTTTCTTC
TGGTTTAGCATTCCAGGAATATTTACATGATCCCATCCACCTTCACCGCCAAGTTTTAAATTATACACAT
CTGGTCTATTTAAAAACTCTTCTGTGACAATATTTTTCTCGGCTTCAAGCATAGATTCTTTATCGTCAAA
ATACTCTAATATTTCTTTAGAAAAATTTTCTATACCATATTTATCTTGGGCTCTTTTTAATAATTTACCA
GAACCCATATATCCATCATCTAAATTTTCGGTAGAATGCACACCAATATAAATTTTATTATTAATTTTAT
TTGTTATTTTATAAGTGTAATAGAACATAAATATCTCCTATTTCTAAGAGTATTTATGTTCTCAAAATAT
GACCCAGACCAGATTTGAACTGGTAACCTTTCCCTTATGAGGGGACTGCTGCTAACCATTGAGCTACAGG
GCCTTGGTGCTGATTGACGGAATCGAACCGCCGACATCCTCATTACAAGTGAGGTGCTCTACCTACTGAG
CTAAATCAGCAAAATTACGGAGGCGATAGGATTTGAACCTATGAGTCGCCGGAGCGACTGCCGGTTTTCA
AGACCGGTGCATTAAACCACTCTGCCACGCCTCCAGTCTCCATACAAGGATTTGAACCTTGGACCTCCTG
ATCCCAAATCAGGCGCTCTACCAAACTGAGCTACACGGAGTAAATTAAATTGGAGCGGATAATGAGAATC
GAACTCACATCATCAGATTGGAAGTCTGAGGTAATACCATTATACGATATCCGCAAATTTGGTGCGAGAA
GTGGGACTCGAACCCACAAGGAAATCATTCCGCAGCATTTTAAGTGCTGTGCCTTTACCAATTTGACCAT
TCTCGCGCTGGGAATAAAGGACTCGAACCTTTGCATCTAGCAGTCAAAGTGCTATGCCTTACCAACTTGG
CTAATTCCCAATTATTAACAAAGGCTCTCTAACAAGAACCCTTGATGATAGAGGGTATTAATCAGTGCGG
TATGAGTTAATAATAACAAATAATTCTTAAAGCATATTTACCATTTATGATGATACGTATTTACGATACA
TTCAAGACCCAAAGGATTCTTGAAAATATCATATTCAAGAGGACCTTTTTCTGTTTCAATAAAGAAATCA
AAATTTACTGTATTAAATTTACGGTCTTCCTTTACTAATTTAACTTGAGAAGATGAACGATCAATGTAAA
CCTTTTCAACTTCAAAACACGTTAAAATGCCATAATCATCAATCAAGGCTTTAGCTGCTTCTTGATCATA
TTTATATCCATTTTCAACGGATGATACTTTCGCATAAAGAATCATCATCAACCTCTATCAACAATAGCAT
GAGTATGGGCATTTACGATTTGCCACCAGTCGAAACGATTGGAACCATAATCTGGTTTATTTTCATTTTC
TTTAATGATATCACGCAGTTTATCTTCTGTTTCAGCATACGCAATTAAATCATCATATCCACCACAAGGA
TAATAATTATCACCTGCGAATAAAAGAAAATTTACCTTAGATGGATTTACGTAATAATGGTCTTTAGGAT
ATTTAGTTCCTCTCCAATCAGTTACTTCAACATAACGGTAAGAAAATCCATTTTTACTTTCAATCCAACT
CCACGCTTCAAAAGGAGTATTAAAAACTTTATCAGGTATTAAATTACCTTCAAAATGAGAAGGATTTGCA
TAATCCCCGGCATAAACATAATATTCGTTAATACTCATTTATTCACCTTTAGAAATTTTATCCATAACGA
TAGCAATTAAACCAATTAAAAATGCTACTACAAGTGAAAACACATTTTCTGCTGTAGTTAATAATCCGCA
TATAAATCCAACAAACATTGAAAAACTAAAAGCAGAAGCAGAAATTGCAATAGCAACATTTCGAATTAAT
TCACAGCGTTTCATTTTATTCTCCTCAGTAGTTGATAGGGTAATAGTATCACAGCTAAAACCCTATGTAA
ACAACTTTGTGAAATATTTATTACAAAAGATTTTTAGCAATAATCTTGAGATGTGCCGCAGAAATGTGTT
TAGCTTTAAACAACGCAGTTTCTTCAGCAGGAGAGATAACGATTGTAGCACCATCCTTTTTAGCAGACCA
CCCATCACCTAGGTAAACAGTACCTTTGATTTCTTCGCCATCAACCAGACTAATCATTGGTTTACCTTCT
CGTCCTTTATTTGCTTTAATAACTTCAGAAGTAAGAGTAGCTTCGGTAATGGTAGAAACCGGGGTAGTTG
TAGAAGTAAATTCTTTAAATGTTTTCATTTTTATTTTCCTAATTAATTTTGATGAGGTAATAGTATCACT
ACCTCATCAGTATGTAAACAACTTTGTGAAATTATTTTAAATCATCTGCCCAATCGAGTTTAAGAGGCTC
TTTGTATTCACGATCTAATACGACCGGAATTTGTACATCACCGCTAAATGATAAGGGCCCAACATTATAA
GACAATGTTATATGCGGTGTGTAATCATCAAAATCATGTGTAGCACCTAGTGCCCGCGCATACATGTGTC
GACAGCGCAGATATTCAGAATCTAGCACAAGTACAAGAGTCGATCCATCTTGTGTTTTCCATACTTCTAA
ATGTCCAGAAGAAGCTACTTCAAAACTTCCACTCGATGGAACATATGGAACATTTACTCTTGAATAACAT
ATAGTCGAATGAATTTTTTCTCTAGGAACTGGATTAGGAACACGTAAAGAGCGCTGAAGTTCTTCCAGCG
CATCAAGTGTTAATTCTGAAAACTTAGCTGCTACATAAAGACCCGTTGAAAAGTCTTTAAATTCCATCAT
TCTTCATCTTTTGCTTCATCTGCAGATTCAGCAGTAAGATTTTTGACAGCTTCAACGATTTCTTCAACTT
TGATAGTATCGCCAGTGATACCTACTGCACGAGCAATTTCAGCCAAAGTTCCTTGCAGAATTTTGGATTC
TTCCATCAGACGAGCAGCTTGATCCTGCGTATCAAGAATGCGAGATTTCAGAGTTACGATTTCAGCAGAC
AGTTTTTGTTCAACAGTTTGTTCAGACATTATAGTACCTTTAGTGTATTTTTAATTTTAGAAAAAAGTTC
TTCAAGAGAACCATCGTTTGTAATTACTAAATCGCCATCACGAATTGGCAATCCAGCTTCTGTAATATGT
GTATCATTGGATTTTTGACCAGGACGAACTACATGAATTACTGTAGCACCCATCGCCCTAGCCGCATCCA
TTTCATGATCTTGACGGGTATCAGGAACGATATAATAATCATAACCTGAGTTAAATTTATCAAGATAATC
TAAAGCAAATAATTTTACCCAGTACATGCGGTCGAAGTTATTAACAATCAAATCCGTACCTAGGGCTTGC
ATCAGACGACGGACTGACCATTGATCTTCAATATTATTTATAACGTCAGTAATCTTATTAAATGCTACGA
AATTAACTGATTCTTTTCCTTCGTCATCAAAAACAAACACACCTTTAATTGGGCTTTTACCATTAAGATA
ACAAAATGCTTGTTCCATAATCGTGATTACTTCTAATTTAGTCAGATTTAAATTAGTCTCACGATCATAG
TCAATTCCTTCAAACTCTTTACGAGTTAAGCAAGGATAGTCAGTGTTTGCTGCAAATACTCCCCATGCAT
AAGCCAATGCATCCTTAATAGGACCAGCAAGTTGGTATTTAACTGCAGAATAATTGCTCATGATAAAATC
AGCAGTAGTATCTTTTCCACTACGCTTTACACCGCTTAAAAAGATTAGTTTCATGTGTTTCTCCTCAAAT
TTAATTAAGATTATAACACACAAAACTGAAGCATTAAACTTCTGCTATAATTTTACCATCTTTTTCTACT
TGAAAATAGGTGTAAGGAATTGTTGCAGTACATACTAAAGCCGGGTCTGAATCTTCCGTGTAGCTAAATT
CTACTTCAGATAGGTCAGAAACCCAAGGCTTATAAAAATTTATTGACATCACGATTTCAGTTTTGCTATT
ATCTAAGATGTAAAGCGTAATGTACTCAGGACCTGTTTTTTGGGCAGTATTTTCACCTGTAAGATAGTTG
CTAGTTCCTAGCATCCATTCATACATTCCTATCCACGACTTAAGTTCTTCATCAACTATAAATCTCACAA
TGAGTGGATCATACTCAAATGTAACACCTGGACGTTGTGCTCGGCCCAGTCCAAACGGCCCAGTCACGGT
ATCAGTAACAGGTATTCTAATTCCTGGAATAGGAACTGACTGAGCATTTAAAGTAAAAGCAGATGTAGTA
TTACTATGTGGTATTGATACTACAAAGTTAGTTGTATTTGCTTGGTTAAAAATTTGTTGCAGAGCTTGCG
ACATATATTCCTCATAATGCTTTATAACTGTTGGTGGTATAATGGGTCTAAGTCCCTTCCATTCAATTCC
ATTTAGAACAAACAACAGAAAAGAATGGAAGATAATAGAATTAGATATTTGACCAGACTTTGTTTGCAGA
GAAACGTTTTCCTTTTGAAACGAACTGCTGAAGTGGCATCAACACAACGTTCGCCCAGTCTTTCGGGGCG
ATTTCAACAAGGCTACCCATAATATTACCAGGTATATATGCCTTAATCATTTGGTCTGCACCCCTAAATC
CTTTCACTTGACTCCAATCAATTTTTAATTTCGTTTTATTAGTAATAGTAGGTGTATTTGCATATTGCTT
TAAAAGCTCTTCTAGGAATTGCTGACGAGCTTTAGGTGGAATATAGTGCAAGTTTAATCCGTACATTAAA
TTATGCTTACCTAAACCAAGGTAAATTATCAAAGGAAATTTATCCCAGTAAGGAAGAGTTTCCTTGTGTT
TAGCATCATAAGCAAAAGCATATATTCGTCCCGGCTGCGGGCGAACAACTTTATGTCCTTTTACTTGCTT
AATAGTTTCAGCAAACCACTTTCTGGTTTTATTATTAATTGCTGCGCCTTCATTACGAATTTTATCACGC
AATGTTTGTCTGAATGAATTTATCATAAGCAGTTGTCTTTCTTGCTTATTGAGTTTATTCATTGGTTTTG
ATTCAAGTTTTTGAATCTTTTCAGCCGTTTTAATTCCTGAAGCATATTTTGACATTGCTGAAGTAAACGT
AGAGTATTTGATTCCTCTTTCTTCAGCAAATTGCTTTCCTGTCATTCCTTTTGCTTTGGCCTTTTTATAT
TCAAGACCTATCTGAATCCATTTCTTTTCGTTTAATGATTGCTTAACCTTTGGAACTTGGGGAGTGCTTT
CATTAATTATTTGAAAAATAGCCATTATGCCCCCTTAAAGCCAAGAGCTCGTAATCCATCTTCTGTTAGA
ATTCTAAATTTTATTCCACGCTTTTCAGCTAAAGATTGTGCTGCTTTCCATTTGTCAGTGTTCACAGACC
AGGTATAAATTTCATTCATAAATCTTTTCTTCGCTGCGGTCGTTAGATGTGCTGGTTTAACTGGTGGTTG
TGTTTCTTTTTTAGGTTTTATTTCAATAAAAAATTCTTGTCCAGAAGAATCTTTCATCCAAATATCCATG
AAGTATCTACGTTTTTTCCCTTCTGCATTACAAAAATAAGGAATTACTGCTGTTTCACTACCCCATGCAA
TAATTTCTGGATTTTTATCTAACCATTCAAAAAAGAATTTTTCCCAATTTGATCTATACGTAATTTTTTT
AGGGTCACCTCTATACTTTGATATATTTTTAGGAACCCATTTTCCAGAATATGCCATTGGATTCTCCTTA
TAAATAGATAATATATTTATAAACAGGAGGGCCCATGCTCTTTACATTTTTTGATCCGATTGAATATGCG
GCCAAAACGGTGAATAAAAACGCGCCGACTATTCCTATGACAGATATTTTTAGAAACTATAAAGACTATT
TTAAACGCGCTCTTGCGGGATACCGCTTACGTACTTATTATATTAAAGGTTCACCACGCCCGGAAGAATT
AGCAAATGCTATATATGGAAATCCACAGCTGTATTGGGTTTTATTGATGTGTAATGATAATTATGACCCG
TATTATGGATGGATTACTTCGCAAGAAGCTGCTTATCAAGCATCTATACAAAAATACAAAAACGTAGGTG
GAGACCAAATAGTATATCATGTGAATGAGAACGGTGAAAAATTTTATAATTTAATATCATACGATGATAA
TCCATATGTTTGGTATGATAAAGGCGATAAAGCTAGAAAATATCCTCAATATGAAGGAGCGCTTGCTGCG
GTCGATACGTATGAAGCTGCTGTTCTTGAAAATGAAAAACTTCGTCAAATAAAAATAATAGCAAAATCAG
ACATCAATTCATTTATGAACGACCTTATACGTATAATGGAGAAATCTTATGGAAATGATAAGTAATAACC
TTAATTGGTTTGTCGGTGTTGTTGAAGATAGAATGGACCCATTAAAATTAGGTCGTGTTCGTGTTCGTGT
GGTTGGTCTGCATCCACCTCAAAGAGCACAAGGTGATGTAATGGGTATTCCAACTGAAAAATTACCATGG
ATGTCAGTTATTCAACCTATAACTTCTGCAGCAATGTCTGGAATTGGAGGTTCTGTTACTGGACCAGTAG
AAGGAACTAGAGTTTATGGTCATTTTTTAGACAAATGGAAAACTAATGGAATTGTCCTTGGCACGTATGG
TGGAATAGTTCGCGAAAAACCGAATAGACTTGAAGGATTTTCTGACCCAACTGGGCAGTATCCTAGACGT
TTAGGAAATGATACTAACGTACTAAACCAAGGTGGAGAAGTAGGATATGATTCGTCTTCTAACGTTATCC
AAGATAGTAACTTAGACACCGCAATAAATCCCGATGATAGACCGCTATCAGAGATTCCGACCGATGATAA
TCCAAATATGTCAATGGCTGAAATGCTTCGCCGTGATGAAGGATTAAGATTAAAAGTTTATTGGGATACC
GAAGGATATCCGACAATTGGTATTGGTCATCTTATCATGAAGCAGCCAGTTCGTGATATGGCTCAAATTA
ATAAAGTTTTATCAAAACAAGTTGGTCGTGAAATTACAGGAAATCCAGGTTCTATTACAATGGAAGAGGC
GACGACTTTATTTGAGCGTGATTTGGCTGATATGCAACGGGACATTAAATCACATTCTAAAGTAGGACCA
GTCTGGCAAGCTGTCAACCGTTCTCGTCAAATGGCGTTAGAAAATATGGCATTTCAGATGGGTGTTGGTG
GTGTAGCTAAATTTAACACAATGTTAACTGCTATGTTAGCAGGAGATTGGGAAAAAGCGTATAAAGCCGG
TCGTGATTCATTGTGGTATCAACAAACAAAAGGCCGTGCATCCCGTGTTACCATGATTATTCTTACGGGG
AATTTGGAATCATATGGTGTTGAAGTGAAAACCCCAGCTAGGTCTCTATCAGCAATGGCTGCTACTGTAG
CTAAATCTTCTGACCCTGCTGACCCTCCTATTCCAAATGACTCGAGAATTTTATTCAAAGAACCAGTTTC
TTCATATAAAGGTGAATATCCTTATGTGCATACAATGGAAACTGAAAGCGGACATATTCAGGAATTTGAT
GATACCCCTGGGCAAGAACGATATAGATTAGTTCATCCAACTGGAACTTATGAAGAAGTATCACCATCAG
GAAGAAGAACAAGAAAAACTGTTGATAATTTGTATGATATAACCAATGCTGATGGTAATTTTTTGGTAGC
CGGTGATAAAAAGACTAACGTCGGTGGTTCAGAAATTTATTATAACATGGATAATCGTTTACATCAAATC
GATGGAAGCAATACAATATTTGTACGTGGAGACGAAACGAAAACTGTTGAAGGTAATGGAACTATCCTAG
TTAAAGGTAATGTTACTATTATAGTTGAAGGTAATGCTGACATTACAGTTAAAGGAGATGCTACCACTTT
AGTTGAAGGAAATCAAACTAACACAGTAAATGGAAATCTTTCTTGGAAAGTTGCCGGGACAGTTGATTGG
GATGTCGGTGGTGATTGGACAGAAAAAATGGCATCTATGAGTTCTATTTCATCTGGTCAATACACAATTG
ATGGATCGAGGATTGACATTGGCTAATATACTTCCAATGAGCGCTGATTTAGGAGAATCCATGGAAGGTT
CTTCTATCGACGTCACCTTTACCGCTCAATTAGAAACAGGTGAAACGTTAGTATCTATAAATATAACTAG
TTACGAAGAAACTCCTGGGGTTTTAGTAGAAGAAAATCGCTTATATGGAACATATGAATCTGTATTTGGT
TTCGGAAATGACGCGTTGAAATATCGTTTAGGCGATGAATTTAAAACTGCTGCTTCATGGGAAGAACTTC
CTACTGATTCTGATACTCAGTTGTATTTGTGGAAAGCTCCTCAAAACCTCCAGAAGACATTCACTTACGA
AGTAACATTAATATATGACTACCAAGAACAAAGTGAATCTGGGGGTTCTGGCAGTAATTCTAGGTCATCT
TCTGATACTACTGAACCGACAGATCCTCCTGCTCCAGTAAGAAAAACTCTAGTTAAAAATTATACTAAAA
CTATAGTTGGAAATTGGAGTCGTTGGGCTAATAAACTGAGAAAATATGCCTATGCAAGACCATAAATATT
TTTATTTGTATTCAATAACTAATAAAACAACAGAAAAAATTTATGTAGGCGTCCACAAAACTTCAAATTT
GGATGATGGGTATATGGGTTCTGGCGTTGCCATTAAAAATGCCATTAAAAAATATGGCATAGATAATTTT
TATAAGCATATTATAAAATTCTTTGAATCTGAAAAAGCTATGTATGACGCAGAGGCAGAAATAGTCACAG
AGGAATTTGTTAAATCTAAGAAAACTTATAATATGAAACTAGGCGGTATCGGTGGCTTCCCAAAACATAA
CACAGCGGGTGCTAAAAATGGATTTTACGGTAAATCTCATTCGCGTGAAACTAGATTGAAAATTAGCATT
AAATCGTCTAGAAAAAGAGGGCCTAGAGGGCTAGAGGTAAAACTCTGAAGATGTGTGGCGCCAATAACCC
AAGGTATGGCAAAATAGCCCCTAATGCTAAATCTGTTATTATCAACGGCGTTTTATATAAAAGTATTAAA
ATCGCAGCTAAAGCTCTTAATATAAATTATAGTACCTTAAAGGGGCGAGTTAAAGCGGGGTATTATAAAT
GTCAGGATTAAGTTATGATAAGTGTGTTACTGCTGGCCATGAAGCGTGGCCTCCAACAGTTGTGAATGCT
ACACAAAGTAAAGTATTCACTGGAGGAATTGCTGTTCTCGTAGCAGGCGATCCAATTACAGAACATACAG
AAATTAAAAAGCCGTATGAAACACATGGCGGAGTGACACAACCTAGAACTTCTAAGGTATATGTCACTGG
AAAGAAAGCTGTTCAAATGGCTGATCCAATATCATGCGGTGATACTGTGGCTCAGGCATCATCTAAAGTA
TTCATTAAATAGGATTTAAAATGGCAAATACCCCTGTAAATTATCAATTAACAAGAACAGCAAATGCTAT
TCCCGAGATATTCGTCGGGGGTACATTTGCTGAAATAAAACAAAACCTCATTGAATGGCTTAATGGCCAA
AATGAATTTTTGGATTATGATTTTGAAGGCTCAAGATTAAACGTTCTGTGTGACCTTTTAGCTTATAATA
CATTATACATTCAGCAGTTTGGTAATGCTGCTGTGTATGAAAGCTTTATGCGTACTGCTAACTTACGAAG
TTCAGTTGTTCAAGCTGCACAAGATAACGGATATTTACCTACTTCAAAATCCGCTGCGCAGACCGAAATT
ATGTTAACATGCACTGACGCATTGAATAGGAATTACATTACTATTCCTCGCGGAACTCGCTTTTTAGCAT
ATGCAAAAGATACTTCTGTTAATCCATATAACTTCGTTTCTAGGGAAGACGTTATTGCTATTCGTGATAA
AAATAACCAATATTTTCCGCGTTTAAAATTGGCCCAGGGACGTATAGTAAGAACTGAAATCATTTATGAT
AAATTAACACCTATTATCATTTATGATAAAAATATTGATAGAAACCAGGTTAAATTATACGTTGATGGAG
CGGAATGGATTAACTGGACGAGAAAGTCAATGGTTCATGCTGGTTCAACATCAACGATTTACTATATGCG
TGAAACTATTGATGGAAACACTGAATTCTATTTTGGTGAAGGTGAAATTTCTGTTAATGCTTCTGAAGGA
GCTTTGACCGCTAATTATATCGGAGGTCTTAAACCTACTCAGAACTCTACGATTGTTATTGAGTACATTA
GTACTAATGGTGCTGACGCGAACGGAGCAGTCGGATTTTCATACGCAGATACATTAACAAATATAACTGT
CATCAATATTAATGAAAATCCAAACGATGATCCAGATTTTGTTGGGGCAGATGGAGGCGGTGATCCAGAA
GATATTGAGCGTATTCGCGAATTGGGTACTATTAAACGCGAAACCCAACAACGATGCGTAACTGCGACTG
ACTATGATACATTCGTTTCAGAGAGATTTGGTTCTATTATTCAAGCTGTTCAGACTTTCACTGATTCTAC
TAAACCTGGGTATGCATTTATTGCTGCTAAACCTAAATCAGGATTGTATTTAACTACCGTACAGCGTGAA
GATATTAAAAATTATCTCAAAGACTATAATTTAGCTCCTATTACGCCATCAATTATTTCTCCTAATTATC
TTTTTATTAAGACTAATTTAAAAGTCACATATGCTTTAAATAAACTGCAAGAATCCGAACAGTGGCTTGA
AGGTCAAATAATTGATAAAATAGATCGCTATTATACCGAAGATGTAGAAATTTTTAACTCGTCTTTCGCT
AAATCTAAGATGTTGACATATGTAGATGATGCAGATCATTCTGTCATTGGTTCATCAGCGACTATTCAAA
TGGTTCGTGAAGTACAAAACTTCTATAAAACGCCTGAAGCGGGTATTAAATACAATAATCAAATAAAAGA
TCGTTCTATGGAATCTAATACGTTTTCATTTAATTCTGGACGAAAGGTTGTAAATCCTGATACTGGTTTA
GAAGAAGATGTATTATATGACGTTCGTATAGTATCAACAGACCGAGATTCTAAAGGAATTGGTAAAGTTA
TTATTGGTCCATTTGCTTCTGGCGATGTTACAGAAAATGAAAACATTCAGCCGTATACAGGCAACGATTT
TAACAAATTAGCAAATTCTGATGGACGCGACAAATACTATGTTATCGGTGAAATAAATTATCCAGCTGAT
GTGATTTATTGGAATATCGCTAAAATTAATTTAACATCTGAAAAATTTGAAGTTCAGACCATTGAATTAT
ATTCTGACCCAACCGATGATGTTATCTTTACTCGCGATGGTTCACTGATTGTATTTGAAAATGACTTACG
TCCACAATACTTAACTATCGATTTGGAGCCTATATCACAATGACAGTAAAAGCACCTTCAGTCACTAGTC
TCAGAATTTCCAAGTTATCCGCAAATCAGGTGCAAGTACGCTGGGATGACGTTGGTGCTAATTTCTACTA
TTTTGTAGAAATCGCTGAGACAAAAACAAACTCGGGGGAAAATCTCCCGAGTAATCAATATCGTTGGATT
AATTTAGGATATACAGCAAATAATAGTTTCTTTTTTGATGATGCTGATCCATTAACAACATACATTATTA
GAGTAGCCACAGCTGCGCAAGATTTTGAGCAGTCTGATTGGATTTATACCGAAGAGTTTGAAACTTTTGC
TACAAATGCTTATACATTTCAAAACATGATTGAAATGCAATTAGCCAATAAATTCATTCAGGAAAAATTT
ACTCTTAATAATTCTGATTATGTTAATTTTAATAATGATACTATAATGGCTGCATTGATGAATGAATCAT
TCCAATTCAGCCCATCGTATGTTGATGTTTCATCAATAAGTAATTTTATTATTGGTGAAAATGAGTATCA
TGAAATACAAGGTTCTATTCAGCAAGTATGTAAGGATATTAACCGAGTTTATTTGATGGAATCAGAAGGA
ATTCTATATCTTTTTGAGCGCTATCAACCTGTAGTTAAAGTATCCAATGATAAAGGACAAACCTGGAAAG
CTGTAAAGCTCTTCAATGACCGTGTAGGATATCCTTTATCTAAGACAGTATATTACCAATCTGCGAACAC
AACATACGTTCTAGGATACGACAAGATTTTCTATGGCCGCAAATCTACTGATGTTAGATGGTCAGCCGAT
GATGTCAGATTTAGTTCTCAGGATATAACATTTGCTAAACTTGGCGACCAATTACATCTAGGATTTGATG
TAGAAATTTTTGCCACTTACGCGACTTTACCAGCGAATGTATACCGCATTGCAGAAGCTATTACTTGCAC
CGATGATTACATTTACGTTGTCGCCAGAGACAAAGTTAGATACATAAAAACGAGTAATGCACTTATAGAT
TTTGATCCATTATCTCCAACATATTCGGAAAGACTTTTTGAACCTGATACCATGACTATAACCGGAAATC
CTAAAGCAGTATGCTATAAAATGGATTCTATCTGTGATAAAGTTTTTGCTCTTATTATTGGTGAAGTTGA
AACATTAAATGCTAATCCTAGAACATCAAAAATAATTGATTCCGCTGATAAAGGAATATATGTTTTAAAT
CATGACGAAAAAACATGGAAAAGAGTTTTTGGTAATACCGAAGAAGAAAGAAGACGTATTCAACCCGGAT
ATGCGAATATGTCAACTGACGGTAAATTAGTTTCTCTGTCTTCGAGTAATTTTAAATTTTTAAGTGATAA
TGTTGTTAATGACCCTGAAACTGCAGCAAAATATCAGTTAATTGGCGCTGTTAAATATGAATTTCCTCGT
GAATGGTTAGCTGATAAGCATTATCATATGATGGCATTTATAGCGGATGAAACATCTGATTGGGAGACTT
TTACTCCTCAACCAATGAAATACTACGCAGAACCATTCTTTAACTGGTCTAAAAAATCTAACACACGTTG
TTGGATAAACAACTCTGATAGAGCTGTGGTAGTTTATGCTGATTTAAAATACACTAAAGTTATAGAAAAT
ATTCCGGAAACATCACCAGATAGATTAGTTCATGAATACTGGGATGATGGTGATTGCACTATAGTAATGC
CAAATGTCAAATTCACTGGATTTAAAAAATACGCATCAGGAATGCTTTTCTATAAAGCCTCCGGTGAAAT
AATTTCTTACTATGATTTTAACTATCGTGTGAGAGATACAGTAGAAATTATTTGGAAGCCAACTGAAGTA
TTTTTAAAAGCATTTTTACAAAACCAAGAGCATGAGACTCCTTGGTCACCAGAAGAAGAGCGTGGATTAG
CTGACCCTGATTTAAGACCATTAATTGGCACAATGATGCCTGATTCTTATTTGTTACAGGATTCGAATTT
TGAGGCATTTTGCGAAGCATATATTCAGTATCTTTCTGATGGATATGGAACTCAATACAATAATTTACGA
AATTTAATTCGTAACCAATATCCACGAGAAGAGCACGCATGGGAATATTTGTGGTCAGAGATATATAAAA
GAAACATTTATTTAAATGCTGATAAACGCGATGCTGTTGCGAGATTCTTTGAATCACGTAGCTATGATTT
TTATTCTACTAAAGGAATTGAAGCATCATACAAGTTTCTTTTTAAAGTTCTTTATAATGAAGAAGTTGAA
ATTGAAATTGAATCTGGGGCTGGTACTGAATATGATATAATCGTTCAATCTGATTCTTTGACTGAAGATT
TAGTAGGACAAACGATTTATACGGCAACAGGAAGATGTAATGTTACTTATATAGAAAGAAGCTATTCTAA
TGGTAAATTGCAATGGACCGTAACTATTCATAATCTTTTGGGACGATTAATTGCTGGTCAAGAAGTTAAA
GCAGAAAGACTCCCTAGTTTTGAAGGCGAAATTATTCGTGGGGTTAAAGGAAAGGATTTGCTTCAAAACA
ATATAGACTATATTAATAGAAGTAGATCATACTATGTAATGAAAATTAAATCCAATTTACCTTCTTCCCG
CTGGAAATCTGACGTTATTCGTTTTGTTCATCCAGTAGGATTTGGATTTATAGCAATTACCCTTTTAACA
ATGTTTATTAATGTTGGTTTAACTCTTAAACATACAGAGACTATAATTAATAAATACAAAAACTATAAAT
GGGATTCTGGATTGCCTACTGAATATGCCGACAGAATAGCTAAATTAACTCCAACCGGTGAAATTGAGCA
TGATTCAGTAACAGGCGAAGCAATTTATGAGCCTGGCCCAATGGCTGGTGTAAAATATCCTCTTCCTGAT
GACTATAATGCTGAAAATAATAATTCAATATTTCAAGGTCAATTGCCGTCTGAACGACGTAAATTAATGA
GTCCTTTATTTGATGCATCTGGAACAACATTTGCGCAATTTAGAGATTTAGTTAATAAACGTCTAAAAGA
TAATATAGGAAATCCAAGAGACCCTGAAAATCCAACACAGGTTAAAATAGATGAATGATTCAAGTGTTAT
CTATCGTGCGATAGTTACTTCAAAATTTAGAACAGAAAAAATGTTGAATTTTTATAATTCAATTGGAAGT
GGTCCGGATAAAAACACTATCTTTATCACATTTGGAAGATCAGAACCGTGGTCATCAAATGAAAATGAGG
TGGGCTTTGCCCCACCTTATCCAACCGATTCTGTATTAGGCGTAACTGACATGTGGACGCATATGATGGG
AACAGTAAAAGTTCTTCCATCAATGCTTGATGCTGTTATTCCTCGCAGAGATTGGGGAGATACTAGATAT
CCGGATCCATACACATTTAGAATTAACGATATTGTAGTGTGTAACTCAGCTCCTTACAACGCTACTGAAT
CAGGCGCTGGTTGGTTAGTGTATCGTTGTTTAGATGTTCCTGATACCGGAATGTGTTCAATAGCATCTTT
AACTGATAAAGATGAATGCCTTAAGTTAGGTGGAAAATGGACTCCTTCTGCTAGGTCAATGACTCCGCCT
GAAGGTCGAGGAGATGCTGAAGGAACAATTGAACCCGGAGACGGGTATGTGTGGGAATATCTATTTGAGA
TTCCGCCTGATGTATCTATAAATAGATGCACGAATGAATATATCGTGGTTCCTTGGCCTGAGGAATTAAA
AGAAGACCCGACTAGATGGGGATATGAAGATAATCTCACTTGGCAACAAGATGATTTTGGATTAATTTAC
CGTGTTAAGGCAAATACTATCCGTTTTAAAGCATATTTAGATTCAGTTTATTTTCCTGAAGCTGCATTAC
CAGGAAATAAAGGATTTAGACAAATATCAATAATCACGAATCCTCTTGAAGCTAAAGCTCATCCAAATGA
CCCAAACGTTAAAGCTGAAAAGGATTATTATGACCCAGAAGATTTAATGAGGCATTCGGGTGAAATGATT
TATATGGAAAATAGGCCACCTATTATTATGGCAATGGATCAAACAGAAGAAATCAATATTCTGTTTACAT
TTTAAATTAAGGGAGCCCATGGGCTCCCTTTTTCTTTATAAATACTATAAACTCATAAGGAAACCGCTAT
GTTCATTCAAGAACCAAAGAAATTGATTGATACCGGCGAAATTGGTAACGCTTCTACTGGTGATATCTTA
TTCGACGGTGGTAATAAAATTAATAGTGATTTTAACGCAATTTATAATGCGTTTGGCGATCAGCGTAAAA
TGGCAGTAGCAAATGGCACTGGAGCAGATGGTCAAATTATCCATGCTACTGGATATTATCAAAAACACTC
TATTACAGAGTACGCAACTCCAGTGAAAGTTGGCACTAGACATGATATTGATACCTCTACTGTAGGTGTT
AAAGTTATCATTGAAAGAGGCGAACTCGGCGATTGTGTTGAATTCATTAACTCTAATGGATCAATATCAG
TTACTAATCCTTTGACAATTCAAGCTATTGATTCAATTAAAGGTGTTTCAGGTAATTTAGTAGTAACTAG
CCCATATAGTAAAGTTACTTTACGCTGTATTTCATCTGATAATTCTACGTCGGTTTGGAATTATTCTATT
GAAAGTATGTTTGGACAAAAGGAATCACCAGCTGAAGGTACATGGAATATTTCTACATCTGGATCAGTTG
ACATTCCATTATTTCATCGTACTGAATACAATATGGCTAAATTGCTAGTTACGTGCCAATCGGTAGATGG
AAGAAAAATTAAAACAGCAGAAATAAATATTCTTGTGGATACTGTTAATTCAGAGGTAATTTCTTCTGAA
TATGCTGTCATGCGAGTTGGGAATGAAACCGAAGAAGACGAAATCGCTAATATTGCATTTAGTATTAAAG
AAAATTATGTAACGGCGACTATAAGTTCTTCAACTGTCGGTATGAGAGCAGCAGTTAAAGTTATCGCTAC
GCAGAAAATCGGGGTGGCTCAATAATGAAACAAAATATTAATATCGGTAATGTTGTAGATGATGGTACCG
GTGACTACCTGCGTAAAGGTGGTATAAAAATAAATGAAAACTTTGATGAGCTTTATTATGAACTCGGTGA
TGGTGATGTTCCATATTCAGCCGGTGCCTGGAAAACTTATAATGCTTCATCAGGACAAACATTAACAGCA
GAATGGGGAAAATCATACGCTATTAATACATCTTCTGGAAGAGTGACTATAAATCTTCCAAAGGGTACAG
TTAATGATTACAACAAGGTAATTAGAGCTAGAGACGTATTTGCTACATGGAACGTCAACCCAGTTACACT
AGTAGCTGCTTCCGGCGATACGATTAAAGGGTCTGCAGTACCAGTTGAAATTAATGTTCGATTCAGCGAT
TTAGAACTAGTGTATTGTGCCCCAGGACGTTGGGAATATGTCAAAAATAAACAAATTGACAAAATTACCA
GTTCAGACATTAGTAATGTAGCTCGCAAAGAATTTTTAGTTGAAGTTCAAGGACAAACAGACTTTTTAGA
TGTTTTCCGTGGAACTAGTTATAATGTAAATAACATCAGAGTAAAACATCGTGGTAACGAATTGTATTAC
GGCGATGTGTTTAGCGAAAACAGCGATTTTGGCTCTCCAGGCGAAAATGAAGGAGAACTGGTTCCTCTTG
ATGGATTTAACATTCGATTAAGACAGCCTTGTAATATTGGTGACACTGTTCAAATTGAAACATTTATGGA
TGGTGTATCACAGTGGAGAAGTTCATATACAAGACGTCAAATTAGATTGTTAGATTCAAAATTAACGTCA
AAAACTTCTTTAGAAGGAAGCATTTACGTTACTGATTTATCAACAATGAAATCAATTCCATTTTCTGCTT
TTGGATTAATTCCAGGAGAACCTATTAATCCTAACTCTCTTGAGGTTCGTTTTAACGGGATTTTACAGGA
ATTGGCTGGCACAGTTGGAATGCCATTATTTCATTGTGTTGGTGCCGATTCAGACGATGAAGTAGAATGC
TCTGTTTTAGGTGGAACTTGGGAACAATCTCATACCGATTATTCAGTTGAAACTGATGAAAACGGCATAC
CAGAAATTTTACATTTCGATAGCGTATTTGAGCATGGTGACATTATCAATATCACCTGGTTTAATAATGA
TTTGGGTACATTATTAACAAAAGATGAGATTATTGATGAAACTGATAATCTCTATGTATCGCAAGGACCT
GGAGTAGATATTTCTGGTGATGTAAATTTAACAGACTTCGATAAAATTGGTTGGCCAAATGTAGAAGCAG
TTCAATCTTATCAACGCGCATTTAATGCTGTTTCAAATATCTTTGATACGATTTATCCTATTGGAACTAT
ATATGAAAACGCTGTTAATCCAAATAACCCTGTTACATATATGGGATTCGGCTCATGGAAATTATTTGGG
CAAGGAAAAGTTTTAGTTGGATGGAATGAAGATATTTCGGACCCTAACTTTGCTCTAAATAACAACGATT
TAGATTCGGGTGGAAATCCTTCACATACCGCAGGTGGAACAGGTGGTTCTACTTCTGTTACATTGGAAAA
TGCTAATCTTCCTGCAACTGAAACAGATGAAGAAGTTCTAATAGTTGATGAAAATGGATCAGTCATTGTT
GGTGGGTGTCAATACGATCCAGATGAATCCGGTCCAATTTACACTAAATACCGTGAAGCTAAAGCATCTA
CTAACTCTACTCACACTCCGCCAACATCAATAACTAACATTCAACCATATATTACAGTTTATCGTTGGAT
AAGGATTGCATAATGAGTTTACTTAATAATAAAGCGGGAGTTATTTCCCGCTTAGCCGATTTTCTTGGTT
TTAGACCTAAAACTGGCGACATTGATGTAATGAATCGTCAATCAGTCGGGTCAGTGACAATATCTCAATT
AGCGAAAGGATTTTATGAACCAAACATAGAATCAGCTATTAATGACGTTCATAATTTTTCTATAAAAGAC
GTTGGCACAATTATTACTAATAAAACTGGTGTTTCTCCTGAGGGTGTTTCTCAAACTGATTATTGGGCAT
TTTCTGGAACTGTAACAGACGATTCTCTTCCTCCGGGTTCTCCTATTACGGTATTAGTATTTGGTCTTCC
AGTTTCAGCAACAACTGGAATGACGGCAATTGAGTTTGTTGCAAAAGTTCGCGTTGCACTACAAGAAGCT
ATTGCGTCATTTACTGCTATCAATTCATATAAAGACCATCCAACTGATGGTAGTAAATTAGAAGTTACTT
ATTTAGATAATCAAAAACATGTATTAAGCACATATTCTACATATGGAATAACTATTTCCCAAGAAATTAT
ATCTGAGTCTAAGCCTGGCTATGGTACATGGAATTTATTGGGCGCACAAACTGTAACTTTAGATAATCAG
CAGACTCCTACAGTATTTTATCATTTTGAGAGAACAGCATGAGTAATAATACATATCAACACGTTTCTAA
TGAATCTCGTTATGTAAAATTTGATCCTACCGATACGAATTTTCCACCGGAGATTACTGATGTTCACGCT
GCTATAGCAGCCATTTCTCCTGCTGGAGTAAATGGAGTTCCTGATGCATCGTCAACAACAAAGGGAATTC
TATTTATTCCCACTGAACAGGAAGTTATAGATGGAACTAATAATACCAAAGCAGTTACACCAGCAACGTT
GGCAACAAGATTATCTTATCCAAATGCAACTGAAACTGTTTACGGATTAACAAGATATTCAACCAATGAT
GAAGCCATTGCCGGAGTTAATAATGAATCTTCTATAACTCCAGCTAAATTTACTGTCGCCCTTAATAATG
CGTTTGAAACGCGAGTTTCAACTGAATCCTCAAATGGTGTTATTAAAATTTCATCTCTACCGCAAGCATT
AGCTGGTGCAGATGATACTACTGCAATGACTCCATTAAAAACACAGCAGTTAGCTATTAAATTAATTGCG
CAAATTGCTCCTTCTGAAACCACAGCTACCGAATCGGACCAAGGTGTTGTTCAATTAGCAACAGTAGCGC
AGGTTCGTCAGGGAACTTTAAGAGAAGGCTATGCAATTTCTCCTTATACGTTTATGAATTCATCTTCTAC
TGAAGAATATAAAGGCGTAATTAAATTAGGAACACAATCAGAAGTTAACTCGAATAATGCTTCTGTTGCG
GTTACTGGCGCAACTCTTAATGGTCGTGGTTCTACGACGTCAATGAGAGGCGTAGTTAAATTAACTACAA
CCGCCGGTTCACAGAGTGGAGGCGATGCTTCATCAGCCTTAGCTTGGAATGCTGACGTTATCCAGCAAAG
AGGTGGTCAAATTATCTATGGAACACTCCGCATTGAAGACACATTTACAATAGCTAATGGTGGAGCAAAT
ATTACGGGTACCGTCAGAATGACTGGCGGTTATATTCAAGGTAACCGCATCGTAACACAAAATGAAATTG
ATAGAACTATTCCTGTCGGAGCTATTATGATGTGGGCCGCTGATAGTCTTCCTAGTGATGCTTGGCGCTT
CTGCCATGGTGGAACTGTTTCAGCGTCAGATTGTCCATTATATGCTTCTAGAATTGGAACAAGATATGGC
GGAAACCCATCAAATCCTGGATTGCCTGACATGCGTGGTCTTTTTGTTCGTGGTTCTGGTCGTGGTTCTC
ACTTAACAAATCCAAATGTTAATGGTAATGACCAATTTGGTAAACCTAGATTAGGTGTAGGTTGTACCGG
TGGATATGTTGGTGAAGTACAGATACAACAGATGTCTTATCATAAACATGCTGGTGGATTTGGTGAGCAT
GATGATCTGGGGGCATTCGGTAATACCCGTAGATCAAATTTTGTTGGTACACGTAAAGGACTTGACTGGG
ATAACCGTTCATACTTCACCAATGACGGATATGAAATTGACCCAGAATCACAACGAAATTCCAAATATAC
ATTAAATCGTCCTGAATTAATTGGAAATGAAACACGTCCATGGAACATTTCTTTAAACTACATAATTAAG
GTAAAAGAATGACAGATATTGTACTGAATGACTTACCATTCGTTGACGGCCCTCCTGCAGAGGGCCAGAG
CCGCATTTCCTGGATTAAAAACGGCGAAGAAATATTAGGAGCTGACACACAGTATGGAAGTGAAGGCTCA
ATGAATAGACCTACGGTTTCTGTACTAAGAAATGTTGAAGTTCTTGATAAAAACATTGGAATACTTAAAA
CATCTTTAGAAACCGCAAATAGTGATATTAAAACAATTCAGGGCATCTTAGATGTATCTGGTGATATTGA
AGCTTTGGCCCAAATAGGTATCAATAAAAAGGATATTTCTGACCTCAAAACGCTAACCAGTGAACACACA
GAAATATTAAATGGAACTAATAATACGGTTGACAGTATTCTTGCCGATATTGGTCCATTTAACGCCGAGG
CCAACTCTGTATACAGAACGATCAGAAATGATTTACTGTGGATAAAGCGTGAACTTGGACAATACACTGG
TCAAGATATTAATGGTCTTCCTGTTGTAGGAAATCCTAGTAGTGGAATGAAGCATCGCATTATTAATAAT
ACTGATGTCATCACTTCGCAGGGAATACGTTTAAGCGAATTAGAAACAAAATTTATTGAATCTGATGTAG
GTTCTTTGACCATTGAAGTTGGTAATCTTCGTGAAGAGCTTGGACCGAAACCACCATCATTTTCACAGAA
CGTTTATAGTCGTTTAAATGAAATTGACACTAAACAGACAACAGTTGAGTCTGACATTAGTGCTATTAAG
ACCTCAATAGGATATCCAGGAAATAATTCGATTATCACGAGTGTTAATACAAACACTGATAATATTGCAT
CTATTAATTTAGAGCTAAATCAAAGTGGAGGTATTAAACAGCGTTTAACCGTTATTGAAACTTCCATTGG
TTCAGATGATATTCCTTCGAGTATTAAAGGTCAAATCAAAGATAATACAACTTCAATCGAATCTCTAAAT
GGAATCGTCGGTGAAAACACTTCATCTGGCTTAAGAGCGAATGTTTCATGGTTAAACCAAATTGTTGGAA
CTGATTCTAGCGGTGGACAACCTTCTCCTCCTGGGTCTCTTTTAAACCGAGTTTCTACAATTGAAACTTC
TGTTTCAGGCTTGAATAACGCTGTTCAAAACCTACAAGTAGAGATTGGTAATAACAGCGCAGGAATTAAA
GGGCAAGTTGTAGCGTTAAATACTTTAGTAAATGGAACTAATCCAAACGGTTCAACTGTTGAAGAGCGCG
GATTAACCAATTCAATAAAAGCTAACGAAACTAACATTGCATCAGTTACACAAGAAGTGAATACAGCTAA
AGGCAATATATCTTCTTTACAAGGTGATGTTCAAGCTCTCCAAGAAGCCGGTTATATTCCTGAAGCTCCA
AGAGATGGGCAAGCTTACGTTCGTAAAGATGGCGAATGGGTATTCCTTTCTACCTTTTTATCACCAGCAT
AACATGGGGCCGCAAGGCCCCAAAGGATTTTAAATGTCAGGATATAATCCTCAGAATCCAAAGGAACTCA
AAGATGTCATTCTAAGACGTTTAGGGGCTCCAATTATTAATGTTGAGTTAACACCCGATCAAATTTACGA
TTGTATCCAGCGTGCCCTAGAATTATACGGTGAATACCATTTTGATGGACTCAATAAAGGTTTTCATGTT
TTTTATGTAGGGGATGATGAAGAAAGGTACAAGACCGGAGTCTTCGATTTAAGAGGTTCTAACGTATTTG
CAGTAACCCGCATTTTACGCACAAATATTGGGTCAATAACATCTATGGATGGAAACGCTACATATCCGTG
GTTTACTGACTTTCTTTTAGGAATGGCTGGTATTAATGGCGGAATGGGAACGTCTTGTAATAGATTTTAT
GGACCAAATGCCTTTGGAGCTGATTTAGGATATTTTACCCAGCTTACCAGTTATATGGGAATGATGCAAG
ATATGCTCTCTCCTATTCCAGACTTTTGGTTTAATTCAGCAAATGAACAGCTCAAAGTCATGGGAAACTT
CCAAAAATATGATTTAATTATCGTAGAAAGCTGGACTAAATCATACATTGATACAAACAAAATGGTTGGA
AATACAGTAGGATATGGAACAGTCGGTCCACAAGATAGCTGGTCATTATCTGAACGATATAATAACCCAG
ACCACAATTTAGTAGGTCGTGTTGTCGGCCAAGATCCGAATGTTAAACAGGGTGCTTATAATAATCGTTG
GGTGAAAGACTATGCAACAGCTTTAGCTAAAGAATTGAACGGTCAAATTTTAGCACGCCACCAAGGTATG
ATGCTTCCGGGCGGTGTTACAATTGATGGGCAGCGCTTAATAGAAGAAGCCAGATTAGAAAAAGAAGCAC
TGCGCGAAGAATTATACTTACTTGATCCTCCATTTGGAATTTTGGTAGGTTAATATGGCTACTTATGATA
AAAATCTTTTTGCTAAATTGGAAAACCGCACAGGTTATTCTCAGACCAATGAAACTGAAATATTAAATCC
TTATGTAAATTTCAATCATTATAAAAACAGCCAAATATTAGCTGATGTATTAGTAGCTGAAAGCATTCAA
ATGCGAGGTGTAGAATGCTATTATGTTCCAAGAGAGTATGTTTCCCCTGATTTGATATTCGGCGAAGACT
TAAAAAATAAATTTACTAAAGCTTGGAAATTTGCTGCATATTTAAATTCATTTGAAGGATATGAAGGAGC
TAAATCGTTCTTTAGTAACTTTGGTATGCAAGTACAAGACGAAGTGACTTTATCTATTAACCCAAATTTA
TTTAAGCATCAAGTTAACGGAAAAGAACCCAAGGAAGGTGATTTGATATATTTTCCTATGGATAACAGCT
TATTTGAAATTAACTGGGTTGAACCATATGATCCATTTTATCAATTAGGCCAAAACGCTATTCGTAAAAT
TACGGCAGGTAAATTCATTTATTCTGGAGAAGAAATTAATCCAGTTCTACAGAAAAATGAAGGAATTAAC
ATTCCAGAATTTAGTGAATTAGAATTAAATGCTGTTCGCAATCTTAACGGTATTCATGACATTAATATTG
ATCAGTATGCTGAAGTAGATCAAATTAATTCTGAAGCTAAAGAATACGTTGAACCTTATGTTGTTGTCAA
TAACAGAGGCAAATCTTTCGAATCTAGCCCATTTGACAATGATTTCATGGATTAATAAATATTATAAACT
AATTAAAGCCCGGATTAGGAGAAGTCATGTTTGGTTATTTTTATAATTCGTCTTTTAGACGATATGCTAC
CTTGATGGGCGATTTGTTTTCAAATATCCAAATCAAACGTCAGTTAGAATCTGGTGATAAGTTTATACGT
GTTCCTATTACGTATGCATCAAAGGAACACTTTATGATGAAATTGAATAAATGGACATCAATAAATTCAC
AAGAAGATGTAGCTAAAGTTGAAACTATTCTACCTCGTATAAATTTACATTTAGTTGATTTTAGCTATAA
TGCTCCATTTAAAACAAACATTTTAAATCAGAATTTACTGCAAAAAGGTGCAACTTCTGTAGTATCGCAG
TATAATCCATCTCCTATTAAAATGATTTATGAATTGAGTATCTTTACTCGCTATGAAGATGATATGTTTC
AAATAGTTGAACAGATTCTTCCATATTTTCAACCTCATTTTAATACAACTATGTACGAGCAGTTTGGAAA
TGATATTCCATTTAAAAGGGATATTAAAATTGTACTGATGTCTGCTGCTATAGACGAAGCTATAGATGGG
GATAATTTATCTCGTCGTAGAATTGAATGGTCATTAACATTTGAAGTAAATGGATGGATGTATCCTCCAG
TAGATGATGCAGAAGGATTAATTCGTACTACTTATACAGATTTTCACGCCAATACAAGAGATTTGCCTGA
CGGCGAAGGTGTTTTTGAATCTGTCGATAGCGAAGTTGTTCCTCGAGATATTGACCCAGAAGACTGGGAT
GGAACAGTAAAACAAACTTTCACTAGTAATGTAAATAGACCAACACCGCCAGAACCTCCTGGCCCAAGAA
CATAGAGGTTATTATGGAAGGTCTTGATATAAACAAACTTTTAGATATTTCTGACCTCCCCGGAATTGAC
GGGGAGGAAATCAAAGTGTATGAACCTCTGCAATTAGTAGAAGTTAAAAGCAATCCACAAAACCGTACTC
CAGACTTAGAAGATGATTATGGAGTAGTTCGTCGAAATATGCATTTTCAGCAACAAATGCTAATGGACGC
TGCCAAGATTTTTCTTGAGACAGCAAAGAATGCTGATTCTCCTCGTCACATGGAAGTATTTGCAACTCTT
ATGGGGCAAATGACTACGACGAACAGAGAAATACTGAAGCTTCATAAAGATATGAAAGACATTACATCTG
AGCAGGTTGGCACCAAAGGCGCTGTTCCTACAGGTCAAATGAATATTCAGAATGCGACAGTATTCATGGG
TTCACCAACAGAATTAATGGACGAAATTGGTGATGCTTACGAGGCTCAAGAAGCTCGTGAGAAGGTGATA
AATGGAACAACCGATTAATGTATTAAATGATTTCCATCCGTTAAATGAAGCTGGAAAAATTTTAATAAAA
CACCCAAGCTTAGCGGAAAGAAAAGATGAAGATGGAATTCATTGGATAAAATCTCAGTGGGATGGAAAAT
GGTATCCTGAAAAATTCAGTGATTACCTTCGTCTACACAAAATAGTAAAAATTCCAAACAACTCTGATAA
GCCTGAATTATTTCAAACTTATAAAGATAAGAATAATAAAAGATCTCGGTATATGGGTCTTCCTAACTTG
AAACGAGCTAATATTAAAACACAATGGACTCGTGAAATGGTTGAGGAATGGAAAAAATGCCGAGATGATA
TTGTTTATTTTGCAGAAACATACTGTGCCATTACTCATATTGACTATGGTGTCATAAAGGTTCAATTACG
TGACTATCAGCGTGATATGCTCAAAATAATGTCATCTAAACGTATGACTGTTTGTAATCTATCGCGCCAG
CTCGGTAAAACCACCGTAGTAGCTATTTTCCTTGCACACTTTGTATGTTTTAACAAAGATAAAGCTGTAG
GTATTCTTGCACACAAAGGCTCAATGTCTGCGGAAGTTTTAGACCGTACTAAGCAAGCAATTGAACTGCT
TCCTGACTTTTTACAACCAGGAATTGTTGAATGGAATAAGGGTTCAATTGAACTAGATAATGGTTCTTCA
ATTGGCGCTTATGCTTCCTCTCCTGACGCAGTTCGTGGTAACTCGTTCGCAATGATTTACATTGACGAAT
GTGCGTTTATTCCAAACTTCCATGATTCCTGGCTTGCTATTCAACCAGTAATTTCATCTGGTCGTCGTTC
GAAAATTATTATTACTACGACTCCTAATGGATTAAATCATTTTTATGATATTTGGACTGCTGCTGTCGAA
GGTAAATCTGGATTTGAACCATATACTGCTATTTGGAATTCAGTTAAAGAACGTCTTTATAACGATGAAG
ATATTTTTGACGATGGATGGCAATGGAGCATACAAACCATTAATGGTTCTTCATTAGCTCAATTCCGTCA
AGAACATACTGCAGCGTTTGAAGGGACTTCTGGTACATTAATTTCAGGAATGAAATTAGCTGTTATGGAT
TTTATTGAAGTAACACCAGATGATCATGGTTTTCACCAATTTAAAAAACCTGAACCAGATAGAAAATATA
TTGCAACTCTAGATTGCTCAGAAGGTCGTGGGCAAGATTACCACGCTTTGCATATTATTGATGTTACTGA
TGATGTGTGGGAACAGGTTGGTGTTTTGCATTCAAACACTATTTCTCATTTAATTCTACCTGACATCGTT
ATGCGTTATTTAGTAGAATACAATGAATGCCCAGTTTATATTGAATTAAATAGTACTGGTGTGTCAGTTG
CAAAATCGCTTTATATGGATTTAGAATACGAAGGTGTTATCTGCGATTCATATACTGATTTAGGAATGAA
ACAAACTAAACGCACGAAAGCAGTAGGATGTTCCACGCTAAAAGACCTTATTGAAAAAGATAAGCTTATT
ATTCATCACCGAGCGACTATTCAAGAATTTAGAACGTTTAGTGAAAAAGGCGTGTCTTGGGCGGCTGAAG
AAGGTTATCATGACGATTTAGTAATGTCTTTAGTGATTTTTGGATGGTTATCAACGCAGTCAAAATTTAT
TGATTATGCGGATAAAGATGACATGCGATTAGCATCTGAAGTATTTTCAAAAGAGCTTCAGGATATGAGC
GACGACTACGCGCCAGTTATATTTGTGGATTCGGTTCATTCTGCTGAGTATGTTCCAGTATCTCATGGTA
TGTCAATGGTATAAATATATTAAAGCATATTAAAGAGGATTAAAAATGACTTTATTATCTCCGGGCATTG
AGCTCAAAGAAACTACGGTTCAAAGCACCGTAGTTAATAACTCTACTGGTACAGCAGCTTTGGCCGGTAA
ATTCCAGTGGGGTCCTGCTTTTCAGATTAAACAGGTTACAAATGAAGTAGATTTAGTTAATACTTTTGGT
CAACCAACCGCTGAAACTGCTGACTATTTTATGTCTGCGATGAATTTCTTGCAGTACGGAAATGACTTAC
GAGTAGTTCGTGCTGTTGATAGAGATACCGCTAAAAACTCATCGCCAATTGCTGGTAATATTGATTACAC
AATTTCTACCCCAGGTAGTAACTATGCGGTTGGAGATAAAATCACGGTCAAATATGTTTCAGATGATATT
GAAACTGAAGGTAAAATTACTGAAGTAGACGCAGATGGAAAAATTAAGAAAATTAATATTCCTACTGGCA
AAAATTACGCTAAAGCGAAAGAAGTCGGTGAATATCCAACACTAGGTTCTAACTGGACTGCGGAAATTTC
TTCATCTTCCTCTGGTTTAGCTGCAGTAATAACTCTTGGAAAAATTATTACTGATTCTGGTATTTTATTA
GCTGAAATTGAAAATGCTGAAGCTGCTATGACAGCGGTTGACTTTCAAGCAAATCTTAAAAAATATGGAA
TTCCAGGAGTAGTAGCGCTTTATCCAGGCGAATTAGGCGATAAAATTGAAATTGAAATCGTATCTAAAGC
TGACTATGCAAAAGGAGCTTCTGCATTACTCCCAATTTATCCAGGTGGTGGTACTCGTGCATCTACTGCT
AAAGCAGTGTTTGGATATGGACCGCAAACTGATTCACAGTACGCTATTATAGTTCGTCGTAATGATGCTA
TTGTTCAAAGCGTTGTTCTTTCAACTAAGCGTGGTGAAAAAGATATTTACGATAGTAACATCTATATCGA
TGACTTTTTCGCAAAAGGTGGTTCAGAATATATTTTTGCAACTGCACAAAACTGGCCAGAAGGCTTCTCT
GGAATTTTAACTCTGTCTGGTGGATTATCATCAAATGCTGAAGTAACAGCAGGAGATTTGATGGAAGCTT
GGGACTTCTTTGCTGACCGTGAATCTGTTGACGTTCAACTGTTTATTGCAGGTTCTTGTGCCGGTGAATC
TTTAGAAACAGCATCTACTGTCCAAAAACACGTCGTTTCAATTGGGGATGCTCGCCAAGATTGCTTAGTA
TTGTGCTCTCCTCCGCGTGAAACTGTAGTTGGAATTCCTGTAACCCGTGCTGTTGATAACCTAGTCAATT
GGAGAACTGCGGCAGGTTCATACACTGATAATAACTTTAATATCAGTTCAACCTATGCAGCAATTGATGG
TAACCATAAGTATCAGTATGACAAATATAATGATGTGAATCGTTGGGTTCCATTAGCAGCTGATATTGCT
GGTTTATGCGCAAGAACTGATAACGTATCTCAGACTTGGATGTCTCCAGCTGGTTATAATCGTGGTCAGA
TTCTTAACGTTATTAAACTTGCTATTGAAACTCGCCAGGCTCAGCGCGACCGTTTATACCAAGAAGCTAT
CAACCCGGTAACCGGTACAGGTGGTGATGGTTACGTATTGTATGGTGATAAAACAGCTACTTCTGTTCCT
TCTCCATTTGATCGTATTAACGTTCGTCGTCTGTTTAATATGTTGAAAACGAATATCGGACGTAGTTCAA
AATATCGTTTGTTCGAATTAAACAACGCGTTTACTCGTTCATCATTCCGCACAGAAACTGCCCAGTACTT
ACAAGGGAATAAAGCTCTCGGTGGAATTTATGAATATCGTGTAGTTTGCGATACAACAAATAACACTCCG
TCAGTAATTGATAGAAATGAGTTTGTTGCAACATTCTACATCCAACCGGCTAGAAGCATTAACTACATTA
CCTTAAACTTCGTAGCAACTGCTACTGGTGCAGATTTCGATGAGTTAACTGGTCTTGCTGGTTAATACGG
TGCATTCTAAAGGCCTGTTTCGGCAGGCCATATAAATACACTATATCCTTAATTCTTTAATTCTATATGC
CCTAGGTTAAACATAGGGATATAAATACTACAGAGGCTAATATGTTTGTAGATGATGTAACACGAGCGTT
TGAATCTGGTGATTTTGCTCGACCTAACTTATTCCAAGTAGAAATTTCTTATCTTGGACAAAATTTTACG
TTCCAATGTAAAGCTACTGCTCTACCAGCTGGTATTGTAGAAAAAATTCCAGTCGGATTTATGAACCGTA
AAATTAACGTAGCAGGCGATCGTACATTCGATGACTGGACTGTTACAGTAATGAACGATGAAGCTCATGA
TGCTCGTCAGAAGTTCGTTGATTGGCAAAGCATTGCTGCGGGGCAAGGAAACGAAATTACTGGTGGAAAA
CCTGCAGAGTATAAAAAGAGCGCTATCGTTCGTCAATATGCTCGTGACGCTAAAACAGTAACAAAAGAAA
TTGAAATTAAAGGTCTGTGGCCTACTAACGTGGGTGAACTTCAATTAGATTGGGATTCAAACAATGAAAT
CCAAACTTTTGAAGTAACTCTTGCTCTCGATTATTGGGAATAAAATGAATGGGGAGAAATCCCCATCCTG
CTTAAAGCAGAGAAGTCCATTATAAATATAACTATAATTCCCATTTGGAGAATACAATGAAATTTAATGT
ATTAAGTTTGTTTGCTCCATGGGCTAAAATGGACGAACGAAATTTTAAAGACCAAGAAAAAGAAGATCTT
GTTTCCATTACAGCCCCAAAGCTTGATGATGGAGCAAGAGAATTTGAAGTAAGCTCGAATGAAGCTGCTT
CTCCTTATAATGCTGCATTCCAAACAATTTTTGGTTCATATGAACCAGGAATGAAAACTACTCGTGAGCT
TATTGATACATATCGTAATCTCATGAATAACTATGAAGTAGATAATGCAGTTTCAGAAATCGTTTCAGAT
GCTATCGTCTATGAAGATGATACTGAAGTCGTAGCGTTAAATTTGGATAAATCTAAATTTAGCCCAAAAA
TTAAAAATATGATGTTAGATGAATTTAGTGATGTATTAAATCATCTATCGTTTCAACGAAAAGGTTCTGA
TCATTTTAGACGTTGGTATGTTGATTCAAGAATTTTCTTTCATAAAATCATTGATCCAAAACGTCCAAAA
GAAGGCATAAAAGAATTACGTAGATTAGACCCTCGCCAAGTTCAGTATGTTCGTGAAATTATAACAGAAA
CTGAAGCTGGCACAAAAATAGTTAAAGGTTACAAAGAATATTTTATATATGATACTGCCCATGAGTCATA
TGCATGTGATGGTAGAATGTATGAAGCTGGCACAAAAATAAAAATTCCTAAAGCTGCCGTCGTTTATGCC
CATTCTGGATTAGTCGATTGTTGCGGTAAAAATATCATCGGGTATTTGCATCGTGCTGTTAAACCTGCTA
ACCAATTAAAATTATTAGAAGATGCTGTAGTCATTTATCGCATTACTCGTGCTCCTGACCGTCGTGTTTG
GTATGTAGACACAGGTAATATGCCTGCTCGTAAAGCTGCTGAGCACATGCAACATGTTATGAACACGATG
AAAAACCGTGTAGTATATGATGCATCAACAGGTAAAATAAAAAATCAACAGCATAATATGTCTATGACCG
AAGACTATTGGTTGCAGCGCCGTGATGGTAAAGCTGTGACAGAAGTTGATACTCTTCCTGGTGCTGATAA
TACTGGCAATATGGAAGATATTCGTTGGTTTAGACAAGCTCTTTATATGGCATTACGTGTTCCTCTTTCA
CGCATTCCGCAAGACCAACAAGGCGGTGTGATGTTTGATTCTGGAACTAGCATTACACGTGATGAATTAA
CGTTTGCTAAATTTATTCGTGAGTTACAGCACAAGTTTGAAGAAGTTTTCCTAGATCCGCTTAAAACAAA
TCTTTTGCTTAAAGGTATAATCACAGAAGATGAGTGGAATGATGAAATAAATAATATTAAGATAGAATTT
CATCGGGATAGCTACTTTGCTGAGCTCAAAGAAGCAGAAATTTTGGAACGAAGAATTAATATGCTAACCA
TGGCAGAACCATTTATTGGTAAATATATTTCTCACAGAACTGCTATGAAAGACATTTTGCAGATGACTGA
TGAAGAAATAGAACAAGAAGCCAAGCAAATTGAAGAAGAGTCTAAAGAGGCTCGTTTCCAAGACCCCGAC
CAAGAACAAGAGGATTTTTAATGGAAGGTTTAATTGAAGCTATTAAATCAAACGACCTCGTAGCCGCTCG
TAAATTATTTGCTGAAGCCATGGCTGCAAGAACGATTGATTTAATTAAAGAAGAAAAAATCGCTATCGCT
CGCAATTTCTTAATCGAAGGTGAAGAACCTGAAGACGAGGATGAAGATGAAGATGACGAAGATAGTGATG
ATAAAGACGACAAAAAAGACGAAGACTCTGACGAAGACGAGGATGATGAATAATGCTTCTGATCCCTGAA
ACTCATGAATTAGTTCTCGAGAATGTCGAAGCACTTATTCCTGAAGCACAGGGTCGCTTTGACGAATTGT
CTTCTGCTTTAAATAAAGACGATATAAATACAATTGTCGAGAATATGCTTGATGATGAAACTGATTTAGC
GGTTGCATTAGCTTCTATTAATGAAAATATGCCGTTAAATGAATTCATCGTTAAACATGTTTCTGCCCGT
GGTGAAATTACTCGCACTAAAGACCGCAAAACGCGTGAACGAAATGCATTTCAAACCACTGGGCTGTCTA
AAGCAAAACGTAGACAAATTGCTCGTAAAGCTACCAAAACGAAGATTGCCAATCCAGCAGGTCAATCTCG
TGCTCAGCGTAAGCGTAAAAAAGCTCTTAAACGCCGTAAAGCATTAGGATTAAGCTAATGAATGAACCCC
AATTACTAATTGAAACTTGGGGTCAACCTGGCGAAATTATTGATGGCGTACCAATGCTTGAATCTCATGA
TGGAAAAGACTTAGGTTTAAAACCGGGTTTATACATCGAAGGAATATTCATGCAAGCGGAAGTCGTCAAT
AGAAATAAACGTCTTTATCCAAAACGTATATTAGAAAAAGCGGTAAAAGACTATATTAATGAGCAAGTTT
TAACTAAACAAGCTCTCGGAGAATTAAATCATCCTCCACGCGCTAATGTTGACCCGATGCAAGCCGCTAT
CATTATAGAAGATATGTGGTGGAAAGGAAATGACGTATACGGACGAGCTCGTGTTATTGAAGGTGACCAT
GGTCCTGGAGATAAATTAGCAGCTAATATTCGTGCCGGATGGATTCCAGGAGTTTCTTCTCGTGGATTAG
GTTCATTGACTGACACAAATGAAGGTTATCGTATCGTAAACGAAGGATTCAAATTAACTGTAGGTGTTGA
TGCAGTATGGGGTCCAAGTGCTCCAGATGCATGGGTAACTCCTAAGGAAATTACCGAATCACAGACGGCG
GAAGCCGATACAAGTGCCGATGACGCCTATATGGCTCTCGCAGAGGCCATGAAAAAAGCGTTATAAATAT
TATTATCTAAACAACAGGACTACAAAATGCTTAAAGAACAACTGATTGCCGAAGCGCAGAAAATTGATGC
TTCCGTTGCTCTTGATAGTATTTTCGAATCAGTTAATATTTCTCCGGAAGCAAAAGAAACTTTCGGCACT
GTATTCGAAGCTACCGTCAAGCAGCACGCCGTTAAATTAGCTGAATCTCATATCGCTAAAATTGCTGAAA
AAGCAGAAGAAGAAGTAGAAAAAAATAAAGAAGAAGCCGAAGAAAAAGCTGAGAAGAAAATCGCTGAGCA
AGCTTCTAAATTCATTGACCATCTTGCAAAAGAATGGCTCGCTGAAAATAAATTAGCAGTTGATAAAGGC
ATCAAAGCCGAACTGTTTGAATCCATGCTTGGTGGATTAAAAGAGCTCTTTGTTGAACACAACGTTGTTG
TTCCAGAAGAATCAGTTGATGTTGTAGCTGAAATGGAAGAAGAGCTGCAAGAACATAAAGAAGAATCGCC
TCGTCTGTTCGAAGAACTGAATATGCGCGACGCATATATCAATTATGTGCAGCGTGAAGTGGCATTGAGC
GAAAGTACTAAAGATCTGACTGAGTCTCAAAAAGAAAAAGTCTCTGCTCTGGTCGAAGGTATGGATTATT
CAGATGCATTCTCAAGTAAATTGAGTGCAATCGTAGAAATGGTGAAGAAATCTAATAAAGATGAAAGCAC
TATTACTGAGAGTATAAATACTCCTGATACTGAAGCAGCCGGACTGAATTTCGTCACTGAAGCTGTAGAA
GATAAAGCTGCACAGGGTGCAGAAGATATTGTAAGTGTATATGCGAAAGTCGCATCTCGTTTCTAATTTT
AAAGGTTAACACAAATGACTATCAAAACTAAAGCTGAACTTTTGAACAAATGGAAGCCATTACTGGAAGG
TGAAGGTTTACCGGAAATTGCTAATAGCAAACAAGCGATTATCGCTAAAATCTTTGAAAACCAGGAAAAA
GATTTCCAGACAGCTCCGGAATATAAAGACGAAAAAATTGCTCAGGCATTCGGTTCTTTCTTAACAGAAG
CTGAAATCGGTGGTGACCACGGTTACAATGCTACCAACATCGCTGCAGGTCAGACTTCTGGCGCAGTAAC
TCAGATTGGCCCAGCTGTTATGGGTATGGTACGTCGTGCTATTCCTAACCTGATTGCTTTCGATATTTGT
GGTGTTCAGCCGATGAACAGCCCGACTGGCCAGGTATTCGCACTGCGCGCAGTATATGGTAAAGACCCAG
TGGCTGCCGGTGCTAAAGAAGCATTCCACCCAATGTATGGTCCAGATGCAATGTTCTCTGGTCAGGGTGC
TGCTAAGAAATTCCCAGCTCTGGCTGCTAGCACACAAACCACAGTAGGTGATATCTATACTCACTTCTTC
CAGGAAACTGGTACTGTATATCTGCAAGCTTCTGTTCAAGTAACAATCGATGCTGGTGCGACTGATGCTG
CTAAATTAGATGCTGAAATTAAGAAACAAATGGAAGCTGGTGCACTGGTAGAAATCGCTGAAGGTATGGC
TACTTCTATCGCTGAACTCCAGGAAGGTTTCAATGGTTCTACCGATAACCCATGGAATGAAATGGGCTTC
CGTATCGATAAGCAAGTTATCGAAGCTAAATCTCGTCAGCTGAAAGCTGCTTACTCTATTGAATTAGCAC
AAGACCTCCGCGCTGTTCACGGTATGGATGCTGATGCTGAACTGTCTGGTATTCTGGCTACAGAAATTAT
GCTGGAAATCAACCGTGAAGTTGTTGATTGGATTAACTACTCAGCTCAGGTTGGTAAATCTGGTATGACC
CTGACTCCGGGTTCTAAAGCTGGTGTATTTGACTTCCAGGACCCAATTGATATTCGTGGTGCTCGCTGGG
CGGGTGAATCCTTTAAAGCTCTGTTGTTCCAGATTGACAAAGAAGCAGTTGAAATTGCTCGTCAGACCGG
TCGTGGTGAAGGTAACTTCATTATCGCTTCCCGTAACGTAGTTAACGTTTTGGCTTCAGTTGATACCGGC
ATTTCTTATGCTGCACAGGGTCTGGCTACCGGCTTTAGCACTGATACTACCAAGTCAGTATTTGCTGGTG
TTCTGGGTGGTAAATACCGCGTATATATCGACCAGTATGCTAAACAGGATTATTTCACTGTAGGTTATAA
AGGTCCGAACGAAATGGATGCTGGTATTTACTATGCTCCATATGTAGCTCTGACTCCGCTGCGTGGTTCC
GATCCGAAGAACTTCCAACCGGTAATGGGATTCAAAACTCGTTACGGTATCGGTATCAACCCATTTGCAG
AATCCGCTGCTCAGGCTCCGGCTTCTCGCATCCAGAGCGGTATGCCTTCTATTCTGAATAGCCTTGGTAA
AAACGCTTACTTTAGACGTGTATATGTTAAAGGTATCTAATCTCTAACGATAGAAACACAATTTTAGGGA
ACCTTCGGGTTCCCTTTTTTCTATTTTATACGATAGCAATCAGGCATATCATCCGCATTTATCCAATTGC
GAATAGTTTTAGGACTAACTTTAAAATGCTCCGCTGCGTAATCAGGATTATCAAATTTAACGCCCTTTAT
ACATATTGGAATAAATTTTTTAATACCACCAAGTTTTTCAGAAATAGCTTTACGATGTGAAATCGATATA
GGTTTGTTTTTTCGTGGATGAACATGTGTTTTATAATATTCATTGCGCCCTTTAACTCGCTTCGCAATAG
TTTCATCAGATTGCTTAACGCCTGTTTTTGCCTTTGATATTTTTCGTTTAGCTTCCACAGTCATTCCTTC
TTTTGTTCGTATTGATAACATATTACGATATGAAGGATCTTGCAAATGAACTATAACTGGATTTCCTCTG
CCACCAATAGCAGCATTATAGGTATCAGTTCTCATAACGAATTCCTCATTAACTAGTAAAGCTTCCATTT
TATACATCTCCTCAGATGAGGAGAAAGAATAAAGAATTTCTTTTTTAAAGTTATGAATACCATATTTTTT
GATGGATTTTTTGATGTTTACGCCAGAACCCATATAACCATCGTTTTCGTCAAGAGTAGCATGAGCTCCG
ATGTAAATTTTTCCATTGATGATATTAGTAATTTGATATATTAAATATTTCATTTTAAACATCACTCCGT
TTGTATATGATTATAATATCATATTACTTTGGTCTTGTAAATAACTTTATAAATAGTATTATATTTCAAC
AAGGAAAATACAATGGCTAAAATCAACGAACTTCTGCGCGAATCAACCACAACGAATAGCAACTCAATCG
GTCGCCCAAATCTCGTTGCTTTGACTCGCGCTACCACTAAATTAATATATTCTGACATTGTAGCAACGCA
AAGAACTAATCAACCTGTTGCTGCTTTTTATGGTATCAAATACCTTAACCCAGACAACGAATTTACATTT
AAAACTGGTGCTACTTACGCTGGCGAAGCTGGATATGTAGACCGAGAACAAATCACAGAATTAACAGAAG
AGTCTAAATTAACTCTCAATAAAGGCGATTTATTCAAATATAATAATATCGTTTATAAAGTATTAGAAGA
TACTCCATTTGCTACTATCGAAGAAAGTGATTTAGAATTAGCTCTTCAGATTGCAATCGTTCTTTTAAAG
GTTCGTCTATTTTCTGACGCAGCGTCAACAAGCAAATTTGAAAGCTCTGATAGTGAAATTGCGGATGCTA
GATTCCAGATTAATAAATGGCAAACTGCAGTTAAATCTCGTAAACTTAAAACTGGCATCACAGTTGAATT
AGCGCAAGATTTAGAAGCAAATGGATTCGATGCTCCTAATTTCTTGGAAGATTTGCTTGCAACTGAAATG
GCAGATGAAATCAATAAAGACATTCTGCAGTCTTTGATTACAGTGTCAAAACGCTATAAAGTTACAGGAA
TTACTGATAGTGGATTCATCGATTTGAGTTATGCATCTGCTCCTGAAGCTGGTCGTTCATTATACCGAAT
GGTATGTGAAATGGTTTCGCATATCCAAAAAGAATCAACTTATACAGCAACGTTCTGTGTTGCTTCAGCT
CGTGCCGCTGCGATTCTTGCTGCATCAGGCTGGTTAAAACATAAACCAGAAGATGACAAATATCTTTCAC
AAAATGCCTACGGGTTCTTAGCTAATGGTTTACCGCTTTATTGCGATACTAACAGCCCATTAGATTATGT
AATCGTTGGCGTAGTAGAAAATATCGGTGAAAAAGAAATTGTTGGATCAATTTTCTATGCTCCGTATACA
GAAGGTCTCGACTTAGATGACCCTGAACATGTAGGTGCATTTAAAGTTGTTGTTGATCCAGAAAGCTTAC
AACCATCTATCGGTTTATTAGTTAGATATGCTTTATCAGCAAATCCTTATACTGTAGCAAAAGATGAAAA
AGAAGCAAGAATAATTGACGGTGGAGACATGGATAAAATGGCAGGTCGTTCAGATTTGTCTGTTTTATTA
GGTGTTAAGCTACCAAAAATTATCATTGATGAATAAAACAAAGGGACCTTTCGGTCCCTTTTTATTTAAC
TTACCAACTCAATCCAAGCTGGACGAAGTACATCTTGTACCATTTTAACTAATTCCTTTTTAATCAAAGA
AGGATTATCTGCTTGAGTTAGAGTAATACCTTCACGAGAAGTTTCTTCCAAAATATCTTGAACAGTTAGC
CCCATCACCTTTCCAAAATCCTTTGGACCAATTTCGCCAATTTTAGAAATAACGTTATTTACGCGGTTCA
GTGTAACGTAACAAGCTAAAATTCCCACCAATTTGTTATCAGCTTCTGATAGCTCAACTTTAGCTTTAAT
AGGCTTATCAGACTTTTTCTTTTCACTAAATTTAGAGTTCTTGCATTTAATCGCTACACGATTTCCATTA
CGAAGCCAAGAAGGATAACAAGGTTTCAATACATATCCTTCAGCAGTAAATACTTCGCCTTTTGCTTCGG
CATTCCAAACGCATTTATTTGCATCAACTAATCCAGCATGGTCTACTGTAAAATTATAATCTTGGACGAC
AGAATCTAAATCATTTGGCAATTTAATAAGCTCTTCAAATTTACCGCGACCTAAAAGTGGAGCCATTTTA
AATTTAAATGTATTACAGAATGATTCCATCATATAATCATCTACATAAGTCACATCACCGCTTTCTGTAG
TAACAATAATGTCAAATACATAAAAATCTTTATCACAATAATCAACATTCTTCTGAATGCCAGGTCCAGC
GAATTCGCCAAAGACTTGATAAGATACAACCGCTGAGGTTTCCATAATATCTTGTACAGCTTTAATGGAA
TCAGCATAATTCTTCAAAATAATTTCATACCCAAAGAAATCTTCAGCAGGAAGAATCGGTCCAGTGCGTT
TAGCGCAAGTCACTTTATCACGCTCAATAATCAATGAGAAATTTGTGCCGTGAATCTTTTCACGAGCTAC
CCACTCCCCACCAGTCAATCCCAAGCTATAAAGTTTTTCAATAAATTTAGAGTTGTAATGATTTTCAAGA
CTGCTATACTTTTTAAACATAATTAATCCTCAAAATGTAATTTCTAACCAATCACCATCACGCTGATCAC
TATTGACTTTAAAGCTGAATCCTTCTTTTCTCAGCCAATCACCAATTTCTTCTGTAATCAATTTATCACG
AGCAATACAATAATAATTAAAATGTGTTTTACCTTGTTCAGCTGCTTTATTAGCAAGTTCTGAAAAATCT
TTAATAAAACACTCTAGCTTAAACTGTTTACTTTTTAATGCTTTTTCGCGTAATTGATTAGCAAAAGATT
CATTTTCATAAAGATCATACTGTTCCATTTTTCACCTTTTTATTGATATGTCTTTTTCTATAGACAACTT
TTTCTCGAGCCCATAATACAGCCACTTCTTTTGCCTGTAAGTTTAATTCACGAGCAATTTCAATGAATGA
CTTTCCAGACTCATGAAGAGTAAACACCACAACCTCAGTTCTCATAATCAATCTCATGTTATCGAGTTGG
TGCCATTATATACATCATTTTCTGATTGTGTTTTGTGTGCTTTCAAAATGAAGAAAGGGGCCGAAGCCCC
TTATGATTATGGATAGGTATAGATGATACCAGTTTCTAAAGCAGTTTTATGAATGATGTATCCATTACGC
GATTCTTGGACATCAACTTCTGGATAGTCTTTCATCATCTTCTGGAGAGTGTAACGATGCAGGTAATATT
TACTATCTGGGTCGTCAGTTTTCCAATCTTTACCTTCTTCGGTCATTTTTTGGATTTCATCCATAACCCA
CCAACCGCACCAGATGTAAGCTGAGCTACGGTGTGGAAGAGGATGAACATAAGGTAATTCACCTTCTGGT
TCTGGAGCAACTTTAGCCGTGACTGTTACATTACCAGTTTTGGTAACGGTTACAGGGTTATAATCTGCAG
CAGTAACAGTTGCAGTAACTTCAATAGTTTGACTTCCAACAGATGAGGTATCGACAGTATATACGTTAGT
TGACCCTTCTACAGGAGAAGAATCTTTCTTCCATGAGTAAGTAATTTGTGCTTCTTCTGGAGCACCCGTA
ACATTAGCCGTAAATGTAGCCGAAGCATCTTGCTGAACATTAATAGAAGGAGGAGTCAATGTAACCTGTG
GATTCATTGTCTTCTTATTAACCGTTAATGATACTTCATTAGAAGTAACGCTTAGTGCATCATAATCTGT
CGCGGTTACTTGGGCTACGCATTTAATTCTTTTTACTCCACTTGTAGTTGGAGTATAGCTAAATGTAGAG
TTAGTTTCTCCACCAACTTGTGAATCATCTACATACCACTGATACGTAGCAGATGCTCCATCAGGTTGAG
AAGCTAAGGCAGCAGTAAATTGAACTGGGGTTCCAATCACTCCAGCCGCAGGACTAGCAGGAGTTACGGC
TAAGGTAGTCGTCTGTGTCTTATTTTTAACTGTGATAGTTGTTGTCGCTTCAGCCGTTTCCGGGCCTCCT
TCAGAAAGTGTATTTGTTGCAACTACTTTAATAGTCTTTTGACCGGCAGGTCCTTTTAGTACATAACTAA
AAGTTGCTTCAGCTCCATCTTGTGGAACATTATCTACGCTCCAAGCATATGTAATAGTTCCGCCTCCAGT
TTGACCACTGGGTGTAGCAGTAAACTGCTTAGTTTCATCAATAACCCCTGTAGGTGTTTTAGGAGTTATA
TCAACTGTAAAAGTCATAAGTTATCCTTATTTTAATGTTACGAAAGAAGAGTTGCGTGTTTCACGAATTA
AAACTGATCCATCGCGATTAATGTAATAAATTAAGCTAAATAAAGTTTGGTGTGCTGACGCATGTTCAAA
ACTAGTTGGGTGAGATTTCCAATCAGGAGTTTCAGCAATCCATTGATAAATCCACCAAGGAACAGTACAG
AATCCTAGATTTTTTCCAATCAGTTGAAGATTCGGACTAAAGTTTTCCGGAAGAGTAAATACAGACGGCT
TTTCAGATTCAATAATCTCAGCTACAGCCTGTTCGAATTTTTCTTCAACAAAAGGAGTATCTTCAATCAA
AACATCGGTATTTTCAGGAATTTTATCCGTTTCTACTACTTCAATTTTAATGTCAGATTTAATCGGAGAA
TCAATCAGAAGTGCTGCTTCTGGATTGACTTCTTCATCGTCATATTTTAATCCCTCTGCGGCATCAGCAG
CATCAATTAAGTCTTTAATAGATAACCCATCAGTCTCTGGCATAGGTTCACTAGCGAGCTTCTGGAGGGC
TTCTTCAATATCAACAACGATATTATCAAAAGATTTATTCTTTTTGACCTTTATACCAAACTGTTCAGCA
TATTCAGCTAATTTAGCTTTAGCTTCTTTGTTATCATCAAGAGCCTTCAGCTCTGCAATATAATCTTTAT
CTATCATAATATTTCCTCAGTATAAATATAGATATATTTATTACTCGGAAAATAGTATGTACCACTTTGT
ATATGAAACAACAAATCTAATAAATGGTAAAAAGTATATAGGAAAGCACTCTACTGATGACTTGAATGAT
GGTTACCTTGGTTCCGGTAAGGCAATTCAGCAGGCTATAAAGAAATATGGTGAAAACAATTTCTCTAGAA
CAATACTAAAAGAGTTTAAAACTTCCGAAGAAGCGTACATGTATGAAGAAGAAATTATAACTCCTGAACT
AATAAAAAGCAAAAATTATTATAATATGAAACCTGGTGGAATTGGTGGAATTGTTATGACTACAGATGTT
ATAGCAAAGATGAAAGAATCTTCCGCTAAAAGATTTGAAAACTCACCGGGCACGGTATTAGGTAAAACTT
GTTATACTAATGGAACTAAAAATATTTTTATTAAACCTGGAGAACTTGTTCCAGAAGGATTTGTAAAAGG
GATGGTTCATCCTAATAGAAAGTCCAGAAAAGGATGTAAAGTCAAACCGACTACCACAGGAACTTTTTGG
GTCAATAATGGCGCAATAAATAAATTAATACAACCAGACGGTATTATTCCCGACGGATTTATTAAAGGTC
GTCTCATGAAAAGAGATTCTAAAGGCAAATTTAGTAAGGCATAATTATGGATATTAAAGTACATTTTCAC
GACTTCAGTCATGTACGCATCGATTGTGAAGAGAGCACGTTCCACGAATTAAGAGATTTCTTTTCGTTTG
AGGCCGATGGATATAGATTTAATCCTCGCTTCAGATATGGCAACTGGGATGGACGAATCCGTCTTTTAGA
TTATAATCGTCTTCTTCCATTCGGCTTAGTCGGGCAAATTAAAAAATTCTGTGATAATTTTGGCTATAAA
GCCTGGATTGACCCACAAATTAACGAAAAAGAAGAATTATCAAGAAAAGATTTTGATGAATGGCTTTCTA
AATTAGAAATCTATTCAGGAAATAAAAGAATTGAACCGCACTGGTATCAAAAAGATGCAGTGTTCGAAGG
ATTAGTTAATCGTCGTAGAATTCTTAATCTTCCAACATCTGCAGGTAAATCTTTAATTCAAGCTTTGCTT
GCGCGATATTATTTGGAAAATTATGAAGGTAAAATTCTTATCATTGTTCCAACAACTGCTCTGACAACTC
AGATGGCTGATGACTTCGTCGACTATCGTTTATTCAGCCATGCAATGATAAAGAAAATTGGTGGCGGAGC
ATCAAAAGATGATAAATATAAAAATGATGCACCAGTCGTTGTTGGTACATGGCAAACTGTAGTAAAACAA
CCGAAAGAATGGTTCTCACAGTTTGGAATGATGATGAATGATGAATGCCATCTTGCTACAGGAAAAAGTA
TTTCATCTATCATATCAGGTTTAAATAACTGCATGTTCAAATTCGGTTTGTCTGGTTCATTACGTGATGG
CAAAGCCAATATCATGCAGTATGTTGGAATGTTTGGTGAAATATTTAAACCAGTAACGACTTCTAAATTA
ATGGAAGATGGACAAGTAACTGAGCTAAAAATTAATAGTATTTTTCTTCGCTATCCCGATGAGTTCACTA
CTAAATTAAAGGGAAAAACTTACCAAGAAGAAATAAAAATTATTACTGGGCTTAGTAAAAGAAATAAATG
GATCGCTAAATTAGCTATTAAGCTTGCGCAAAAAGATGAAAACGCTTTTGTCATGTTTAAACATGTATCG
CATGGTAAAGCTATTTTCGATTTAATTAAAAATGAATACGATAAAGTTTATTACGTATCAGGGGAAGTTG
ATACCGAAACCCGCAATATAATGAAAACCTTAGCTGAAAATGGTAAAGGAATAATTATAGTAGCTAGTTA
TGGTGTATTTTCTACTGGTATTTCAGTTAAAAATCTGCATCACGTTGTTTTAGCGCACGGTGTTAAATCA
AAAATCATTGTATTACAAACAATCGGTCGTGTATTACGTAAGCATGGTTCTAAGACAATAGCAACAGTCT
GGGACCTCATAGATAGCGCAGGCGTCAAGCCAAAATCTGCTAATACGAAAAAGAAATATGTTCATTTGAA
CTATCTTTTAAAACACGGCATTGATCGTATTCAGCGCTACGCAGATGAAAAATTTAATTACGTAATGAAA
ACAGTTAATTTAATAAGCTTCGGCCCTTTGGAGAAAAAGATGTTACTAGAATTTAAACAATTTCTTTATG
AAGCTTCTATTGATGAATTTATGGGTAAAATTGCCTCTTGTCAAACATTAGAAGGTTTAGAAGAACTTGA
AGCTTATTATAAGAAAAGAGTCAAAGAAACTGAATTAAAAGATACTGATGACATCTCTGTGAGAGATGCT
TTGGCAGGAAAAAGAGCTGAATTAGAAGATTCAGACGATGAAGTAGAAGAAAGCTTTTAAATTAAAAAAG
GCCCAACCAAAAAGGAAGGGCCAAAACTATAGACTAAAGGTCACACTATAGCAAAAGTTGTGTTTCATTT
AATTGTTCTTCCGAACTTTCTGAAACTGGTAGTTCTTTAATGTAATTATAGCAAGGCCCAGGATGTACAG
GACCTTTGTCTGTTTCAACAACCAATGCAGAATCGATTGGAGTTTTACAGACAACACAAATCTTATCTGA
CATGATTGTCTCCTCTGAATTATATCTATTTATACAACTCTCATATGCATATCAATGCCCATATCTTTAG
AATAAAAATATTCATCAAGATATCCGGCAAATTTTCCTTTAATATAAAGGACATCTTCACCACACGGGTG
GTCGGCCAGGATACGAATATCCTGACGCTTAAGATTATGCTTTTTCATTAAGAATTGAATTTCCGTTTCA
AATTCTTCTTCATAATTAAAAGCATCATCAATGCTATATCTCATTATTTTCCAGCCTCAAATGCTCGCAT
GTCTTGAATATGCTTAATAGCAAATCCACGTGATTTAATAGCATCAAGAGCTCCGCTACAGAAATCTAAT
AAAATCCCCCAATACTGCAACGAGGTATCAACCTTTAAAACATCCTTATCCGCTGATAGAACTGTCTTCA
TTTCTGATTTCTCGTAACGATCCATACTAAATTCATCACCATCTCCTCGTCCCGAGTAGTAGTCTAATCT
AGCTTTAAGAGCAACTTTTTTCTGTGCTTCAATTCTAAGCATTTCCTTTTTAATACTTGAATGCTTATTA
AGCCATTTACTATATAACATCACATTATTAGCTGCTTCATACTGTAATTTAGTCGAATCTATAAACACAT
CTTTCTTCAATTCTTCTTGAAGATCTTCTAATCTCATATTGTTCTCTATTCAATTGTTATTGGTTGTTAT
TGGATGGACTTAGATTCATTATACCACGTTTTAACGTGAAGCATTATACTCTATTACTGGAAGCCAGCTG
CAGTTTTATCTGCTCAATATCATCAGGATTATCGATGACCGAAAAGCGTATTTCTACTATCAGAGTATAA
TCGTCATAAACGGGTATCACATTAACTGCTAATTTATCAATACGTGGCTCATAGTTTCTTACTGCGCTTT
CGATATTGCGTTCAACCGTGTCAGCAGTAAGAGGAGTCATATTTTCAAAAAGCTGGTCTGATAAATCACA
TCCAAATTCAGGGTCAAACGGTCTTGAACCTTTTCTTGTTGTAATAATTCCCAAAAGACTGTTTTTAATT
GACCTTAATCCAAGCGATCTGGAAACGTCTTTGTTCCAATCCATTTTCATTTCCGGGTCAATATCAGAAT
AAAGCTTATTAATATTTGCCATTATAGTAACTCAAAGAACTCTTTGAGGCCTCTTATTACGTGAGCATGG
GTTTTTCCACACTCTGGACACTTAATTGGAACAGCCAAATAAACGGTAGGCTTTAAAAGCATATCTTTTA
TAGCTACAATATCTGACTCTGTGATGATAGAATATAAATCTTCTAGTTCCTTTTCATTTAAGTCTTCAAC
TGGAATGCTTTCCCCGTTAGCATGAATCGTTTCTATACATGATACTATCATGTGGGCTATATTTTTATCA
TCAAAAATTTTAGGGTATCGGAATTTAATTTTAATGTCACCTAGTGTATACCAGAGGTCTTCTGGTGCAT
CTATTTGTGTATGTAATAGATTTATATGGGTTGGTATTTCAGTTCCACAGGTGCACTTCCAGGAGTTTTC
GTGATTAACTTCACCGAGAGAATGTGCCCATAAATGAATCAACAATAGTTCTGATTCTTGGCGGTTTAAA
TCTTTTGCATTTGTGCAGTCTTTGATTAGCTTTTTAACAATTACTTCTACGGAACCATTATTTTTGGCAG
TAATAAGTTCTAGATATTCTTTAAGCGTGAATGCGCGACAATTGATTATTTTAGAACCAACTCTCACATC
AAATTTGTATTCATACATATTTAGCTCCTTTATTTATCATATTTATAAATAGAATAAAAGGAGCATCTAT
GGCAAACATTATTCGTTGTAAATTACCAGATGGTGTTCATCGTTTTAAACCATTTACGGTAGAAGATTAT
CGAGATTTTTTGTTAGTTCGAAACGATATAGAACATCGGTCACCACAAGAACAAAAGCAAATAATTACTG
ATTTAATTGATGATTATTTTGGAGACTATCCGAAGACTTGGCAACCATTTATATTTTTGCAGGTATTTGT
AGGGTCAATAGGTAAAACTAAAAGTACGGTCACATTTATATGTCCAAAATGTAAAAAAGAAAAGACAGTT
CCATTTGAAATATATCAAAAAGAATTAAAGGACCTTGTTTTTGATGTAGCTAATGTTAAAATTAAATTAA
AGTTTCCTTCTGAGTTTTATGAAAATAAAGCAAAGATGATTACTGAAAATATTCATTCTGTTCAAGTAGA
TGAAATATGGTATGATTGGAAGGAAATTAGCGAGTCCAGTCAAATAGAACTAGTTGACGCCATCGAGATA
GAAACATTAGAAAAAATTCTCGATGCAATGAATCCTATTAATTTAACTCTACACATGTCATGCTGTAATA
AGTACATTAAAAAATACACTGATATAGTAGACGTGTTTAAGCTATTAGTTAACCCAGATGAGATATTTAC
TTTTTATCAAATTAATCACACACTCGTAAAAAGTAATTATAGCTTAAATTCAATAAGTAAAATGATTCCT
GCCGAGCGCGGATTCGTATTAAAACTGATTGAGAAGGATAAACAATAATGAGTATGTTGCAACGCCCCGG
ATATCCAAATCTCAGCGTTAAATTATTTGATAGCTACGACGCTTGGAGTAATAATAGATTTGTTGAATTA
GCTGCTACTATTACCACATTAACTATGCGGGATTCTCTTTATGGCCGAAATGAAGGAATGCTGCAGTTTT
ATGATTCTAAAAACATCCATACAAAAATGGATGGAAATGAAATAATTCAGATTTCTGTAGCTAATGCAAA
TGATATTAATAATGTTAAAACACGAATTTATGGATGTAAGCATTTTTCCGTGTCAGTAGATTCAAAAGGT
GATAACATCATTGCTATTGAATTGGGAACTATTCATTCTATAGAAAATCTTAAATTTGGTAGACCATTTT
TCCCTGATGCAGGTGAATCTATAAAAGAAATGCTTGGTGTCATTTATCAGGATCGCACATTATTAACTCC
AGCAATAAATGCTATAAATGCTTATGTTCCTGATATTCCATGGACTAGCACATTTGAAAACTATTTGTCA
TATGTAAGAGAAGTTGCTCTAGCTGTAGGAAGCGACAAATTTGTATTTGTATGGCAAGACATCATGGGCG
TTAACATGATGGACTATGATATGATGATAAATCAAGAACCATATCCAATGATTGTCGGTGAGCCATCTTT
AATAGGTCAATTCATCCAAGAATTAAAATATCCATTAGCATATGATTTCGTTTGGTTGACTAAATCGAAT
CCTCACAAACGTGACCCAATGAAAAACGCTACTATCTATGCGCATTCATTTTTAGATTCTTCAATACCAA
TGATTACTACAGGAAAGGGTGAAAACTCTATTGTGGTGTCAAGGTCAGGTGCTTATTCTGAAATGACTTA
TAGGAATGGATATGAAGAAGCTATTCGTCTTCAAACTATGGCACAATATGACGGCTATGCTAAATGTTCT
ACTATCGGTAATTTTAACTTGACTCCTGGTGTTAAAATTATTTTTAATGATAGTAAAAACCAATTTAAAA
CAGAATTTTACGTTGATGAAGTTATCCATGAATTATCCAATAATAATTCAGTAACTCATCTATATATGTT
CACTAATGCAACGAAACTGGAAACAATAGACCCAGTTAAGGTTAAAAATGAATTTAAATCTGATACTACC
ACTGAAGAAAGTAGTTCTTCCAATAAGCAATAAAGAAGTTTCTATTCCTAAAATGGGTCTTAAACATTAT
AACATTTTAAAAGATGTTAAAGGTCCTGATGAAAATTTAAAACTTCTCATTGATTCTATTTGTCCGAATT
TATCACCGGCAGAAGTTGATTTCGTTTCTATTCATTTATTGGAATTTAATGGAAAGATTAAATCTCGTAA
AGAAATAGATGGTTATACTTATGACATTAATGATGTTTATGTATGCCAAAGATTGGAATTTCAATACCAA
GGAAATACATTTTATTTTAGACCTCCTGGAAAATTTGAACAATTTTTAACGGTGAGCGATATGTTATCTA
AATGCTTACTTAGGGTCAACGATGAAGTTAAAGAAATTAATTTTCTTGAGATGCCAGCATTCGTTTTAAA
ATGGGCAAATGATATTTTTACAACTTTAGCAATTCCTGGCCCTAATGGTCCAATAACTGGAATTGGCAAT
ATTATTGGATTATTTGAATGAAAAAGCCACAAGAAATGCAAACGATGCGTAGAAAAGTTATTTCAGATAA
TAAACCAACACAGGAAGCGGCTAAATCCGCTTCTAATACTTTATCTGGGCTTAATGACATATCTACGAAA
TTGGATGATGCTCAAGCTGCTTCTGAATTAATAGCTCAAACTGTCGAAGAAAAATCGAATGAAATAATTG
GAGCAATTGACAATGTAGAAAGCGCAGTGAGTGATACATCTGCCGGTTCTGAGTTAATTGCTGAAACTGT
CGAAATTGGCAACAATATTAATAAAGAAATCGGTGAATCGCTCGGAAGCAAATTAGATAAATTAACAAGT
TTACTAGAGCAAAAAATCCAGACAGCTGGAATTCAACAGACTGGAACTAGTTTAGCTACGGTTGAAAGCG
CTATTCCTGTTAAAGTCGTTGAGGATGATACTGCTGAATCTGTGGGTCCTTTATTACCAGCTCCTGAAGC
AGTTAATAATGATCCTGACGCTGATTTTTTCCCTACCCCTCAGCCAGTTGAGCCAAAGCAAGAATCACCA
GAAGAAAAACAGAAAAAAGAAGCATTTAACTTAAAATTATCTCAAGCTTTAGATAAATTAACGAAGACTG
TTGATTTTGGATTTAAAAAATCCATTTCAATTACTGATAAAATATCAAGCATGCTATTTAAGTACACCGT
CAGTGCTGCTATTGAAGCTGCTAAAATGACTGCAATGATATTGGCTGTTGTTGTTGGAATAGACCTTTTG
ATGATTCACTTTAAATACTGGTCAGATAAATTTTCAAAAGCCTGGGATTTGTTTAGTACAGACTTTACCA
AATTCTCTAGCGAAACCGGAACTTGGGGTCCTTTATTACAGAGCATCTTTGATTCTATTGATAAAATTAA
ACAACTTTGGGAAGCGGGAGATTGGGGTGGATTGACAGTAGCTATTGTTGAAGGGCTTGGAAAGGTTCTT
TTTAATTTAGGTGAACTTATTCAATTAGGTATGGCTAAATTATCTGCAGCAATTCTTCGAGTCATTCCTG
GTATGAAGGATACTGCTGATGAAGTAGAAGGAAGAGCATTAGAAAATTTCCAAAATTCTACTGGAGCATC
TCTCAATAAAGAAGACCAAGAAAAAGTAGCAAATTATCAAGATAAACGAATGAATGGAGACCTTGGCCCA
ATAGCAGAAGGACTAGACAAAATCTCTAACTGGAAAACTCGTGCATCTAACTGGATTCGTGGTGTAGATA
ATAAAGAAGCGCTGACTACCGACGAAGAGCGTGCGGCAGAAGAAGAAAAATTAAAGCAACTTTCACCGGA
AGAAAGAAAAAATGCTTTAATGAAGGCTAATGAAGCTCGTGCTGCGATGATTCGTTTTGAAAAATATGCC
GATTCAGCTGATATGAGTAAAGACTCAACGGTTAAATCAGTTGAAGCTGCCTATGAAGACCTTAAAAAAC
GGATGGATGACCCGGATTTAAATAATTCACCGGCAGTTAAAAAAGAACTTGCTGCTAGATTTTCTAAAAT
TGATGCTACTTATCAAGAGCTCAAGAAAAATCAGCCTAATGCCAAACCTGAAACTTCTGCTAAATCACCA
GAAGCGAAACAAGTCCAGGTGATTGAAAAGAACAAAGCACAGCAAGCTCCTGTTCAACAAGCATCTCCTT
CGATCAATAATACTAATAATGTTATTAAGAAAAATACTGTCGTTCATAATATGACACCTGTAACGAGCAC
GACTGCTCCTGGTGTATTTGATGCGACTGGAGTTAATTAAGGAATAATATGGCAATTGTTAAAGAAATAA
CTGCTGATTTAATTAAAAAGTCCGGTGAGAAAATTTCAGCCGGACAGAGTACTAAATCAGAAGTAGGAAC
TAAAACATACACAGCCCAGTTTCCAACTGGGCGTGCTAGTGGTAATGACACTACAGAGGACTTCCAGGTA
ACAGATCTATATAAGAATGGATTATTATTTACTGCATACAATATGTCATCTAGGGATTCTGGAAGTCTTA
GATCGATGAGATCTAACTACTCTTCTTCATCTTCGAGTATTTTACGTACAGCTAGAAACACTATTAGTAG
TACAGTATCAAAACTATCAAATGGATTAATATCAAATAATAATTCAGGAACAATAAGTAAATCTCCTATC
GCAAACATTCTTTTACCGAGATCTAAATCTGATGTTGATACATCATCACATAGATTTAATGATGTTCAAG
AAAGCCTTATCAGTAGAGGCGGAGGTACTGCTACTGGTGTGCTAAGTAATATTGCTTCAACCGCAGTATT
TGGGGCACTGGAAAGTATAACACAAGGTATAATGGCTGATAATAATGAACAGATTTATACGACAGCCAGA
AGTATGTATGGTGGTGCTGAAAATAGAACTAAAGTGTTTACATGGGATTTGACTCCACGTTCAACAGAAG
ATTTAATGGCTATTATTAATATCTATCAATATTTTAACTATTTTTCTTATGGTGAAACGGGTAAATCTCA
ATATGCTGCTGAAATAAAGGGGTATTTAGATGATTGGTATCGTTCTACGTTAATTGAACCTTTATCTCCG
GAAGACGCAGCTAAAAATAAAACACTATTTGAGAAAATGACATCGAGTTTAACTAACGTTCTAGTAGTTT
CAAACCCGACAGTTTGGATGGTGAAAAACTTTGGCGCAACATCTAAGTTTGATGGAAAAACGGAAATATT
TGGTCCATGTCAAATACAGAGCATTAGATTTGATAAAACACCTAATGGTAACTTTAACGGATTAGCTATT
GCTCCAAACCTCCCTAGTACATTTACTCTCGAGATTACTATGAGAGAAATTATCACGTTAAACCGTGCTT
CTTTATATGCGGGGACTTTTTAATGTATTCTTTAGAGGAATTTAATAATCAAGCAATAAACGCAGATTTC
CAACGTAATAATATGTTTAGCTGCGTTTTTGCGACAACTCCATCAACTAAAAGCTCTTCGTTGATAAGTT
CAATTAGCAACTTTTCTTATAATAACTTGGGCCTAAATTCAGATTGGTTAGGATTAACTCAAGGTGATAT
TAATCAGGGAATTACCACGCTAATTACAGCTGGCACACAAAAACTGATAAGAAAATCAGGAGTCAGTAAA
TATCTTATTGGTGCCATGAGTCAACGTACAGTTCAAAGTTTATTAGGCTCATTTACAGTTGGTACATATT
TAATTGACTTCTTTAACATGGCATATAACTCATCTGGATTGATGATATACTCTGTAAAAATGCCAGAGAA
TAGATTATCCTATGAAACTGACTGGAACTATAATTCTCCTAATATTCGTATAACCGGAAGAGAATTAGAC
CCTTTGGTTATTTCATTTAGAATGGATTCAGAAGCTTGTAACTATCGTGCAATGCAAGACTGGGTTAACT
CCGTTCAAGACCCAGTAACTGGACTGCGTGCTTTGCCACAAGATGTCGAGGCAGATATTCAGGTTAATCT
TCATTCTCGCAATGGATTACCTCATACTGCGGTGATGTTCACGATGCATTCAATATCAGTGAGCGCTCCT
GAGTTATCATATGATGGAGATAACCAAATAACTACATTTGATGTTACTTTTGCGTACAGAGTGATGCAGG
CTGGAGCAGTTGATAGGCAACGTGCGCTTGAATGGCTTGAATCTGCTGCTATAAATGGTATTCAAAGCGT
TCTCGGAAATAGTGGAGGTGTTACTGGACTATCTAATTCGCTTTCACGACTTAGTAGATTAGGGGGAACT
GCAGGAAGCATTTCAAACATTAATACTATGACAGGAATTGTCAATTCGCAGAGTAAAATATTAGGAGCAA
TATAACAATGGGGACCGAAAGGTCCATATTTTTATTTACGGAATGAAATGAAAGCAGCAACTGAAGCAAC
TAAACTGTCTTCAATATAAACTTCAATTTTTACAGGAGCTTCTGACTCAAATTTACCTGTTACTACACCC
TGAAAAATACTTTCAGTCTGTTCTGGCTTTGAAAAATTTTCAGAAGGAAAAATTCCGAACTTTTTATCTG
TTCCAAAAATTTTGATAAATTCATCGTAAACCGCTTCGTTAAAAGCATTATCAGCAGGAATAACGCCTTC
AACTACAAGTTCTTGACCTAAAAAGCGTAAAGAAGATTTCATTTTGTGTTCCTCATGTTATGTTAGTAAG
ACTACTATAACACAACACGAGGGACTTGTAAACTACATTTTGAACTTTTTAGTACGCGTAATAGGCATGC
GTCGATTTTATACTGTTTCATTGTTTGAAGAGCAGTATCAAAAACAGCATTAATGACACCAATTGGATTT
CCACCCAAGTTTTTAAGCTTAACTAATGATAGTTTCTCATTAACACCTATCATTACGATATGCATCATTT
TATCGCCTGGTTTAACGTTTTATTTGTATCACCGCCAGAGGTGTATGTACAAAATCTAAATCCAGGAAGT
ACACTTTCTTCACCGGCTTGAATAGCAAAAATTTGTGGTATTTTATGCTTAGGATTTAATGTAACAACCG
GCGCCGAAGCGCCGTCAAATAATTCTGTAATAAGTTCCATGATTTATCCTTGAACGAACTTGTAAGGCAT
GTTTGCAATATCTATGCAAGACGCAATAATTCCAAGAGATGATTCTACTTTCTGTTTAACATTTGATGAA
ACAAATGATGCAAAGTCAACTCTATCTTCTATATCACCTGTCATTGTAACCAATTCACCAGTTTCCATTA
AATGGTCTCCGTCATAGATTACCGATTCGGTGAGTTCTTCTGTGGTCATAACTTCAGCTTGAATAAGCTT
GTTGTTAGTTTTAACTGTTCCATCATTGTAAGATGCATCGGTTATTTTATTAACTTTGACCATTAATCCA
CGTGGCAAAATGACTTCCATTTCATTTGAAGGCGCTAAACTTCCACCGGGTAAAACAACATTGACCTTAT
CAGCCCCAGTAATAACCCATCCAATTCCAACTAAATTATCGCTAGAATTTACAAGTCCTTCATCAGTTTT
ATCAATAGAAACGCTTAAACGCTTTTCGTCTGGTAAAACACCTATAGATGAATCAGTCATCCAAGTACCA
AAAATATTTGGATATAATGATGTTGACACAAAGTTTCTAAAATAAAAAACTCGATTTTTTACCATTGCTT
CGTATATTGAAGGTAACATTCGTTGTGAACGATACAAAGTAATACCTTTTGGTAATCGTTCACCATTTTT
AAAGGCTGAATCTAAATTATCAATAGCTTTTTCTATGTCAGATGCTGTCAAAATACTTGTACGCTCATCT
GGATTATATAATCCCAAAAGAGCATTATTTATGTCTACATATCCTGAACCTACGTATTCACGAATTCCGC
GTTTTTGCGCTGGTGTGTATTTAGATGAATCTTTATTTTCGACTATAGGATGTAATGACCATCCAGCGGT
AAGAGCATATCCACGTAATTCTCTTCGAATAATCTTTGTTTTTATTGCATTCCAAGAATTTTGTACTAAC
TCATTTGCAGCATCTTGGTTTAAATGCGAATGTTTGTTAATATTTCTTTCAAACCAAGCACCCTTATATT
TTTCTAAAGTATCATCGACGATAGAAGCAATAGTTTCTAAGGTTTTTATTGAAGTAATAGATTCTTTTCG
TAATCTTTCTAAAGCTTTATTTTTTATTTCTGCGTTAATAATGGATTCTTTATCTTCAGGAATTATAGAA
GCTTCTCTGAATGCCATCCCAGCTGTAACACTGCTAAGAGCATTCTCTAGTGCAAATCCTGAAGTAGAAA
TTACCGTTAATTCATTAGAATCAGAAATTAAAGGCGCGTCCGCTGGTTTATTTAATTCGGCCGCTGAAGC
CTCAAATTTTTGAAACATTGGTGTTTCAAATCTAGAAGATTCCAATGACTGACTTTGCGCAATTGCTCTA
CGGGAAATTTTAACTTTAACGATTACAGCTTGGTCAGAACGTTTATCATTTTCTTGCGCAATAGATGCTG
CAATTGCCTCATTTTTAGTTACTTGAGCCCCAGTATCTTTATTGATATAAACATCACCGACCTTCGATTC
AACTTTAGTAAAGAGCTCGGTACTAATTTCCGGAACTCCTGGAATGTCTTCTAGTGATACATTTTTGCGA
TGTATAAGAATATATGCATACTTTTTATCGTAATCCCAGAGTTCCTTAAGAAGGACGTATCTACCACCTG
AACGACTACGGATAAGTCTATCAGCAATAACTTGAATTTGTCGAGCTTGGCCAGCAGTTTTAGACTTAAG
AATACGGAGCATACAGGCATCAATTTTATACTGGCGCATTGTTTGCATTGCAACAGTAAAAACTGAATTG
ATATAATTAATTGGGCTTGGACCAAGACCTTTTAATTTAGCAATTGAACCTTTAGCAGTTAATGTAAAAG
GAACAATATGCATCATTTTATCGCCCATCTTTAAATCACGATTAGTATCACCTCCAGATGTATAGGTACA
TAAACGAAAGCCTGGTTGTTCAATTGCATCATCAACATGAACTGAAAAAATTTGCGGTATTTTCTTCTTT
GGATATAAGTTTGTAATTGGAAGAGTAGTATCTTCGTCAAATAATTCTGTAATAAGTTCCATCATATCCT
CTCTAGTGTTTATTCTATTCTATTTATAAAATTAAAGGCCCGAAGGCCTTTAATAATCTATTGGTAAGAG
AGTACGATATATTTCAAACTTTGGACCTTTTTCATAAGCATCAAATGTTTCTGTGAATTTATTATAAGCA
TATGCATCTATAAATTCAATCATGATTTGTGATACAGAAGTAGAAACATCTCCACCTTCTTTTTGAGCCA
CGACAATTGTTTCTAAGTAAGCTTTCATAGACCAGTTACCTCATGAAAATCACCAAATACATCTTCGAAT
GTATTAGCTTTAGTTTTATCTTCACGTAAACGAATCGCAATCGGAAGAAATAATTTAACGTAATCAGTGC
GGCCATCAGATTTTAACCAACCGTTGCATTCGCACTCTAGAATTTTTCCAATATAATAATTTTGGTTTTC
CATAATGCGAGTACGGTCAAGTTCATGCGATTTTACACCGGCTTTATCTTTTAAGCCTGAACCAGCATTT
ACCTTAATTTTTCCACACTCTGACTCAAGAATAAATCCACCCGCTTTAGTAGGGTCTTTACGGTGAGGAT
AAATTCCTACAATTTTTAAATCAACATCAATTACTTCTTTAAATTTATAAAGATTTTTTGAACGAGCATT
TTCCCATAATCCATCGATATTTTTGAGAATAATACCTTCAAGACCTTGGTCAATATACTTTTTATAAATT
ACCTTAGCTTCATCTAGGTTATTTACTACCTGGTTTTCAATTAAAATTACTTTATCATATCCAGATGTCA
TTTGTTCTAGTTTAGAAAAACGTACATCATATTTCAAACGAAATGCAGGAAGACTGTATATTTCTACCAA
CGGGACATAATCCCAGACCTGAAACTTCATGCATTGTGCTTCTTTTTCAGAAATGGTTCCCTTTAAAGAT
TTATTGGCGATTCCATTAGAAGCAGTACGTGATTCAGCTACTTCGGCGAATTCTTTAGCTTTACTGTTTT
CAGGATAAGCATCAAAAAGAAAATCTAGGCCTTCTGGCTCCTTTTTAACTTGCTCATGGTATACCAATTC
GCCATCAATCAACACACCTTCTGGATGAATCTGGCGGGCTTCAGCGGTCATTTTAATTAACTCTTCCTTA
AGAAGATCTAATCCTAGATATTCATTACCAGCTCGTGATAAAAGACGAACATCATCTAATTCATCACCTC
TAACTTCAGCAAAACACCGAGCTCCATCAGCTTTTAACTGAGCAAAGGCTGGAAATTTGATATTCTTATT
AATGCCTTTTTCATCATAAGAACTTGCGAGCATTTGAGGTTGTTCAGGAATTAAACCTGGCCAAACTTTG
TTTGCAATAGATACTGAAGCACCACATTCAAGGTCTCGCATCATCACTCGACGCAAAACTTCAACATCAT
CTTTTTTACCATCGGTGATATATCCAGTTAATTCCTCAATTGCTGCATTTCCAGTCAATTTCCGAGTAGC
TAATGTGAATTCAATGAAGTCAAGCATATCGGTAAGAGTCAACATTCCAAAACTCTGGGTAGCAATACCA
GGTTTAGGCCATTTCTTGATATAATACTGTAACCCACGAGAATAAGTCAGACGATATACTCGTTTAAGCA
ATTCATTATCTTTATTCTTTTCAAGAATTGCTTGCTTCTGTTTAGTTGAACCAATAGATGCTATTTCGTT
CAGAATTTTAAGAATCATTGTTCATCCTTTAGAGTTTGGTTTACAGCTCTATTATAAATCAATTCATCAT
TAAGCTCAGTCAAAGACCTGTGGTACGTGGTTCTAACTTTATTTCCTTGCATCCAGTGCTTGATATAAAT
GAAACCTTGCTCTACACATTTTTTAAAAATTCGTTCGTCTTTTTGAGCTCGGAATTCTGGATTGCATCTA
AAAAATTGATTTACGTGACCGTAATCACGTGTAGTATTACCTTCATTTTCATAAATAGTGTGAACAACAA
ACATTAGAATGCTCCTTGGAAAATATTATCACCACAAGTAGGTCTATTATACAAATACTCTATACCGCCG
GGCTTAATATAGTTCCATGTCTGAAACGGATGCGTCTGATATGGATGATATGGATTATAAGGATTAAATC
CAGGAGTTCTCCAGGTAGTGTTCCAAGGAAAAGAATCGTTTTTAATCATCTTTTCAATTACATCTTTTAT
AGCTTTTTCACTATCAAAACTTTCTTTATTTTCCTTTGGTGAAAAAAGCTTATTCTCTACATCGTTCCAT
GTATAAACTCGCTGAGCAGTTTTTGGAATATTGTCACGCTCTCCTCGAGCCATCCAATACACAGGAACAC
GTAATATTTCACTCGCGTGATCGCAGTGGTGAGCGAGATCGTCAATATAACAAATTACGTTATATTTCTC
TTTTGCTTTTTTGAACAACTCTTCTTTTGAAGAATCATGACTACACATCAGTACTTCTGAGAAAGCACCA
GGAAAAAGAGCATTCAAATTAAATTGACGATTTAATAGAGCGTCAATAGAATCACCCAATGCTGTAACAG
CAACAAAATTATAATCTTCTTTTAATTTGTTAATTACACACAGAGCATCTTTATATGGAGACAAGTAACG
AATAAAATCCGAACGATTGTATTTTTCAATTAACTTGACGCCAAGTTCTTCATCACAGTTAAAGAGTTTA
CCAGGAGAAATAAATTTCTCATCTTGGATCATTTTTAAAATATGTTCTAACGGAAGATTATATTTCTGAG
CAAAATAAGGAAGGCCTGATTGCCAGCTTAAACATACTCCATCAATATCAGTTAAAATAGTAGGCTTCAT
AGAGAGTCTCTTAATAGGTTTAACACATCAATAAATTCAGCTTCGGTTAGTATTGTATCATCTTTTGTTA
GACCACTAGCAATGCTGTGCTTCAAAACTTTTCCTTTCGAGGCTTGTAATGCATCACGAAAGCCCTTGTT
TTGAATCGCTGCTTCAAAATATGCATTTGTGTATAATTCTTTCCACGCCGGGGAGTATCTTGAAAACGGA
ACTCCAAGCCAAAAGAGGGTCCCACGGTCCTGAGCTCTAGCATAAGACCTTCCAGCTTGTTGGGCGGCAA
GCCCGGATAACCCAAATATACGACGTTGTTGTTCAACATTTTTCACCTTACACCCTTGGAGGAATCCTTC
AAGACCTCCAAATTGAATACCATCCATAACGAAAGGCCATTGGGCGAAATTACTTAATGCACATGATGGC
CACCTAAAATTGCTTCTAATCTCTAACTCAGACATTTTCAATGCTTATAATTTCAACATCAGCCCAATGA
CCATAGCAAGGAAGACGAAATTCAACTGGCCAGTTAGGGTCCCTTTCTAATATAATAGACTCAACTTGTG
GTTCTTCGTGTTCATCAGTATACGGATTTTTATCTGTTACTTTATATGTGACCTTAATGTACTGAATTCC
AAAAATCTTATTAATTATATTCATACTAATTCCTTTAATCCGTAGATAGGAGATAATTCATCACCCATAC
GAAGGTCTTCATTTCCATCTACCCAGGAAACAATATAAGCCTCTTTTATTTGAATACCACTCCATTTAAA
TGGAGGTAGAACCTTAGAAATTAATCCAGGTATACCAACTCCCTTTAATTCAACAGTTTGACCTAAAAAG
AATTTCATTAGAACCTCATCTGAAAACCGTGCGATTTAACATTACCGCCGCCAATATCAGAAATGTTAAT
TTCACGCGCAATTGAAGGGTCAATGTCAATTTCACGATTGAGTTTAGTGATAGCTAAAGTATCACGCCCA
TTTACAGTACGGAACTCTAAAGGACAAACCACATCAACGTAGTTTTTAATCGATTCGCTAGTAAGTTGCA
AATGTGCAGGAAGGTCTTTAGGTGCCTTTGAGAAAACCACTTCACAGAAATTTTTGCGGGTATCAAAATA
AGTAGTCATAAACATAATATTTTCCTCAGTAAGGGGCTGAAGCCCCTCATTTTATTTTAAATATCAAATT
CATTAAGAACTACATCAAAGATTGCTTCAAGATGCTCAGGTTTAGCTCTGTTACTCAGAATATGACGAAT
CCAAGTTTTAACTAAGAGTTTACGATTAGCACCATTCCAGCAAGGATGAGTCCCTAAATCGCGTTGGCGG
AAATCATCATCCAGAGCGATTTTGAAGTTGGAACCTTTCATCGTGATTGAAACCGTGATACCGTTTTCAA
ATCGCATATAAACGTAGTTAGGAGTCATATACTGTTCAATTTCGCATACTGATCCATTTTGATGTTTCCA
GAGGCAAATAGTATCAATAGAACCTGCAATACCATTAGAAACATATTTACGTTCAAAGTTAATGTAGTTC
ATTTTTATTCTCCGAGATGTTTAATTGCGGTACAGGTATATAATATCATATCCTGTACCAAAGTAAACAA
TTATTTTACTACTTTCCAATGCTGCATGTCAAGTTTACCAACTTTTTTCATCTTCTCAATTAAGCGTTCT
GCACGTTGGCGAGCTGTAACATAATGCCATTCGCCTAATTCATTTTGTTCAATTTTTCCAACGATTACTG
TATTCAATTCATAAATCCAACCAGTAAAGAAATTATGAACTTGAATTGTAAAGGTGAAATCTGTTCCCAT
ACCTTCTGTTGTTTCTACTTCAATAATATCACCTTCAACTGCCATTAAGAACCACATAGTTTCATCATAT
TTACCATTGAAGCATTTAGTTTTAACTGCAGCGTTCAGATTAATCGTTTTCATTTTATTCTCCTTTGTTT
GTGTAAGATAATACTATCACAAAGGAACTATACTGTAAACAACTTTGTGCAATCTTTGGAAAATAAAAAA
GGACTCCCGAAGGAGTCCTCAACTTATGCTTTCTGCTTACCAAAACGAGAAGCATCATCTCGAAGAACCG
CACGTGCTCGGCGCATGATCTTCTCAACAGTTTGATTGATACGAGAGTTCGACCCACGCTTGTAGCCAGC
GCGTTTAGAATCACCAACTTTCTTTTCAACTGCTTTCTTTGCTTTAGCTTGTTTTGCCATTATAAATTCT
CTTTTAAATGAAAATGCAGGACTTATTGGCATTGCCTGCGCAAGCCCTCAAGGGGAACATAGGTTTTTGG
ATATTTAACGACCAGGATAACCATAAACCCGTCATCATTCACATTCAAGAGGTACACCGTAAAACTGTCG
GGGTCTTAAAACTATAATGATTCGCAAATCATTAATCAGACAGTTCGACGGCTCCTCGATTTAGCTCACA
CTAAGGCAGTGAATCTCCAATAAATTACTTCAGTGTTACCACAAAGTGACGAACTGCTTTTCGTGCAGCA
GAAGCCAGAGGCTTAGCATATTTAAGTTCATCTTTTTCCTGAAGCTCAGCAGCTAATGCAGTTTGAGCAG
GATTCAGATGTTTGAAATAACGCAGGATTTCAAGAGCTTCGGCTTCAACATCAATAGATGCGCCATAGTT
TTCGTGACCATTATTCCATGCGTTTCGTTGCAGTTCAAGAGCGTGTTGTAATTGTTTAATCATTTAAAAA
TTCTCGTTAGAGATTAAAACTCGGTAGTCACGTTCTTCTGAATTTCATCTTCTTTCGACAGATCTCTCAG
TTGTAGACTACCACATAGAATTGTTCGGTTAACTTATTATTCCGACACCCAATTCATATTATTATTTATA
TCACTTATAAAGACACGGAATAGCTTTATAGTGACAGGTAACGAATTTTTGTTTAATTTCTTTTGGCTGC
TTAAGACCCAGAGCTACAAAAGGATGCGGAACATTTCGAATTTGACCAACTGGAAGAGAAGTCAAATCAC
CAACTTCGCAGAAACCTTCAGGAACATCAGGACCGACAGAGTGAACTACACACAGTTCAGGAACTTCACC
TTGAACACGTTTACCGATAATAAGTCCTGATTCTGTAACTTCTTCATCACCGGCTTGTGCAGGTTCAGAA
ACTAAAATAACATATTCACCGACAGCACGAATTGGTAGCTGTTGTACTTCAGACATCGTTTTTCCTTTTT
GTTAACAGATGAATTAATAATAACAAATAGTTCTTAAAGCATTTATTTACCAATAAATTGAAGCAAATGC
TCAACTTTCATACCATTAACGGAAATCAATTTGTCAATAGAAAAACCTCGCCACGCACCAAGCTCAACAT
CAAATACTGGAATCATGTCAGTAGATTCTTTCCGAGTAGATTCAGTCAATTTGCCAGTTTGCATGGTTGG
CATAAAGTCTGCATCACGAGTACCTTTCATAGTACGAATAGTACCATCAGACTTTTCAAAAACTACGTTT
GAAACACCCATGGACAATTTAGTTTTCAAAATTTCACGAATTGCTACTTTCTGCTCAGTTGTCAGTTTCA
TTTATTTACCTATTACAGTTTTAATATGAGTTGTTCCACGTTCTTTAAGGGTGGAAAGTAATTTTTGGCA
TTTTTCTAAATCAGATTTCCAACTATATGGTCTATCAATACAAACCCAATTTGTCTTATAATACTGTTTC
CATTTAGAGAAAAAATATTTCTTATACTCTACAGCAAATGAGATGTTCTCGTTAGAATAAGAACTAATTG
CTGTGAGTTTTACCAAACGAAATTTCATTATTCACCACAGAATTCGTTGATATTTTCCCAGTTTAACTTA
TTCAAGTTTTTCTTAGGAACATTAAACACTTCAATACCTGCATTTCGCAGAATATCATCCCAACCGGGTT
TATTTTTGTCGTATGTTTCACAATAAACCAGCTTTTTAATACCAGATTGAGCTATCGCTTTTGCGCAATC
TGGACAAGGAGAAAGTGTTACATACATAGTAGCACCTTCAATAGAAGAACCATTTCGTGCAGCAAACAAA
ATTGCATTTAGTTCAGCATGAATTTCATTTTTAGATGACCATTCCGAGTGAGCACTACGATGTTCTTTCG
CCAAAACAAAACGATCAGTTGAACCAAATGATACGCATTCAGGCTTATGACCTTGAATGATAGCATGTTT
AGGCTTATTCAACAACCATCCTTGCTCAGCAGCATAATCACAACAGTTCACACCCCCTGCGGGTGAACCA
TTATACCCAGTAGAAATAATACGTCCATTCTTTTCAATTACTGCTCCTACCTTCCAGGAGCAACATTTTG
ATTCCTGCGATACTAAATATGCAATTTGAAGTACTGTACTCGCTTTCATTTCATAATCACCAGATAAGCA
GATTTAGCAGTTTCAACACGATAAATTTCGTGACGAAGTTTAGTTATACTTTTAATAACAGAACTAATTA
TATTCTGCCCATCTTTAAAGCGGTTTTTCTTATCAATAAAAACTGCGCCAGTCATCTTTTTGTGAAGCTC
AACTGGATACTTCGTCACAATAATAGCATCATACACAGAAGGATGAATACTATTCACCAGAGTATCATTC
ATTAAAGTTATTCTAATGAACTGTGCTGTTTCAGAATCAAGCGCTCTATGATCGCCAGTATCATTTTCAA
GACAATTATCAATTATATCAGTTAAATTCATCATAGTACGCCATACACCCTTTGTGCTTCAACTAATCCA
TCAAAATCCAGTTTAAGATGCGATATTTGATCGCCATCACCTGGATTCACAATTACTAATACTGAACGAG
GAGTTTCGGTAATAACACGAACCGATGTTTCAGGAAATCGTTCAGAAACCTTATTTACTAATTCCTGCGC
AAATAGTTTAACTTTTTCTTGGAATTCCTTTAACAGTAATCGGTTTTTCACTTAGCAACATTTTGTTTTC
CTCATTTGTTTTGGTAGAGCTATAATATCACAACTCTACCGTAAAGTAAACCATTAAATCGCTTTGAATT
CCGCAGTTTGAGATTCAAAGCGAATATCGCCTTTGATAACAAGCTCAGCATCAAGACCAAATACGACAAT
GATATGCGCGGAACCTGGATACAGTGTAATGGCAATAGAATCCACCTGGTCTGGAAGCAAAGTGTTCAAT
ACATGAGTCACTTGAGCATGGATTCGAAGCTCAGCTGCGTTATCAAGTTTTTCAAACATATTATTAGCGA
TAATTTGGCTAAACACTACTTCTACGATTTTAGAGTAAGTCGGAAACATATTTACCTCACATAATTTTCT
TCAAGCCAATCAATAACATCCAACGCATTATCAAAAGTTGAACCATCTACTCTGTCTTCTGTTTCATAAT
CAAGAACATCTAGGCCTACTCTTCCGTCAACAATAGGCCATAGACAAAATAGATATTTCTTTTCTTTTTC
AATTTTATCACAAAGACGATAAATCTTTTCTAGGTTATTCATAAGTTTTCCATGGTAAAGGCAGTTTAGT
TTTCTTTACTACTAGTTCAACATCGGGATTCTTTTCTCTTAATTTAAGACATTCCTCCCATGCTCTATTT
TCACTAGTAAATACACAAAATTGCCCATTACTAGTACCAACTAAACCGCTATTTACAATAACAATAGCCC
AAGTTTCATGGTGCCAAGCCATTAAAAATCTCCCGAAGCGACTTGCCAGCATTCAACACCGATACGACGC
CACATTTCAACTACTTGAGTTCGGTCATCAATAGCTAATTTCACGTCAAAATGCGGTGCAATGTGTTTCC
AGAAAATTTCTTCTTTAACTACATCGTCTTTACGGGTATCGCCTTGTTCGCGCTGACATTGCATAACTAA
TGGAACGCCAGCAATGTCCTCAACCCATTTACGGGTCATACGATAATATTTCGTTGGGTCTTCTTTAGTT
CCACTTTCACGACCTGAAACGACTACGATTTGATAACCCATAAGAGCATACATCTTAGACAGTTCAACAA
CCATAGGATTGATAACATCGGTATCGCATTTTTCAAGGTCATAAGGACCACGACCATTCATTTTAGCTAG
TGTACCATCAACATCAAAAATAACTGCTTTTGGTTTACCAGGAGTCCCATTATATACTGGAAGACCGAGA
TACTCTCGCATGCTTTTATACATTGAACGTAAAACATCAATTGGTACTGCTTTAGTTCCGCGTTTTGAGT
TACGTTTAACCAATTCAGTCCAAGGAACATCAAACACTTTATGTTCAACTTTCCAGCCGTATTCTTTGGC
AAAAGTTTCCCATGCTAGGCGACGTTCAGGATTCAGGTTAGTATCTGAAATGATTACTCCCTTAACAGAA
TCGCCACCGTACAGAATACTTTTAGCTGTATCAAACTGCATACCAGTTACGATACCTTCTTTCTTTTTGG
TATACTTGTACTCATCGCGTTCTTCATGCGCCATAATAGATTGGCGATAGTCATCACGATTGATATTATA
AAACCCGGGATTCTTAGCAATAAATTCACGAGCCCAAGTACTCTTACCAGAACCAGGACAGCCAATAGTC
AAAATAATCTTTTTCATTTATTTTTTCTCAACTAATGATTGAATATAATCATGTAGGTCTTTAGATGCTT
TACCCCACTTATTTTGATATTCATTTTTGAGATTAGCACGTGATTGAGCTAATAAAACATCATTAGTTGG
AGGTAAAGATTCTAACCGCTGAATCTGGCGTCCATAAATCATTGCAGCCATCTCGGATTCATAAATCAAT
CCTTTGAGATGTTCAAATTGATGCCATGAAATCATTTACATTTATCCTCTTTTAGCTCTTGACGATAATA
ACATATCATAGTTTTCTGGTCATGTACATATCGTTTTACATCATTAAGCCAAATACGAAATTCCTGAGAA
TCTTCAAATGGCATACCGACCCAGGCTTTACCATCAATAACTTTAACTTGCCAAGATAGTTTAGCTTCAT
CATATGACTTTATTTGCACAGGCCAATTAGGATGAACTGTTTCTTTCTTTACTTCTAGAGGCTTTGTCGA
ACAACCAACTAGAAGACCAATAGATAATATTACTGCTGATAGTTTAATCATTTAGAAAGGTCCTGGATGT
CTTCTGCGAACTTGTTGAAGGAGTTGTTGATTTGTTTTTCAACCAATCCTGGCTTATGAGCCACCACATC
CGCCTTCTTTGCATCTTTGCGCAGTTTTTCATTTTCACGCTCAATAGCAGCAATTGCCTCACGATTTTTA
TTATTCATCGCATCAATATAATTATACTGAATTCGCAAATTATTTAATGCTAAGGCGTTTTCATTGGCCG
TTTTTGTAATTTCAGTAACTGATGTTTCTAATCTTTCTACCTTATGTTTTAAAATAATAGATGTTCCGCC
AAATGCTATTACAAGTAATAGCAATCCAGCTGTAAAATTACTTACATGCATAAAGTTTTAATAACCTCTA
CAATATCGTCTTGAGAAAGACCGTTAATTAAAATATGATGTTCAGCTGGAGATTTAGAAATTTTAAAGCA
TGCCTCAACATCTTCTGCCATATCCGATGCACTACGATTTGGATTACTAATACCAAGGCGATGTTTTCCC
GTCAAAGGATTAACGATGATATAGCATTTGCAGCTATTGATATGAACATTGGGTTGAGTCTGATTAATGA
ACACTTCACAATCATACTTAGCAAGTTGATTTTCTAAAAAGACTTTCATCTCTTCAACCGCATCAGGAAG
CATATCACGGGCTTGCTCAAGACGACGATTTCGATATTCTTTAATGGTCGTTTTCCGCTTGACTTGCTTA
GCTAAATCTTTCTTAAGACCAGTGATATATCCAACTCGACGATTTCCTTTGAATACAGAAATCCCATCTG
TAGTATCACCGTATGCTTCAACGACCATTTCAGTAGTAATAAGTTGTAAATCCATCATAAAGTCCTCATG
TTATGTCAGTAAGACTACTATAACACAACACGAGGGACTTGTAAACAACTTAGTATCCTTCTGGGATAAA
TTTTTTATAATTTTTCAAAAAATTCTGTTCGATTTCACACATGACCTTTTCTTGACTATCGTACCCCTGG
TATAAGCTCATGATGATACCGAACAGGTGATCCATTCCAGCACCTTTAGCAACACCTTGTGCTTCCATTG
CATAAGTCTTTCTATCCTTACCGCAATGCTTATTATGACAGTCAAGAACTAAAAACAGAGCTCGGTCTAA
GTACTTCAGATAAGTCGTTTCAAATGCTTCAATTTTTCTGTATGAATATTCATCGTCAGCATACATTGCT
TTAAGATCATCTGATGCACCATCAATAATAGTCTTAAACAATTTTTCTGGATTATCTAATGAACTTTTTG
TACTATGAAGAGACACGTACCAGTCAGACTTAATTTTAAAATGAGAACCATCTTTCATCACAGCAACATA
GCCTTCGATGTTTTCTGCATTTTTAGCTTCTTCTATCCATTTAGGGCTATCGATTTCGTATCGTTCAACT
AGATACGGACGAAGAGTAGCATCTTTATAAATATCATCGTATGAAATGTATTCACCCGTTTCGTTTTCAC
GAACATTCAGTAAAATAATTTTCATCTCTTGATAAGCAAGAACGATTCTATTCGTCGGGGCAACGAATTC
GAAGTTAGCAGTAAATCCATCTTCAGCTAATTCTTTAAGTCTATCACGCAACCGATGGTGATTAATATTC
ATCAAAATTCCATTAGCCATTAAAGCCTGCTCAGATTTGATTGAACCCTTTGATTTGAACAGAATTTCAT
CACCGTCTAAATAAGTTGATACCAAAGACCCGTCTTCTTTTGTTAGAATATAATCAACATCGTTTAAATC
GATATTCATCGTGAACGGATTTTCATTCAAGTTAAAAAACTTTTCCATAGGACGAGAAGCAATTCTTACT
GGTTTTTCTCCATCCATTTCAAACATAATTCCACGACATTCTAGTGCATCTGGAAGTAACCAATCAGAAT
AAGATGCATAATTATATGAGAAAATTCTGTAAGTTCTTCCAGATGCACTTACATCATCTGAGTAAAAAAA
CTTACGCTGCGAATCCTTACATAGTTCCATTAAATTGTTAAAAAGTTCTTGCATTGTGTATCCTCTTTTG
TGTTTTGAATATAGTACCACACTCCATGTGGAAGCATCATTTTTTCTTGTGTTGAATATTCCAAGGCGGG
TTAAACAGTTTAATGAATAGAGGCTCCTCTAAGTCAATCGTTGCGATTGTCATTGTACCTAACTCATTTG
TCATAGAAAGATTAAAACATTGGCGGGCGTAAAATTCAACTTTGCTTCCTTCCTTTAGCGCAGAATGAAT
TAATGCAGATTTAGTAGAATCAGACGTTTTGTCTTTGCGGTTAATAGCAGTTCTATAATAGTTTATTCTT
TTACGTAAATTTTTAGTTTTTCCAATATAAACAAGCTCATCATTTATAGCAATAGCATAAATTACGTTAT
ACTTGTTTGGAATAGATAATTGTTTTATACTTCCATTGTCGTCTAATTCTAGCTCAGTATATTTAATAAA
TGAATATTCTGTTGCAATTTCTTTCATAATAAAATGGGCCTTGCGGCCCACTCCTTAAAAGTATTTTTTA
AAACTCATCATAACTTTATCATCAACATCATTATCAATCTGTGCAACAAGGTAAGATGACAGTTCTACTT
CTTGCGGCGCGGATTGAACATTATCAGAATTAAGATATTCACGAATCCAAGGATATGGATGTTTAACCGG
AGCATCGGTAATTGGGCATGGAAGACCGCACTGTTTCATACGAGATACAGTTAAGTAATCAATAAAGCTC
CACATGCTATTTGTATTTAATCCAGGAACATCACCATCTTTAAATAAATGAACTGCCCAGTCTTTTTCCT
GGCGGTTAACTTCCATGAAAATATCAACTGCTTCTTGTTCACACTCTTGGGCAATTTTAACCCATTCATC
ACCATCAGTACCGGATTGAAGTTGACGAATAATATATTGGGTGCCTTTAAGGTGAAGCTGTTCATCACGT
GCAATGAACTTCATAATCTTGGCATTACCTTCCATGATTTCCATGTTCTTATGGAAGTTAAAGGTACCGT
ATGTTCGATTGAGCTCGCTAATTCTCAACCCGTTCTCTTATGAACTGCTGCATGTTACCATGCAGTCCAG
ACTATATCACAATCCCGGAGGGATTCTCCCCATTTCGAGTCGCTTGACCCTACGTCCGAAGACTAGTCGT
TGAACCTTGTTTTGCAATTGTCGCCGTGCCATCGTTTATATGTTGATGGTGATAAATCTTTCTTATCACA
ATATGGACAATTCAATTGAATTCTATTTGGATTTAATTTACATCTGTCATTATGTTTTAGATAAAATCCA
GGACCTTTACCAATGTGACCACAGAAATCACATGTGATTTCTTTTTGTGCAGGATGGGTCCCGTTCTTAA
CCATTTCTAATGTTTTTGCTGATGTTCTTTTCTTATGTTCTTCCATCAGAATCTTTAGGTATAGGTCCGT
TAGCATCTATCCAAATTTTTCTATAGTTCAATTGTGCATCCTCTTAAAAGTATAATCATATTTATATTAT
ACTAATTAAAGGTGCAAGCAAAACCTTGGCTGCTAGTTTTCCATAAAGGACTTTCCAGCAATTAAAGGAG
TTTTCGATAAACGTTACCGTTTAAAGGCGCATTTTACGCAAAAGATACATAAAAACGAATAGCTTCTAAG
GCATTAATAACGTGCAAACAGAGGTAAAGAGATTTCATCAGATCTCGTTTAGCTCTTTGCTCAACGTCTT
TATCAGCTAGAATTAGACCTTGGTCTTTATAATATTCAACCATGTCTTTAGCATTTTCCCATTCACGGGT
TTTAACCAAGACATCATCGTAATATCGCCCAATGGACTCAGCACGTTTCATAATAGCTTCATCTAATACA
ATCTCATCAAACACCTTCGATGGATCAGTATAAAGATTTCGCATGATATGAGTATATGAACGACTGTGAA
TAGTTTCACTAAAAGTCCATGTAGCAACCCATGTATCAAGGCTTGGGTCTGAAATTAATGACATAAGTAC
AGCAGATGGCGCACGACCCTGAATGCTATCCAAAAGTGATTGATACTTCAGGTTGTTAGTAAAAATATTT
TGCTGATACTGAGGAAGCTTATTAAATTGCGCAGCATCCATCATTAAGTTTACTTCTTCAGGACGCCAAA
AAAAACTGATCTGCCGCTCAATGAGTTCTTCAAATACTCGATGTCGTTGAATATCATATCGAGCTAAACC
TAATCCAGAACCAAAGAACATCGGTTCATTCAAAACATCAACTGGATTTGTATTAAAAACTGTACTCATT
TAGAATCCTTAAATTTACATTTATCATAATGCCATCTTAAAGCATTGCCTTTATTAACTTTCTTACCACA
GTGCGGGCAAGGTGGATACTCTGCTCCTTGTTTAGTTCTCAAAGAAATCAAATCACGGGTTTCTTTAGTA
TGAGGAACATCTCTTGTCGGAGAAATTGTACCATACATCGGATTAAGCACGCCGACTTTAGCAGCAGCTA
TATTCTTTTTAGCTTCTTCAGTTTTAGGCTTTCTCATCTTTTTCTTAGGTTTCTTCGCTTTTAGCCCTTC
CTTTGGTTGCCGCCGATATTTTGTGTTTGGTTTCTTCTGTTAAACGCAGACCTGTTTTCGCTTTTTGACA
TTTTCAGTCTCGTTTCTTCAGAAATGACCGTACCTTTGCGAAATTGCGAATTTAACTTCTTTGCATGGGC
ATATATTTTAGAATGAACTTTATAATGACGTTTCTTAGTTCCTTTCATATTGCACATCATGAAAAATGCG
AATATAACAGATTTTACCGGATAAATCTTTGATAAAATAGCATGCGCTATAAAATGCTCTCTAGCTGTTA
ATTCAACTAAATTTTCTTTATCATCAGAACCTCCCATGCATCTAGGGATTATATGATGTGTCTCTTTATA
TTCGGATAAAGGTTCCCGAGCCTGAGCTCGGGAAATTAGGTCGTTATAGATTTTTTGATAATTCATTACA
ATTTACACGCTGCACAATCATCGGCTTTAGGAGTTTCTATTTCATAATCATCAGTACCAGAACCATCACG
GGTATTATGATAATAGAAATTTTTAATGCCATAATACCATCCGTATAGCATGTCATCAATCATTATTGAC
ATTGGAACCTTTCCTTTTGGAAAAATCTGCGGGTCATAATATGTATTCGCTGAAGCTGATTGACATACCC
ATTTCAGCATAATAGCTACCTGCGTAAGATAAGGTTTATTACCTTTCTTAGCTAATTTCCATGTATAATC
ATAGAGGTCTATGTTATGCTCAATATTGGGCACGACTTGATTAAAGGAACCCTCTTTTGATTCTTTAACA
CTTACCGGTCCACGTGGAGGCTCGTAGCCGTTTGTACTGTTAGAAACTTGGGAAGATGACTCACATGGCA
TAAGTGCTGATAATGTGCTATTACGGATGCCAAATAGCTTAAGGTCTTCCCGCAGCGCCGACCAGTCACA
AACGTATTTTGGAGCTGCGATTTGGTCAATCTTTTTATTGTACCAGTCGATAGGTAATTCGCCTCGAGAC
CAACGAGTGTCTGAATAATATTCCGAAGGTCCTTTTTCTTTGGCGAGCTTAATGGATGCTTTAATGAGTC
CATACTGTAATCTCTCAAATAGTTCATGTGTTAAATCGTTAGCATCTTCATAAGAAGCAAAGTTACTTGC
CAGCCAAGCTGCATAGTTGGTAACACCTACACCGAGGTTACGACGCTTTTTAGCTTTTTCTGCTTCAGGA
ACCGGATATCCTTGGTAATCCAACAGATTATCAAGAGCACGAACTTGAACTTCTGCCAATTCATTAATTT
TATCTTGGTCTTGCCAGTCAAAATTATCTAATACGAATGCAGAGAGAGTACACAATCCAATTTCAGCATC
AGGGCTATTCACATCATTTGTTGGAATAGCAATTTCACAGCACAAGTTACTCTGACGAATAGGTGCCTTT
TCACGAATAAACGGAGTATAGTTATTCGTATTATCAATGAACTGCACATAAATCCTTGCTGTTCCTGAAC
GTTCAGTCATGAGCAATTCAAATAGTTCACGGGCTTTAATACGCTTTTTACGAATATTAGGGTCTTTTTC
TGCTGCTTCGTATAATTCACGGAAACGGTCTTGGTCTTTAAAATAAGAATAATAAAGCTCGCCACCCATT
TCATGCGGACTGAACAAAGTAATGTAATCGTTTTTTCCAAAACGTTCCATCATCAAATCATTCAGTTGAA
CACCATAATCCATATGACGAATGCGGTTTTCTTCTACGCCTTTGTTATTTTTCAAAACGAGAAGATTTTC
AACTTCCAAATGCCAAATAGGATAATAAGCAGTAGCAGCGCCGCCACGAATTCCACCCTGTGAACATGAT
TTAACTGCAGTCTGAAAATGTTTCCAAAAAGGAATAACACCAGTATGGCGTACTTCACCCATGCCAATCT
TAGAACCTTCGGCACGAATCATACCAACGTTAATACCAATTCCAGCGCGTTTAGAGATATATTCAACAAT
TGAAGCAGAAGCCTTATTGATAGACTTCAATGAATCTCCTGCCTCAATAACAACACATGAACTAAACTGT
CGAGTCGGAGTACGACAACCAGCCATAATAGGAGTTGGCAATGAAATCTGTCGAGTTGATACTGCTTCAT
AAAAACGGATAACATGTTTTAATCTATCAACAGGTTCATCTTGATGCAGTGCCATTCCAATAGTCATAAA
TGCAAACTGTGGAGTTTCATAAATTTGACCAGTGGTTTTATCTTTAACTAGATATTTTTCTTTTAATTGC
ATTGCCCCGGAATAAGTAAATTCCATATCCCGTTCGTGCTTAATTTTTGATTCTAAAAATGTAATTTCTT
CTGCTGAATATTTTGACAATAATTCAGGGTCGTATTTACCTGCATTTACACAATAAGAAATATGGTCAAT
AAATGAACGTGGTTCATACTGCCCATAAACATGCTTACGAAGAGCAAACATTAAACAGCGTGCAGCTACA
TATTGATAATCAGGTTCTTCAACCGAAATAGAATTCGCAGCAGCCTTAATGACAATAGTCTGAATATCAT
CAGTTGTCATTCCATCACGGAGATAAGATTTAATATTTTCATATAATTCATAAGGATCTACTGATGTTCC
TTCAGCTGCCCAAGATAAAACTTTAATAATTTTTTGTGGGTCAAAGCTCTGAGAAACACCACTACTTTTG
ATAACATTAATTAATTGCATAAGTCCTCAACTTGAAAATCGTCTTTAAACAATCGGTTAACTATATGAGC
TATTATATCACCATGACACGGCTTTGGTTTACATGTGCATCCTAGCCTCATTCCACGTAAAGGCTCTAAA
TGTGCTTTAGTTATTTCTCCGGATTTAATTCGACGTATAAAATCTTTTTTGAATAATTCAATGGCAGCCT
CCCGGCTGCCAGCATCTTTACCGACGTAATTTCCCCAAAATGTACCGCGGTGAATATTAACATCAAAGTC
GGATTTATATTTATTCACTACCCGGCATAGACGGCCCACGCTGGAATAATTCGTCATATTGTTTTTCCGT
TAAAACAGTAATATCGTAGTAACAGTCAGAAGAAGTTTTAACTGTGGAAATTTTATTATCAAAATACTCA
CGAGTCATTTTATGAGTATAGTATTTTTTACCATAAATGGTAATAGGCTGTTCTGGTCCTGGAACTTCTA
ACTCGCTTGGGTTAGGAAGTGTAAAAAGAACTACACCAGAAGTATCTTTAAATCGTAAAATCATATATCC
TCGCAATAATAAAATTACACCGCCATCTTTCCTTTAATAGGAGGGTGTGATACATAGTTGTTAAGAACGA
AATCTTTAGGCCTAAGTTTAAGAACATATTTTAATTGTTCTTTAGTAGAAAGATATCGGAATTTATAAGG
TAGACCACTTATTACCAGCTCACAAAGCTCTTTAGGTTCACGCCTCAAAATTTCTTTACATTGTTCTACG
TGATTCATATAGATATGAGTATTACCACCAGAAAATATCAAATCCCCTGGAATAAGATTACACATCTTAG
CTACAATATGAACTAACGTAGCATATGACGCAATATTAAACGGTAGCATTATGTTCAGATAAGGTCGTTA
ATCTTACCCCGGAATTATATCCAGCTGCATGTCACCATGCAGAGCAGACTATATCTCCAACTTGTTAAAG
CAAGTTGTCTATCGTTTCGAGTCACTTGACCCTACTCCCCAAAGGGATAGTCGTTAGGCATTTATGTAGA
ACCAATTCCATTTATCAGATTTTACACGATAAGTAACTAATCCAGACGAAATTTTAAAATGTCTAGCTGC
ATCTGCTGCACAATCAAAAATAACCCCATCACATGAAATCTTTTTAATATTACTAGGCTTTTTACCTTTC
ATCTTTTCTGATATTTTAGATTTAGTTATGTCTGAATGCTTATGATTAAAGAATGAATTATTTTCACCTG
AACGATTTCTGCATTTACTACAAGTATAAGCAGAAGTTTGTATGCGAACACCGCACTTACAAAACTTATG
GGTTTCTGGATTCCAACGCCCGTTTTTACTTCCGGGTTTACTGTAAAGAGCTTTCCGACCATCAGGTCCA
AGTTTAAGCATCTTAGCTTTAACAGTTTCAGAACGTTTCTTAATAATTTCTTCTTTTAATGGATGCGTAG
AACATGTATCACCAAACGTTGCATCAGCAATATTGTATCCATTAATTTTAGAATTAAGCTCTTTAATCCA
AAAATTTTCTCGTTCAATAATCAAATCTTTCTCATATGGAATTTCTTCCAAAATAGAACATTCAAACACA
TTACCATGTTTGTTAAAAGACCTCTGAAGTTTTATAGAAGAATGGCATCCTTTTTCTAAATCTTTAAAAT
GCCTCTTCCATCTCTTTTCAAAATCTTTAGCACTTCCTACATATACTTTATTGTTTAAAGTATTTTTAAT
CTGATAAATTCCGCTTTTCATAAATACCTCTTTAAATATAGAAGTATTTATTAAAGGGCAAGTCCTACAA
TTTAGCACGGGATTGTCTACTAGAGAGGTTCCCCGTTTAGATAGATTACAAGTATAAGTCACCTTATACT
CAGGCCTCAATTAACCCAAGAAAACATCTACTGAGCGTTGATACCACTGCAAATCCAAATAGCCATTACG
CACATTAAACTGATAGAACATATGACAAGGCGGTAATGCCATATATTTAAGTTCAGCTGGATTCCATGCA
GAAACAATTTGACGCCTATCATTTGGCAGTTTTTTAATACGATCAATAACTTCTATAATTTGGTCTACAC
CACCAAAATCACGCCACTGTTTTCCATAAATTGGACCAAGTTCACCGCTATGGTATCCTAAATCTTTTGC
TTGATTTTCGTAATTTTCATCCCAGACTGTTTTGCCTTGGATTAACGAATCGTGTTGAATTAATCGTAAA
TCATTGACATTTGTGCTTCCTGATAAAAACCATATTAGCTCAGCAATGCAAGCTTTCCAGGCGAGCTTCT
TAGTTGTTACCGCAGGAAAACCTTTAGTTAAATCCCAGCGTAATTTAGATCCGAACAGAGCAATTGTTCC
TGTGCCTGTACGATCATCGGTTTCATAACCATTTTCAAAAATGTCTTTAATTAAATCTTGGTATTGTTTC
ATTTATATACTGATTCCGTAAGGGTTGTTACTTCATCTATTTTATACCAATGCGTTTCAACCATTTCACG
CTTGCTTATATCATCAAGAAAACTTGCGTCTAATTGAACTGTTGAATTAACACGATGCCTTTTAACGATG
CGAGAAACAACTACTTCATCTGCATAAGGTAATGCAGCATATAACAGAGCAGGCCCGCCAATTACACTTA
CTTTAGAATTCTGATCAAGCATAGTTTCGAATGGTGCATTAGGGCTTGACACTTGAATTTCGCCGCCAGA
AATGTAAGTTATATATTGCTCCCAAGTAATATAGAAATGTGCTAAATCGCCGTCTTTAGTTACAGGATAA
TCACGCGCAAGGTCACACACCACAATATGGCTACGACCAGGAAGTAATGTAGGCAATGACTGGAACGTTT
TAGCACCCATAATCATAATTGTGCCTTCAGTACGAGCTTTAAAATTCTGGAGGTCCTTTTTAACTCGTCC
CCATGGTAAACCATCACCTAAACCGAATGCTAATTCATTAAAGCCGTCGACCGTTTTAGTTGGAGAATAA
CGGAATACCAATTTAATCATTACGTAAATCCTATTTTAATTGAAAACGAATGCTTACTTGGATAATTTCA
ATGACATACATAATATTTTCCTCAAACAGACTTTTTCACAATTTTCCAATCAGCTTTAAACTGCTCGACG
TCAGAATGGTAAATCCAAAATCCTGCGCTTTCTCCGTCTTCATAAAGAGGACATCCATCGCATTCATCTT
CCCATCCCATATCACGTAAAAGATGTTCAGCTTTTTCAACAAGTTCAGAATCTTTACCGATGATATTAAA
ATACCATTTACCTCTAACTTCTGAATCTTTGATGCTCTGGCGTTGTAATCTCATTTTATTCTCCTTAGCA
AGCTTTAATCAAAAGATATAAACAGACCAACATAACTGCTGCCATAATATAAGGTGCGAACATTTTCTTT
TCTCCATTAGTTTTGATAGGGTAATAGTATCACACTACTACCCTGATGTAAACTACTTTTTGAAAGTTTT
TCGCAAAAGTTCAATGATTTCATCTACATTGTTTTCGTCAACAATGCAGTGAATTTTTGTTACGCCAGAA
ACCTTGTCTTTGACTTCATCTTCTTCAGAAGTCGGTTCTTTATATTCGCGGAAACAATAAAACTCTTCTT
CACTAAGTTCAAAATAATCATCACCCATACCATCATCATTATAGATTTCACCATTAGCACAAATGATTTC
GGTTACATAATCAAAGCCATCTAAACTTGATATTGATTTAACTTCAAACCAACCGCCATTTTCTTGAATG
ATGCCGACCATACTAGCATTTGATGAACTAATATCAATGAAAGATTTAATACGATGTGGATTTAACTCGT
ATTTTTTGCCGATTTCCATTTTGATTTTCCTCATTTTAATAGGGGCTTGATAGCCCCTTGATAATTATTG
TTCAATCAGTCCCATGTAAAATTCTGCGTCTTCAGAATCCATGCCATCACAATATTCATTAGCCATAAAG
CGGGTGAGGTCTTCAAGAGGACCTTCAATAACGATTTGAATACTCCAAAACTTAGAATCTTGCACGCTTG
TGATACTAAGTTCAGGATAACGATTACGAATAATCTCTTCAATATATTCAAAATCAACGATGTCAATATC
AACTTTAGCCATATTATTTTCCTCTTTAATTATTAGCAGTATTGCCGATAGTTGTATAGTACCATAAAGC
TTTATGCTTGTAAACCGTTTTGTGAAAAAATTTTTAAAATAAAAAAGGGGACCTCTAGGGTCCCCAATTA
ATTAGTAATATAATCTATTAAAGGTCATTCAAAAGGTCATCCAGGTCCGTGTCATCAGCACTAGATGAAC
TACCAGAGCTTGAGCTCATAAAATCATCTTCAGTTTTTGTATTGAAGTCATCAACATTGAATGCATCCAA
ATCATCAGCCACTTTATCAGCTTTCTTAGCAGCAGTTGCAGCAGCACCGCCCATCACAGCAGTTCCCATA
ACTTGACCGAATTTAGTATTAAGTTCTTCAAACGATTTGAATTTATCTTTAGAAGTCATTTCAGAAAGGT
CAACCATTTGTTCGAACAGTTCTTTCTGGAAAGATTCATCGTCAATGTTTGGAATCGCAGATTGATTCAG
GAATTTAGATTCATCGTAGTTACTAAATCCAGAAACTTGTTTAACTTTCAGTACAAAGTTAGCACCTTCC
CACGGACAAGTTACATCAACTGGAGTTTCACCCATTTCAACATCAACCGCAATCATTGCATTGATTTTAT
CCCAGATTTTCTTACCAAAGCGGTATTTAAATACTTTACCTTCGTTTTCTGGAGCAGCTGGGTCTTTTAC
TACAAGAATGTTAGCCCAGTAAGAAGTTTTACGTTTAACAAGACTGTACTCTTTATTGTCAGTGTTGTAT
AGATCATTTTTACTGATGTATTGACATACTGGGCAAGAATCGTAATCACCATGGGTAGATGAACATGTTT
CAATATACCATTTACCATTTTTCTTGAAACCGTGATTTACAAGAATTGCGAATGGTGCTTGTTCATCATT
TTTAGACGGAAGAAAACGAATTACTGCTTGACCGTTACCCGCATTATCGAGTTTCAGTTTCCACTCGCCT
TTATCTTCAGAAGAAAAACCTTTATTGCCATTCAGTTTAGCCATTTGTGCAGCGAGTTCAGCAGTAGATT
TACGTTTAAACATTTTTATTTCCTTTTTAATTTAATTTAATTAACAGTTGGTGCTATGACACTTTACCTC
ATAGCTGGCATAATTCGCAATACTCTGGGTCTTCGAGAGGTATCCAACCTGAGTTGAAATACTTTACCAT
CGATTTAGCAGTTGTATCAGTTATATTTATATTACCTTTAACTCTTCGCCATCCAGGAGTTTTACCGTAC
AGATTAGAGGATAATAATAACACATAATTCTCGTAAGCAATATGAGATAATTTCCAAGACTCTATATTAG
CTCGTGATGTTTTCCAAGGTCTAAAATCGTCACGGTTCATATAATTAGCCAATCTCATATGCTCTCTAAC
TTCCGGGTCTTTGGCTGGATGAGTTTCACCACTCACACCAAATCCACCACCAGCATATACCAGATTAAAA
TAGTCTGGATTATCTCTGGCATTTACTTCAAGTTGGTATTTTCGTTCTGCTTCAATAACATCCAAGTCAT
CATCAATTTGAATTATTTTAACGCTCGGTTTTTGAAGCATTAGCGCATTCAAAAATCTTTTTTGTTTACA
TGAGCTCCAGTATTCCTTTCCGGAAGAGTCATATATTATTCCGTTCTCAAATGAGCAATTTAATTTACTA
CCGATATAGTAGTATGGCGGAGTCTTATTTTTGACACGGTCTTCAAATGTAAACCAATATACTATATTCA
TATCAATACTTGCAAGATTTCACAGTTTCAATGAAAACATTTTTAGCTTTCTGTGAATCAATATTTAAAA
TTTTTCTATAAGCCTTTAACTTTATAGAATAATTATTCCAGACTAAATTATCAGTCTGTTCATCATGTTT
ATCAATTATATTTAAAAACGAATCAAGCAAGATAAACGTCTCAAACGAAATTATGTTCGATTGCAGAAGT
TTAAAAATATAACTTGATTGAACTTTTGGATTATACTCAAAAATTTCTTTAAAAGCAGAAACTTCAACTT
TTTTACTAAAATAATAAATGTTGCGAATATCTTCTTCAAACTTAAATTTAATTTGCTTTAAGCGTCCGAT
ATATTCACGATAAAACACAAGTGCATCAGCGTCAGAGATGTCACCAATCCAAGCATCTTGGTTAGCAACC
AAATTGCTTATAAAGATTAAAGCAAGTTCCTTTAATTTATATTTTTCTGATAACTTCTGGAAAAAATACT
TATCCCTTCGCTTTTGATAAGCGGCATCAGACACCCGCATGCACCAATTATACTTAATTACATCATACTT
TCCATTCATATGTTGTTTTATCATTAAGTATAATTTATAAACTGATTTACCATCAATGTATCTTTCACCA
CCAGCAGGCATGCGGAGTTTAATCATAGTAGAAAATCTAATGTATTAGTTTTTTCACAACGAACAACAGA
AGGACGTAAAAGATTTTCGTCAATAGCTTCTGACTGAATTTTTTCAATTATACCCGAAGGAATAAATTTA
GCAAATTGAGTTTCAGGAATAGAATTTTCTTCTAAGAATGCTGTTGTAGCTTCAAGATAACTCATTCCAA
ACTCTTCTACCATTTTTTCAATAATAAATCCATTTTCTTGGCGGTCAAGAAGCTTTGCTATTTCATCCTT
TTCTTTCTTAATTGAAAGTTCTTTTTCTGAAAGACCGGTCTCATCGACCGGACGAATATCATTTAGAGAA
AACTGTGTCATAAAGTTCAACTACCTCTTCAGTTTCAGCTTCAAACACATCACGGTTATCTTTATGATAC
AAAGCTAATAGACGATTAAACATCTTACCATCAACGCCAAGTTCATCTTTAGCACGAATTCGAATATCTT
TAATCAGTTCATTATAACCGGAAATTTTCAGTTTATGATCAGATGCTTCTTTAATAAATTTAGCCAAGTC
TTCGCCATGGATAGCTTCATCAAATTCAACCATTTCTTTTTTAGCCATTATTCACCTCAAAATTCATTAA
TGCTATTAGTTAATTTAGAAAGACCCGCTTTTACAAAATATGAATAAATTTTGCCACGCGGTGGTAATTT
ATATGAATTATAGTAATTCACAATGTTTGAAGCAATATTATCAGGAATATAATCAAAATCAATTAGAACT
AAATTTTCTTTATAACGATTATATTCAGATTCAGTGAGAAGCACCTTAGCTTGCTCACGGTCATTAGCAA
TAGCTTCAACGATTGAAGTTTTCATTGAAGGAGTTCGTTCACCTTCAACTCTGGTAAACCAAAAGTCAGA
TCGTACTTTAACTGAAGCAACGTTATCCTTTTTGTCGCCTTTAAGGATTTTAGTCATACAGTCAATTTCA
GCAGAACCGCTTTTAATTTTAACCCATTTCTTATGCATCGGAGACCATTGCTTAACATTTGGATATTTGT
GAAGCTGAGTAAAGTCACCATCTGACGAAATGATTAAAATCTTATGTCCTTCTAAAGAGAACTTTTTAAC
AAGAACAGCAATGTGGTCATCTGCTTCATACTTATCAATATCCATAACAATGTATGGCATATAAGCTTTC
AATTCATCTATAACTTTATGGCTGGATTCAAAATAACCTTCCCAGTCCCAAGTAGATTCTTCTCGTGCTT
TTCCACGGTTTTTCTTATAATAATAAGCGAAATCACGACGCCAATATCCAGATTTCGCGTTATCAATACA
CAGTACAATTTTAGTGTATCCAAGCGTTTTTGCTTTTTTGACATTAAACTTAATTGAGTTCAATATCAAA
TGACGAACCATTGATAAATTAATTTTTTCTTTATCTGGGAAGTTTACCAAAGCAGTTGAAAGCGCAATTT
GACTAAAGTCAATTAAGCAGATTCCTTCTTTGTAATCTTCATCCAACATCATTTCTAAATCCATATGAAC
CTCGTTCAATTAGTGAGATTTCTATTATATACCATCCAAATCTTAAAGTAAACAAGTATAAATACTTATT
ATTGAAAACACAATAGGAGCCCGGGAGAATGGCCGAGATTAAAAGAGAATTCAGAGCAGAAGATGGTCTG
GACGCAGGTGGTGATAAAATAATCAACGTAGCTTTAGCTGATCGTACCGTAGGAACTGACGGTGTTAACG
TTGATTACTTAATTCAAGAAAACACAGTTCAACAGTATGATCCAACTCGTGGATATTTAAAAGATTTTGT
AATCATTTATGATAACCGCTTTTGGGCTGCTATAAATGATATTCCAAAACCAGCAGGAGCTTTTAATAGC
GGACGCTGGAGAGCATTACGTACCGATGCTAACTGGATTACGGTTTCATCTGGTTCATATCAATTAAAAT
CTGGTGAAGCAATTTCGGTTAACACCGCAGCTGGAAATGACATCACGTTTACTTTACCATCTTCTCCAAT
TGATGGTGATACTATCGTTCTCCAAGATATTGGAGGAAAACCTGGAGTTAACCAAGTTTTAATTGTAGCT
CCAGTACAAAGTATTGTAAACTTTAGAGGTGAACAGGTACGTTCAGTACTAATGACTCATCCAAAGTCAC
AGCTAGTTTTAATTTTTAGTAATCGTCTGTGGCAAATGTATGTTGCTGATTATAGTAGAGAAGCTATAGT
TGTAACACCAGCGAATACTTATCAAGCGCAATCCAACGATTTTATCGTACGTAGATTTACTTCTGCTGCA
CCAATTAATGTCAAACTTCCAAGATTTGCTAATCATGGCGATATTATTAATTTCGTCGATTTAGATAAAC
TAAATCCGCTTTATCATACAATTGTTACTACATACGATGAAACGACTTCAGTACAAGAAGTTGGAACTCA
TTCCATTGAAGGCCGTACATCGATTGACGGTTTCTTGATGTTTGATGATAATGAGAAATTATGGAGACTG
TTTGACGGGGATAGTAAAGCGCGTTTACGTATCATAACGACTAATTCAAACATTCGTCCAAATGAAGAAG
TTATGGTATTTGGTGCGAATAACGGAACAACTCAAACAATTGAGCTTAAGCTTCCAACTAATATTTCTGT
TGGTGATACTGTTAAAATTTCCATGAATTACATGAGAAAAGGACAAACAGTTAAAATCAAAGCTGCTGAT
GAAGATAAAATTGCTTCTTCAGTTCAATTGCTGCAATTCCCAAAACGCTCAGAATATCCACCTGAAGCTG
AATGGGTTACAGTTCAAGAATTAGTTTTTAACGATGAAACTAATTATGTTCCAGTTTTGGAGCTTGCTTA
CATAGAAGATTCTGATGGAAAATATTGGGTTGTACAGCAAAACGTTCCAACTGTAGAAAGAGTAGATTCT
TTAAATGATTCTACTAGAGCAAGATTAGGCGTAATTGCTTTAGCTACACAAGCTCAAGCTAATGTCGATT
TAGAAAATTCTCCACAAAAAGAATTAGCAATTACTCCAGAAACGTTAGCTAATCGTACTGCTACAGAAAC
TCGCAGAGGTATTGCAAGAATAGCAACTACTGCTCAAGTGAATCAGAACACCACATTCTCTTTTGCTGAT
GATATTATCATCACTCCTAAAAAGCTGAATGAAAGAACTGCTACAGAAACTCGTAGAGGTGTCGCAGAAA
TTGCTACGCAGCAAGAAACTAATGCAGGAACCGATGATACTACAATCATCACTCCTAAAAAGCTTCAAGC
TCGTCAAGGTTCTGAATCATTATCTGGTATTGTAACCTTTGTATCTACTGCAGGTGCTACTCCAGCTTCT
AGCCGTGAATTAAATGGTACGAATGTTTATAATAAAAACACTGATAATTTAGTTGTTTCACCTAAAGCTT
TGGATCAGTATAAAGCTACTCCAACACAGCAAGGTGCAGTAATTTTAGCAGTTGAAAGTGAAGTAATTGC
TGGACAAAGTCAGCAAGGATGGGCAAATGCTGTTGTAACGCCAGAAACGTTACATAAAAAGACATCAACT
GATGGAAGAATTGGTTTAATTGAAATTGCTACGCAAAGTGAAGTTAATACAGGAACTGATTATACTCGTG
CAGTCACTCCTAAAACTTTAAATGACCGTAGAGCAACTGAAAGTTTAAGTGGTATAGCTGAAATTGCTAC
ACAAGTTGAATTCGACGCAGGCGTCGACGATACTCGTATCTCTACACCATTAAAAATTAAAACCAGATTT
AATAGTACTGATCGTACTTCTGTTGTTGCTCTATCTGGATTAGTTGAATCAGGAACTCTCTGGGACCATT
ATACACTTAATATTCTTGAAGCAAATGAGACACAACGTGGTACACTTCGTGTAGCTACGCAGGTCGAAGC
TGCTGCGGGAACATTAGATAATGTTTTAATAACTCCTAAAAAGCTTTTAGGTACTAAATCTACTGAAGCG
CAAGAGGGTGTTATTAAAGTTGCAACTCAGTCTGAAACTGTGACTGGAACGTCAGCAAATACTGCTGTAT
CTCCAAAAAATTTAAAATGGATTGCGCAGAGTGAACCTACTTGGGCAGCTACTACTGCAATAAGAGGTTT
TGTTAAAACTTCATCTGGTTCAATTACATTCGTTGGTAATGATACAGTCGGTTCTACCCAAGATTTAGAA
CTGTATGAGAAAAATAGCTATGCGGTATCACCATATGAATTAAACCGTGTATTAGCAAATTATTTGCCAC
TAAAAGCAAAAGCTGCTGATACAAATTTATTGGATGGTCTAGATTCATCTCAGTTCATTCGTAGGGATAT
TGCACAGACGGTTAATGGTTCACTAACCTTAACCCAACAAACGAATCTGAGTGCCCCTCTTGTATCATCT
AGTACTGGTGAATTTGGTGGTTCATTGGCCGCTAATAGAACATTTACCATCCGTAATACAGGAGCCCCGA
CTAGTATCGTTTTCGAAAAAGGTCCTGCATCCGGGGCAAATCCTGCACAGTCAATGAGTATTCGTGTATG
GGGTAACCAATTTGGCGGCGGTAGTGATACGACCCGTTCGACAGTGTTTGAAGTTGGCGATGACACATCT
CATCACTTTTATTCTCAACGTAATAAAGACGGTAATATAGCGTTTAACATTAATGGTACTGTAATGCCAA
TAAACATTAATGCTTCCGGTTTGATGAATGTGAATGGCACTGCAACATTCGGTCGTTCAGTTACAGCCAA
TGGTGAATTCATCAGCAAGTCTGCAAATGCTTTTAGAGCAATAAACGGTGATTACGGATTCTTTATTCGT
AATGATGCCTCTAATACCTATTTTTTGCTCACTGCAGCCGGTGATCAGACTGGTGGTTTTAATGGATTAC
GCCCATTATTAATTAATAATCAATCCGGTCAGATTACAATTGGTGAAGGCTTAATCATTGCCAAAGGTGT
TACTATAAATTCAGGCGGTTTAACTGTTAACTCGAGAATTCGTTCTCAGGGTACTAAAACATCTGATTTA
TATACCCGTGCGCCAACATCTGATACTGTAGGATTCTGGTCAATCGATATTAATGATTCAGCCACTTATA
ACCAGTTCCCGGGTTATTTTAAAATGGTTGAAAAAACTAATGAAGTGACTGGGCTTCCATACTTAGAACG
TGGCGAAGAAGTTAAATCTCCTGGTACACTGACTCAGTTTGGTAACACACTTGATTCGCTTTACCAAGAT
TGGATTACTTATCCAACGACGCCAGAAGCGCGTACCACTCGCTGGACACGTACATGGCAGAAAACCAAAA
ACTCTTGGTCAAGTTTTGTTCAGGTATTTGACGGAGGTAACCCTCCTCAACCATCTGATATCGGTGCTTT
ACCATCTGATAATGCTACAATGGGGAATCTTACTATTCGTGATTTCTTGCGAATTGGTAATGTTCGCATT
GTTCCTGACCCAGTGAATAAAACGGTTAAATTTGAATGGGTTGAATAAGAGGTATTATGGAAAAATTTAT
GGCCGAGTTTGGACAAGGATATGTCCAAACGCCATTTTTATCGGAAAGTAATTCAGTAAGATATAAAATA
AGTATAGCGGGTTCTTGCCCGCTTTCTACAGCAGGACCATCATATGTTAAATTTCAGGATAATCCTGTAG
GAAGTCAAACATTTAGCGCAGGCCTCCATTTAAGAGTTTTTGACCCTTCCACCGGAGCATTAGTTGATAG
TAAGTCATATGCCTTTTCGACTTCAAATGATACTACATCAGCTGCTTTTGTTAGTTTCATGAATTCTTTG
ACGAATAATCGAATTGTTGCTATATTAACTAGTGGAAAGGTTAATTTTCCTCCTGAAGTAGTATCTTGGT
TAAGAACCGCCGGAACGTCTGCCTTTCCATCTGATTCTATATTGTCAAGATTTGACGTATCATATGCTGC
TTTTTATACTTCTTCTAAAAGAGCTATCGCATTAGAGCATGTTAAACTGAGTAATAGAAAAAGCACAGAT
GATTATCAAACTATTTTAGATGTTGTATTTGACAGTTTAGAAGATGTAGGGGCTACCGGGTTTCCAAGAG
GAACGTATGAAAGTGTTGAGCAATTCATGTCGGCAGTTGGTGGAACTAATGACGAAATTGCGAGATTGCC
AACTTCAGCTGCTATAAGTAAATTATCTGATTATAATTTAATTCCTGGAGATGTTCTTTATCTTAAAGCT
CAGTTATATGCTGATGCTGATTTACTTGCTCTTGGAACTACAAATATATCTATCCGTTTTTATAATGCAT
CTAACGGATATATTTCTTCAACACAAGCTGAATTTACTGGGCAAGCTGGGTCATGGGAATTAAAGGAAGA
TTATGTAGTTGTTCCAGAAAACGCAGTAGGATTTACGATATACGCACAGAGAACTGCACAAGCTGGCCAA
GGTGGCATGAGAAATTTAAGCTTTTCTGAAGTATCAAGAAATGGCGGCATTTCGAAACCTGCTGAATTTG
GCGTCAATGGTATTCGTGTTAATTATATCTGCGAATCCGCTTCACCCCCGGATATAATGGTACTTCCTAC
GCAAGCATCGTCTAAAACTGGTAAAGTGTTTGGGCAAGAATTTAGAGAAGTTTAAATTGAGGGACCCTTC
GGGTTCCCTTTTTCTTTATAAATACTATTCAAATAAAGGGGCATACAATGGCTGATTTAAAAGTAGGTTC
AACAACTGGAGGCTCTGTCATTTGGCATCAAGGAAATTTTCCATTGAATCCAGCCGGTGACGATGTACTC
TATAAATCATTTAAAATATATTCAGAATATAACAAACCACAAGCTGCTGATAACGATTTCGTTTCTAAAG
CTAATGGTGGTACTTATGCATCAAAGGTAACATTTAACGCTGGCATTCAAGTCCCATATGCTCCAAACAT
CATGAGCCCATGCGGGATTTATGGGGGTAACGGTGATGGTGCTACTTTTGATAAAGCAAATATCGATATT
GTTTCATGGTATGGCGTAGGATTTAAATCGTCATTTGGTTCAACAGGCCGAACTGTTGTAATTAATACAC
GCAATGGTGATATTAACACAAAAGGTGTTGTGTCGGCAGCTGGTCAAGTAAGAAGTGGTGCGGCTGCTCC
TATAGCAGCGAATGACCTTACTAGAAAGGACTATGTTGATGGAGCAATAAATACTGTTACTGCAAATGCA
AACTCTAGGGTGCTACGGTCTGGTGACACCATGACAGGTAATTTAACAGCGCCAAACTTTTTCTCGCAGA
ATCCTGCATCTCAACCCTCACACGTTCCACGATTTGACCAAATCGTAATTAAGGATTCTGTTCAAGATTT
CGGCTATTATTAAGAGGACTTATGGCTACTTTAAAACAAATACAATTTAAAAGAAGCAAAATCGCAGGAA
CACGTCCTGCTGCTTCAGTATTAGCCGAAGGTGAATTGGCTATAAACTTAAAAGATAGAACAATTTTTAC
TAAAGATGATTCAGGAAATATCATCGATCTAGGTTTTGCTAAAGGCGGGCAAGTTGATGGCAACGTTACT
ATTAACGGACTTTTGAGATTAAATGGCGATTATGTACAAACAGGTGGAATGACTGTAAACGGACCCATTG
GTTCTACTGATGGCGTCACTGGAAAAATTTTCAGATCTACACAGGGTTCATTTTATGCAAGAGCAACAAA
CGATACTTCAAATGCCCATTTATGGTTTGAAAATGCCGATGGCACTGAACGTGGCGTTATATATGCTCGC
CCTCAAACTACAACTGACGGTGAAATACGCCTTAGGGTTAGACAAGGAACAGGAAGCACTGCCAACAGTG
AATTCTATTTCCGCTCTATAAATGGAGGCGAATTTCAGGCTAACCGTATTTTAGCATCAGATTCGTTAGT
AACAAAACGCATTGCGGTTGATACCGTTATTCATGATGCCAAAGCATTTGGACAATATGATTCTCACTCT
TTGGTTAATTATGTTTATCCTGGAACCGGTGAAACAAATGGTGTAAACTATCTTCGTAAAGTTCGCGCTA
AGTCCGGTGGTACAATTTATCATGAAATTGTTACTGCACAAACAGGCCTGGCTGATGAAGTTTCTTGGTG
GTCTGGTGATACACCAGTATTTAAACTATACGGTATTCGTGACGATGGCAGAATGATTATCCGTAATAGC
CTTGCATTAGGTACATTCACTACAAATTTCCCGTCTAGTGATTATGGCAACGTCGGTGTAATGGGCGATA
AGTATCTTGTTCTCGGCGACACTGTAACTGGCTTGTCATACAAAAAAACTGGTGTATTTGATCTAGTTGG
CGGTGGATATTCTGTTGCTTCTATTACTCCTGACAGTTTCCGTAGTACTCGTAAAGGTATATTTGGTCGT
TCTGAGGACCAAGGCGCAACTTGGATAATGCCTGGTACAAATGCTGCTCTCTTGTCTGTTCAAACACAAG
CTGATAATAACAATGCTGGAGACGGACAAACCCATATCGGGTACAATGCTGGCGGTAAAATGAACCACTA
TTTCCGTGGTACAGGTCAGATGAATATCAATACCCAACAAGGTATGGAAATTAACCCGGGTATTTTGAAA
TTGGTAACTGGCTCTAATAATGTACAATTTTACGCTGACGGAACTATTTCTTCCATTCAACCTATTAAAT
TAGATAACGAGATATTTTTAACTAAATCTAATAATACTGCGGGTCTTAAATTTGGAGCTCCTAGCCAAGT
TGATGGCACAAGGACTATCCAATGGAACGGTGGTACTCGCGAAGGACAGAATAAAAACTATGTGATTATT
AAAGCATGGGGTAACTCATTTAATGCCACTGGTGATAGATCTCGCGAAACGGTTTTCCAAGTATCAGATA
GTCAAGGATATTATTTTTATGCTCATCGTAAAGCTCCAACCGGCGACGAAACTATTGGACGTATTGAAGC
TCAATTTGCTGGGGATGTTTATGCTAAAGGTATTATTGCCAACGGAAATTTTAGAGTTGTTGGGTCAAGC
GCTTTAGCCGGCAATGTTACTATGTCTAACGGTTTGTTTGTCCAAGGTGGTTCTTCTATTACTGGACAAG
TTAAAATTGGCGGAACAGCAAACGCACTGAGAATTTGGAACGCTGAATATGGTGCTATTTTCCGTCGTTC
GGAAAGTAACTTTTATATTATTCCAACCAATCAAAATGAAGGAGAAAGTGGAGACATTCACAGCTCTTTG
AGACCTGTGAGAATAGGATTAAACGATGGCATGGTTGGGTTAGGAAGAGATTCTTTTATAGTAGATCAAA
ATAATGCTTTAACTACGATAAACAGTAACTCTCGCATTAATGCCAACTTTAGAATGCAATTGGGGCAGTC
GGCATACATTGATGCAGAATGTACTGATGCTGTTCGCCCGGCGGGTGCAGGTTCATTTGCTTCCCAGAAT
AATGAAGACGTCCGTGCGCCGTTCTATATGAATATTGATAGAACTGATGCTAGTGCATATGTTCCTATTT
TGAAACAACGTTATGTTCAAGGCAATGGCTGCTATTCATTAGGGACTTTAATTAATAATGGTAATTTCCG
AGTTCATTACCATGGCGGCGGAGATAACGGTTCTACAGGTCCACAGACTGCTGATTTTGGATGGGAATTT
ATTAAAAACGGTGATTTTATTTCACCTCGCGATTTAATAGCAGGCAAAGTCAGATTTGATAGAACTGGTA
ATATCACTGGTGGTTCTGGTAATTTTGCTAACTTAAACAGTACAATTGAATCACTTAAAACTGATATCAT
GTCGAGTTACCCAATTGGTGCTCCGATTCCTTGGCCGAGTGATTCAGTTCCTGCTGGATTTGCTTTGATG
GAAGGTCAGACCTTTGATAAGTCCGCATATCCAAAGTTAGCTGTTGCATATCCTAGCGGTGTTATTCCAG
ATATGCGCGGGCAAACTATCAAGGGTAAACCAAGTGGTCGTGCTGTTTTGAGCGCTGAGGCAGATGGTGT
TAAGGCTCATAGCCATAGTGCATCGGCTTCAAGTACTGACTTAGGTACTAAAACCACATCAAGCTTTGAC
TATGGTACGAAGGGAACTAACAGTACGGGTGGACACACTCACTCTGGTAGTGGTTCTACTAGCACAAATG
GTGAGCACAGCCACTACATCGAGGCATGGAATGGTACTGGTGTAGGTGGTAATAAGATGTCATCATATGC
CATATCATACAGGGCGGGTGGGAGTAACACTAATGCAGCAGGGAACCACAGTCACACTTTCTCTTTTGGG
ACTAGCAGTGCTGGCGACCATTCCCACTCTGTAGGTATTGGTGCTCATACCCACACGGTAGCAATTGGAT
CACATGGTCATACTATCACTGTAAATAGTACAGGTAATACAGAAAACACGGTTAAAAACATTGCTTTTAA
CTATATCGTTCGTTTAGCATAAGGAGAGGGGCTTCGGCCCTTCTAAATATGAAAATATATCATTATTATT
TTGACACTAAAGAATTTTACAAAGAAGAAAATTACAAACCGGTTAAAGGCCTCGGTCTTCCTGCTCATTC
AACAATTAAAAAACCTTTAGAACCTAAAGAAGGATACGCGGTTGTATTTGATGAACGTACTCAGGATTGG
ATTTATGAAGAAGACCATCGCGGAAAACGCGCATGGACTTTTAATAAAGAAGAAATTTTTATAAGTGACA
TTGGAAGCCCGGTTGGTATAACTTTCGATGAGCCCGGCGAATTTGATATATGGACTGATGACGGTTGGAA
AGAAGACGAAACATATAAGCGAGTTTTAATTCGTAATAGAAAAATTGAAGAATTATATAAAGAGTTCCAA
GTTTTAAATAATATGATTGAAGCTTCTGTCGCCAATAAAAAGGAAAAATTCTATTATAAAAACCTTAAGC
GGTTCTTTGCTCTTTTAGAAAAGCATGAGCATTTAGGTGGTGAATTCCCTTCATGGCCTGAAAAAGAACA
GAAGCCTTGGTATAAGCGTTTATTCAAGCATTACGTATAAATATCTTAAAAGGAGGGTCTATGGCAGCAC
CTAGAATATCATTTTCGCCCTCTGATATTCTATTTGGTGTTCTAGATCGCTTGTTCAAAGATAACGCTAC
CGGGAAGGTTCTTGCTTCCCGGGTAGCTGTCGTAATTCTTTTGTTTATAATGGCGATTGTTTGGTATAGG
GGAGATAGTTTCTTTGAGTACTATAAGCAATCAAAGTATGAAACATACAGTGAAATTATTGAAAAGGAAA
GAACTGCACGCTTTGAATCTGTCGCCCTGGAACAACTCCAGATAGTTCATATATCATCTGAGGCAGACTT
TAGTGCGGTGTATTCTTTCCGCCCTAAAAACTTAAACTATTTTGTTGATATTATAGCATACGAAGGAAAA
TTACCTTCAACAATAAGTGAAAAATCACTTGGAGGATATCCTGTTGATAAAACTATGGATGAATATACAG
TTCATTTAAATGGACGTCATTATTATTCCAACTCAAAATTTGCTTTTTTACCAACTAAAAAGCCTACTCC
CGAAATAAACTACATGTACAGTTGTCCATATTTTAATTTGGATAATATCTATGCTGGAACGATAACCATG
TACTGGTATAGAAATGATCATATAAGTAATGACCGCCTTGAATCAATATGTGCTCAGGCGGCCAGAATAT
TAGGAAGGGCTAAATAATTATTTGTTCGTATACATCTCTAGATATCGATATACACCCTCAAAACCCTCGT
TGAATTCGTCGATGAGGGTTTTCTTATCTTCTTGAGTTAATTCAGAAACAATTTTACGGAATGAATTTTG
ATTTAACTTTCTACCTTCATGCGTTACTCCAATCTCATTTAGAAATGCAATAAAATTAGCACGATTCTCA
ACAATATCTTCTCTGGAAAATTTAATCAAAATAGATGCAACAGTAATAATTTCACGAACTGTATCAATGT
TTTTATTCATTAACTATACCACTCAATTAGTTGACTTTGTTATAATATCATCAGACGCTTGATTTGTAAA
CTGGTCTGTGTAATTTTCTTCAAAAATTTTTTCTACGAATTCCTTGAACGATTCACGTTCCTGAGCTACA
TTATGCTCGATTACCTTTTCAAGATTATGACTCATTCGAAATAATCTTCAATTTCATAATCATGGACATA
AATCATTATAGTTTTTAATACATCATCAATATTTTTTCCTGGAGCTGGAATTACGTAAAAATACCCTGCT
TTTGAGAGGTCTTTATAAGTTCCAATCAAGAAATCATTATTCTCAAGATGTAACTCTTCAACTAATTCAT
TGACAATTGAATGGTATAGGTTTGGCAGAAACTTATATAGCTTTTCTAGAATATCAATTTTGAATGTATA
TTGAACCACGGACTGAGAATCAATAATCATAGACCTTCCCCTTATGTTTCTGTTTGCGATTAGATTCTTT
AAACGCTTTCTTCTTATCCTTATGAACAGAAGCTTTATTAAAATTATGCTTTGCGACTAAATTGTTCATA
GTGCTGAATTACCTCTCTTAAACATTTGCATGTGAATGAAAACTTTTTAGCTACACCACATTCAAATATA
TGTTCTCTTAAATCGCGTGTATCGGTATATCCCATCTCAACAATAAAATGCCGTATTAGATTTTTATCTT
TATCGTTTAGAGAATTAAAATAATCAGATTTTGAATTAATTTCCCTGGCCAAATTGAATCACCTTCAGTT
GACGTTTTAACTCTTTTATCATCTCTTCGTTCATCGCAATATAAAGATCGCGTAGAGCAGGTTTTAGCAT
TCCATTTACTGGAGAACTAAATGGACATACATAATCTTTTCCTACGAGCTTTTTAGTGAATTCCATATCA
CAGAACTGAAATCCCGGCTCATTGGTATAAATTCCCCAATTAGTTGACATCATTTTATTGGCATATTCCA
GTGCCTGGATTTGATTCATAATTCCATCAATTTGAAACTTTTTAATATTCATTAGTAAAGGTCCTCAGAG
TAAAGTTCTTTTTCACTACCACGTTCAATACTTACTTGTCCAGCGTAAGTTGCAATAATCATTGCTTCTT
CACGTGTCCAATAATTACTATATTGGTCAATAAACCCTTGGTCATCATCACAAACTTGCTGAGTAACTAA
TTGAGGTTTAACTACATCTAAAACTTCTGCCATATCTTTAGAATAATGACGAGCACCTGGAATAATAAGA
GTTCGTCCATCTTTTAATTTAAAACGGTTGGCTGCGCAAACAATTCGTCGTTGATACTTTTGGTTTTCAT
CCCAGTACGCAGTCTGCCAACAGATTTCAGGAACTTCTTTCAGAATATCTTCTTCTGTGCATTTATAACC
ATGCGCTTTAAATTTTTCAACAAGACTTTCTGGAGTTTCACGAGATAAAGGAACATTCAGCATTTTTAAA
CGATTGATAAATGGGTTCATTTAAACCATCCTTTAATACGCTGCCACAAAGTTTTCTGTTGAGCTTTGTT
GACGCCAATTGAGCGAATAACCGGTTGAGATTCCTGGAATTCTTTATAATCAGCAAGGTAAATTTCGTAA
GCTGCATCCGTAAATGAACTTATCGCTGCCATAAAATTATTGCGAATACCTACTGGAGCATCTTTACTTT
CACGAATGATCATGTATTTACCAGTCTTAATCTTTACGATAGTTCCAAGATAAGCTCCATGGTACCAAAT
ATCCCAACCCTCTTGAGTAGGTTCTGCGCAACGACGAAGTTCATTGACAATTTCTAACTTGTTTATTATT
TATTCCTCACAGTTCAGATGCTACAGTGATTACAGCTTCAATGTTTTCTGCCGAGCGTTTAATGTCAAGA
TACACATTACCGTTTTTAGCGATTTTACATGACATTCCGATGTCAGTAAATTTCTGAATATGATGTTCCA
TCATTTTGTATCCAAAAATTCGCATATTTCCATTGTTATTAATTTCAAAATTACGAATTCCGTTAGTGCG
TTTTTCTAAAATAGCAAGATAATTACTACGATAAATTTCAACCTTTTTAAGAACAAATCCATTTTTATCT
AAAAGTTTTAACATGAGGTCTTTATCTTCTTCCATATCGGAAGTAATCTCGCGAGCTTTACGAGTTGCTC
GTTTTTTCAGCAGTTCCGGAGCATTTTCCTGTGCATATAAAGTTGCTGCATTTGAAATAATATCCTGAGC
TTCACCAGTAATGATTAATCCATCACCAGATTTCTCCACCAGGCCTTTTTTAATCAATACCCCAATATTA
CTATTAACTACTGCGTTACCTAAATCTGGATGCACCTCACGAACTTCTGCAGCTGTAATGAAATCTTTCT
TAGCAATGGTAATTAAAATCGTAGCAGTTTTTTCATTCAGAACATCGTTAGAAGCTTTGATGATGTAAGT
TACTTTAGACATTTTCTAATCTCCGTAATTCTGTATCAGTAGTTGATAGTTGTATAGTACCACAGTATGC
TTTGGTTGTAAACCGTTTTGTGAAAAAATTTTTAAAATAAAAAAGGGAGAGCCTCGGCTCTCCCTAAAAT
TACTGCATGACTGTGATAACTGTCATGATAACACGTTGAATTCCGAACGCAAGAAGACCTCCTGCTACGG
CTGGAACAACGCCTAAACCCGCCAGTAAAATGCTACCAGATACTAATGCAGCGCTTGTAATACCAATGAA
TGGACTCATTTGATTTCCTCTAAATCTTTGGTGTATTCAGTAACTACATCAGTAGTTTTCCAATATTCGT
TTTCTTCTTTTTTAGCTTTAGCTTCTTCAGCAAGTTTCTTTGCTTCATCGGAAGTCATATGAAAAATATT
CATTCCAACTAGTTTATCAACATAAGAAGAATACATATCGATTTTCGAAAGTTCTTCGGTCAGTTCTTTA
CGAGTTTTACCCTGTACAACAATTTCACCTGAAATTACTTTCTTAATGAAATGTGCTTTGGCAAAGGCTA
AACGAAAAGCTGACTCAGTTTCTTTAATTTTGTTATCAATTCGTTTTTGGACATAAGTTTTACGAACTTC
AACAAAGTCTTTAATTAAATCAACTACGTTATCGTAAACTTGCAGCTTTCCTTTCTCATTAATAACCGTA
ATATTCTGGGAACGACGCTCAATCAGTCCGAAGTCTTTCATAATTTTTGCATGGCGTTCTTCTTCGTTAT
CGCTCAAAGAATATTCTTTGCGGAATTTAACTTTGAAGCCAAAACCATGCTCACCACAAGCATCATCCCA
TGTAATGAAGCCTTTATTTTCAAGTGGGTCTAAGATTTTACTCACATAAGTTTCACGATCATACTTATAC
GGAATCTCAGTGATATGCATTTGAGTTCGTGAAGTAAACTTATATGTTCCACGAATTTCATATTGCCCAT
CAATTTCAACGACTTCACCACGAAATTCTGGGAATTCTACTTTCGGTTTAGTTACTTTCTTTCCTTGAAG
AGCTTGCAGTACAGCTTTCTTGACAGAAGAAACACTATGAGGAAGAATGTAAGTTGCATAACCAGTTGCA
ATACCGGAAACGCCATTAAGAAGAACAGTAGGAATAATAGGCAAATAGAAAGCAGGCGGAATGTGTTCTT
TATCTTGATGTACCGGAGCATATTCAGTATCTTTATATACGTTATAGAAATTTTTACTTACACGAGCAAA
AATATAACGACTTGCCGCTGCCTTTTGGACAGTACGAGAACCAAAGTTTCCTTGACCATCTAACAGAGGA
AAGTTATTATTCCAAGTATTAGCCATCAAAGCACCTGCGTCTTGTGCAGAGTTTTCACCATGATGATATC
CAAGGTCCGCTACACCACCTGCAATAGAAGCGAGTTTGTGAAACTTATCTTTATTTCCTCGTGCCAAATC
AAGAGCTCGAGCAATAACAAATCGTTGAACTGGCTTAAATCCATCAATCATATTTGGGATAGCACGATTT
TCAACCGTGTACATAGCATAAGCCAATGCTTCATTATCAATGATACTTTTTAAATCGCGATTATTCAGTT
GCATAAATTTACCATACTAGTGAATGTAGTGCCATAATAACATCAGAAATGAAAAGCACGACTTGAATTA
ATCCGAACATTACTCCGTAATATAGTGCTACCAATAAAGCAGCAAGGGCTAATGAATAGCCCAAGATTTT
CTTAATCATTAGATAACAACACAAATGTTAAATATGCACACATACCCTGGGCTAAAGCTTGTGAAAACAC
ACTGCTAGCATCGATACAGATAGTTAAAACACATGCTACTATCCAACAAATAAATGAAATAACTCCTAAT
AATTTTGCAATATTCATATTTTCCTCACTGGCGTCCGAAGACGCCTTTAGTTTTAAGATTGTTACGATAG
AACTGCATCACGTGTTCGTTATGGAAATTACTCATTAATATGCCTGTAAAACAAATTTAAAGTTATCAGC
CAACATACGGTTCATTTCTTCGAGTGTTTGATACTCAGAATGATGATTACGAGTAAACGCCAAAGCTAGC
TGACCTTTTCCAAATCCCGTCGTTAGAGGTTTCATCTTAGAAGCAGGCAGATAAAACACTGTGTATGGAA
CATTGTTATTTGCAATAGTACGCGCAAGTTGAGACCGGCGTTGACGAATATGACTTAAAACCGCACTGAA
TCCTTGCTTAGAACGCTGATTACCTACATAAAATCGTGCAGATACGCATGGATTACTAAATGGACCACCG
AGTTTACTCACTAAAAAGTAAAATCCAGGTTTAGATAAAATATCTTTATGCGGAGTTCCTAAAAACCATT
CACCACCCTTGATTGTACCAATAACAGTAGCGCCTGCATCATTCAGATCAGTAACAGTCATATATTTCAT
ATTAATTTCCTCTAAATTATTTTCTACTCCAAGGCCGCATGAATACACACGGCCATTAAATTACTCGTCG
CAGTCGACGCTCAATTCCCAAAACTCTTCTACAGTATAAGTTTCAGTATCATTTTCAATACAGAAACGTT
CATTACTATTATTTGCTAAAGTAGCATTAACTGTCATTTTTTCGCTAGTGCTCTTAAGAGGTGAAATACG
AATTAACTGATCACCGTTATCTAAACAAAAAATTTCACCAACTTTTACATCTTTAAAACTTTTCATAATT
CACCTCAAGGAGTATAAAATCCAAATGCAGTTGTTGACCATCCCATCCAATATGGAAAATTTACACCAAT
GTAAAACATAAGAATATAAAACCAACCGCTCAGCAAATTCATCATTTTACACCATTCCAAATTGTTTCAA
CCACGGATTTTAAACCATTTTGATGAATATCCATTCCTACTACCGCCATCAAATAAATTCCAACTACAAC
TGAACCTAAGGCAAAAATCAGCATGAAAATGAATAAAGCCGGAAAAATATTATCGAAAAACCATTCAATA
AATGTAAAAGCACTGCGTTTACGTTCATATTTTCCTCACATAAATCCAAAGTAAACGTTTAATACATCAA
TCATTAAAACGATTGGGAATATACTCAAAACTATTAGTATTATAACTACATTCCATATAGCTTTAATAAT
CTTTTTCATTTTCTGTTCCTCCATAGTTGATAGGGTAATAGTACCACGGAAGAACAGTCTTGTAAACAAC
TTTTTTAAAAATATTCGTAATAAATGTGAATACCAACTACTACCGCTGAAACCTGTGCAACCCACCACGC
ACAAGCAATAAGTACAGAATTCAAAATTTTCATAATAACCTCATTACAAAAGTAAATGTTAAACAAATTA
CTGGAATACTAATTAACCAAACAAAACACCACCATAATGAACTCATAGTTCAATCTCAGCGATTTTCATT
TTATTCTCCAAATCCGTATCAGTAGTTGATAGTTGTATAGTACCACGGTCCTTGTGGTATGTAAACTGTT
TTGTGAAATTTTTTAAATGGAAAGATACCATCCGTTGTAGTTGCTTTTTCTTACAACTTTACGAAGGTCT
TCTCTGTCACCGATGAACTTCGGAGTGTACTGGATGACACCTGGATGAATTTCTTTAGTGTTGAATATAA
TTATACAGTCAGCGACTTGATGATTTAGAATGGGCCCTAGATTTATTCCAGAACCATATGGATACTCTCC
GCTGCATCCCGTTGTTACCGAAATCCAACGTGAGTCAGTTTGATGTGTCTTAACTTCTACACGAAGCCCG
CAGTATTTTGGATGAGCCAATACATCCCATGCATATGTGTACGGATCATCGACATCCTCTTGGCCTTTAT
TGACATATCCACTCAACCAATCTGCCACAAAAAACTCTGCGTACACAGCGATACGGCATCTTTCGATAAC
TTCTGCCTTATCTTGATTTGGGTTTTGTTTTAAAGAGTATCTTGCAGTATCAGCAATTTTGACCTTCATT
TCACAGGTCAAGTCACTGTTCGATAGGGTAAATGTCGGAATCTGAAATAGTCTCTGTAACCCAGGATTCG
TTTTCTGCATTTAAACTTTCCTTTATGTCGGAATCACCGATATTCATATAAATCATAATTTCTCTTAAAA
CAAAAGGCCGAAGCCCTTTATTTTACTTGAATTGTGCAATTCTTTTCTCTAGACATTCAGCATAAGATTT
CATTGAGATGAACTGCGAAAGTAGCAGTTCTTGCTCAACTGCGCTAACTGTTAGAAACTTTGCGCTTTCT
AAAAATTTGCTCAGTGCATTAATTTTGAGCATTAATTGATCGTATTCTTCTTTTACTCGTGCTTGATAAG
CTAACATAATTTTCCTTAGTTAAGGGCCGAAGCCTTATTTAAATTGTTCAGTAACGTCTTCAACTACTTC
ATATTGGCAGGTACGCATTTTAGCATCGTTGTAATCAATCGGAATTGATACTACATCGCGAGGATGAACT
TTAACTTTTACAACTCGGCTGGTTGAACTACCAAAGTGACGAATATAAGATTTAGAACACACATGCAAAC
CACGAGAACAAGTTTGTGTATCATCGTCATTCACACGAGTACGTGGCATTTTAACTACTTTACCCGGACT
GTTATCAAAGGTGTTTGAGTGACAGTCAAAGTAATTGCTGCGAACTACTTTCCAAGCATAGAAGTAACCA
TCTTCTGTAATTTCAATATCGTTTGCTACCAAGAAATCAAAGAGTCGAGATACCGCTTTTTGGCTTGGGT
TTTCCAACAGATTTTCCAAGAACGGAAAATAAAATTCAAAGTTTTCGCCTTTTTCCATCGAGTCAAGAAT
ACGATCAACCAAACCAGACCGCAATTCAATATTTTGATAGAACAAGCTTCCACCTTCAATTCGAACATCG
CCGGAAATATATTTTTCAACAGCACGACGAACATTAATTTTTTGTGCCGCTTCTTCCAACTTATCCGCTA
CAAGCAGATTAAGAATTTCCTGGAAGTTTGAATGAGTATTAGGAGTTGCGTTATAAGTTACGCCATCAAC
AGTAATTGAAATGAATTTTTTAGATGCATTCCAAATAATGTCAGATTTAGCAACTGGAGCAATAACTGCA
TCGCTATTAACTTTAACTGTAATATCACCGCTAATAGTAACTTTAGGGCGTTTAGCTTCTTCAGCATTTT
TCAAAACACGACGGATTGTGTCAACCGATACACCTTGCCAATCAGCCAATTCCTGTTGGGTGTAATTACC
ACTTGAATACAGTTTAACAATTTCAGCTTGTTCGTTTTTGGTCAGGCATTTAATATTGTACAT
Spacer targeting E coli fimH
(SEQ ID NO: 6)
CGAATGACCAGGCATTTACCGACCAGCCCATC
Spacer targeting E coli bolA
(SEQ ID NO: 7)
AGTGGGAAGGGTTGCAGGACACCGTCTTTGCC
Spacer targeting E coli rpoH
(SEQ ID NO: 8)
CCGATGTTACCTTCCTGAATCAAATCCGCCTG
Spacer targeting E coli IptA
(SEQ ID NO: 9)
TGATTGACGGCTACGGTAAACCGGCAACGTTC
Spacer targeting E coli murA
(SEQ ID NO: 10)
GCTGTTAACGTACGTACCGCGCCGCATCCGGC

1. A composition comprising a plurality of transduction particles, wherein (a) each transduction particle comprises a nucleic acid encoding a Cas nuclease and a nucleic acid encoding a crRNA or guide RNA that is operable with the Cas nuclease for targeting the genomes of Escherichia coli (E. coli) cells,
wherein (i) each transduction particle is a phage and the nucleic acid encoding the Cas nuclease is integrated into the genome of the phage, or (ii) each transduction particle is a phage capsid and the nucleic acid encoding the Cas nuclease is a phagemid contained in the phage capsid,
(b) the E. coli cells are cells of E. coli phylogroup B2; and
(c) wherein the plurality of transduction particles comprises a first type of transduction particle and a second type of transduction particle, wherein the first type of transduction particle comprises a first adhesion moiety that is capable of recognizing and binding to LPS displayed on the surface of phylogroup B2 E. coli cells, and the second type of transduction particle comprises a second adhesion moiety that is capable of recognizing and binding to Tsx displayed on the surface of phylogroup B2 E. coli cells.

2. (canceled)

3. The composition of claim 1, wherein the E. coli cells comprise an E. coli strain selected from the group consisting of ST131 and ST1193.

4. The composition of claim 1, wherein the E. coli cells comprise a plurality of different phylogroup B2 strains of E. coli.

5-6. (canceled)

7. The composition of claim 1, wherein the B2 E. coli cells comprise a strain of E. coli that causes sepsis, septicaemia or diarrhoea in humans.

8. (canceled)

9. The composition of claim 1, wherein each particle comprises a phage capsid containing the nucleic acid.

10-13. (canceled)

14. A composition comprising a plurality of transduction particles, wherein
(a) each transduction particle comprises a nucleotide sequence (N1) encoding a Cas nuclease for targeting the genomes of E. coli cells and a nucleic acid encoding a crRNA or guide RNA that is operable with the Cas nuclease for targeting the genomes of E. coli cells,
(b) the E. coli cells are cells of E. coli phylogroup B2; and
each transduction particle comprises an adhesion moiety that is capable of recognizing and binding to a cognate moiety selected from the group consisting of a LPS, LamB and Tsx displayed on the surface of phylogroup B2 E. coli cells,
wherein each transduction particle is a synthetic T-even phage comprising an insertion of N1 into the genome of the phage, wherein the insertion is between the pin (protease inhibitor) gene and the iPII (internal protein) gene.

15. (canceled)

16. The composition of claim 1, wherein the nuclease is a Type I, II, III, IV, V or VI Cas nuclease.

17. The composition of claim 1, wherein the composition comprises at least 1×10⁷ PFU of the transduction particles are administered to the subject.

18. (canceled)

19. The composition of claim 1, wherein the E. coli cells comprise a strain or at least one strain that is an antibiotic-resistant or MDR (multidrug resistant) strain; and/or wherein the E. coli cells comprise a strain or at least one strain that is a B2-I strain.

20. The composition claim 19, wherein the antibiotic-resistant strain is resistant to fluoroquinolone, carbapenem or vancomycin; and/or wherein the E. coli are a beta-lactamase (ESBL)-producing E coli.

21-33. (canceled)

34. The composition of claim 1, wherein each transduction particle is a lytic phage.

35. The composition of claim 4, wherein the plurality of different phylogroup B2 strains of E. coli comprises E. coli ST131 and ST1193 cells.

36. The composition of claim 9, wherein the phage capsid comprises capsid proteins of a T-even or lambda phage.

37. The composition of claim 1, wherein the crRNA or guide RNA is operable with the Cas nuclease for targeting the genome of an E. coli strain selected from the group consisting of ST131 and ST1193.

38. The composition of claim 1, wherein the plurality of transduction particles comprises a third type of transduction particle, wherein the third type of transduction particle comprises a third adhesion moiety that is capable of recognizing and binding to LamB.

39. The composition of claim 1, wherein each transduction particle is a synthetic T-even phage.

40. The composition of claim 1, wherein the Cas nuclease is a Type I Cas nuclease.

41. The composition of claim 14, wherein each transduction particle comprises a nucleic acid encoding a crRNA or guide RNA that is operable with the Cas nuclease for targeting the genomes of E. coli cells.

42. The composition of claim 14, wherein the plurality of transduction particles comprises a first type of transduction particle and a second type of transduction particle, wherein the first type of transduction particle comprises a first adhesion moiety that is capable of recognizing and binding to LPS displayed on the B2 E. coli cells, and the second type of transduction particle comprises a second adhesion moiety that is capable of recognizing and binding to Tsx.

43. The composition of claim 14, wherein the nuclease is a Type I Cas nuclease.

44. The composition of claim 43, wherein the plurality of transduction particles comprises a third type of transduction particle, wherein the third type of transduction particle comprises a third adhesion moiety that is capable of recognizing and binding to LamB.

45. The composition of claim 1, wherein the crRNA or guide RNA targets a chromosomal sequence of the E. coli cells.

46. The composition of claim 45, wherein the plurality of transduction particles comprises a third type of transduction particle, wherein the third type of transduction particle comprises a third adhesion moiety that is capable of recognizing and binding to LamB.

47. The composition of claim 45, wherein the nuclease is an endogenous nuclease of the cells and is not encoded by the nucleic acid comprised by the transduction particles.


SEQUENCE LISTING (10 sequences)

SEQ ID NO: 1 - protein, 446 aa, Escherichia coli
MMITLRKLPLAVAVAAGVMSAQAMAVDFHGYARSGIGWTGSGGEQQCFQTTGAQSKYRLGNECETYAELKLGQEVWKEGDKSFYFDTNVAYSVAQQNDWEATDPAFREANVQGKNLIEWLPGSTIWAGKRFYQRHDVHMIDFYYWDISGPGAGLENIDVGFGKLSLAATRSSEAGGSSSFASNNIYDYTNETANDVFDVRLAQMEINPGGTLELGVDYGRANLRDNYRLVDGASKDGWLFTAEHTQSVLKGFNKFVVQYATDSMTSQGKGLSQGSGVAFDNEKFAYNINNNGHMLRILDHGAISMGDNWDMMYVGMYQDINWDNDNGTKWWTVGIRPMYKWTPIMSTVMEIGYDNVESQRTGDKNNQYKITLAQQWQAGDSIWSRPAIRVFATYAKWDEKWGYDYNGDSKVNPNYGKAVPADFNGGSFGRGDSDEWTFGAQMEIWW

SEQ ID NO: 2 - protein, 294 aa, Escherichia coli
MKKTLLAAGAVLALSSSFTVNAAENDKPQYLSDWWHQSVNVVGSYHTRFGPQIRNDTYLEYEAFAKKDWFDFYGYADAPVFFGGNSDAKGIWNHGSPLFMEIEPRFSIDKLTNTDLSFGPFKEWYFANNYIYDMGRNKDGRQSTWYMGLGTDIDTGLPMSLSMNVYAKYQWQNYGAANENEWDGYRFKIKYFVPITDLWGGQLSYIGFTNFDWGSDLGDDSGNAINGIKTRTNNSIASSHILALNYDHWHYSVVARYWHDGGQWNDDAELNFGNGNFNVRSTGWGGYLVVGYNF

SEQ ID NO: 3 - DNA, 1341 bp, synthetic construct
  source 1..1341: mol_type = other DNA
atgatgattactctgcgcaaacttcctctggcggttgccgtcgcagcgggcgtaatgtctgctcaggcaatggctgttgatttccacggctatgcacgttccggtattggctggacaggtagcggcggtgaacaacagtgtttccagactaccggtgctcaaagtaaataccgtcttggcaacgaatgtgaaacttatgctgaattaaaattgggtcaggaagtgtggaaagagggcgataagagcttctatttcgacactaacgtggcctattccgtcgcgcaacagaatgactgggaagctaccgatccggccttccgtgaagcaaacgtgcagggtaaaaacctgatcgaatggctgccaggttccaccatctgggcaggtaagcgcttctaccaacgtcatgacgttcatatgatcgacttctactactgggatatttctggtcctggtgccggtctggaaaacatcgatgttggcttcggtaaactctctctggcagcaacccgctcctctgaagctggtggttcttcctcttttgccagcaacaatatttatgactataccaacgaaaccgcgaacgacgttttcgatgtgcgtttagcgcagatggaaatcaacccgggcggcacattagaactgggtgtcgactacggtcgtgccaacctgcgtgataactatcgtctggttgatggcgcatcgaaagacggctggttattcactgctgaacatactcagagtgtcctgaagggctttaacaagtttgttgttcagtacgctactgactcgatgacctcgcagggtaaaggtctgtcgcagggttctggcgtcgcgtttgataacgaaaaatttgcctacaatatcaacaacaacggtcacatgctgcgtatcctcgaccacggtgcgatctccatgggcgacaactgggacatgatgtacgtgggtatgtaccaggatatcaactgggataacgacaacggcaccaagtggtggaccgttggtattcgcccgatgtacaagtggacgccaatcatgagcaccgtgatggaaatcggctacgacaacgtcgaatcccagcgcaccggcgacaagaacaatcagtacaaaattaccctcgcacaacaatggcaggctggcgacagcatctggtcacgcccggctattcgtgtcttcgcaacctacgccaagtgggatgagaaatggggttatgactacaacggcgatagcaaggttaacccgaactacggcaaagccgttcctgctgatttcaacggcggcagcttcggtcgtggcgacagcgacgagtggaccttcggtgcccagatggaaatctggtggtaa

SEQ ID NO: 4 - DNA, 885 bp, synthetic construct
  source 1..885: mol_type = other DNA
atgaaaaaaacattactggcagccggtgcggtactggcgctctcttcgtcttttactgtcaacgcagctgaaaacgacaaaccgcagtatctttccgactggtggcaccagagcgttaacgttgtcggaagctatcacacccgtttcggaccgcagatccgcaacgatacctaccttgagtacgaagcattcgctaaaaaagactggttcgacttctatggttatgcggatgcgccggtattcttcggcggtaactccgatgcaaaaggtatctggaaccacggttctccgctgtttatggaaatcgaaccacgtttctccatcgacaagctgaccaatactgaccttagcttcggtccgttcaaagagtggtacttcgcgaacaactacatttacgacatgggtcgtaataaagatggtcgccagagcacctggtacatgggtctgggtaccgatatcgacactggcctgccgatgagcctgtccatgaacgtctatgcgaaataccagtggcagaactatggcgcagcgaacgaaaacgagtgggacggttaccgtttcaaaattaaatactttgtgccgattaccgatctgtggggcggtcagctgagctacatcggcttcaccaacttcgactggggttccgatttaggggatgacagcggtaacgcaatcaacggtattaagacccgtactaataactctatcgcttccagccatattctggctctgaactacgatcactggcactactctgtcgtagctcgttactggcacgacggtggtcagtggaacgacgatgcagaactgaacttcggcaacggcaacttcaacgttcgctctaccggctggggtggttacctggtagtaggttacaacttctga

SEQ ID NO: 5 - DNA, 168903 bp, T4 phage
  source 1..168903: mol_type = genomic DNA
aattttccttattaggccgcaagggccttcatagttttagcgatttgggaaacttcatcatcacttaaagagttgcgataaccgatgaagtcggaaacaatacggaatttcttggtaaactcagcaaccattttatcactgttttttgaagcattatttgataatacatcaaaaagattagttactgtccaaatgtcatgaccgatggtatcttttccaccattaaaatatacaccctgtaatgaactaaccatattagcgagtcgtgtatattcttcagaaacttcatctatactgaagtacttcatcataaaatctaactcaggatacttgataattttatcaatatatcgtttagctgaacttgaataacctacatacttatcataatctacatcatcaaaagcatctacatataaatcacgcaaagcttcaaaaatacattggcactgaccgagttcttttacctttttctgtaaaagcggacgaataacataaaattcattaatgccaataagattagccatacgaatcaaaatattcatagatggatgacaaagagatgtagtaccatccatagagaaaatatcagaacgatgcatatacgctacataaccagtaatttcatctgcttctgatgtgaggcgtaaataattcctcttttcccagcgcccgtctttaatttcaaacttaaacgctgtagcagctttaggacgaggagctttacttttaactacctttggaatataactttttactaaagcttcaatttctgacaaataatgaatgttaacttcatcactttcaaacatcgccataatatcaggaagcaaatcaatctgcgattctacttctggattaataaacagaagacgttcgttatgatgaatattcaaagtgttattaaattcactatcatctaacgcacgtgctaatccacggacaatattaacacgatttttaatattatcaataacgatattaatttttgttgtattaataccaaacagacgataacttgatgcaacggctgaagtttcatgactttgcttaatgcgcttcagtcgagggtcaagatttacttcatacacaactcccgcgttgcataacttactgtcaggttcaaacatgctctgcatctttttatatgacagatttttagtcgtgaatttgactgaattactaatcatataatctcgagcagaataccccatcttcatcaattcacgatatgtgtgacgaggagatgtagattctttaaatcgttttacatcttcattaaatgctttctcactgagttctttaactcgttcaataatatttttacgagtgcgatcatccagtgaaagagcctcgcgagatggagcaatatcaagtgaacccattggaaacttaatgtaattcacttcattgcgaatgcttagccagttacggtctctaataacaccatcgataggataaacaataccaccgtagatagcatataatccaccacgatcaggccagtatctttctggatttacaccgtaatagtcatcaaaatccggaaaataatcaatttcgcggtcaagaccattaatgatagccaaatctttgaacggtcgcatgatataagaaacttcataagcaaagtttctaaagtctttttcttcaactggaactacgatttcaataccagttttatcatctggacccatttcttttacgaatgtaggtttaatctgtggaccatcaccatccatgtaagctacataaccacgaatttcacctttatgatacgaagtaatactaaacgtatcagtataactaaacggagatttagaacctaaaccaaatccgccaataaagtcattagattcagctttagatgaactgaagtatgaattatacaacccaggagaattatcatcaccttgaatatcaaaatcactcatacccggaccaaaatctcgacaaacaaatcgtgggtctaaacgtccaggaacttgtatgataaatttttcaggatttccattaagtgcatgagcatcaatcatgttagtaatcaattcacggactactgcgcgaatcttgtttgtatacaaatcagatgacagaattttaaatactttaggagatgctgtgatgctaaatgcttttgatttagaaccattaccaagaattgtttctttttcagtggtgataatcataatttcctcattaattcatattacgcttaataacttcagcaacttctagtagttcatctttagttgcggtgtcggattgaattttatctctaatatctttaaagcgggttttaaattcttcggcttctcccatatcgaaaaagcgttgaatgattctatattctcgatgaactgctttatcaaaaagttctaaatttactttatatgatttcatttcaatatcctcatttgcccaattaattataccacatccttgtggtaaagtaaactactggctcatccattctttacgaaggtcagcattatctcccatgagcatttcaaaaagctctttccagttctcaggaagtttaacaacatcatatactgggttttgaatcatctcacgatattcagatttttccaaagagccaagtcccttaatataacggatgctatgtttaggtagagcatctttggcactctcatattcagcgactgtataaaaccattcttgttttttaccgacctgagcgatgattacaggagttttgacaaagcgaattcgtccttgctcaaacaattctggccaattactaaaaaatccgagcagagaaggataaatagaacctaatccaataatctcttaattatgaggtatttctatagatagcccgaaggctatccatcgtgatctgcgtctgtcataatagcgacattcgcatagttcattgaagaggatttaatagaacgacgaacattgttctgcaatttatattttttcatatcaacgctagaagaatcaatttttacaaatttcattatacacctcatagaacttttcatcaggaatccaaccgcgtttaaattcattaaatgctcggccgaataattttgaattcacagttatattattaactgatttccatttagcaactcccgttcgtttataatgatcggggtcatatttcgtgacgtaccattcatataaatttggtattaattttacagcctctggatttgtcgctgatttattataccatggtttcgctttttcaagttcagctgcttttgcagtagcagcaacagtatttccaacaccaactaatttttcagttttaatgcgtggatcattaacatgaagacgaaaaacttcgcccttttcatttttaaagcatgtcatgcctttaattccaggaagcttaacacctttattaacaccgcatcattcctttgggttaaatgatcctttaattaataaggcgcatttacccgatttaactacttctcattcaacaactttatctttcataacgttttttgaccattcagatactgctctttgatggctaaattctagcaatttcactataatttgcactagaacgtaaactatttttcagtgtttcaacattctattcatcgcatatgccatttcacgattacgatgaattttatatagtagaaaatagtgctaaaaagtgttcacgaaaagtcatgtttcaccaaattttcgttatcatcagaacctcccattgatcttggaaggatatgatgaatttcacccttaaatttagaaacgcgggttttaccccgcactattaagtcattatagattttttcgtaattcacctactgttatccatttaccattaatctgtacttcatcattttcatttacgataattgtatcgccatttagctcgaaagtaaaccactcgccatcttctttttcttcaaacgctttttcaccgagaactagaccagtgattgcgcaaatatcaaatagttctttgtttttaagcatatctgcataagacataccccaactgttgagaactttaccacgcaatggataaccaccgtgaagttctttatcacgaacatcaataagatatccgatagccgaatcaccctcagtcaagaaaagagtagtatcagcatctttaccgcaaagattcgctttgatatgtttatgaaccttagctttagaagcctttttagctgctttagtttctgctgctttttctgccgccaatttacgagccaaagcagcttcaataatcggcattagaattgcttcattatttagaatatcacgtgaaatctttttagcatcaagttgaatatgactacgaatttcgccaaatggagaagtcaaacgctctttagtttgacgaatcaatcgcatgtttttcatatcacgaacaaacataacgatagtcaaacattctttgacacgtgctttagtcacatcaattttgaacttacgtttgatttgtggaataaggtcttcacaaatatcatccatagcgcagtcaatgtgatggccaccattcttagtatgaatgttattgacgtatgttaattgacgaaaaccatccggtgaacgaccaaccgcaatagaacaattttcttgctcttgaacaatagcatgttcatcatactgccgtgcatatttcttaaaattgccctgaacctttttaccattaaaggtaaattgaatatcaggataaactacagcaagtgtctggagacgatccagtgtaatgtcaagataaacttgggacagctcattagtttcaaatgacataaaatcaggaatgaaagtaacacgagttcctttccattttccaggaatatcttcccatgatttattttccatgccatttgaacaacgaactacaatattattttgaccgtcgccagtttcaccgacaaacatcacagaaaaaatgtttgtcaaactagaaccaacaccgttcataccgccggtgacgcgttctttatcatcaccaaagttaccacctgcttttggaatagtccatgcggcaacaggaccaggaatttcttcaccggtaggtgttttaaccatcgcttgtggaataccgcgaccgttatcttcaactgttacttgattgtttttaatagtaacattaattttattcgcgaatttaaacttagtacgaataccttcatctactgagttatcgataatttcatcaataagcttaacaagaccaggtacatactgaacactttcccatttaccaaacataaagcgctcatgcgtttcattagcagaagagccaatgtacatgccactacgctttttgatatgttcaatatcgctcagaattttaatttcattcttaatcatcacttatcctcgtttggtttcgggaatattatactccggtaatcataaagctaaaggcccgaaggccttttatttaaaacgaatagttgaatccttaaagaacagcccagaacatactgttccttctactttctgcccggtaggtccaatagcacgaaatccagtatgctggaaatcattttcagagcaaccgaaccaattatatccagtgatttcaatattagtaaaaccacttgaagacaaaactttggttgcattaatcagcatcagtactattaattaaagacactgctaatactaatgctgcaattgaacgactaatatatttcataactaccctttaagcaagtcgtaaaatccattattcccatgcttaggaagcggaaactaaccgaacagccagccgatgacaatcaggacatacaccagtatctcttccagaaattttcttgattttttcgtattcttttgcacagtctttggattgacatttataatcataaagcggcataattattccttaaagtaagctttcaacatctgatataaagaccacgcctgatcattattttcaatagtaactttcatgactgggaattctgtgaaatcttctatttgttcttgctctttctcttcctgctcttgctcttcaaccgcctgatatggattttccacttcatcaaagaacccagcttcgttagtagagagccagataaagttttcgtcaaggatatcaccgccggcacaacgtttgagtacacctatggatgtcataattttagtaggacgcccaagataatcagcatctaaaattttaaaaggctccatacctaaacgtcgtgcatagattccgttatcagtatggtctttaataaaattttcttgagcttgtttatttttaaattgataccatttattaacttcaaatttaatagccattaataaatttccttccagtaagttgtgccgtcttcagtaatttcacgaaatacaccataaattggctgtttatcaccgactttctcatacacataaacagaagtcaagtgagtaaacttgctagtatgttccttttgaactactaccaaatttggatcaaataatacatcttcaaattcatcattagtgcaattctgaacaattttacgtttcattacaatttcctcattaattgaacagtggagcgatacgtttcagaagagtatcaacacctttagcgaattttccattttattctccaagttgttttctgtatcagtagttgatattgatatagtaccataatcaactactgatgtatatagttttatgaaaaatttaaactttatgcatagagagcattgctatagtgtttaatccaactttcaggaatgactttgtatgttcctaaaaataccacgttgtacaacttaacaccatcttctacccattgatcggtaatgtatccacacatagcgcgagtataaacaacccttccatcatctttaataaagttaaattcacaaggagcaatgaacttgatagcctgaccgagtttccacttaaagtctacacctacatgcgaagtatcaatcgtttcaattcctttagcaggaacagcttttaaaaacgcagactcaagaaatttcgcacgaacatagccaaactggggtttagactttccatctttaggaatgatacgcacttttacttcagaatcttcatctttaacaccatgcttaagctgaatgcttacaacttcgaccaattttcctgctgctttagaacgggatttatcagatacacgagctaattcaccaatattaataatcatagttatctctcacttgttaaaaagattttatactccacgggaccattatactctggtcccaagagtttgtaaactattaattcaaaatagctaccactgcactacgaggaactacggagtactctccagcatgaactacgttcagaagttcaacgccatcttccaatccattggtcagttacccaaccaccaatcggattcgcaaatggacgacgaatgtaaactgccttacacaacaaatcagtcgggtcttcaggtttttcaatttctgtcaatagattaaaatcttcttcatagatgatgaatgatgatacccttccataatagtttctaatttccatgtacactgtaccaatgagaattccactattaacactaatgacagtaaaaggatatccgccagttgtaccaagaagttcccaaaatttgctatcagtcgtattcgacattgtctgaaaatcaatttcagatggttttttcatttgatacgcagtattaattttaatcataattttctctttagtttaaggtaataaagccttttagttcggcataggatttacggaacattacttgatgcccgccaatgataacttggtcatctggtacttcgtatacagcaagataaaatcctttcgaagctaattcttcacgctcttctcgtgtgaaccatttcatcatatcatattcgctagcaaaagcaaaatgataaagagctacaaaccatccgggaatatgatattctactccaacataatctttcttgaacttagtattaattacgatattagcatttttaactaatagtttgtcttcgtgcggcacaggaattcttttattatcattactatgatgcataaaattaggtctgtcataacctacatgtaataaccactcttcactccatgaatctattatacttctatacggcgttatttgaacacaaagatctcggcgtattgttatagcgtcttcataattaagaatactaaacgatgattcaacacgataaattttcattttattatcctcagtagctatggtgttatagtaccacaactaaccgaggaagtaaacaactttttatcgttttgttggaagagatagaggatcgcattcttcctctgatggagcatcttcaagacccatagcatatcgcaaagcatacttcatcatcaggatgtctttcgcacagtcatgaatagaatcatgtgcaacgaatccatctaaagttccctttggaagaggacacgttgtcatatcacgaacaagcagaagtgcttcaattctagtacgaatatcacgctgattccaaaatttacaaggttctaacttaaatgtatcaagctcattctcggaaacgccgttaagacgttgaatatcgcgaataagatcgactaaaattggaaaatcaaacgacattccacggcaccagccttgagatttccaaggatcgatattatgtgcattgatgtaatcattaaattttgcaataccgtcgatagtgcttacatcttcatcggatggtgcaatattttttcgagcttcaggagattgattcttccaccattcgatagtacttttagtaaaaagacggtgtcctttttggctttttaaatcaaatttgattttaatgccacgtgaaactaattcatcgaatgtttcaactacttctggattagggtcaaaagcaattacagccaaatcaataaccgctgctttttcaccacttcccattgtttcaaaatctataataaaatcaaacattaaattttcctcgctaaatcacgaatttgacctacagtatagtcttgaatataaactttattaataggctcatcaataaattttgccatagattcaatatctttttgtatttcttcaagactgtatactatctttgaagctttttcgcgaatagtgatattttcaggacccggattttcttgaatgacaactttaacatttgtcataagagatttaaactggtaccaacttaattcaatcattaataatcgcctcataaagatagctaatttcgcctaaaacataatcattgattgtaacagttttaacttcaccgcaaaagaattctaacgcaattaaatctcgttcaatttcttctaattgaagcatcaacttactagattcaatttttacagtttcacgatttttgctataagctatttcataaatttcgcttactttatcttgaagaagataaaactgatctttagttatttccacgaatagcttcctcaaatttaatcatacataaaacacatcataacgaccacgggtgacaccaacataaagaagttgttgagctaattcaacatctgcataatgaatacaaggcgtataaatgaaagcacggtctacagacataccctgcgctttatggaatgttgatgcaggaagtgctttcactttactaaactgtgatttagcatcccaaaaatcactccacggagcttttccgcctttgttccaatttttataagtttctgctgttttagctaaaaataggttaaacttatacaattcttcgtcagatgaaattattttaatcttttcacgataatattcatcatcgccataagtttctactgttaaatcccaatgacgaattagatattctccaggaacaccacgggctttaacaaacgttgatgtatactctgcttctataatacgaactaattgtccgttattaaaaataatttctgacacaggctttccatcaattttatatgttttaaataatggttcctgcattacaataatttcaccgacaataaaatctttatcagtttcaaaaatctttttacgaataatgctatttaacttgtcaacagatttattcgtaaatgccattacgcgattttcaaacaaatcatctagtgatttgacgattgaaaaataatttaccataaaatcgcgtaaagcggtatcaccagtaaatccacgtactccatgcccgtcaacaactttatcataattccacttaccgttgcgaacgtcagtagctacatcaataataggagcattactgcgtttaacttcagtgagttcacactgataaaaatctttatgtgtaaagaatggactgatataagcagtattttctcctggttcaacaggtctgatttgcttattatcccctattccaattatagtacaccaaggtggaatagttgaaagcagaattttaaatagctttctatcatacattgacacttcgtcgcagattaatactctgcatttggctaaatcaggtacttctttttgttcaaaaagaacattttcttcatatgttactgggttaattttaagaatactatgaatagtactcgcttctttccctgatagttttgaaagaatctttttagctgcatgtgtaggagctgctaaaataataccagttccacccgtagatattaaagcttcaatgatgaacttagtaagagtagtcttaccggtaccagcaggtccattaatagttacatgatgtttcttttctttaatagccttcataacaatgttaaaggcatttttctggccttcggtcaaatcatcaaatgtcatcgtaaattccctgcaattggtatactaacaatacgcccagtatctaaaattcgctgatataatctttgcgtgtctacgtcaggcttaacatgtttaacttctattttattaaaccaaaatttacgtggagtctcaactaatcttggaattcccttacctaaagctaatcgatactgctctttaagagtggtaaatactttatcagcaatcttccattcaaaaaatacagcaggacgatgttcatcaagcggaactggcgctgtaaatccgtctttgtctcggtaaactatcgcatatacataaaccatattatcctcggataagtttaaaaattgaacaatttagcggatatcctcttttcagtttaagtttatcaataaaagacaaattttgataccgctctacaccttgaataattttatcacacatatcatattgcatttctgcttctgacaactttttcacaattttccaatccgagcctttaagaagaacgttcaatttaacaacttcagcgccttctgctatgcgagaaccatcaatacgtgctttaagtgctataattctcagcttaatgtcagaggtctgttttgatttagaaagctgagaaatgtgttcaattcgattttcacgttttttctgtatagctttaatttgattataagtctttttgattttagcccatttcttttcatctaaatttagtttatgaacttttttcgcagatgaacgaccaattcgcaaagcaaataaatcacgcttttcaatcaactcttctaaagtataatcagaacgaaatgtattatactttttctttactgcaataacattccctttaatgtatccaacgttattatcaaaacgttctaatgataatttctctccttcaatacgattatcaaaaggttctcccgagtaagcacaaactttttgatctaaaatgttcttaatgtaattgaagtctaagttaaaatctttagaacgtctttttgcagatgcctgagtatgctctaaacgacgtttaattttacgaatttggttattagacagcttcatatttttctcacatcttacggacggttaactacttatactataacatttttactttaacttgtaaacaactttatgaaaaatgctttaaaactttcatggtataatgaatctaagtccttccattatagattaaatccttcaaaatcaagagtatagatagtgtatgttgaacactttttatactcatatctatctgcaattctaaatacacttccagctggtatcattacttcttgttcatctgaaactaattccatattacgataacgatgactatccggaaacttaaagtttggattgtattctttacagcgtagagcttttatagcatactcctggaaattgaataccataggagctttgaattcaaaaataacttgtgtgttatactctaaaccagaagcaaaatgtagagctatatttttatcatatgaagctgatacgactttatcaaatgtaataatatcaattccttgatttaatacttgtttagtctcagctggaacacctctccaaagaggtttatcgtttggaaccaaacgagatttgattatttcatttaaccaagaatggtcatctggtttattagtaatacaatgaattaaaagttcaatttcagataaattaaacccttcagaaagtaattcttcacgaatagaagcacgcaccgatgcatccattgattttattttaaaatcttttagttgcattactgagtatttcattcaactacctcaatatcataaactttaaatgttccaaatgaatcgtgtaatttttcttttgaaatagaagttattttatactttccaattggaatcatccattcttgttcacgcacaatcatcattaagttatcagtacgctctgaatctaatccatcagtatcttcatacgtgtacttaaactcagtattaggagaagaaagtataatatcgctgatatggtcagaataattaaaagctttatcagtttttaaacgaagtattgtttcagtgaaatattcagcataagaaaaagaacacgctgtatgcaaactagtagtaaatgaatctaccctgttcgttgaaaacacttctccaacttgtaaatctttaatgagttcttttgtcgattttgatataccacgatataattgataaggcgatttagttaaatgctttttaatgatttcatttaaatgcttatgaagagcttcattctttttggcttccatacattgccaaagaacagactgctcaaagtcagtaaatttttcacagacctttttatacatatcatattgaaaatcaacgctttcagcttttatagataactgttcaacatctgcaagattaataatcatgatagcctccgtatacttcagaagctatcatatcatcgttagaaaggaaagtaaacaactttttgaattattttgcccagggagcccaaggcggagggtcaagatggtatgaagctagttcttctagaagagcatctggggcttcaattccataattctgtaatactatacggtactctttcttataatcactagaatcattctggttattcgtagaatgattatcttctaacatctcaaataaatccatattaattcctagcgataaaaaccaaatttacgattagtttcaatgatctttctttcttcttcggacattctccagcgtagtccaacatcaaaatgagcccagaccatcctaacaaatgcatttatatctttaatatcttcaataatgaatgttttacttttagaaggttgactcgctaatttaactttatcgtcttcaaacatgtcaaaaagaccatattcatgagctcttttatagcctttaatagttaaagcttcagaagaagaaaatggtgaatctgtattctgcaaaatatcttcagtatgctttataattagaataatattttctggatattttctttcttttatatctttaattaaaaaatccggattttctgctaaaggaataattaatgagcaatttttatcaagtgtatttactgatttaccttctttagacataaattctattgaatataattttgctacttcaatcatgtgatttccttttgcctactaatggaccgtcaggaattttattttcctggatatatttctcattttcttccatcattttactgccaattttaagaagcaaatccattgcttcatttgcttttgctttagcttcttctaacgtcatatctttgttcatgatttatcaccatagatgtctctcatcaatttaagcgctgagcgttctagtttcttttctttctcagcactaatcattgatttcatccattcttctgattcattctgcatttctttatttgcttgttcaacccaaccgtcatcaatatacattgagtttggtctattgaaccattcaagcatcttcttcagaactttcattcgttttacctaaaacaatagtaggagcatcgtcaaatttatgaatttttagcaaatttggatttaaattattccataaagaggtaataaaatatgatagcgcactttcgtccgtaattataaatttatttcctttatctattttccaatcatatattgaatcatatgaatagaaggataacggtttattattataatatgcttcagcaacatcaatatagttagatttagtaaatgcttgaattgccataaatggagaattttgtacagtttcaataattccgatctttttaagttttatttcaacatcttctggaattggcattgaaataaaatcttctactagatacatattattgtcatatcttttatcaatcattactgctacagtaattggaacatccttgacaaacgccatactaatactattgatagacatttcaaacaaaattgcttccataattttcctcaatcacaagatgtagatgaacaactagaatcacaagaacttccacatgaatcacctgcccatacatgaacaggaacattagtatcatatgaatcagaactagactgtgtattctgtgtgttagatgatgtagtaggtgttgaccagcgccaaggatttttataatattcttgggcttcttcataagtcatagtaactgcttctactgttccatctcccatataaacatattcaactacagttaaaggaaggtagtcatttgaaataggaactacaccttccccaggagttgtagagaaaaaatccgtaaagaaacttttaaaccaattaaagataaacattacaaaaagcctcttttgaattcgacttgcttctcaccataatcatatcgaatctctacattaaattcgacagaaccatctgcgtacatcataaatgaatgcacaacaacttctgtagaccatggttgtagttcatatttcttcattacatgtcgtgaaatgataatatctaaatcttcatttggtttaatccaacgatttaacatagtactctcctctataagataattctattataccatactcattttggaaagtaaaccatttaaatgaaaaaaggactcccgaaggagtccttgagttattaaccagttactttccacaaatcttcatttgcagcaatccattcagtacgttgattttcttcatatactgtagaatatgctgctttttctgaagggaatgtctggtaatgagcgccagaaattttgtgaacgtcagaataaagagggaaagctacagaaatttcctttccttcaattttcttatttccatctaatttctgctcaaatgttttgatattaacataaccgcgagtactagccatgtaattctcctttatttaaattacatgattatttatacatcttcttttctgaataagtaaattaaattcttaagagccgaacttgttacatcatattttcctttaagcgcctttacaaccgggcctgttgctggtttacctaaagaaacccataactcgtgtatttcgcttttaagtggttcatgccaatgcggtgcttttctttgcgcccttgaagttccttctgaaatctttttatttccgccattcgaataaaactttttcattacttcagattgccgagcttttctttctgccctattttgggctatacgttgtgaattcttcatccgagtttttgtttcaggattgtttaatctaagtttatgttctaatcgttgttgctcagtccattttcttccttctccaccctgaccaccaggagaaatattaatgcaagtatctgggtgtttacgttttaatgcagatattagctcacgttcaacttcatatgatttttctctagaaccatggcattttgaccatcgtattttataattaaatccatacttacgatatatgttccatagtattttacctgaacccggatatttatcattatatggatttacaataaatgattcatgtttcccagcgtaccaaaagacgcctttcggcgtcctaacttttactatatatgttctataaaatttttgtttaatatccattatcttgacgttcaaaattatgtttgttcttcagataataaagtttaaagatttcttccgcattcattccaaggccaacaaacatatttaatacgaaatgaaatatatccacaagctcaaatttaatttcgagctggtcttcgggggacatttcatcaatgcgtttttcttgcgcttcaatataacgtgctttccattttttccatacagcagaagcttctttttcaccacgtgacatttcaccaagagaagtcagaagttcgcggaattcatcatcaatacagtctttttgttcacgcatccaagaaacaacatcaccggcagtttctaatttatctggatgatagcagtattcgcggacattagccaaacgaatctgtaaaaaccgctgcatatcaagcataacttgcagcggatctttttcatcaccgagaatatcccagtattcattttgagctttatcaacaccttcgatcaaatgagcacattcattaaagtgagccattagttttcctttcaattcattaataagttaaataattatatcatttgagtatgtaagcaattaattaaaaatatatacttcatcagttccattcttttctttggaatgatatatgttaaagacgtattttttattaagatgctttacattatattttttagaccattctttaagaagagtgttttcctttccgtggtgttctaaaacattcgactgcccaaattttattcctctatcatttaaagaatctaaaagatttaaaaggtctttttcttcatcttctgaccaaaatttattataatcagcaactgttatgagatacggaggatctacatatacaaaatcgccgtctaaaattttaacatctttaaaatgcaatgaactaaagattattttatcacaattttgtttaaagtgattatattgtttttcactatttttgtttatagttctttttccaaacggagtagtaaaatttcctttatcgtttatacgaatcatattactaaatccgtgaaaatgaagaacataaagtaaaagaggatctctagttttattataatcttcacgtaatttcaaaaactcttcttttgatgtttttgatagtttgtattgctttattacttttaaaacgtcatcccatgatacattaataagacgcttatacatttcaataattggttcttgaatatcattggccaatacagggccattaacattcaaagacactgataaacctccacaaaataaatccacgaatctgttatattttggaaagtgagatttgagttcaggtaataatgattgtttattacctgtatacgcgatagctcctagcattatattctctcatttattgcagcaaaaatgaattatacaattcttcatcatatgtatttgatagtaatactaacacgttttgcgataaatattaatttaaaggaggatatatatggtacaaaaattaatggcacttgttaatgccataaaaggtaataagaaacgtatagcttttactatttctactatggtaggaattttactctggaactttattttatcacctgttgcaattgcacatggtgttaatattccagtagttactcttgatacattcgtagatttagcatttgctttagttgggttaatttaaatcttagcatatttagatagccgcattttagccattaacccctgggcaatattatttttcatatattccataatttgttcaggggttgcaccttcctttctaatcatatcattaacatcttttgatttccagggagatttatcccaaaacataaccctttctcctgcatcaactaatttagtcattcgtttaatagtgtcagggtgacgaggttcattatctaagacccacacacgtctatctttaaatggaacaacttctaggtctaattgaccgcccgtaatagctataccattttcaataaaaagtgaatctataggtccttctagaacatatacatcaccatctttaactcgttcgactccatagatttttgttgcctcaggataagcttcgatggtgatatatttttgaggagcatctttctttaatgcacgtccttgaaaagactcagcttttccattagcattataaattggaataacaagacgaggctcagaaatttcctttttgtatgttcccggtgctatgctattaactaatttaggccattcggttgtaaaccaaagatatttccatttatcctttggaatacaacgagcttttacgtattttataattggatggtcttccgccagtttatctaatctaacacatgacggaagagatttaattattttcttctcgggttgtttaggaagttctttaggtttttctattggacgacttttacctttttcttttcttatttcaaagatatactcacgatataaatcgggttcaaactcctttaaatatattccgattggtgcatgatagttacagttataacaatgaatatttccttcattattatcaccataataccatccacgggctttattctggtcggtttttgaatctccacaaacagggcatctaaaccgtaatttaaaagttgaactattatttacttgtgtgaatttaggtaaatgagctaatgcacggtatgcaaactcattatcaatccaaggtattgatgacatttttactcttctttttctttagattcctcttttttctttttaggaatctgttcaggacctttatttactacagcgcctgatgttgttccaatagagatattttcaggattaccacctgaatctccagcgaccatatcttctttgataaattctttaaatgttttcatattaacctctattcataaaagcattaaaaatttggtcatcaatagaaacatttactttaggctgtttttcagatggcaattcatatccacatattacaattttatgatcaatatcaaaatacacagaagcaatatgattaatgatattttcagtaaagtctaaatcaacatcaatatctttttgaccaaagcccaaaggataaataatgcgagtaattcgattatctttaacaaagattccaccgacatactctgtgctgcgtttaaagtctacacgccgacgaaatgaaaaatattcaggctctttatgagctcggctcataggacacaacgaataactagaataagagatgtcaaatcctacgccttcaatatgaactaaattgtcatgattaaaccaattataatcatatgccaagtccattagattgtcatatgtgaaaagcaccggattaacatcattggtcacaagcatataattagcaactgctataatttcattatttttaacgaatacaaaccgtgattgatgcgaatggcctggacctgcgttttcacgattaatgatataacactgggcacctttatatttgtacgtgtctttacacttgtgcatttgataaagcattattcacctaccacttcagcgatgatatttttgttattaaagtttttatcgcaatacagaacataattatactgcattacaccaccagacttaagctgtttttgcacttcagctttcatttcaggacgatcacgcttaacgatattcataatatctgcttcaatttgagtttcaacctcagtctgatccgcagtcatagaccattcgcacaaatctttatcataacctgccatagcaggctgagcagcacaagaagctaaagcaaaaattgtagcaaagatgaattttttcatgataatctcctcagtagtttatgtttatatagtatctcaatttccaacaaaagtaaacagttattttaaaatttctgcgtaatcacatgttacaaactgtttctctaacttgacgattttacgaaagtatcttttgcattggcgaatctgcctcttcgtagggcgaacagcaaacttaataaattccactcgaccaaatggagggctttcttctgctggaatatctaacaccaattcccacgtatctgcaataagtgctttgaattgcgtatttttcctgacgttatacggagtaggtttaaataaaacaatatgcatattatcctcggcaatccacttcacatactttcttgtcatcaatgaaagctttaactaatgctttattaacttcagcatattgagtagtagcccattgaacgtcatctttcatcattgtgatttctttagtaaacatgctttcattcttaaaccaccccataaaaactacctttaccaattccataacaatctcctcatttaaccgacaagactactataccatagtcttgtcagcttgtaaactaaaattttaattcattcgccaaagcatctaactgagctcgagtcgattcatttctttgatagcgattctgctcagcctgaatctgttgtgaacctgctacttcgttcacttcagttggagtagaatcttgttcaatttctacccatttttgatttcctttttgaacacccatcaaaaacttattccacttattcttatcaccatatcgtgatttgatttgcttaatgagttgttgttcagcagctgctagctcctcggtttcaatgaccgcaagcataaaatcggctgttgctggaagaccggcagattctgcaatatcgctcatgttaacatcggaagagtcccaagcttgtttaccaacctgtgctgcagtccaaagaacagtttcggtttcaacagccagagcacgtaattcctctgcaatagctttaacagttgtgtaactattttctgaataaactctaatgcggcaagatttacaaatacctagatagtcgacaataatgattgttggaacaaaattcttcttgagcttcaattcgtttaaaagtgatcgaaatgtattagcgtctgctccaccagtaggatactgtttaacgattaaacgaccaagagtagatttctcacgccatttttccatttttcctttatactcagcgtaagaaatatgcccatcatcaatgtcatcaagagaaacatcaagcatattagcgtcaatacgtttagcacagacttcttctgccatttccatggaaatgtaaagaacattatgtccgagctgtaaataatctgccgctaatgaacacaatcctaatgatttaccaacgttaacgccagccattaaaacgttcagtgttccagtttcagctccgcctttcgtaattttgttcagaattctgagtttaaatggaaccttacgagctttattcatataagatagccaacgtgcttcgtagtcatccatccaatcatgaccaacgtaactatcaaatgaaattgataatgcctggcgcatgatgtcaggaatagcaccaacatccggcattttcttatttcgtttttccggaggaagctcagcattagtttgaatttcgattattttagacgtagcattaaacatcgccctttgctgaacatatttttctgtttcttttactaaccagctgtggtcttccggagaatcagccagttttgaaataagtgtttttacaccagaatattctgtttcagtaaatgaactattttctaatgcaacatttaacgcattaatagatggaacgctatggtactcattaacatgagatttaattaatttgaatgtatttttagctggaccactttcaaaatattctgaatccatatatggccaaacttttgaaaaataagcttgatcaaatatgagatgagaaagaataatttctaccacacttactccttaaaagaatttaaattttttctttgaccttttattaaatgcatcttgcagttgcattgtaatacatttttctacatgaggagctaactcagcttttctttcttggtcaagaacagcaaagtccattacaacctttccatcaacccaatccagtttagttacatacactatatgcgtagaaccatcttctagtttaatgacaatctcctggataacattttccatagcagatttaattatcttaagagactcattaaaaagacgttcttttctttcttcttccccctccgaagagggggattcatcgataatttctagatctaaatctaaatcatctttattcattaaattcttccatatcacttagctgttcgaggtcagtttctaaatcagcagctgatttacttttactttctggagatttaaatttttcaacctttgagttaatcaattcatcaacttcagcttcaacaatttcattactatcaatagcacctaactgataagcacgtttaatagcatctcggaatggttgatgcttaaataaaggaccccagaatgtagtgcagttggtatcttttgcacgccaagatttttcttcgcgaatcatctcgccagtttcttcgtcaagaaattcacgagcataccagccatttttaggttttaccacgaatcctaattctagagccatatctaacaatccagaataaggatcgataccaccgtcaaatttaacatcaataaagaatttacttttttctttaacggtacgagatttttctacatttagaacaaattgatacccctgaagatcagaaccatctttaatctggcgtttaccgataatgaatacggtatcagccgaatacatcggtccagtaccacctcccataactgttttactaaacatttcttgtgtttcgtatgtatggttaatagcaatacatggaatatttttagtactaaaatagggagttacgatacgaaataagcttttcattgttttagctctagtcatatcactaacaactttttcatttaaagcatcttcagtttctttcttagaagctaagttaccaagtgaatcgataaaaacgactaccttttcgccgcgttcaattgcatccaattgattaaccatgtcaatacgtaattgctcaagtgattgaaccggagtatgaattactcgttctggatcgactcccatagaccgcaaataagcaggagtaataccaaattcactatcataaaacaaacatactgcatcaggatattgacgcatgtaagatgacaccattgttaatccaaagtttgatttaaatgattttgatggacctgccaaaattaacagaccagattgcataccaccagtaatttcaccagaaagtgcaatattcatcataggaatttttgttcgaactacatctttttcattaaagaatttagatgctgttaattctgcagtcaatttagaagtagaagctttaatcaaacgagattttaaatcagacatataataccttatagtagtgttcttgttccacgaaatacttcgagcattcttgcataagatttatttggaaatccagcttcaattgctaattttttaagcttaatagcaccggattttccagagttttcccatatttctttaatcttttcaatgttatcccacattattgggtcacgtttatgtgatggtttatttctattataaccatatggattattaagcaaagcttcttttcgttttgctttagtttcttctgactgctttttgcctagctgggctttccggcaaacatcactaaatccaagatgttttggttttcctttttgagcttttgaaattttttctttggtttctgatgaaactctataaccaattcttctaccaccctctccaccaatttttaaattataagtcataggatcatttaccacatctattgttactaattctctttcagcatcacgggctgatttaaaatctttataaaacccaagaatgcttaaattaaaatttttcttaccatactttttcttggcttgtgctaataaagttcctgatcccatataaccatcatttaaatcatcggttgaatgagttccatagtaaattttattattaactaaattagttataacataagtataattatatttcttttctttatgtcttttcatgtcattaatcaactgctattcgatgaacttctctcttttctaatcaatataacaaaagaagtcccaaaaagcaaacagacctaaaccgataataagcaataaaggtcctaacatttattccaccggttaaagataaataactttctaataatagttcataatttttataaatcaatagcttttttgaacgcatcttgccattcggctttctttgcacgggttttatttaaaatatcatgttgaatagaaagcatctctttacgcaaaacatcactgtgttttaactcattgactctatcaatgagttcagcacgattatttacataaaaacgagcatcattaataattcgatgtttggtatcaaattcttcgtcaattagcatcactgcatcagatgccattgtttcccagacgcgtaaggtaataaagttgtcattataattcttgtcaccaataattaatgcagcaatagcttgactattcttttcagataccatgttcataggaatttttccagtgaacaccggagctttggtccaaggatatttaggatttttaaactgtttttctcgtgcattgccaaaaaactcaatatttaaaccggtgtcaaataagaattctaccatcttggattcgcgttgaccggaccgaaatgaaccgccataaataacatccaaagttttcttggtaggcttagataattgaaaatcgttcatatgaattttatattgttcaataggaaaatattcaaattcaataacattatcaactttcttatgcgcagccttagcaatgtctaaatttataccttgggaaatcactttaattggtgatttaattaatagctcttcttcagtgtacaaatatgcccatggtctatttttaacatttggccaagactgcgaaaacggcaaacgtatatctgtaaataaataataaattttacttttgtattttgccataaatttttgcgcagataaaattgctaaattaggtttaccgccaaaaaagttaatagaagaattaacaactatcaaacggtcataatcattaacatctacttcatcaaaagatttagtgtaaacaccatttttaagagaaataatgtcgacattaagacccatttcagaaataactttaaaaagataaatagtttcagaagatggaacagttttaaaattaataacattattacccatattaattatagcaattttcatattattccttttatgttaaacgattaagcgtattttcctacataatcttttttcgaaatatgtgtttcgccagttttccaccaatgatcaaccaaataaaaatgacgagaatacacatgtaggcttccaacattccatataatggaacctgctttatactggcgagttgaatcacctgcattcaaatcagatactaatttatctaatacgtatttttgccatgcataatcattacggaatccgaagaccacgtcatttgagcgcatgttaacaaccgcattgattttcttgtcacgaatcaggtattgtactgtattcgtgcacatgaaatctgacataccatctttattatagtcaaactgcatagatggacgagtataaatcatgataccacgtcgagaatcaggattttgaccaagttcagctaaacacatgtcatactgagcatagttatcttctgaccagatagcccaaccataattcgagttaatttcacctttagaagatgctacttgttgccaaatcttcggtgtttcacccggaatatctttaacgaacaagcttttagatttataccattcaagttcacgctgaatgtattcatcattaagagcgccaaaaataaacggttcatctgctacaaatgatgcgccaataatttcaatagttttaacacctgttttatcaactacgaaatctttttcttttaatgcaagccccaaatgaagacggatttcttcaactgtcatagagtcactaatcatttaaacctcaattgatacattcatatttaacttgtaacagtaataaactccaacctaaaataatagttggaatcataagaggaaccgttacactatagtatatacttattataatcatcaagattaaaagcaatgctgctataattttgcttttcattccttctctctgatgataattacctgatttggctgcgcagactttttagtttcacctgcaattgaccaaataaatgtaataaaccaaccaataattgaccagttaaacagtaaagatgcgaaaaagattcctactgtcgattttgacccacgcatcaaggcgataaaccatggaagcatgtatataataatagccaacacgcctgaaactaaaaccataaaaattgaacctgctactaaagtttccatgttttcctcacttaggtcaaattttttacacatgaattataagaattcactacatactccatcggagcgtttttacctgtacgccactggtaattattagcccaatttgcccaaagctcagcgcagtagttttcaattttttcttcgcgtgtaattacatctgaattacgatatgcttgagcagattcatctggacgaatagcttcgtcaaaattcgcctgcatttgttctactgtctgttttggagcttctttataacacttgacattaggattataaaatttgcttgaacagtttacaatttttcctacatcagactgatttactaccggtccttgagctacacaaccagcaagacctaatgcaataaccaaaatagcgattttcatttcattctccaaatccgtatcagtagttgatagttgtatagtaccatggaagaacagtcttgtaaacagttttgtgaaaaaatttttagggaatccaaagggtccagaatcatctctttttcataagtatagatttatattacttgtatgaaaaagggacctggaggtcctagatttattctatcagccaaacaggaagtctaacgaagctttttcttcatagtccatgccagccgattcacacatacccgcaagcggtttaacaaacgatttttggaacaaagttgagtggtcaatccaagatagcacatcagaacgaatttcttttggaagttctgtacccgatggccaagcaatgcacttgtcaccaaatggatttccttcacgtaatggaagaaccattactttatttccatccaaaattggagctacacctaaaccgctaacagctcgacgataagttagcacaccacgaatatggaacgggcatttaaatcctggccaacctttatcatcatatttcgctatatcgttcgcagtttttacttcagcaataactttatagtcaagttgacgatattctttctcgaagttcttgtagtattcttggacagactcttcaccttcctgaagaatacgacgaatactttcttcgagagcttcttgcactgcttttggtgttgaactctgctgagtttccatacccatgatttttagatgcggttcagcaaatcgcttatcttccatatcataaacgttcagagcataacgctttttcgctttccaaaatccaccaacgccctttgaaccaagcggagggcaagaaatagcttcacggtccatatgcatcagatgctcgcggttattcatataatcacataactcacgatatgcaacatcaatcataggttccatctttttcttaccgaactgattcatgaattcaaccaaatcgttctgctctttgaatcggtcaagaccaactttttcaataactttatctacgcaaacatataccgaatcagtatcacctgctgcaatgaaatcttcatcattagttccgcatactttattcagatattcattaattttacgagcaatccactgaataccgacttggccgaaaattgtgatagcagtagcatttcgcaaatcatagtaacggaaatgaatattaccaagagcaccataaagactgttaatgagaattttacggttcagctgatttgtattagcaagtgtagctgctttttcacattcttcaatcagactattgagaacagattcggtgtaattcgatagttcatttaagaaatcatcactgaacttaacatatcgttcaacttctggtttagttgaacaagaccctgcgcctttcataataatctttttaatagcttcggcattcatttcttcagcgaacattttctttttccagtctttacgctggaaaaatactttagcgatttcctttggaatgataccttcttgatgtttatcatacatccatccattcggagaacaagaatattcatcactcggtttaggagctgttcctgcgatatattcatgaattggatgaactttaaactgaccacgaatagtttcaggactaatgttaacctggcgaataatgctcggatacagagacgtcaagtcaaaactcataatgtatcgacgtgcaattggtttaggttcaaacacaaatgcacccggaaaactctgtttaacgtgcgaaccttgttgaggaataaccttatgttcacctttcaatgagttaaaaataatagcatcccaagttttaataggactcattacaccagaaaaaggcattttagcgtaataagacatacttaaaactagatcgataaacccacgaattttatcgattgcttgaactgattctacgtcaatgatgttataactaatgtatcgttgatgattagtctcacgaagtttattaataggaccgtcgtatggtaatttaccttttttggtttcatgttgagcaactgattccaaagagaatgacggcaaattagtaaaagcgaatttcttgtacaaatctaaataatcaagaatagatacgccatcaatagaataaatttctttgctaccgtacatattttgaattagtttagattttacccgaccgattggagagaaacgtttcatactacgttcacccagaatcattttaacacgattcatgatatacggaacgtcaaacccctcaatattccaaccagtaaaaatagcaggtcgtttctgttcccaaagattgatatattccatgagcatatcacgctcattatcgaatggcatataaattactcggtcaagaatttcttgaggaacttcatcaccaccttcacagtcaagcttagcagctaactttgcatcccattttgatactgaaccgtacattgaattcaaaaggtcgaaaacataaaaacgatcgtcaattgaatcgtaatgagtgatagcatcaatttcatattctgctttcattgggtcaggaaatttatcaccagtaacctcaatgtcacagttagctacacgaacaaattttcggtcataaacaatttctgaaccatatgtatcacttatataagcgagtttaaaatcgttcataccgagagcttcgagaccgatgtcttccattcgcttcatccaatctcgagcatctttcattgatggaaatttttgaggagcgcagtttttaccatagatgtctttgtattttgactcttccttacaatgcctaaacatagttggaagatattctacttcacgggtacgttcctttccattttcatcaatataacgttcaacaatgttatttccgactgtttcaatagagatataaaattctttcatagatattccttagtttatagcccgagttattaggctcttgatatattatactccaaataaggggccgaagccccttgcttaattaccaatcgtatatttaggaacgagcttccattcatgtttttgtttaaaagaaataactcggaagttattagttaaatctttcataaaagttctttgaccaggaacgatttcaattagtccccaatcttctaatagccatgcaatcgaatcacgacgaacttcatcttcttctgtcatttcaacttgacgaccatccatacgaagcatttctttaaaatgaacgatatagtatagtccttttttctgaagaatatgacaggactgatatagaactttatctttattattagcaattcccatacgagtcaaagtttcttttactttcagaaaatcttcaggttttttaagagtaatttcaatcattttaccattccaatgctagttttttgagttgtttctgttcttttacgttcttagtcacttctttcaaaaaatcatccgtgactaaaccttttagttcttttaatactaaaggaagttttccatttttagtaagaattgatttatagttaattgcatcatttgtattaacttgataccgcttagcaagtaacttaataatcaatacttcggtggaatcttcaaccagttttgcccatttaccatatcttttaccacgaggaactgcagccattagataattaaaatgagcttcatcacttaagcctgatccaattaaattcatagcatatacagctggcatacactctggaaattgtgataatgcattttcaaccatgaattttgaataatctttttgagcaatagagcatttagttttattattaatagctccaattatttcaaaaaattcattttctgctttttctttaaaagaatcagcagcggattggacagctgtccaatcttttgaataccaagcaacttgatgctcgtttaattgaatatcatctttaaataagctcatatcacttccactgcatttcgcatgctaattgaatgaaaagataagctaaatgcaattcagtattagctgcaataccatgatactgattattttcgccgacaatttcgtacatacgaataatactttgtggagttacacgtgaatagatttcttcggcaagtttacccacgaaccacgaataatcagccgcatattttggtgctaaagctctgagttgtttaacatctttatttttgagagactcaagaacatcatcaatagcaccacgatcgttagtaaccagtgataaaataccagcatccaaaacacctttagacgaataactatcgagctcgccaatagttttacgaaaatcaggaaaattctttttaaccaaagctgctacaactttcatatcagctatagcaattccttcatgcttgcagatttcagtcaatcgacgaatcatctgcttcatcatttcaattttatcttcatcagttggttgaccgaatgtaataactcggcagcgtgactgaagcggtttaataataccatcaatattattagcagtaataataatactacagtttgaactataagcttccataaaggaacgaagatgtcgctgagactctgctaaccctgaacggtcaaattcatcaataacgattactttttgacgaccatcaaatgaagcggcgctggcaaaattagtcaaaggaccacgaacgaaatcaattttacaatctgacccattcacaaacatcatatcagcatttacatcatgacataatgcttttgctacagttgttttacctgttcctggagaaggagaatgaagaataatatgtggaatcttacctttacttgtaatagatttaaaggtttctttatcaaaagcgggaagaatacattcatcgatagtagatggacgatatttctgttcaagaatgtgttctttttcatttacagtaatcataatttcctcattcaagttttagtgtaaattataaaggccgaagccctctattaaaaatcgtgggtagaatcagcttcaagagctaccacataattcgcgtgttcaccttcaaatttagcagcaccttgtttaccttttgcccaaagcagaagtttataatttcctggttgcattttcatatttgccatattgataatgaaattaaatgtattttcaccatcataatcaccaagagtcaaagaatatttaacacgggtcagagcagaatcttctactttattaaaaccgttaattacgattttaccttcttttaccgtgatagcaattgtatcaatttgcagaccacgagatacacgcaacagctgttgaaggtcttcagctttaatttcagtaacagcagatgctaccgggaatggaattggtttattaggagcaactactgtactcggatcggctgctggccaaaaaattgttgagcgggcatcagcaattttaatatttccatcttctgactgggaaatttctgcatcatcattaactaaagacagaataccgagaaaaccgttcaaatcgtaaattgctacatcaaaatcaataacgtcagaaatatttgcttccgcataagttgtaccattaactgcgcgagtcataataaattgaccggatttaagcataataccagagttaatagtagcgaaatttttaagcagagcagtagtatctttagacagtttcatgtaatttccttcaattcaaatgagatttaattttataactaatttaataaagcaattaacgattaaaatcagccgcaattgtttccgcaacaatttgagcagcaacaattagacgttcatctgcattaccgcaataatcatcttcaaggcgttcaccacatgaagtcataataaatttagcaccggcgtttagggattctgtagtatgtttgcgcattagttcaatccatttattacttacttcacgatcgatagcttcataatacgcatgacgagcaggtgcagatttaattttgttctgaataacttccattgcgttatcagaaagagacaaaacccatgctcgacgaattttattttggttttgtggatttgattcagaacgcacgtgttttggctgaatatcttttacatcaacagtataattcacagtaattttagtcataatacacctttagtcataataatcagtaacagtccaagcttcatttctattggacattatttttgtatattctgctttaaatgcattcctaagcatagattcagtaactatatgctcttcattagaaaaattatttctcagaatatatcgttttatttcaggaatagttaatagatgctgtccagttgaatattccatgtttttcctccatagagattatactctaataaattaaagcataatctcttataaattaaaccattacagtaaatcgaccaactttcttcatttgaagatgctgaccatattcttgcgggtcatggtctttatgcgaaattataaaaacgttagtgtttttcattgaatttataatattagctacacctttaataccttcggcatcaaatgacccatcaaacacttcatcaagaattaatgtactaatactaacaccagatacgatagaagcaatatcacgccaagtaaataaaagagcaatatcgattcgtgccttttcaccttcactaaatgaagcataactaaaatcttcacgaccacgggatttaattgtctcattaaattcttcatctaatgtaaacacataatccgcttccattattttaagataatggttaatctgcttattaaataatggaatgtactttttaataatagcacctttaataccagaatctttgagcatatcagtcaaaattcctcggtggtatttttccattactaaattagtttttgtcttaacaattttatcaagttcttcttgaagcagtgctatttcatcagcatggtcaataaactcagaagatgctttttctatagccgctttaacttttttagctttatctactgctgcgatcagagattgctttttattgcgaatatcatttgccaacgactgctgggttttaatattatctcggtattcatcaacaagaacttttaaattatcacgatgtgttgaaagctgttcaaacgaatgtgtgcattcagaaactttatctttaattttagaaacaactttatcaccggaactcaattgtgacaaacaggttggacataatccaccttcgtgatacatattaatgactttattatacgagtcaatttttgatttaattaaaaatgcttcttgaccgattttattaaatgcatcagtcgggtcttcgtccaaaacaatattaactaatctttcgttagcttcttctatttccgattttagcgttctagcttcttttgccaaatcatcatacatattttgtagacgagtaaggttgtcacccgttaattttttctggcgttcaacattatcattatatattttaatttgttggataatactatcttttttaacatcaagcacttggttctgcgaatttaattcacgtattagtgctttattaagcttatccatttcagctaatgttcctacctcaagcaggtcttccacaagctttcttcgcgcaggggtcgacaaacccatgaaaggggtataccctgctgtaccaaggacaacaatctgcttgaaactggcatatgacattccgataagctgttcaaattctgcttggaaatctttactgctggcagattcattaagacgtgtaccgttaacggtgatttcgaaaacgtttggtttttgtcctcttttgatatagtactttttctcatcatattccatccacagttcaactaaaagttctttcttatttgtgctgtttattaattgacctttctttacatcgcgaaatggcttaccaaaaagcccaaatgtgatggcttctagcatagtagacttaccaccgccatttcgtccagtaataagagttttttgaaccttatctaattgaatgtcaatcccattttgaccaactgacattatatttttatattttactctattaagtttaaaattcttcacaaaagattccttttaatgtatcttttagaccattctatcatatcatcataatctaaaaagtattcatcaaattcagccatgcaaacaacgccttgtgctgtttttgatgtgatataaattattccaacatatctagaatcttcttcggtataatcaatatttgctatgaattcatcattaatgtcaaatgtcgaaaacttcacagtatgcatccttaatacaagatacagccatatctcgtaatgatttaggtgtgtcattatctaaaatgttgaagttaaaagatacagcccagtcagtgcagacagtaacctttttaatacaattatcttcaacctctaacggttcaaaccagtattcaataatttctttatgaccaatatcatcttttacttcacatttaaaatgctgactcatcataacatttttaaattcatcaaaagtcattgtgttgcctctacatatagctgatttgcatattgaataagtgcttcacggtcagaatcagtgatgtctggaattgcattaatatactcttccattaatgtctgaagcgattgaacttcaacttcttcactgtcatctgactcgacagagttatcaatctttgacacaactcgtaatgaatgcacaactttttctagttcagattcgaacttcgtcagatttttgtctacttcagttactataacacgtactgatagatttgtaaaatctttatagtcaatttttcctttaaatggataatgaattctacgatgccaggtagtattgtttggaataaattccgttcgttctgtttctgtatcaaacatccagaacccacgagggtcattctcgtcacctgcggttagtgtccatggtgtcccaatatatctgacgtttgcagcctcagaaatagtatggaagtgaccagaccacacttctttataagtcttaaggaaatcgggttcaagaccatgagatttcattcctttataaaaataaaatccattcagttcccagtgaccaacacaaaaagaagcagatgaagttttgatatgctcaagaatttcaccagtattttcttcgcacatccaaggaatcaaatcaatcaaacacccgtcaaaatctactgtagtaggcttatcatacactttaacattaggatatttagccaaaagctcagtagaagcatttggatgcattacatttttatagtggagatcgtgatttcctacaatagtgtgtaatgtaattccagcatcatcaagcgtttgaactatttcacgggcaaactccatagttttatgtgtgatcgcttttcgcacatcaaaaatatcaccgtattgaatccaggtagtaattccatttttcttagaatattctatcgcttgcttaattccatcaatttgaataccgcgaatccactcatcatcagctttaacgcctaaatgccaatcacctaaatttaaaattttcatatatcaagaaccgtcattgaaatgcaaaataaaattattgaaataaatccatctggagtgctaaagaacccaatccaacatgctctagtgaatagataaaatgcaagaaaaagtatcacatatccaagaaatatcattatatcaaactccgtataaagctaaagggccgaagccctttattttgtaataatgtcaaactgttctttaaagcagaagcttgaatcttgatgctgatacaaaaattcatatgctttttctcgctcacggtcataaagagctcggtcagatgacagttctttaatacgttcaaatgttgattccatatcattttcatcaaaccaaatgataccgctatcatgcgaggtcaaaggagtattatcaacacggaattttaaattttcgccagtagatttccaaaataccggaattgttccacatgcaccaagctcgagatgagtatattcgagtgagcgttgtaagtatttctggttaagtttactcaactgatatccaaagccagatttactcattcgttcaagcatttcactattaatataacaatctaggatttgtgccggttgattcggcgcgagattcattttatcaatctcacgattaccgtaatattcatacggaatacctttttccttaattgcaataaaagcaggggaacgttccagaccttccattacagtggatttaccagcaggttttaagaatttttcatgaaaatcaaacatctggtaaaaacctttccatgtagtcgtacgaccaatccaacggttgatattcatgttaatttcagaaacatctttccaataagttgaccgaaccttcacaatatccataggaggctgaaaattatatactgtcggtgcttcttcaatatcatcaaacagagaaacagtttctggataccattctttcatcagaactttattaaaatcaccattatcagaatggctaaaaataacatcagctcgacgaacagtttcttctaatcccaaatttcgacgcaaagaaagaacagaatgatcatgctgataaactacaacacgaatagaaggtttaatattatctaaaagttttttatagttattaatcgtagcttcttgaacggaagtagcaggaacagaattaataattagaatatcacaatcatttactagcttaagtgctttatcgtattctttagctaaaataactggaattgaaaatgatttgtggtcatgagaacttgtacgagtaaatgatttatctttagcataaaccaaagttacttcatgaccatttttaataaaccaatcacgttgctcgagtgagaattttgttacaccacaaccttcaagacctcgagccataaaaatgcaaatacgcatagttttcctcttttcatttaataaatcatgtaaataatattttattttctataaaacgctacgaataggcccaaacatatccataagcaattttgctttttccagatatgcactttttaatattagacatgcatcctttaacggatacagctgcgtctttaattctaggaaatactcgtaataattttccagtagtatcatattgaaatactggcacatttcttttctgagttatcccacgtttagatttagacattttttgtttagttgcattagacatcctagagggctttcctatgttatcagaataataatatttccattgaaatcctccagcggttttccttttaccatctacacattgtttaattgaagttgaacagctatatgacatatcttctgcagcatctgtaatacatctatatttgcgaataaaatttccatttaaatcatattgataaattggttttccagcatttcgtctagcgttagacatgtattttttattatcatcatctttccagaactctttcatgctttccgatattgaagaagatacagctgttttaatccatccatacattttattatttagtcttgttccgtcagaactataacacatcatacgaatagctaaagccaatttaggaagtctataaattttaaataataataaatgcgcggtaaaatgttcttctggtgtcaaaagaactaaattagttttatcatctgtaccacccatacatctaggaattatatgatgtgtttcagtatagtatgtcaaaagacttttatcattgcctctgtttagtcctttttcgatcagtaaattatatatgtttaaataattcattttagtttattttaccaaaaaatttataaagcaatataggagccgaagctcctatccacataatacgccatacagaggctcgttagaacttttaaattttatgcgcttatatgttatagttccttctgctttagctttatcatgagcctctttaaagcgtctcatcatttcctctctagaggaacgaattttattataatctatttcagaagtcggatgttcatctttcacagttgccaccattttttgacttgataagaatcaacccacactttcatattagggtctgctcttacaataggaggattaacttctttgattgaactatcacggtattctttttcagaaattttcacgcaaagatgaccattcaaagattcagtaaatcctgcattaattttaagacgcttgaattttacgtgtgtaagcatcaatcatatcctcaatctgcgatctagtagtcttccaaagaatactgatgagttcatcgttatatggctgttttagaatatcccgacttttcttgatagcatattcgtattgagcaaaattattattttcagctgcgatctgagcgtgcttataaagacgattcagttcgcgtttgtttttagacaataacttatttgcttttctttctgcttcaagacggttcttttcttcaatagaagaaataagcttttccacttcatcattaatttcgggtttatcagtcatattatttctctaatataaaataaaaatcatcatctgttaaatgataccgatagtttaattctacaccattagatttaaaagcggtatcatacggattttctggatcaatatcaatgtcaagagctaaaacttccctgagatacattttaagtaaatagggaatagcttcaacttcaggtatttcttccaagaatccggagaggttaatcgttagcctcatataaaaaatccaaactaggagaatcatctacaacacttttcttttcagcccccggtgttctataggttgattcttcgtaatgcgtcattttatcatagatgtcttgaataaaagtttcatctactaacgcaaccatatcgtcgtcacggctgtcatagacattgtgaacgaagtaactatatttctttgcaacttccttacgttcttttttaatacgttggacgaatgcattaaaacaagcttgagttatatacgcatgtgggtttttatatttcgtttcatcaaaattgtgaagccccttaatagaagcttctataccatctgcaatcatttcttgtttccaagactgggtgtatcctgaaaagttgaaacgtttagataagccttctgcaataagcataatggctaatccgatagtatcattctgacgaactactttatttgggtctttattatttgctaattctgttttccaatcaataatagcttgtaaaagctctttattgtttacgtaattatatttaggcttagtttctgacattttcacctcttagctcaattcatagatctattatatcataatatttgaagacctatcttaaagcatagaggatgaatcaatttcaagcacttcatcagattagccgctccaagagctgcatctagtgaatcaaattggtcaacatattcaattaattcgccgtaattagcgtataaccaccattggctaaattcatactcaaggatgaatccatttccttcaatttgagttaaaccaatgccatttgtatttacttcatacccagcgagacgtaaatcgttaataagagcttcgttcataattataccttagtaattttcaggtctgcaaattttttcttgcgttgatttttcatgcgacgaatagttttatcggaaatttcatgcttttgataagctttagattctacaccaaaagctttaacatcaaattctgacaagatatattgaaccaacaattcacggacagtattgcgtccaatcttctgatcattctgtttcatcgtttgatgaagctctttttcccatttatccaaaatttgaggagttacaatatcgcctttttctagcaaagaaactactttatcatatgcgtaagaattgatggcgtttttaatttgaatagtcatacattatcctcaattacgttaaaattttattatccaaaaaggccgaagcccttaggctaaactttttggcacccttccagccttcgtacatcattgcgactgacaatgacaaagctccttcacatgctgcatacttattattccagaaccaatttagaaaaacttcatcctcaataccgttgtgtttcatgttctggaaaaatttggcgcgttcttcagcaattcgctgcgataatttagattcaggattcatttaaatttcccaattgccattttcatcaataaatttaatccagtcatttactgaccacttggtcgtatcgccttttggagtaacatttaaagtgtatagcccttgcttaaaaagcatgcgtttgatattcatattttcctcagctgtaacgataacactcgtttgatttacgtttagcaactcgttgagaagtattataatcaaaatcatcatcaatgtaaactgattttttcaactttcttacttcaccgcgtaattgacgatttaactcatctttaacttctgaatcaatacctttcattctacgccatttatctgagcgaaaaatgttttcaaccatatctttatgacttaccccatcaggagctttacgctttccgaaatagtcataatcacgcacttttaagtctttacgacgatacgttttacccatggagtttaatttccttagcaactgaactaaatacagcacgatcacaaatcatacgtttatgtaacttgagtataagatagtaaacatcaaaaccatttacataagtaacacgaacaacattaccattcacgagataatactgtccctttttaatctctttatcaaccacaaccatatcaattcctcaaaggtaattcatatgttaataataccacggtttgaacttgttgtaaacaactttgtgaaaaatattttagggaatgataagaaaggaacgatagcttagaatggtaatatacagaatgtgagaaagaaaggcccagagggcccgtcttaatcttctatgatatctctatcatatccaagtgaaatgagagtttctttgaagtgtttaatgttcttttgtctagaatcattaatgaaaatgactggataacgaatgttaagagatgtgaatccagcgcgtttagcaagagatacaatcagcggacgatcatactcaatcttaccattatttgtaagaactttatagaaagtaaaaggagcattgagctcctttagaagttttgtaactgattgacatccaggacaacgacctacttcatctggaattccatagacttcaatcttattttgtttcacaactattccttactaagagcagcattcagcttgttagtaattttatccagacgctcattgaattcactagaagataagcccttttctggagaaatcaaactaatcacgaaaaatatagcaataaaaggaataaggaaaatagctccaactgccataaacagaaagaatgttacagttgtaagaaaatcagctaaacctttacgaaatttatacatttacatttacctttaattgattaaccaagcattgataagcactaaactatactgcgaataaaattctggaccaaaatgaaaatcatatcatttatagtatccataatgtaattcaatttaatcatgtttccacaccccatcggtatttgaccaaagtcgctgattatctgatcctcgccacagctttttggtcggaagatttttctcatacttcccatcaataataacatcaacatatttaagcatttctagttgtttaatatcttcaaacttatatcctgtccacaaccagatatctttctccggaaatcttgctttaacccaagaaactaaatttgaaatctcttctcggttctgtggataaagtgggtcaccgccggttaaggtaaggccttggatatacgatttgcttaaatgggacgcaagttctttaacggtattcatagtgaataactgtccgttacgagcattccaagtactacgattataacaaccttcgcatttatgcaaacatccagtgacgaagagaacgaccctacatccagggccgttaacgaaatcgcatggataaattctatcataattcatctttatatttccaagtatatcccttatgaatagattgaattcctttgcaacatttataaacagcagaatgaaaaaatcctgcatttcttatttcagttgctccagttagttctattgtatttccgtctggtgaatagccaataaccgttttggtgttaaagcgatttctagaactatttttgatatgctctttatgattagtagacaaagttcttcctgttagcccatcagatatcctttttccaaaatcatcaggaagagttactttagacctagctatgctcattagcttcttagttttatcgcttctttttgcgccacgctgattttcaaatttcttagcttttgcatacgcgtattgtgaagaagtaacttttctatgcttactattactcattatccaatatgcatcatacatctttccaccatatattttagccaataaatggtgagcagtatagtgggccttataagttaaaaatactaagttgtctaaatcatctgaaccacccatacttcttggaattatatgatgaagttcatacccggctttagtttgacggggcttagatgggtgttcagcagaattaactagatttgaatagattctgtcataattcattggtgcttaaccctatgcatgatttctttatttttaccgagattaaatccgcgttcgttcggatttcccaaataaccacatgttcttcttatggtattcatctttttaggatcagtttctccacaaatagaacaaacaaatccgttttcagtaggagtcatttcatgggtacttccacatgtaaaacatttatctactggcatattaacaccaaaataatctaaatgctgtgcagcataatcccacacggcctcaagaccttttaagttatttttcatatcaggaagttcaacataagaaatgtgaccacctgtcgcaatgaaatgatatggcgcttcacgagaaatcttttcaaacggagtaatattttcttctactgaaacatggaaactgttagtgtaccagcctttatcggtaacatcttttacgcttccatatttttctgtatcgagtttacagaagcgataacacaggttttcagcaggagtcgaatataaactaaaagcaaatccggttctttcagtccactgtttaagatgagcattcattttagttaaaatttctcgtccaatatcacgaccgacaagaatattcaattcgtgaataccaatgtatcctaaagacactgaacttctaccgtttttaaataactcaattatgtcgtcatcaggtttaagacgaaccccgaatgcaccttcttggtaaagaataggagcaacagtagctttaactccttttaaggaactaattctacacatcaaagcttcaaaacataaatccattcgttcattaaatagctcaacaaatttctgttcattgaactgtgttccaatataagaatctaacgcgatgcgaggaagattcagtgttacaacaccaagattattgcgtccatcaagaatttcattgccagtcgaatctttccatacgctcaagaaactacggcaacccatcggagaaacaggaacagatgaaccagtgatagctttattgttcttagctgaaataatatcaggatacatccttttgcttgcgcactctagagcaagctgtttaatatcatagttcggatcgtctttataaagattaacaccttcttcaacgaacataacaagcttagggaaaataggagttatcccatcacgaccaagacctttaatacgatttttcagaattgctttctgaatcattcgttcagtccagtcagttcccgtaccaaatgtaattgttacaaaaggagtctgtccgtttgaactgaataacgtgttcacttcatcaatgtgttcagtagagttcgctacttctctacccgtttatatgaaacaatgtaatttggataattgtcatctaaacacaaatctttaatacgacttgggtggagttttaaatctttagcagcatccacaaatgatttatagatattgtcatttattttaacatattctgggacgggatgaattgttatttttatatctataacatttggatgattactaacctgttcttcagagcatttaagaaattttgcagctgatttaaaacttctaaatttatttccggattttaaagctatagaaacagtctttttagccgttctactaccaatattatttttaacatgagcttcacttctactattatttttataatgctcaatcaatttctctcgtatcttatctttatgttttaaagataagattttacctttatgggcattactaagtttttgcttatgttcttctgaatccggatatttgttaaatttatatccacctatagatttattaagaataaattcgttattaaaatatttcctaataagcatttcttcatgtttaagggccgattcataagaatcaaaaacttgaagaattatccacttagctttataatctttaagcttttctttaacaagcttagatgacgaattgtattctttccaatttgtatctttaccatatatagttttgaattttttaaaacctatataaaaagacttatcaggaaatcttaccatataggtaaatgctacagaattggcaatttttaagctttttcttaatttccatttcatcgtttcacctcgtattcatatttatacgaggataaacagctgcatgtcaccatgcagttcagactatatcttcaactcttagagttgtctgccgtttcgggtcgcttgaccctactcccttacattcatcagggatagtcgttgggcatttacagctactgctgatttagcaacggattgtctcagtgagagtttcccgttttaggcagattttacatgagcttgacttacgttaactcataagcttggaatgcatcgtatacgtctttttctgttttagattgagcataattcaacgcatcagcgatttgccatttttctgcatcctcaatatgttttgcataggtgcgtttaacataaggagaaagtactttatctacattcgcaaaagtcgttccgccgtattggtgagaagcaacttgcgcagtaatttgtgccataattgcagtagcaactccaattgatttaggagtttcaatctgcgcattaccaagcttaaatccgttttcaagcattccttttaaatctactaaacagcaattagtaaatggaagagcaggggaataatcaatatcatgcacgtgaataattccgctttcatgcgctttcataataaaagacgggaccatatttttggcaatgtgtttagacacaataccagccataaggtcccgttgagttggaaaaacacgagaatctttattagcattctcgtttaaaaggtctttattagttttatgaattaatccttcaatttctttttcaattgtcattttaaactctttctaagctgcttcttgaatgaagctattaattgtgttttggtgtcagattcattatattcaaatcctctttgaagcatctcggccatcatttcctcttttcctaaacgagaaaattcctttgatttatctccaacaaagttagggtgaatattattttgggtgtaatcggattttaaataagtaagtaaattttctaaccattcaagataatcaacaccttgtccctttaagccagaacgattaaatttatgcttcatttgaccttctgcagcattgcatagattacaaagcaatccacgcacctttcctgcttttggtccatttaattcatggtcatggtcgaggtgattagcttgaacatcaggatttagttctcgttggcaaattaagcatttaccgttttgtgcatcataaaatttctgtttttcttctttgtataatttgccagtcaataacataataaacccttaccttaaatagataagggtatttattattttcaagtattgtaaaacatttgatgcaatcgcttatattgctgaatcattcggtcagaaaaagaaatttgagtttcaagccattcaatgtactctgcggcagcttgcattaaatttccttcatagccatcgttattttcttgtgcagctaatttagctaatgcgtatgaaatacgttcaccttgaaaatcggctttaggcttctgaacaacttgattagttcgctctacaacttcttcaatttcgccattttctactgattcagtattccacaaacaccaatacgtaattggcttatcgtagatgttaataatctttccatcagaaagttcaatttcaataattccagtgtcaggctctatatcatcttcacattcttttacaagttcacgaactttaaagacagtacctgcactaagttccggccagtaattacacagccctgtatcagcacgattaattctaaaccatttatctactgtaatcatgtcccatctccatatcaattaagtcatttatcgttggttcattatacaccgtttcttcatcagtgtaaaccggttcttccggctctggctctacagtttcccatctagccgcccaccaaggttttatagcgtaatccattctcgtactgtctgagttactacacgttctcgaagctcaaccagttcaacagtaggaactgcataataccagtcagaatggtaagaacctgagcgagattcatttacagccacatgtacattatgttttggactaaaataaacacactgacgatattggcatttaccatcttgcacccagtcttctatttcgataggtttaagatagtcggaaaaatcgaaatcatagttttccgaaaatgcatcatggtcttcaatgatttcgctcagaactgcatcaacatctaatttattttcaatattcatttttcacaccaccaactgcttacttcaaaatcgggttcgccatgttcccagaaccaggggccgataggaatccacttaccttcatcggtgacgtcatattcttcggatttaagcccaacgtatcgccattcgacttcataagcggtaccaccgattacaactgtgttctcgccatacgggttagacacaacaaagtctataccttctgctttagctaaccaaatcatgtattcatagagtttatattccaggtcgccttctgttccatcgcctagaaaaataatttgttcatttaattctatcatttaaagtattcccgcaattggtcaaatccaccaatatgacttccatcaggagcaaatacctgaggcattgttaagccgatttgagtatcacgacctagtttagtcagaagctcagcaattttctcatcatcaaaaacacctttttccggcataatgttgataaattcaaacggctgtttcttcacagtcaaaagacgttttgcattatcgcaatacacacatttatgaatgttgctatcataaccatatactttaaacatattattccttaattcctagtacttgtttaaaagtctcgtcgtaatcaagactttggcccgtttgttctttatgcttgtatataatatcacttacttctgatagcatatttttatatgaacgagttaaagcagatttaagcacgctgtatctatcaggaaatttaccgtgttcattataataagctattgctaattcacggactgccttttcagcagtctccatatattctttacgctttgtcattttcttctcggtcaaatcggtgtttacaatggcgacatttataacgaagattattagtctgccaatggaccaattgcacctgttcagttccacattcagggcaattaggaacgtttttagaagccctttcgcgacgttcaaccatgaccattacagaatcccaattaacaggagggctataatcatcacaaccataaatctttccacgcatttccagatcgtcttcttcaccagccataataattttaattagactagaattacttgaagctgcaatgtcttctaatagacgctttttcatttcaatacctcaatagcattacgtaaaccatttgctttggcggttaaatccttaagaactttagtatgctcttcaatctgtttttcaacttcaatcaaacgggtactgagatattcgcgttcttctttcatagtatcattcccatgattgggctttggcgtaggtttaaatttattagggtcctttaacttaataacaactttagtttcaaacagtgaaagaccataattcgtattatcatcaaaggattcaactacgtccattttagacaataacgtacgaagtttataagtcaaagaacgaatcgttttattatgtttattagcttcacatggtttcatatatggaagatttttatatatttcatgcggagcacaaacaataattaacaattcaaatggtccattctgagatgatggaatgaatttagccgtaacccaatcttttacatttacattaatagtacgatcatcgccatgacaaaacagtctacgagaatgcttaaaaagcatattcacattattattaaattcgtgtgaaaactcactggcaaatttttctttactatctttagaaaaccattcttctaaataagttcttttcacgtcgttaataatattaaatgtaacaacattatcatttgatacattagtctcaatgtagcttggatacataaattttttaacattattaacagctatcacagaagatagtaatcgggaacgtttgaaccactcgagcaaagaaccaagagaatgtgaatacgcattttgtgtatctttacaaatatggttttcccatccaatatagtctaaaaaatcacgaagaatattattgattacttctctatgtcctttctgataatcacgatgtttagtaataagactgtcaaaataatcaatataatgtttacgagttttcatgttcttctcacttggttaatgatttatactccgagccatccttggctttaaattttacttaattaactgcaaagcttgttctagacgatcaagacgattaacggattcttcccaaatctttttagcctgctcatattctttctgcgctttattagaaatttctagaacttctttataagctttttctagtgcaataacttctggacgaatttctatcggttcaggtgaatcatcaagacattccattagttcctcaagggtagtttcttctttaggagtattcacaatttcatcacatttttgttggtaaatttctttactagtaggtgaataagcacatttcacttcacgaaggctaattacgagtttatatccttcccaaccgcatatattcttaaaaggatatgtatgaatattttctactgtttccatacaaagtaatgcggtctctaattgacgagttatagtgctagcaattgagaaaaatttattaatattcttttggttagtttctggtgtaaaaaatccataattcacaaaaatagatactttatttttatcaagttcatattcttttatgataatcatatcagaagccaaaggatgaatttgacgataatcgccataacataatttagaagctgattttagaatttgcttcttgaaaagtctgaaattactaatccaacgacgagtaaaaatattctcagggtcttctttattattgagatgataagaattaacatcaccgaaccaattatatcctactacatctttagttcgtttaatttctttcccaaaattaccaagaatatcccgattaaccaaatatgaaagaagacgagactctttaaaggttttcttgataacatctttatctacacggctagaaattttacgaatttcatgaataaatttagtagaagaaacaatacttgcatcaacactattgagccactgattgataatagaaagaacgctattttggccaaacatcggtatagctttatcatcaataacgctattgaatgatttgatatattcgttacgagtcatattaatctcctcagtagaaagtaagaacattataccacatccttgtggcaaagtaaactagttcagtgcatttagtgcattgttcagtttagaacgttgctttgtcagattttgaacccttgactgagcttgttctaacgccttttcagcttctagcacttcattggtcgctttaactaattcagcatctactgccttaagagacttctcaatcgcatccgcgtgccatttttcaacaggtttaagacttggattttcaacaggaagaaaattcactttattgaatttccatgcatctttattacctgagctatacatccaaaatttaggatcgtttgataagtaattagataaatttaagttatcttgcacttcttgctttttctcttgtgtgacttcgtcctttctcaaaaattcatatttaaatgaaacgatcatatcaagttcatatgatgttgtatctaacttaaatcgttcaaaacgaggtaaaatcttagattgaacagcagcaacaacatccatatacttaaatgcttctgtcaactgcgatttaaggcattcgcaaattgaaagagaattttttgtattaggtttaaagctaattcgtgcagttctattattttcttttaacggtcttacttccatctgaagagtataaccatcaaatttcagattctttaagttaatgtcagagccttttaatcgactagcagtagacaaaatttgctttaattgtctacggaacagagcaataaatctccattcaatacgataatgatttggattgaaaaatccagatgataaatcgaccttactttgaccaacgccttgaacaaatagaggatttttataatcaatagttttacagaaatcggtgagctgttcacgagcagtcatttgagtaatataccctgctttactaaaattacgcacccattcactgctattcgtatacttaaagtgatgaataacacgattaactttatcagggtctaaattattttcacacaaaaatgtcataacatcacgagtatagctggcattacgaaccatatcttcaatttgagaacgagttttcatggtgttccttaagatttaagtaaatcaacaattttaattaacttttcacgctcagatttagctttactactcaatccagatagtctgaaaatttcatcatcatattgttgaatagaaatatcagctcttcaatttgcttattaaaataatcaatttgttcagaatgtttttcgttactacgaactggtacaggttttgtaggcaatttagttgaactggattcattcgggcgataaattaaaatgcaatttgaaccaatcgggcatttagcaccagatgaatattctaacgttccagcttcttttaaaacttcaaaagctaaacagagatgatgccccatattaacataatcagtagagcgagctctgatgtaataatcatctccacgagtgctaaatttaaagcatttaaggtcttctgtattattagttttaaaaatcatttgtttatctagacctttagccaatcgagcacctaatgctaataatcgtcgttgattttcccacaaatctgcgatcatttggtcaaatgataatttcggcattagccgaccataaaggtcatatcctttattataattgcgaaggatttcactcgcattaatacgagacaacgctccaattttattaagggctttcataatacgattagataaactcattccagacccgtttgttcgctgtaaatgtttttctaatccaaatccaatatcaactttaaatttatctaaaatttcagcatgaacatctctgtcaataacattcaaatccaaagttgggttaaatctatgaaaaaatttatctggctctccacgacgtaaaacagtccattcattcatcatttttttattaactaaagatttaattactgcgttgacattattaattactactgacatattttcctcactcaattttaccaattacgcggaataagattgaacagactatataagtaccacatatagattgaactaatgccattccaaagaaccaaacaatattatcaaaccatgtctgttttacgtcgaaaggacgtaaacttacagtaatattatcacctttttctattgaagaatacgtctctggggaaatatattcactaaatctataaccgtctttgagttcatatacagcaataaacgataaactagacccctttccttgagttcctgtaagggtattaactacagtaacatcataatctttataatgcatataatcattaattgcgtaataaccatatgcaattactatacataaacaacatatcaataaattcaatcttttaattatcaactgtttcataataatctcaattaaaagggcttagaaccattataccatccttggtataaagcggttatgcgagtaccgtatttaaccgttcttcaaacttccgaagagtgttctggcgttcagctctttgctttttgtaagtttcaatacgctctgaaatgagagtgtatcgttcatttactgattctttcataaaatcaggaatttctcgagaagctttaatctcgtcaaatttatcaataacagcttgctcttcagcaattaagttatcatacatcaaaatatctttcttgatgaactcaatatcttcttgagttacacgagataatttagatgctttatcctttttgtactgttcgttagtatcacgagaccagtgtaatgtacgatttttattcgtattcttgtaaatttctacaataccaatctcatcgataataacgatccaattccaacgggatttgtaaatttgtcctccattaacagtgatttcacctccgattgagatgtcattaaagaacttgctttgtgcttctgatttaaatttaccatcgttgtaatttaccaggttgaaaatatctttagcgttcattttgtgttcctccgtagttgatagttgtatagtaccacagaggaacggtcttgtaaacaactaaaagaaacttctttcacaattttttccactgaaccaagcgctcactgctttcttagtttcaggagcagtgttatccataaaccattcaaaggcagcctttttatgattctggagggcttctcgggctttaatctgctcacggtctattaacactaacatatgagcctttcttgtcaccaggggcttcttatgattttttgaatactcccaatcatttgtccatcgcatcgttgttgcgaattgaaatacagcttctttaatcttagtttcgtaaatttcacgagcctttgagtataacatcattacctccatttaccagtttaattctagtcatctttttgatggcagtccatataatctatttctgaactgcctttttgtcttagaaggcctcttatgaatttatttcagaagagtaacccgtagcgatttcttcccaaccgtttttgtcggtcataataaagtcagcaagataaagtgcagtacgcagtgaaacattacgtaaacggttaacattgactttcatccatgataatgctttataagtttcttcatcagaaagaccacgcttttgcatcatgtcggttgaaagaataacatcttcaaccctgaccataatttcttcattagtgtgaacacccaaatccaaataaactgagcgggacactaatgcttgtaaatgtggagcaagtttagtaccacggtctaattcgcggtcaatgtcaacgtttgtgataaaaacaattgttcctttaaattcgagctcacgctcaatgcctttttcttctaagtaagaagatgcagtgctccagcagactttacgggtctctccagtgtccagagcagctttcagaagattaagaatgtccatatcagagaaaacatccacatcatcaatcaaaaggacagaattctcttcacgattattccaaagctgttcataaagaccgataccagagattttaccgttaatgcttttatactcaatgtatccaatatcatttgctttattcaaagctttatctaaagaatatgttttaccaatacccgccgcaccagagataattaatgaacgaatatttccgttaataataccattcgtcatcattcccataacattaaatcttttattaatgcgggttttcatatcttcatatgattctttaacttcttcaacttttacaccatcatatgaaatgtctgatttgtaaacccaaacaccgcgacgtttaccgtcaatttcaacaaaaactttaccatctccttgtgcatctaccggagcattatcagggaaccattcacctaagagctcaaaagttccagagatttctttaccgaagtacatacccttattgatagttacagttttcattttattctccaaatccgtatcagttgatagttgtatagtaccataaagctttatgcttgtaaaccgttttgtgaaaaaattttgaaataaaaaagggagcccgaaggctccctatcatttataataacttcgatggttttcaagataaaccctctcaaggaagtcatcccagaaactcatgtctactttttgctgcataccgttcttagaagcttcagtagatgctgcttctacttgatcgaccacatcttccaaaaactcttgaacggttttaaatggatgcttacccaacttcacgtcgagaataaatggagcatcttggagtggataaaccaagtcaccagttttgtaaatttccaatagttgaagtccaccacgacaagcatggctcagagctttccagtcaatgccttcattggcttcggccttacgagcacgttcgccgtattcagcatctaatttgttcagtgactgcttaagctcaataagagaaagcgttgtctgatatttacgacccaacactgtgtaaaacgtttgtgggcctgttttctcatgattatggaacacccattcacagaattcgttttctggaagacgatgcttaatatcttcaactttagtacgacgctgcttaatggaaccatcttcttggtaatcaacccattgctcagggatttgattaactactttcaatacatcgcgtaatgcagccaaacgagaacccttgacgccgtatttagaagcttgcttgcggacatatcctaaatatgatttcatgttagtcgtataaaaacgagaacggttgtcttgaataaacttccamacatcaggcaaatcggatttaaccactagttcaggtggagtgtgaagcatatccaatgctacaggttcaccatctgctgctaatttaaagaaatacttaagactatacaattcgtggtcaatattatctttagtgtttttagatgatgtgttgttggtatttttactcatgtgctctttgacgtttccaataagaatatcgcgagcaggaggaacaaagatttctttaaaatctacatcagattctggagtagaagttccataaagatgactaccaaaataagacttaactacagttttcattattagacctttcataatcttcattaaattgtacaatcaatcgatgataattagattttggatatcctaatttacttatataagttccgaatgacccacgtttaggtctatttaatttaatccataacttataaagtaagtcatagtcttgccaatgtttaccagttctttgtctaattttagatgaatttgattgtttctttttagcttcaaaattattcaaagattttttgactgcagcagagtgtttttctttaactcccggtcgtttaaaagcagctttcacccctttagatattttttctttaacttccggtttgttttgtgcttctaactgggattctgaaattttagcccttatttcaggaagacttaatgttatcgaacgtttttctctgtattcattagacttccacatttctttcattgtattagaatgtatttttgaaaatttttctctaactaactgatatcctcttgaagttaatttgagatttcttcctaaagaatcttctccaaaattataaaatgaccaccatgcataaattaatccaggcgaattgtaatgaattttagctaaaagccaatgggctataaaatgctctctagctgttaataaaactagattatcagaatcatcattaccaccaatacaagatggaataatatgatgcttttccgtataaaaatttaatttagatttatctaattttctgagttttcctttcttaattaaattattatatactttagtataattcattggttcttcgtctcatttaattttgctttgcatttataacacatgtctgttccacttgaaataaacatatttcctcactttgaaatcatagttggaataacagaatcaagataagtctttagtgcaatagcttcctctttctttaatgtaatgatatgtgatcgaaaatcatcaatttgacgaatagatactacatctccctcttcatagcattttgaaacatttaaaatagtttcatcattctgattacaggagttagtaataatagcattacattttttaaaccattttaaattatgttttcttttagtagaatcataaaaatatttaatgttagttataaaattatcccaattattaattgataagcacattgactcgcttttaatattaaatcctgggcatgaagaataaaaatgaattttatgctcatcattaatgcttacaattttatcagtgtaagcatattcaatttgggttaaacgaacaatttcgctaggcgtaaaatataacatgtcatcttcttgcgtcaaacgatacatgttatttactttttctatagccaattcaccaaaaagtggactaacttctaatactagtcgtttcgcctcgctcatcattacatactcctctgaatcatattaataatgttattcaccagattataagtaaacattgggtaattatattgaatcatcacatatataacaaacaaaactttcattctcttctccttggcagttgacaagattactataccataatcttgtcaacttgtaaaccattaaatgacgttttcgataaaattttgaagctttgtatgagcatcaaccatgattttcatttcttccttggaaaagctgtgcctctttcccgagaagaacattcatagtcagaaactgcattagcatattcttcaattagcttcattaaaaacatctgtttttcagttttcattattccacctaatcatttcaagatattgaactaacttagctttggatttatccaaatcccttttagctgcttctataccgtcgtatgaatatccttcacaatgctcaactgctaattgatatgaatctatttcaatatcatgcgctaatttaatgattttttcaaactgttcgcgtgttagcatacttaaactctcgtattatgatcgataatttcatcaagaaacatatctaacgcttctacagcattattaactttagcttcaaattcttcaatacctgataaggcaaagttaatgcgttcctcatctgctttacgaattaaagctaccaattccttaattttatccgcttgttcgatactaatcattattccaccacatatgaaagagagaatattgcacacgccatgtgagttgcaacttcatcacacatattataacgtttcttaagaagttctacaagttcttcactagtaacttcatccatgtcgacgaaaaaatcaccattaatgatgacgtagatatttccttcttgattgagtgcttcaattttcatgatgttctcctctttatccgatggttgtatagtaccacagctcaaacggaaagtaaaccggtaaaatgaaaaaaagtctcccgaaggagactaatgttattcgagggaaagaagatacttactctggtaaaacattccagtaatatcatctatcgtgctttggatggctggaggcatttctttataaatgctgttagattggtctagtatgcgatcaatcattttaattgtgtcggtaggaagtttactggcatctggaattgaaggcgtgtattttcgaccagaataccccaaatattgctcaccaaatttatcaatcaaatctggcaactcggaaaaaataaaatcgtatgctttgtgtctagcataacttttagtttcaaaatgtgcagaatgaaaataagcttgtgcagccattaataaacctaagtattcatctgcctttgaaggttttccactttgtgaaaagtcgctgaatttcattcagtctccaatttaatgttcataattctagcgtatgattgtgccatctccgcgcctcgctctatacattcaaaatcagaagagcacgggtcatttttataggtcgttctcataaaactatagaattgttcagacgattctacgcttttattttcaaaaagcatataaacgtgcctaataccagattccataaatttatcaaaatgaggatcgacattcgcttcaatcgatggagataaaacaaatgacaatcctagcatggcaaaaagtgctgttgcttttaaggccataaaggcctcctatcatttttgtcctgtatttactttgtgccgatgcacggccttaactttatcaaggtatttttcaaaatttcgcaatctagtatagtctgccggagattggttgagtgatacttctcgacgcaaagctgaaatgatatttccaacttccctacgaatttcatctaattgaagaacagtaagattgcgaagttgcttttcagttaattgtagcatatatacccctttagttagataaacctatttataacttttgcactaaccgagctttttagttaattcattccaatgttttctacacaaagaaacataaatttcatcaccaatacaaatttgattaccttctttaactggtgttccatcttccattaatcgagctgtcataatcgcttttttaccacaatgacaaactgcttttagttcaataagtttatctgcaatcgctaaaagttctttagaaccttcaaataattttccagcgaaatcagtccttagcccataagccataacaggaacattatatgtatcaacaattcggctcaattgatgcacctgttcagtttttaaaaactgagcttcatctacaaatacgcaatgaatatctttttgtgcttcagcccatttatagaactcgaaaatatccatatcatctgtaataatattcgcttcctgcttaattccaatgcgagaaacgacttcacagacagaatcgcgagtatcaatagcaggcttaagaactaatacactcattccacgttctttataattatgtgcagcaatcaaaagagaagcagattttccagcattcattgctgcataagtaaaaattaaactcgccatcttagtccttagttaaattttctaaatatgtttctaaatcattttcagctttatcgatagattttactaattcgtaatatgtttcggcatctccatattcagaagatatttcaaaagacaaatccttttctaaactaataatttcaccaactaaaaataatatttcgttcttttgttcgcgagtaatcataaggaatttatataatcaatgagttcttgttctttattatcgaattctttagaaagttcttcgtactcgtttgcgctaaaaggaccgcattcattacaaactttttccaattcactatttttatccataacttcgtggataagagaaaagagtgtgtctttttgttctttgcttaaactcataaccatgtcacctttaagcagtattcttctacatgctgtttacgacctttcttatcaataaaggtatattcaacgaatgttccaatgtagtctttatctacatcatgtggactattaattggacatttagtgcgacaaatgcgttcccattgacgaataattactgccttattctttgggtcatatggatgtggataatgtatattcataataatggttcccaatcaacaatcacaatttctaatttagaggaatatgtatctaaaatcccctcaataatatcccagttccctttacctatgcctgcaccaatcctaggcatatagattgtaggtttaatcagtttattttcaccaaactcatttaattctaacatacaattcattaaagcggaatactcaaaatttggccctggttgaaattgagtataaagattgaagcagtaagctttatgagtcctaaagtatttttcatagactgagtaagaaccgagtttagttacatcaccccattcagtctgtaatttatcagcttccaaaattttagggaaagctttggttaattgacccgctacgcctgaacccatagtatgaaaacaattacatccatgtgcaatatttttaccttcagcgaaaagggcgacaatatcgcccttgatatattttacaatcatctagtactcaatcctcgattataagaatctaccaaacggtcaaccattgaatgacaagcggctttatctttctcctccgcaactgaacattctaaggtattccactttttagcatatcgttttaacaatgtatcgtttttgtatctgcttgatttatctctttctccgtctttatatgcatatattaatttctgtgcaaattcagcttggcatgccttatttttcccacaataatctgccgcagtgcggtttacatattctctaatttcagtatatgatgtatctgctgacgcagaagcagaaaatgaaattaatcctatacataaaaccaaaattttagtcatttactatttccaaaagtttattatttttaaggtaattagccttttctaggacttcagaagcatatttagaacctgctttaacattccatcccgaattataagaggatattgcttttcttatatcgcccttatgtatatttaaccaataagaaagttcaatgtacgcccaggaagctgaattggatcgtttattcaacattctttttatttcagcatcggtcatattataaccaagttccttaactcttgctcgcatagtaggcaaataattttggaacattccgtaggcgtgatgctttggtttagattttaaattaactccgccagagctttcttgccataaaatggcagccattatatgacctaatccgctcttgtggatatttttgtgtgttttatattttccatccttagaaaattgttccccgaattgatacgcgtaacgcatgttatcgagttggacattactgaaagtatgctcggagctatgtgccatcattgaaatggccaatagaccagcgagtagtgcttttctcatgcttacctcattgagttttaattactgctttagaagcctttcctggtaaacgacgactgttgataattgccatcctacattgaagtgacgggtctttgaacttctcgttaggtttacaaactgtaaatccaagccaaagatttccatctgtgatttctaaacgtccaggacgatattcaaccccatcaataaaatcctcgtcaatgtcaggacgcggaggcatactcaggaattcattaacttctaaaacatggtcttttattttatggaataattcaaaaacgtatgtctcatcaatctcccgttgaattgcgcgatcaagaagatgttgagaatattttagatgaaacgatgagactcctgctgcttttgatgcctcacgaatctcattgttaatttgacgaaactccgactcaaagtgacgacgaagcttatttcgacggataaaaacttctgtattgatagtcatgttattctcctcttaactgatagaaaaattataccacagtcaagaggaaaagtaaacagttattctttaaatctaatcaatttattcatagactttgaaacttcggcacgaacctcatgtagatttttgagctgttcaagacgctgcgtatagtaagcaatttcatcttcttcgagacagtcctgcgaatcttctttaagataacgtgcatagtcctggaaagcgttacggactacttcctggaagtcatcaagactttgaattttcttaggagcaacagatacacgacgaggggcagtataatactcataaccaaaccctgcgcttaattgagccattagtatttttcctctggttggaacactgcacgacaagcccacatactggcttctttgagtttcgttttagcaatagttaactgatcgagactttcagcataattcttcgcgaattcacagtcttcgcaattatctagtgcttcccagaattcatcatataaagcatcaaagataagtcctaaacgaacttcagcgtctttaatagcatttactttaccgattttctcttcagtatgtggtttataacccttaatatcttcaatcatatttgacttcctcaccagtacataatacgtattcaactaaacgaataggttcatgaatgccatagccttgaacagaaatttctgtcgtaggataaattccatcaatatcacccatattccacgcttcattaaattgctgttcgcctgagttactaaaccactcgcgaaagcatttagcacatcttcagaaccttcaataattatctttgccattacaaactttcagtaaaggtacgagcgataacgtcgcgctgctgttccggagtcagagagttaaagcgaactgcataaccggatacacggatggtcagctgcggatatttttccggatgcttaactgcatcttccagagtttcatgacgcagaacgttaacgttcaggtgttgaccaccttcaattttaactgtaggttgtggctcaatttcaatttcacgggcatgcaaaccatagaaaatttctgggtctacaaaagagtcctctttaaaggttttagagacaataattcgtgcttgaataccatcttcaaaataaatagtacctttatgtgtgccttcaagaatttgatatgctttcatataaacctcaattagaaaataaatttatccaagattgttctttaattaaaaatggctcagaatcatatgccattaaactttgcgtaattaatcctttaaaaggtccatcaataaattccatggtaaaatatggaattttattcattagccgtgcattaggagcagtgcacaaaactctgcatcctttgaatacgcctttttgtaatttgtattgcttgggataaaattcgctcaaaatgttattttttgccaaaatttcaaaatgattcaccaatttatttttaatagtttttggcgaaaaataaagatattcgaaaagctgagtgtctgtcatcattgcattccgattacgaaaaactgtggacgagtaataccaccaatgcaacatttactattacagcagtagtgtacggtgtcaatatggacactataaatcttatccatatcaggagatttgacaggctcatcaattatatacaaaattcgcgaaagctttaaacctctgaacttgcttcctttattaccaataaaactgcgtacagaatcagtaaataaacgaaaacgtatatcatcattagaataacgcgaaaattcctttttgatgttatttgcagaaattttagcataagctgaagtattagaaagaacaataactgttccgccatcatacaaccaattagcagcaaagttagtcacagcaattgatttaccggattgacgtccaccatctagtcgaagtgtacaatactgtttaagtaagtcttcaaatggcgggatatattcgtttttacaaatttcttctactctagcatcagaatggtgtgtaaaagcattcatcagggatagataaggaccagttaaaaatgttctcatttcttctctataagctctataagtttgggccattccgtggcacatgaattgtccatttctgtatttacccattaccgcgcttgggctcgaccttattacaggttggcgggaatccctcatataatcatgaggtccaggttgttcccttatgcataaatcgccttaccgtagtatttgtaccaagtaggacgttgtgcaattttttcatctaaacgagcttgtgatatagcaatagaagcttcatggggaatataatcaccacggaattcctgaggaatatcactaatatcctggactgtagtatccttgatattaaaaccacgttttaaacattcagctataagctcaatttgacgtttacgtaagaactcgagcttatcgtaaaagaatgtaacatgacctgcgccaaggataaaagtaggactgattttaaaatcacgaacacgtttaccgttagcaacatgcttacgaactgcaccaaaaacacgcggcaattcacgatattcagccattaagtgttggtcagccaattcagatactaaagtaaggttgatacgagtcattttagtgttctcctgtagttgataggtctatagtatcatacctacaggagatgtaaactgttatttatctttaattgctttagctgcttcgatagccgcttgctgaaggtcatccatagacatgccgaacttagaagcaaagttatcaattttcttttcgacggcattcaaaggcttggcctgtttaccttcattagcgccaggaagagcgagaatacgctctctgtcaatataaaggcttacaagtttcttacggtctttatcgggcaaatcatgaaatgaatgggcctttttatttacagcggcttctaatttacctgcgcccactcgcgcttcggcaataaattcttgatatgttttcatatgtttcctttaaatgtaaatatttttattattctatcctagaattgtgataatatattcacaattctaggagttgtaaactgcttttatttaagcgtcccaagtataagctttattaagaattaccacgggctgcattagcaacggcgtaagcgtactgaatattagcgtctttaaacttacctttagaggtatctatttctgccttaaagccgcctttagtcataacatcggcaaattctttacggaaagccattgcatcaagacctttccatgatgatttatgcttggcaaattcaagacctgcgaagttgacagctttagccaatttattatcaatgacccatttaccggctttaggcacaaacttcgggcctttctgtttagagaacagttttaaattccagcggcgaaggtctgcatcagctttaacaaattctgagtctaagttactagcaacaacatcttcaatatgagcaaatttaagtccgtcaacttcaatatttaaatcggtacgccagcgaagtccttcccaagcgaaggctttaaagtcggaagcctttgtagctaagtaccgttcaataggagctgttttagggtcaaagccgtttcctgatcggtaggtccactcatctttgttaatgcctttggcctttactacagaagcttcggcaataaattcttgatatgttttcatatgtttcctttaaatattttaattagtaattgtctattcaagtaattgtgaatatactatcacaattccaagagaaagtaaacagctttatagatttttatacgcgtcccaagtgccagttctaaacgttgtaatgactcgttttgcgcgattaggtgtttgattataccatatacttttagctaagttaactgctgcttcatcccagcgtttttgttgaagcatacgtaaagagttagtaaatcctgccacaccggtttctcccatttggaaaaccatattaatcaatgcacagcgacgaaccgcatcaagagaatcataaaccggttttaatttagcatttctcagaattccgcgaacagcagcatcaacatcctgattaaagagtttttcagcctcatcttttgtaattacaccattgcaattacgcccaatagctttatctaattcagatttagcagcattaagtgatggactttttgtaagcaaatgaccgatgccaatagtgtaatagccttctgtgtctttatagattttaagtctaagacgttcatctatacgtaacatttcaaatatattcataatacctcctaagtatttatagaaggtatttataaaattaaaagaggttgttcattattcggtaaagtgaaggacccatcacatattgccactgagtacgaggaataagagcaaaagcgtccatctctggaatcataacgccatctttattttcaaaataagactcgcaacggcaatttctgaacatctcatgctctactggaatcgtgtaataaaataactgtaggtctttattactagaatatttaaatacacctaggtcttctagaaggtctggattataattgctaaaaccagtctcttctaaacattctcttcgtgctgcatctaatgcgcttaaatcagaattttctacacggccctttggaatatcccaacgatgtgccatcattccagtcttacgagaaccagtaacccgacccataaataaatctttatcttctgtcataaagataataccagctgataatgttttcattttaatttcctgcattcagtgataaagttatttaaattttgagcatatttcttttcatcaaaaatcttttgctgtctgcgtaaccgccatggcatttcaatgaacatacgccatatccctagataataccgctgctgtaaaaatattaacaagtatagttaaaagaatccaatcgcctattctgtccattggatttttataaaaaagtaaaatacgaatgatgatataggaagactaatgatataccacagaagaaccttcttatctgtgaaccaatcagcattcgttaacttagcgcgaccattttgaatacacacgaatttatcatctgttacagtaaatggcttagctgcttgatatcccattctaaactccctaattaatcgtttctttgtatcttcggaacaaccattccaatcaactctatcaactggaatgccatcatccccatcatctaaatcataccagcgagtttttaaaatcatttaattttcctgcaatcaatcacaaactctttcattgattcattttcaatataagacatgtagctattatattcttttaattgtattttgtaatccttttttctttgccaatttattttaaaattatcataatgaaaatataaaatgataccaaagaatgaaaacaatgaaataattttagtataacaaagctcgctccaaacttctattatatctactgtaccactgatttttaaaataaaacagtcaattaatagtccaataagactacctgtaagagctgcagccaacgccacagcaaaaattaaaaatgactcagaaaacgaatatttgactttatttagctttggcttttgcatcgtgattccttaacaaatttcataatttcattaaattcatactcagcaagtttaagctggtgttcctttttaatctttttgcactgggctttccaatcacgtacgcgtttacgataatgtcttccttgataccagtatcctatccaatttacgggtactaataaaaatggaactaccaatggaagagttagcattaacataattattacgccagaatcaatatcagtcataacatctaaaacacctccaataatcaatagaattacaaatgatatagctatcacaggacctattaatacatcagtagaaattatctggcgctttaattcatacttcaaaggtttacttggaaggtatagtgatggctttgacatattctctacattccttaacaaatttttctagtaataaatcactttcaaaattaggattttccactaatttatcaaaaagatcatcaacaatattcaagatatttcttttactaagaatacgtttattttcatgcttcgtttcagaatcaactataagagtaaagaaatatttctttccctgaaattttaccgtagtatcaatataaaataaatttgacttttgtaaattacgtttaaaccatgcgtcacttaaactataaacaccgagataatcaaaatcgtcgtttaaataacaaactgaccattcaggagaaatgaaatcagtaaattcaacatcaaaatcacatgtcaatgaatgaattgattcaatactgttaataagtattccaggacgtattaaagactttttacctctggaaaatcttccagaaagactttcatcagtttcatatgaagaaccccaataataattacgtccttttgccatatgtttaagagcatttagtaattggtctggaacatcaacgtgtctttggaactcttcaaacattgaattgaaatcactttgcattttcattcctatttactccaagtaataggggccgaagccccttatcattatttcagagaattaatatattcctgaacatcggcagaggtagtttcaaccccagaaatattaccattaaaggtttcaactcgagcaagagtatcttcaatatcaaccttagtcagtgctgcaatttcaactacatcatcagcagtactaattccaagggcatttgctgcacgagtttcacggatatattccaatttaactgcaagttcttggcgagcatcatctaactcaactactttcttggcgatttcaattcgcatttcagcataaccatcagctttagtagtcagctgttcagctgttcgacgatatagcaagccgagtttagcatgcattgttacatcttgaccttcggaaagaagcttgcgaatttcacgctcttttgattcggcctgtttattcttttcaacaataagttcacgaatacgtttttcttcattaatagatttaacagaagcagtttttaggtctttaattttatcaagtagttttgctgctgcagcagtatactgttcttcaacagatagatttttagccattgcagaaccaagtttagtgcgaataaactcaacaattttcttcagtgtgttcatagtatttccttaggttggtataattagataatataatatcacgtttctaatagattgtaaacttattcttcgtctagctcgtcgataaaggcgttgatggcctcgataatggcatcattgatagccaataaaataaaatcatcgtcagtacctttagaagattctaaagcattgatatatgcttggttgacgagttcccaagcctttttaaaataaggagcttcatcatcaggacaaatatcccgcacgccttcaaagatacgtttggcataatctaacacccattgtacaggcatgttttgagaacgttcgttaaactctttaaagtccttagattcaaaaagctcttcaggataattatttctattacaaaaagctttactaaagttacgtttcataatgttttcctcatttgtataggctcataatatctcaatcatsagcctatgtaaacttatttcatattattgaaatattcttctgcgatttcgtcgttatcatggtaaactttagaagacagtttaacataactttcagcagtgaacatgttaatcacaacctttacagtataccactgaccgtcttcattacccattactgcgtaagtttcaaacatcggatggtcaggaccgataactttaatatcattcaccgtacgaccgaaatcttctgaaacacatttcataaagaagttgaaaagttcaccgtaattatccattttattctccaagttattttctgtatcagtagttgatagttgtatagtaccatggaagaacaaggatgtaaacagttttgtgaaaaaatttttaaaaagttttagggaattctagggcggagaggggcaattaaaagataggataatatattataaagggtataaactaaatgatgcctagagaggtctggaaaggcttagataccaaaaagccccaacctttcggtcggggctaaccgttgcggcaaccttgtcggggttccacctgccaaggcaagtgtttgtacgaaacgccgggattcgaacccggttattaagtagttgacgctactcaatatttttaaaaggccatatctcaaccatatccgaacgttccgtcaaaaacgctactcggcttacggcaaagatatttcctcgaatcgataatttggtgcgccgtttctgctgtgatgtaagagggcatcaataaacgcaaagattattaacgcaattccttactcagggaaccatcagtccgacgacttaccggtagcgacccggtttctcatttggtatcccgccctgggatcgaaccaggaccgcaaacttagaaggatcgtatgctatccattacaccagcgggacgtaatttaaaatttcatttttcgacctttaaaccatccttctggaattgggtcagttttcttaatacgtttagaaactttttcatctaatgaatgaatccacatcataccgaattgggaattcttttcacctttctggtgattatttttggcgtgagattctttcattttattaatagtttcaggagtatgatgcttatttagaaatctgctattatttaaaaatttttccctgtattcaggagttgaccacaaacgtttaaatacatttgaaccaattttacgatatttttcttgaagtaaaatatcattttcaaaacgtgacttaaacgatttagctccttttaagctagcatctttcttctggtttagcattccaggaatatttacatgatcccatccaccttcaccgccaagttttaaattatacacatctggtctatttaaaaactcttctgtgacaatatttttctcggcttcaagcatagattctttatcgtcaaaatactctaatatttctttagaaaaattttctataccatatttatcttgggctctttttaataatttaccagaacccatatatccatcatctaaattttcggtagaatgcacaccaatataaattttattattaattttatttgttattttataagtgtaatagaacataaatatctcctatttctaagagtatttatgttctcaaaatatgacccagaccagatttgaactggtaacctttcccttatgaggggactgctgctaaccattgagctacagggccttggtgctgattgacggaatcgaaccgccgacatcctcattacaagtgaggtgctctacctactgagctaaatcagcaaaattacggaggcgataggatttgaacctatgagtcgccggagcgactgccggttttcaagaccggtgcattaaaccactctgccacgcctccagtctccatacaaggatttgaaccttggacctcctgatcccaaatcaggcgctctaccaaactgagctacacggagtaaattaaattggagcggataatgagaatcgaactcacatcatcagattggaagtctgaggtaataccattatacgatatccgcaaatttggtgcgagaagtgggactcgaacccacaaggaaatcattccgcagcattttaagtgctgtgcctttaccaatttgaccattctcgcgctgggaataaaggactcgaacctttgcatctagcagtcaaagtgctatgccttaccaacttggctaattcccaattattaacaaaggctctctaacaagaacccttgatgatagagggtattaatcagtgcggtatgagttaataataacaaataattcttaaagcatatttaccatttatgatgatacgtatttacgatacattcaagacccaaaggattcttgaaaatatcatattcaagaggacctttttctgtttcaataaagaaatcaaaatttactgtattaaatttacggtcttcctttactaatttaacttgagaagatgaacgatcaatgtaaaccttttcaacttcaaaacacgttaaaatgccataatcatcaatcaaggctttagctgcttcttgatcatatttatatccattttcaacggatgatactttcgcataaagaatcatcatcaacctctatcaacaatagcatgagtatgggcatttacgatttgccaccagtcgaaacgattggaaccataatctggtttattttcattttctttaatgatatcacgcagtttatcttctgtttcagcatacgcaattaaatcatcatatccaccacaaggataataattatcacctgcgaataaaagaaaatttaccttagatggatttacgtaataatggtctttaggatatttagttcctctccaatcagttacttcaacataacggtaagaaaatccatttttactttcaatccaactccacgcttcaaaaggagtattaaaaactttatcaggtattaaattaccttcaaaatgagaaggatttgcataatccccggcataaacataatattcgttaatactcatttattcacctttagaaattttatccataacgatagcaattaaaccaattaaaaatgctactacaagtgaaaacacattttctgctgtagttaataatccgcatataaatccaacaaacattgaaaaactaaaagcagaagcagaaattgcaatagcaacatttcgaattaattcacagcgtttcattttattctcctcagtagttgatagggtaatagtatcacagctaaaaccctatgtaaacaactttgtgaaatatttattacaaaagatttttagcaataatcttgagatgtgccgcagaaatgtgtttagctttaaacaacgcagtttcttcagcaggagagataacgattgtagcaccatcctttttagcagaccacccatcacctaggtaaacagtacctttgatttcttcgccatcaaccagactaatcattggtttaccttctcgtcctttatttgctttaataacttcagaagtaagagtagcttcggtaatggtagaaaccggggtagttgtagaagtaaattctttaaatgttttcatttttattttcctaattaattttgatgaggtaatagtatcactacctcatcagtatgtaaacaactttgtgaaattattttaaatcatctgcccaatcgagtttaagaggctctttgtattcacgatctaatacgaccggaatttgtacatcaccgctaaatgataagggcccaacattataagacaatgttatatgcggtgtgtaatcatcaaaatcatgtgtagcacctagtgcccgcgcatacatgtgtcgacagcgcagatattcagaatctagcacaagtacaagagtcgatccatcttgtgttttccatacttctaaatgtccagaagaagctacttcaaaacttccactcgatggaacatatggaacatttactcttgaataacatatagtcgaatgaattttttctctaggaactggattaggaacacgtaaagagcgctgaagttcttccagcgcatcaagtgttaattctgaaaacttagctgctacataaagacccgttgaaaagtctttaaattccatcattcttcatcttttgcttcatctgcagattcagcagtaagatttttgacagcttcaacgatttcttcaactttgatagtatcgccagtgatacctactgcacgagcaatttcagccaaagttccttgcagaattttggattcttccatcagacgagcagcttgatcctgcgtatcaagaatgcgagatttcagagttacgatttcagcagacagtttttgttcaacagtttgttcagacattatagtacctttagtgtatttttaattttagaaaaaagttcttcaagagaaccatcgtttgtaattactaaatcgccatcacgaattggcaatccagcttctgtaatatgtgtatcattggatttttgaccaggacgaactacatgaattactgtagcacccatcgccctagccgcatccatttcatgatcttgacgggtatcaggaacgatataataatcataacctgagttaaatttatcaagataatctaaagcaaataattttacccagtacatgcggtcgaagttattaacaatcaaatccgtacctagggcttgcatcagacgacggactgaccattgatcttcaatattatttataacgtcagtaatcttattaaatgctacgaaattaactgattcttttccttcgtcatcaaaaacaaacacacctttaattgggcttttaccattaagataacaaaatgcttgttccataatcgtgattacttctaatttagtcagatttaaattagtctcacgatcatagtcaattccttcaaactctttacgagttaagcaaggatagtcagtgtttgctgcaaatactccccatgcataagccaatgcatccttaataggaccagcaagttggtatttaactgcagaataattgctcatgataaaatcagcagtagtatcttttccactacgctttacaccgcttaaaaagattagtttcatgtgtttctcctcaaatttaattaagattataacacacaaaactgaagcattaaacttctgctataattttaccatctttttctacttgaaaataggtgtaaggaattgttgcagtacatactaaagccgggtctgaatcttccgtgtagctaaattctacttcagataggtcagaaacccaaggcttataaaaatttattgacatcacgatttcagttttgctattatctaagatgtaaagcgtaatgtactcaggacctgttttttgggcagtattttcacctgtaagatagttgctagttcctagcatccattcatacattcctatccacgacttaagttcttcatcaactataaatctcacaatgagtggatcatactcaaatgtaacacctggacgttgtgctcggcccagtccaaacggcccagtcacggtatcagtaacaggtattctaattcctggaataggaactgactgagcatttaaagtaaaagcagatgtagtattactatgtggtattgatactacaaagttagttgtatttgcttggttaaaaatttgttgcagagcttgcgacatatattcctcataatgctttataactgttggtggtataatgggtctaagtcccttccattcaattccatttagaacaaacaacagaaaagaatggaagataatagaattagatatttgaccagactttgtttgcagagaaacgttttccttttgaaacgaactgctgaagtggcatcaacacaacgttcgcccagtctttcggggcgatttcaacaaggctacccataatattaccaggtatatatgccttaatcatttggtctgcacccctaaatcctttcacttgactccaatcaatttttaatttcgttttattagtaatagtaggtgtatttgcatattgctttaaaagctcttctaggaattgctgacgagctttaggtggaatatagtgcaagtttaatccgtacattaaattatgcttacctaaaccaaggtaaattatcaaaggaaatttatcccagtaaggaagagtttccttgtgtttagcatcataagcaaaagcatatattcgtcccggctgcgggcgaacaactttatgtccttttacttgcttaatagtttcagcaaaccactttctggttttattattaattgctgcgccttcattacgaattttatcacgcaatgtttgtctgaatgaatttatcataagcagttgtctttcttgcttattgagtttattcattggttttgattcaagtttttgaatcttttcagccgttttaattcctgaagcatattttgacattgctgaagtaaacgtagagtatttgattcctctttcttcagcaaattgctttcctgtcattccttttgctttggcctttttatattcaagacctatctgaatccatttcttttcgtttaatgattgcttaacctttggaacttggggagtgctttcattaattatttgaaaaatagccattatgcccccttaaagccaagagctcgtaatccatcttctgttagaattctaaattttattccacgcttttcagctaaagattgtgctgctttccatttgtcagtgttcacagaccaggtataaatttcattcataaatcttttcttcgctgcggtcgttagatgtgctggtttaactggtggttgtgtttcttttttaggttttatttcaataaaaaattcttgtccagaagaatctttcatccaaatatccatgaagtatctacgttttttcccttctgcattacaaaaataaggaattactgctgtttcactaccccatgcaataatttctggatttttatctaaccattcaaaaaagaatttttcccaatttgatctatacgtaatttttttagggtcacctctatactttgatatatttttaggaacccattttccagaatatgccattggattctccttataaatagataatatatttataaacaggagggcccatgctctttacattttttgatccgattgaatatgcggccaaaacggtgaataaaaacgcgccgactattcctatgacagatatttttagaaactataaagactattttaaacgcgctcttgcgggataccgcttacgtacttattatattaaaggttcaccacgcccggaagaattagcaaatgctatatatggaaatccacagctgtattgggttttattgatgtgtaatgataattatgacccgtattatggatggattacttcgcaagaagctgcttatcaagcatctatacaaaaatacaaaaacgtaggtggagaccaaatagtatatcatgtgaatgagaacggtgaaaaattttataatttaatatcatacgatgataatccatatgtttggtatgataaaggcgataaagctagaaaatatcctcaatatgaaggagcgcttgctgcggtcgatacgtatgaagctgctgttcttgaaaatgaaaaacttcgtcaaataaaaataatagcaaaatcagacatcaattcatttatgaacgaccttatacgtataatggagaaatcttatggaaatgataagtaataaccttaattggtttgtcggtgttgttgaagatagaatggacccattaaaattaggtcgtgttcgtgttcgtgtggttggtctgcatccacctcaaagagcacaaggtgatgtaatgggtattccaactgaaaaattaccatggatgtcagttattcaacctataacttctgcagcaatgtctggaattggaggttctgttactggaccagtagaaggaactagagtttatggtcattttttagacaaatggaaaactaatggaattgtccttggcacgtatggtggaatagttcgcgaaaaaccgaatagacttgaaggattttctgacccaactgggcagtatcctagacgtttaggaaatgatactaacgtactaaaccaaggtggagaagtaggatatgattcgtcttctaacgttatccaagatagtaacttagacaccgcaataaatcccgatgatagaccgctatcagagattccgaccgatgataatccaaatatgtcaatggctgaaatgcttcgccgtgatgaaggattaagattaaaagtttattgggataccgaaggatatccgacaattggtattggtcatcttatcatgaagcagccagttcgtgatatggctcaaattaataaagttttatcaaaacaagttggtcgtgaaattacaggaaatccaggttctattacaatggaagaggcgacgactttatttgagcgtgatttggctgatatgcaacgggacattaaatcacattctaaagtaggaccagtctggcaagctgtcaaccgttctcgtcaaatggcgttagaaaatatggcatttcagatgggtgttggtggtgtagctaaatttaacacaatgttaactgctatgttagcaggagattgggaaaaagcgtataaagccggtcgtgattcattgtggtatcaacaaacaaaaggccgtgcatcccgtgttaccatgattattcttacggggaatttggaatcatatggtgttgaagtgaaaaccccagctaggtctctatcagcaatggctgctactgtagctaaatcttctgaccctgctgaccctcctattccaaatgactcgagaattttattcaaagaaccagtttcttcatataaaggtgaatatccttatgtgcatacaatggaaactgaaagcggacatattcaggaatttgatgatacccctgggcaagaacgatatagattagttcatccaactggaacttatgaagaagtatcaccatcaggaagaagaacaagaaaaactgttgataatttgtatgatataaccaatgctgatggtaattttttggtagccggtgataaaaagactaacgtcggtggttcagaaatttattataacatggataatcgtttacatcaaatcgatggaagcaatacaatatttgtacgtggagacgaaacgaaaactgttgaaggtaatggaactatcctagttaaaggtaatgttactattatagttgaaggtaatgctgacattacagttaaaggagatgctaccactttagttgaaggaaatcaaactaacacagtaaatggaaatctttcttggaaagttgccgggacagttgattgggatgtcggtggtgattggacagaaaaaatggcatctatgagttctatttcatctggtcaatacacaattgatggatcgaggattgacattggctaatatacttccaatgagcgctgatttaggagaatccatggaaggttcttctatcgacgtcacctttaccgctcaattagaaacaggtgaaacgttagtatctataaatataactagttacgaagaaactcctggggttttagtagaagaaaatcgcttatatggaacatatgaatctgtatttggtttcggaaatgacgcgttgaaatatcgtttaggcgatgaatttaaaactgctgcttcatgggaagaacttcctactgattctgatactcagttgtatttgtggaaagctcctcaaaacctccagaagacattcacttacgaagtaacattaatatatgactaccaagaacaaagtgaatctgggggttctggcagtaattctaggtcatcttctgatactactgaaccgacagatcctcctgctccagtaagaaaaactctagttaaaaattatactaaaactatagttggaaattggagtcgttgggctaataaactgagaaaatatgcctatgcaagaccataaatatttttatttgtattcaataactaataaaacaacagaaaaaatttatgtaggcgtccacaaaacttcaaatttggatgatgggtatatgggttctggcgttgccattaaaaatgccattaaaaaatatggcatagataatttttataagcatattataaaattctttgaatctgaaaaagctatgtatgacgcagaggcagaaatagtcacagaggaatttgttaaatctaagaaaacttataatatgaaactaggcggtatcggtggcttcccaaaacataacacagcgggtgctaaaaatggattttacggtaaatctcattcgcgtgaaactagattgaaaattagcattaaatcgtctagaaaaagagggcctagagggctagaggtaaaactctgaagatgtgtggcgccaataacccaaggtatggcaaaatagcccctaatgctaaatctgttattatcaacggcgttttatataaaagtattaaaatcgcagctaaagctcttaatataaattatagtaccttaaaggggcgagttaaagcggggtattataaatgtcaggattaagttatgataagtgtgttactgctggccatgaagcgtggcctccaacagttgtgaatgctacacaaagtaaagtattcactggaggaattgctgttctcgtagcaggcgatccaattacagaacatacagaaattaaaaagccgtatgaaacacatggcggagtgacacaacctagaacttctaaggtatatgtcactggaaagaaagctgttcaaatggctgatccaatatcatgcggtgatactgtggctcaggcatcatctaaagtattcattaaataggatttaaaatggcaaatacccctgtaaattatcaattaacaagaacagcaaatgctattcccgagatattcgtcgggggtacatttgctgaaataaaacaaaacctcattgaatggcttaatggccaaaatgaatttttggattatgattttgaaggctcaagattaaacgttctgtgtgaccttttagcttataatacattatacattcagcagtttggtaatgctgctgtgtatgaaagctttatgcgtactgctaacttacgaagttcagttgttcaagctgcacaagataacggatatttacctacttcaaaatccgctgcgcagaccgaaattatgttaacatgcactgacgcattgaataggaattacattactattcctcgcggaactcgctttttagcatatgcaaaagatacttctgttaatccatataacttcgtttctagggaagacgttattgctattcgtgataaaaataaccaatattttccgcgtttaaaattggcccagggacgtatagtaagaactgaaatcatttatgataaattaacacctattatcatttatgataaaaatattgatagaaaccaggttaaattatacgttgatggagcggaatggattaactggacgagaaagtcaatggttcatgctggttcaacatcaacgatttactatatgcgtgaaactattgatggaaacactgaattctattttggtgaaggtgaaatttctgttaatgcttctgaaggagctttgaccgctaattatatcggaggtcttaaacctactcagaactctacgattgttattgagtacattagtactaatggtgctgacgcgaacggagcagtcggattttcatacgcagatacattaacaaatataactgtcatcaatattaatgaaaatccaaacgatgatccagattttgttggggcagatggaggcggtgatccagaagatattgagcgtattcgcgaattgggtactattaaacgcgaaacccaacaacgatgcgtaactgcgactgactatgatacattcgtttcagagagatttggttctattattcaagctgttcagactttcactgattctactaaacctgggtatgcatttattgctgctaaacctaaatcaggattgtatttaactaccgtacagcgtgaagatattaaaaattatctcaaagactataatttagctcctattacgccatcaattatttctcctaattatctttttattaagactaatttaaaagtcacatatgctttaaataaactgcaagaatccgaacagtggcttgaaggtcaaataattgataaaatagatcgctattataccgaagatgtagaaatttttaactcgtctttcgctaaatctaagatgttgacatatgtagatgatgcagatcattctgtcattggttcatcagcgactattcaaatggttcgtgaagtacaaaacttctataaaacgcctgaagcgggtattaaatacaataatcaaataaaagatcgttctatggaatctaatacgttttcatttaattctggacgaaaggttgtaaatcctgatactggtttagaagaagatgtattatatgacgttcgtatagtatcaacagaccgagattctaaaggaattggtaaagttattattggtccatttgcttctggcgatgttacagaaaatgaaaacattcagccgtatacaggcaacgattttaacaaattagcaaattctgatggacgcgacaaatactatgttatcggtgaaataaattatccagctgatgtgatttattggaatatcgctaaaattaatttaacatctgaaaaatttgaagttcagaccattgaattatattctgacccaaccgatgatgttatctttactcgcgatggttcactgattgtatttgaaaatgacttacgtccacaatacttaactatcgatttggagcctatatcacaatgacagtaaaagcaccttcagtcactagtctcagaatttccaagttatccgcaaatcaggtgcaagtacgctgggatgacgttggtgctaatttctactattttgtagaaatcgctgagacaaaaacaaactcgggggaaaatctcccgagtaatcaatatcgttggattaatttaggatatacagcaaataatagtttcttttttgatgatgctgatccattaacaacatacattattagagtagccacagctgcgcaagattttgagcagtctgattggatttataccgaagagtttgaaacttttgctacaaatgcttatacatttcaaaacatgattgaaatgcaattagccaataaattcattcaggaaaaatttactcttaataattctgattatgttaattttaataatgatactataatggctgcattgatgaatgaatcattccaattcagcccatcgtatgttgatgtttcatcaataagtaattttattattggtgaaaatgagtatcatgaaatacaaggttctattcagcaagtatgtaaggatattaaccgagtttatttgatggaatcagaaggaattctatatctttttgagcgctatcaacctgtagttaaagtatccaatgataaaggacaaacctggaaagctgtaaagctcttcaatgaccgtgtaggatatcctttatctaagacagtatattaccaatctgcgaacacaacatacgttctaggatacgacaagattttctatggccgcaaatctactgatgttagatggtcagccgatgatgtcagatttagttctcaggatataacatttgctaaacttggcgaccaattacatctaggatttgatgtagaaatttttgccacttacgcgactttaccagcgaatgtataccgcattgcagaagctattacttgcaccgatgattacatttacgttgtcgccagagacaaagttagatacataaaaacgagtaatgcacttatagattttgatccattatctccaacatattcggaaagactttttgaacctgataccatgactataaccggaaatcctaaagcagtatgctataaaatggattctatctgtgataaagtttttgctcttattattggtgaagttgaaacattaaatgctaatcctagaacatcaaaaataattgattccgctgataaaggaatatatgttttaaatcatgacgaaaaaacatggaaaagagtttttggtaataccgaagaagaaagaagacgtattcaacccggatatgcgaatatgtcaactgacggtaaattagtttctctgtcttcgagtaattttaaatttttaagtgataatgttgttaatgaccctgaaactgcagcaaaatatcagttaattggcgctgttaaatatgaatttcctcgtgaatggttagctgataagcattatcatatgatggcatttatagcggatgaaacatctgattgggagacttttactcctcaaccaatgaaatactacgcagaaccattctttaactggtctaaaaaatctaacacacgttgttggataaacaactctgatagagctgtggtagtttatgctgatttaaaatacactaaagttatagaaaatattccggaaacatcaccagatagattagttcatgaatactgggatgatggtgattgcactatagtaatgccaaatgtcaaattcactggatttaaaaaatacgcatcaggaatgcttttctataaagcctccggtgaaataatttcttactatgattttaactatcgtgtgagagatacagtagaaattatttggaagccaactgaagtatttttaaaagcatttttacaaaaccaagagcatgagactccttggtcaccagaagaagagcgtggattagctgaccctgatttaagaccattaattggcacaatgatgcctgattcttatttgttacaggattcgaattttgaggcattttgcgaagcatatattcagtatctttctgatggatatggaactcaatacaataatttacgaaatttaattcgtaaccaatatccacgagaagagcacgcatgggaatatttgtggtcagagatatataaaagaaacatttatttaaatgctgataaacgcgatgctgttgcgagattctttgaatcacgtagctatgatttttattctactaaaggaattgaagcatcatacaagtttctttttaaagttctttataatgaagaagttgaaattgaaattgaatctggggctggtactgaatatgatataatcgttcaatctgattctttgactgaagatttagtaggacaaacgatttatacggcaacaggaagatgtaatgttacttatatagaaagaagctattctaatggtaaattgcaatggaccgtaactattcataatcttttgggacgattaattgctggtcaagaagttaaagcagaaagactccctagttttgaaggcgaaattattcgtggggttaaaggaaaggatttgcttcaaaacaatatagactatattaatagaagtagatcatactatgtaatgaaaattaaatccaatttaccttcttcccgctggaaatctgacgttattcgttttgttcatccagtaggatttggatttatagcaattacccttttaacaatgtttattaatgttggtttaactcttaaacatacagagactataattaataaatacaaaaactataaatgggattctggattgcctactgaatatgccgacagaatagctaaattaactccaaccggtgaaattgagcatgattcagtaacaggcgaagcaatttatgagcctggcccaatggctggtgtaaaatatcctcttcctgatgactataatgctgaaaataataattcaatatttcaaggtcaattgccgtctgaacgacgtaaattaatgagtcctttatttgatgcatctggaacaacatttgcgcaatttagagatttagttaataaacgtctaaaagataatataggaaatccaagagaccctgaaaatccaacacaggttaaaatagatgaatgattcaagtgttatctatcgtgcgatagttacttcaaaatttagaacagaaaaaatgttgaatttttataattcaattggaagtggtccggataaaaacactatctttatcacatttggaagatcagaaccgtggtcatcaaatgaaaatgaggtgggctttgccccaccttatccaaccgattctgtattaggcgtaactgacatgtggacgcatatgatgggaacagtaaaagttcttccatcaatgcttgatgctgttattcctcgcagagattggggagatactagatatccggatccatacacatttagaattaacgatattgtagtgtgtaactcagctccttacaacgctactgaatcaggcgctggttggttagtgtatcgttgtttagatgttcctgataccggaatgtgttcaatagcatctttaactgataaagatgaatgccttaagttaggtggaaaatggactccttctgctaggtcaatgactccgcctgaaggtcgaggagatgctgaaggaacaattgaacccggagacgggtatgtgtgggaatatctatttgagattccgcctgatgtatctataaatagatgcacgaatgaatatatcgtggttccttggcctgaggaattaaaagaagacccgactagatggggatatgaagataatctcacttggcaacaagatgattttggattaatttaccgtgttaaggcaaatactatccgttttaaagcatatttagattcagtttattttcctgaagctgcattaccaggaaataaaggatttagacaaatatcaataatcacgaatcctcttgaagctaaagctcatccaaatgacccaaacgttaaagctgaaaaggattattatgacccagaagatttaatgaggcattcgggtgaaatgatttatatggaaaataggccacctattattatggcaatggatcaaacagaagaaatcaatattctgtttacattttaaattaagggagcccatgggctccctttttctttataaatactataaactcataaggaaaccgctatgttcattcaagaaccaaagaaattgattgataccggcgaaattggtaacgcttctactggtgatatcttattcgacggtggtaataaaattaatagtgattttaacgcaatttataatgcgtttggcgatcagcgtaaaatggcagtagcaaatggcactggagcagatggtcaaattatccatgctactggatattatcaaaaacactctattacagagtacgcaactccagtgaaagttggcactagacatgatattgatacctctactgtaggtgttaaagttatcattgaaagaggcgaactcggcgattgtgttgaattcattaactctaatggatcaatatcagttactaatcctttgacaattcaagctattgattcaattaaaggtgtttcaggtaatttagtagtaactagcccatatagtaaagttactttacgctgtatttcatctgataattctacgtcggtttggaattattctattgaaagtatgtttggacaaaaggaatcaccagctgaaggtacatggaatatttctacatctggatcagttgacattccattatttcatcgtactgaatacaatatggctaaattgctagttacgtgccaatcggtagatggaagaaaaattaaaacagcagaaataaatattcttgtggatactgttaattcagaggtaatttcttctgaatatgctgtcatgcgagttgggaatgaaaccgaagaagacgaaatcgctaatattgcatttagtattaaagaaaattatgtaacggcgactataagttcttcaactgtcggtatgagagcagcagttaaagttatcgctacgcagaaaatcggggtggctcaataatgaaacaaaatattaatatcggtaatgttgtagatgatggtaccggtgactacctgcgtaaaggtggtataaaaataaatgaaaactttgatgagctttattatgaactcggtgatggtgatgttccatattcagccggtgcctggaaaacttataatgcttcatcaggacaaacattaacagcagaatggggaaaatcatacgctattaatacatcttctggaagagtgactataaatcttccaaagggtacagttaatgattacaacaaggtaattagagctagagacgtatttgctacatggaacgtcaacccagttacactagtagctgcttccggcgatacgattaaagggtctgcagtaccagttgaaattaatgttcgattcagcgatttagaactagtgtattgtgccccaggacgttgggaatatgtcaaaaataaacaaattgacaaaattaccagttcagacattagtaatgtagctcgcaaagaatttttagttgaagttcaaggacaaacagactttttagatgttttccgtggaactagttataatgtaaataacatcagagtaaaacatcgtggtaacgaattgtattacggcgatgtgtttagcgaaaacagcgattttggctctccaggcgaaaatgaaggagaactggttcctcttgatggatttaacattcgattaagacagccttgtaatattggtgacactgttcaaattgaaacatttatggatggtgtatcacagtggagaagttcatatacaagacgtcaaattagattgttagattcaaaattaacgtcaaaaacttctttagaaggaagcatttacgttactgatttatcaacaatgaaatcaattccattttctgcttttggattaattccaggagaacctattaatcctaactctcttgaggttcgttttaacgggattttacaggaattggctggcacagttggaatgccattatttcattgtgttggtgccgattcagacgatgaagtagaatgctctgttttaggtggaacttgggaacaatctcataccgattattcagttgaaactgatgaaaacggcataccagaaattttacatttcgatagcgtatttgagcatggtgacattatcaatatcacctggtttaataatgatttgggtacattattaacaaaagatgagattattgatgaaactgataatctctatgtatcgcaaggacctggagtagatatttctggtgatgtaaatttaacagacttcgataaaattggttggccaaatgtagaagcagttcaatcttatcaacgcgcatttaatgctgtttcaaatatctttgatacgatttatcctattggaactatatatgaaaacgctgttaatccaaataaccctgttacatatatgggattcggctcatggaaattatttgggcaaggaaaagttttagttggatggaatgaagatatttcggaccctaactttgctctaaataacaacgatttagattcgggtggaaatccttcacataccgcaggtggaacaggtggttctacttctgttacattggaaaatgctaatcttcctgcaactgaaacagatgaagaagttctaatagttgatgaaaatggatcagtcattgttggtgggtgtcaatacgatccagatgaatccggtccaatttacactaaataccgtgaagctaaagcatctactaactctactcacactccgccaacatcaataactaacattcaaccatatattacagtttatcgttggataaggattgcataatgagtttacttaataataaagcgggagttatttcccgcttagccgattttcttggttttagacctaaaactggcgacattgatgtaatgaatcgtcaatcagtcgggtcagtgacaatatctcaattagcgaaaggattttatgaaccaaacatagaatcagctattaatgacgttcataatttttctataaaagacgttggcacaattattactaataaaactggtgtttctcctgagggtgtttctcaaactgattattgggcattttctggaactgtaacagacgattctcttcctccgggttctcctattacggtattagtatttggtcttccagtttcagcaacaactggaatgacggcaattgagtttgttgcaaaagttcgcgttgcactacaagaagctattgcgtcatttactgctatcaattcatataaagaccatccaactgatggtagtaaattagaagttacttatttagataatcaaaaacatgtattaagcacatattctacatatggaataactatttcccaagaaattatatctgagtctaagcctggctatggtacatggaatttattgggcgcacaaactgtaactttagataatcagcagactcctacagtattttatcattttgagagaacagcatgagtaataatacatatcaacacgtttctaatgaatctcgttatgtaaaatttgatcctaccgatacgaattttccaccggagattactgatgttcacgctgctatagcagccatttctcctgctggagtaaatggagttcctgatgcatcgtcaacaacaaagggaattctatttattcccactgaacaggaagttatagatggaactaataataccaaagcagttacaccagcaacgttggcaacaagattatcttatccaaatgcaactgaaactgtttacggattaacaagatattcaaccaatgatgaagccattgccggagttaataatgaatcttctataactccagctaaatttactgtcgcccttaataatgcgtttgaaacgcgagtttcaactgaatcctcaaatggtgttattaaaatttcatctctaccgcaagcattagctggtgcagatgatactactgcaatgactccattaaaaacacagcagttagctattaaattaattgcgcaaattgctccttctgaaaccacagctaccgaatcggaccaaggtgttgttcaattagcaacagtagcgcaggttcgtcagggaactttaagagaaggctatgcaatttctccttatacgtttatgaattcatcttctactgaagaatataaaggcgtaattaaattaggaacacaatcagaagttaactcgaataatgcttctgttgcggttactggcgcaactcttaatggtcgtggttctacgacgtcaatgagaggcgtagttaaattaactacaaccgccggttcacagagtggaggcgatgcttcatcagccttagcttggaatgctgacgttatccagcaaagaggtggtcaaattatctatggaacactccgcattgaagacacatttacaatagctaatggtggagcaaatattacgggtaccgtcagaatgactggcggttatattcaaggtaaccgcatcgtaacacaaaatgaaattgatagaactattcctgtcggagctattatgatgtgggccgctgatagtcttcctagtgatgcttggcgcttctgccatggtggaactgtttcagcgtcagattgtccattatatgcttctagaattggaacaagatatggcggaaacccatcaaatcctggattgcctgacatgcgtggtctttttgttcgtggttctggtcgtggttctcacttaacaaatccaaatgttaatggtaatgaccaatttggtaaacctagattaggtgtaggttgtaccggtggatatgttggtgaagtacagatacaacagatgtcttatcataaacatgctggtggatttggtgagcatgatgatctgggggcattcggtaatacccgtagatcaaattttgttggtacacgtaaaggacttgactgggataaccgttcatacttcaccaatgacggatatgaaattgacccagaatcacaacgaaattccaaatatacattaaatcgtcctgaattaattggaaatgaaacacgtccatggaacatttctttaaactacataattaaggtaaaagaatgacagatattgtactgaatgacttaccattcgttgacggccctcctgcagagggccagagccgcatttcctggattaaaaacggcgaagaaatattaggagctgacacacagtatggaagtgaaggctcaatgaatagacctacggtttctgtactaagaaatgttgaagttcttgataaaaacattggaatacttaaaacatctttagaaaccgcaaatagtgatattaaaacaattcagggcatcttagatgtatctggtgatattgaagctttggcccaaataggtatcaataaaaaggatatttctgacctcaaaacgctaaccagtgaacacacagaaatattaaatggaactaataatacggttgacagtattcttgccgatattggtccatttaacgccgaggccaactctgtatacagaacgatcagaaatgatttactgtggataaagcgtgaacttggacaatacactggtcaagatattaatggtcttcctgttgtaggaaatcctagtagtggaatgaagcatcgcattattaataatactgatgtcatcacttcgcagggaatacgtttaagcgaattagaaacaaaatttattgaatctgatgtaggttctttgaccattgaagttggtaatcttcgtgaagagcttggaccgaaaccaccatcattttcacagaacgtttatagtcgtttaaatgaaattgacactaaacagacaacagttgagtctgacattagtgctattaagacctcaataggatatccaggaaataattcgattatcacgagtgttaatacaaacactgataatattgcatctattaatttagagctaaatcaaagtggaggtattaaacagcgtttaaccgttattgaaacttccattggttcagatgatattccttcgagtattaaaggtcaaatcaaagataatacaacttcaatcgaatctctaaatggaatcgtcggtgaaaacacttcatctggcttaagagcgaatgtttcatggttaaaccaaattgttggaactgattctagcggtggacaaccttctcctcctgggtctcttttaaaccgagtttctacaattgaaacttctgtttcaggcttgaataacgctgttcaaaacctacaagtagagattggtaataacagcgcaggaattaaagggcaagttgtagcgttaaatactttagtaaatggaactaatccaaacggttcaactgttgaagagcgcggattaaccaattcaataaaagctaacgaaactaacattgcatcagttacacaagaagtgaatacagctaaaggcaatatatcttctttacaaggtgatgttcaagctctccaagaagccggttatattcctgaagctccaagagatgggcaagcttacgttcgtaaagatggcgaatgggtattcctttctacctttttatcaccagcataacatggggccgcaaggccccaaaggattttaaatgtcaggatataatcctcagaatccaaaggaactcaaagatgtcattctaagacgtttaggggctccaattattaatgttgagttaacacccgatcaaatttacgattgtatccagcgtgccctagaattatacggtgaataccattttgatggactcaataaaggttttcatgttttttatgtaggggatgatgaagaaaggtacaagaccggagtcttcgatttaagaggttctaacgtatttgcagtaacccgcattttacgcacaaatattgggtcaataacatctatggatggaaacgctacatatccgtggtttactgactttcttttaggaatggctggtattaatggcggaatgggaacgtcttgtaatagattttatggaccaaatgcctttggagctgatttaggatattttacccagcttaccagttatatgggaatgatgcaagatatgctctctcctattccagacttttggtttaattcagcaaatgaacagctcaaagtcatgggaaacttccaaaaatatgatttaattatcgtagaaagctggactaaatcatacattgatacaaacaaaatggttggaaatacagtaggatatggaacagtcggtccacaagatagctggtcattatctgaacgatataataacccagaccacaatttagtaggtcgtgttgtcggccaagatccgaatgttaaacagggtgcttataataatcgttgggtgaaagactatgcaacagctttagctaaagaattgaacggtcaaattttagcacgccaccaaggtatgatgcttccgggcggtgttacaattgatgggcagcgcttaatagaagaagccagattagaaaaagaagcactgcgcgaagaattatacttacttgatcctccatttggaattttggtaggttaatatggctacttatgataaaaatctttttgctaaattggaaaaccgcacaggttattctcagaccaatgaaactgaaatattaaatccttatgtaaatttcaatcattataaaaacagccaaatattagctgatgtattagtagctgaaagcattcaaatgcgaggtgtagaatgctattatgttccaagagagtatgtttcccctgatttgatattcggcgaagacttaaaaaataaatttactaaagcttggaaatttgctgcatatttaaattcatttgaaggatatgaaggagctaaatcgttctttagtaactttggtatgcaagtacaagacgaagtgactttatctattaacccaaatttatttaagcatcaagttaacggaaaagaacccaaggaaggtgatttgatatattttcctatggataacagcttatttgaaattaactgggttgaaccatatgatccattttatcaattaggccaaaacgctattcgtaaaattacggcaggtaaattcatttattctggagaagaaattaatccagttctacagaaaaatgaaggaattaacattccagaatttagtgaattagaattaaatgctgttcgcaatcttaacggtattcatgacattaatattgatcagtatgctgaagtagatcaaattaattctgaagctaaagaatacgttgaaccttatgttgttgtcaataacagaggcaaatctttcgaatctagcccatttgacaatgatttcatggattaataaatattataaactaattaaagcccggattaggagaagtcatgtttggttatttttataattcgtcttttagacgatatgctaccttgatgggcgatttgttttcaaatatccaaatcaaacgtcagttagaatctggtgataagtttatacgtgttcctattacgtatgcatcaaaggaacactttatgatgaaattgaataaatggacatcaataaattcacaagaagatgtagctaaagttgaaactattctacctcgtataaatttacatttagttgattttagctataatgctccatttaaaacaaacattttaaatcagaatttactgcaaaaaggtgcaacttctgtagtatcgcagtataatccatctcctattaaaatgatttatgaattgagtatctttactcgctatgaagatgatatgtttcaaatagttgaacagattcttccatattttcaacctcattttaatacaactatgtacgagcagtttggaaatgatattccatttaaaagggatattaaaattgtactgatgtctgctgctatagacgaagctatagatggggataatttatctcgtcgtagaattgaatggtcattaacatttgaagtaaatggatggatgtatcctccagtagatgatgcagaaggattaattcgtactacttatacagattttcacgccaatacaagagatttgcctgacggcgaaggtgtttttgaatctgtcgatagcgaagttgttcctcgagatattgacccagaagactgggatggaacagtaaaacaaactttcactagtaatgtaaatagaccaacaccgccagaacctcctggcccaagaacatagaggttattatggaaggtcttgatataaacaaacttttagatatttctgacctccccggaattgacggggaggaaatcaaagtgtatgaacctctgcaattagtagaagttaaaagcaatccacaaaaccgtactccagacttagaagatgattatggagtagttcgtcgaaatatgcattttcagcaacaaatgctaatggacgctgccaagatttttcttgagacagcaaagaatgctgattctcctcgtcacatggaagtatttgcaactcttatggggcaaatgactacgacgaacagagaaatactgaagcttcataaagatatgaaagacattacatctgagcaggttggcaccaaaggcgctgttcctacaggtcaaatgaatattcagaatgcgacagtattcatgggttcaccaacagaattaatggacgaaattggtgatgcttacgaggctcaagaagctcgtgagaaggtgataaatggaacaaccgattaatgtattaaatgatttccatccgttaaatgaagctggaaaaattttaataaaacacccaagcttagcggaaagaaaagatgaagatggaattcattggataaaatctcagtgggatggaaaatggtatcctgaaaaattcagtgattaccttcgtctacacaaaatagtaaaaattccaaacaactctgataagcctgaattatttcaaacttataaagataagaataataaaagatctcggtatatgggtcttcctaacttgaaacgagctaatattaaaacacaatggactcgtgaaatggttgaggaatggaaaaaatgccgagatgatattgtttattttgcagaaacatactgtgccattactcatattgactatggtgtcataaaggttcaattacgtgactatcagcgtgatatgctcaaaataatgtcatctaaacgtatgactgtttgtaatctatcgcgccagctcggtaaaaccaccgtagtagctattttccttgcacactttgtatgttttaacaaagataaagctgtaggtattcttgcacacaaaggctcaatgtctgcggaagttttagaccgtactaagcaagcaattgaactgcttcctgactttttacaaccaggaattgttgaatggaataagggttcaattgaactagataatggttcttcaattggcgcttatgcttcctctcctgacgcagttcgtggtaactcgttcgcaatgatttacattgacgaatgtgcgtttattccaaacttccatgattcctggcttgctattcaaccagtaatttcatctggtcgtcgttcgaaaattattattactacgactcctaatggattaaatcatttttatgatatttggactgctgctgtcgaaggtaaatctggatttgaaccatatactgctatttggaattcagttaaagaacgtctttataacgatgaagatatttttgacgatggatggcaatggagcatacaaaccattaatggttcttcattagctcaattccgtcaagaacatactgcagcgtttgaagggacttctggtacattaatttcaggaatgaaattagctgttatggattttattgaagtaacaccagatgatcatggttttcaccaatttaaaaaacctgaaccagatagaaaatatattgcaactctagattgctcagaaggtcgtgggcaagattaccacgctttgcatattattgatgttactgatgatgtgtgggaacaggttggtgttttgcattcaaacactatttctcatttaattctacctgacatcgttatgcgttatttagtagaatacaatgaatgcccagtttatattgaattaaatagtactggtgtgtcagttgcaaaatcgctttatatggatttagaatacgaaggtgttatctgcgattcatatactgatttaggaatgaaacaaactaaacgcacgaaagcagtaggatgttccacgctaaaagaccttattgaaaaagataagcttattattcatcaccgagcgactattcaagaatttagaacgtttagtgaaaaaggcgtgtcttgggcggctgaagaaggttatcatgacgatttagtaatgtctttagtgatttttggatggttatcaacgcagtcaaaatttattgattatgcggataaagatgacatgcgattagcatctgaagtattttcaaaagagcttcaggatatgagcgacgactacgcgccagttatatttgtggattcggttcattctgctgagtatgttccagtatctcatggtatgtcaatggtataaatatattaaagcatattaaagaggattaaaaatgactttattatctccgggcattgagctcaaagaaactacggttcaaagcaccgtagttaataactctactggtacagcagctttggccggtaaattccagtggggtcctgcttttcagattaaacaggttacaaatgaagtagatttagttaatacttttggtcaaccaaccgctgaaactgctgactattttatgtctgcgatgaatttcttgcagtacggaaatgacttacgagtagttcgtgctgttgatagagataccgctaaaaactcatcgccaattgctggtaatattgattacacaatttctaccccaggtagtaactatgcggttggagataaaatcacggtcaaatatgtttcagatgatattgaaactgaaggtaaaattactgaagtagacgcagatggaaaaattaagaaaattaatattcctactggcaaaaattacgctaaagcgaaagaagtcggtgaatatccaacactaggttctaactggactgcggaaatttcttcatcttcctctggtttagctgcagtaataactcttggaaaaattattactgattctggtattttattagctgaaattgaaaatgctgaagctgctatgacagcggttgactttcaagcaaatcttaaaaaatatggaattccaggagtagtagcgctttatccaggcgaattaggcgataaaattgaaattgaaatcgtatctaaagctgactatgcaaaaggagcttctgcattactcccaatttatccaggtggtggtactcgtgcatctactgctaaagcagtgtttggatatggaccgcaaactgattcacagtacgctattatagttcgtcgtaatgatgctattgttcaaagcgttgttctttcaactaagcgtggtgaaaaagatatttacgatagtaacatctatatcgatgactttttcgcaaaaggtggttcagaatatatttttgcaactgcacaaaactggccagaaggcttctctggaattttaactctgtctggtggattatcatcaaatgctgaagtaacagcaggagatttgatggaagcttgggacttctttgctgaccgtgaatctgttgacgttcaactgtttattgcaggttcttgtgccggtgaatctttagaaacagcatctactgtccaaaaacacgtcgtttcaattggggatgctcgccaagattgcttagtattgtgctctcctccgcgtgaaactgtagttggaattcctgtaacccgtgctgttgataacctagtcaattggagaactgcggcaggttcatacactgataataactttaatatcagttcaacctatgcagcaattgatggtaaccataagtatcagtatgacaaatataatgatgtgaatcgttgggttccattagcagctgatattgctggtttatgcgcaagaactgataacgtatctcagacttggatgtctccagctggttataatcgtggtcagattcttaacgttattaaacttgctattgaaactcgccaggctcagcgcgaccgtttataccaagaagctatcaacccggtaaccggtacaggtggtgatggttacgtattgtatggtgataaaacagctacttctgttccttctccatttgatcgtattaacgttcgtcgtctgtttaatatgttgaaaacgaatatcggacgtagttcaaaatatcgtttgttcgaattaaacaacgcgtttactcgttcatcattccgcacagaaactgcccagtacttacaagggaataaagctctcggtggaatttatgaatatcgtgtagtttgcgatacaacaaataacactccgtcagtaattgatagaaatgagtttgttgcaacattctacatccaaccggctagaagcattaactacattaccttaaacttcgtagcaactgctactggtgcagatttcgatgagttaactggtcttgctggttaatacggtgcattctaaaggcctgtttcggcaggccatataaatacactatatccttaattctttaattctatatgccctaggttaaacatagggatataaatactacagaggctaatatgtttgtagatgatgtaacacgagcgtttgaatctggtgattttgctcgacctaacttattccaagtagaaatttcttatcttggacaaaattttacgttccaatgtaaagctactgctctaccagctggtattgtagaaaaaattccagtcggatttatgaaccgtaaaattaacgtagcaggcgatcgtacattcgatgactggactgttacagtaatgaacgatgaagctcatgatgctcgtcagaagttcgttgattggcaaagcattgctgcggggcaaggaaacgaaattactggtggaaaacctgcagagtataaaaagagcgctatcgttcgtcaatatgctcgtgacgctaaaacagtaacaaaagaaattgaaattaaaggtctgtggcctactaacgtgggtgaacttcaattagattgggattcaaacaatgaaatccaaacttttgaagtaactcttgctctcgattattgggaataaaatgaatggggagaaatccccatcctgcttaaagcagagaagtccattataaatataactataattcccatttggagaatacaatgaaatttaatgtattaagtttgtttgctccatgggctaaaatggacgaacgaaattttaaagaccaagaaaaagaagatcttgtttccattacagccccaaagcttgatgatggagcaagagaatttgaagtaagctcgaatgaagctgcttctccttataatgctgcattccaaacaatttttggttcatatgaaccaggaatgaaaactactcgtgagcttattgatacatatcgtaatctcatgaataactatgaagtagataatgcagtttcagaaatcgtttcagatgctatcgtctatgaagatgatactgaagtcgtagcgttaaatttggataaatctaaatttagcccaaaaattaaaaatatgatgttagatgaatttagtgatgtattaaatcatctatcgtttcaacgaaaaggttctgatcattttagacgttggtatgttgattcaagaattttctttcataaaatcattgatccaaaacgtccaaaagaaggcataaaagaattacgtagattagaccctcgccaagttcagtatgttcgtgaaattataacagaaactgaagctggcacaaaaatagttaaaggttacaaagaatattttatatatgatactgcccatgagtcatatgcatgtgatggtagaatgtatgaagctggcacaaaaataaaaattcctaaagctgccgtcgtttatgcccattctggattagtcgattgttgcggtaaaaatatcatcgggtatttgcatcgtgctgttaaacctgctaaccaattaaaattattagaagatgctgtagtcatttatcgcattactcgtgctcctgaccgtcgtgtttggtatgtagacacaggtaatatgcctgctcgtaaagctgctgagcacatgcaacatgttatgaacacgatgaaaaaccgtgtagtatatgatgcatcaacaggtaaaataaaaaatcaacagcataatatgtctatgaccgaagactattggttgcagcgccgtgatggtaaagctgtgacagaagttgatactcttcctggtgctgataatactggcaatatggaagatattcgttggtttagacaagctctttatatggcattacgtgttcctctttcacgcattccgcaagaccaacaaggcggtgtgatgtttgattctggaactagcattacacgtgatgaattaacgtttgctaaatttattcgtgagttacagcacaagtttgaagaagttttcctagatccgcttaaaacaaatcttttgcttaaaggtataatcacagaagatgagtggaatgatgaaataaataatattaagatagaatttcatcgggatagctactttgctgagctcaaagaagcagaaattttggaacgaagaattaatatgctaaccatggcagaaccatttattggtaaatatatttctcacagaactgctatgaaagacattttgcagatgactgatgaagaaatagaacaagaagccaagcaaattgaagaagagtctaaagaggctcgtttccaagaccccgaccaagaacaagaggatttttaatggaaggtttaattgaagctattaaatcaaacgacctcgtagccgctcgtaaattatttgctgaagccatggctgcaagaacgattgatttaattaaagaagaaaaaatcgctatcgctcgcaatttcttaatcgaaggtgaagaacctgaagacgaggatgaagatgaagatgacgaagatagtgatgataaagacgacaaaaaagacgaagactctgacgaagacgaggatgatgaataatgcttctgatccctgaaactcatgaattagttctcgagaatgtcgaagcacttattcctgaagcacagggtcgctttgacgaattgtcttctgctttaaataaagacgatataaatacaattgtcgagaatatgcttgatgatgaaactgatttagcggttgcattagcttctattaatgaaaatatgccgttaaatgaattcatcgttaaacatgtttctgcccgtggtgaaattactcgcactaaagaccgcaaaacgcgtgaacgaaatgcatttcaaaccactgggctgtctaaagcaaaacgtagacaaattgctcgtaaagctaccaaaacgaagattgccaatccagcaggtcaatctcgtgctcagcgtaagcgtaaaaaagctcttaaacgccgtaaagcattaggattaagctaatgaatgaaccccaattactaattgaaacttggggtcaacctggcgaaattattgatggcgtaccaatgcttgaatctcatgatggaaaagacttaggtttaaaaccgggtttatacatcgaaggaatattcatgcaagcggaagtcgtcaatagaaataaacgtctttatccaaaacgtatattagaaaaagcggtaaaagactatattaatgagcaagttttaactaaacaagctctcggagaattaaatcatcctccacgcgctaatgttgacccgatgcaagccgctatcattatagaagatatgtggtggaaaggaaatgacgtatacggacgagctcgtgttattgaaggtgaccatggtcctggagataaattagcagctaatattcgtgccggatggattccaggagtttcttctcgtggattaggttcattgactgacacaaatgaaggttatcgtatcgtaaacgaaggattcaaattaactgtaggtgttgatgcagtatggggtccaagtgctccagatgcatgggtaactcctaaggaaattaccgaatcacagacggcggaagccgatacaagtgccgatgacgcctatatggctctcgcagaggccatgaaaaaagcgttataaatattattatctaaacaacaggactacaaaatgcttaaagaacaactgattgccgaagcgcagaaaattgatgcttccgttgctcttgatagtattttcgaatcagttaatatttctccggaagcaaaagaaactttcggcactgtattcgaagctaccgtcaagcagcacgccgttaaattagctgaatctcatatcgctaaaattgctgaaaaagcagaagaagaagtagaaaaaaataaagaagaagccgaagaaaaagctgagaagaaaatcgctgagcaagcttctaaattcattgaccatcttgcaaaagaatggctcgctgaaaataaattagcagttgataaaggcatcaaagccgaactgtttgaatccatgcttggtggattaaaagagctctttgttgaacacaacgttgttgttccagaagaatcagttgatgttgtagctgaaatggaagaagagctgcaagaacataaagaagaatcgcctcgtctgttcgaagaactgaatatgcgcgacgcatatatcaattatgtgcagcgtgaagtggcattgagcgaaagtactaaagatctgactgagtctcaaaaagaaaaagtctctgctctggtcgaaggtatggattattcagatgcattctcaagtaaattgagtgcaatcgtagaaatggtgaagaaatctaataaagatgaaagcactattactgagagtataaatactcctgatactgaagcagccggactgaatttcgtcactgaagctgtagaagataaagctgcacagggtgcagaagatattgtaagtgtatatgcgaaagtcgcatctcgtttctaattttaaaggttaacacaaatgactatcaaaactaaagctgaacttttgaacaaatggaagccattactggaaggtgaaggtttaccggaaattgctaatagcaaacaagcgattatcgctaaaatctttgaaaaccaggaaaaagatttccagacagctccggaatataaagacgaaaaaattgctcaggcattcggttctttcttaacagaagctgaaatcggtggtgaccacggttacaatgctaccaacatcgctgcaggtcagacttctggcgcagtaactcagattggcccagctgttatgggtatggtacgtcgtgctattcctaacctgattgctttcgatatttgtggtgttcagccgatgaacagcccgactggccaggtattcgcactgcgcgcagtatatggtaaagacccagtggctgccggtgctaaagaagcattccacccaatgtatggtccagatgcaatgttctctggtcagggtgctgctaagaaattcccagctctggctgctagcacacaaaccacagtaggtgatatctatactcacttcttccaggaaactggtactgtatatctgcaagcttctgttcaagtaacaatcgatgctggtgcgactgatgctgctaaattagatgctgaaattaagaaacaaatggaagctggtgcactggtagaaatcgctgaaggtatggctacttctatcgctgaactccaggaaggtttcaatggttctaccgataacccatggaatgaaatgggcttccgtatcgataagcaagttatcgaagctaaatctcgtcagctgaaagctgcttactctattgaattagcacaagacctccgcgctgttcacggtatggatgctgatgctgaactgtctggtattctggctacagaaattatgctggaaatcaaccgtgaagttgttgattggattaactactcagctcaggttggtaaatctggtatgaccctgactccgggttctaaagctggtgtatttgacttccaggacccaattgatattcgtggtgctcgctgggcgggtgaatcctttaaagctctgttgttccagattgacaaagaagcagttgaaattgctcgtcagaccggtcgtggtgaaggtaacttcattatcgcttcccgtaacgtagttaacgttttggcttcagttgataccggcatttcttatgctgcacagggtctggctaccggctttagcactgatactaccaagtcagtatttgctggtgttctgggtggtaaataccgcgtatatatcgaccagtatgctaaacaggattatttcactgtaggttataaaggtccgaacgaaatggatgctggtatttactatgctccatatgtagctctgactccgctgcgtggttccgatccgaagaacttccaaccggtaatgggattcaaaactcgttacggtatcggtatcaacccatttgcagaatccgctgctcaggctccggcttctcgcatccagagcggtatgccttctattctgaatagccttggtaaaaacgcttactttagacgtgtatatgttaaaggtatctaatctctaacgatagaaacacaattttagggaaccttcgggttcccttttttctattttatacgatagcaatcaggcatatcatccgcatttatccaattgcgaatagttttaggactaactttaaaatgctccgctgcgtaatcaggattatcaaatttaacgccctttatacatattggaataaattttttaataccaccaagtttttcagaaatagctttacgatgtgaaatcgatataggtttgttttttcgtggatgaacatgtgttttataatattcattgcgccctttaactcgcttcgcaatagtttcatcagattgcttaacgcctgtttttgcctttgatatttttcgtttagcttccacagtcattccttcttttgttcgtattgataacatattacgatatgaaggatcttgcaaatgaactataactggatttcctctgccaccaatagcagcattataggtatcagttctcataacgaattcctcattaactagtaaagcttccattttatacatctcctcagatgaggagaaagaataaagaatttcttttttaaagttatgaataccatattttttgatggattttttgatgtttacgccagaacccatataaccatcgttttcgtcaagagtagcatgagctccgatgtaaatttttccattgatgatattagtaatttgatatattaaatatttcattttaaacatcactccgtttgtatatgattataatatcatattactttggtcttgtaaataactttataaatagtattatatttcaacaaggaaaatacaatggctaaaatcaacgaacttctgcgcgaatcaaccacaacgaatagcaactcaatcggtcgcccaaatctcgttgctttgactcgcgctaccactaaattaatatattctgacattgtagcaacgcaaagaactaatcaacctgttgctgctttttatggtatcaaataccttaacccagacaacgaatttacatttaaaactggtgctacttacgctggcgaagctggatatgtagaccgagaacaaatcacagaattaacagaagagtctaaattaactctcaataaaggcgatttattcaaatataataatatcgtttataaagtattagaagatactccatttgctactatcgaagaaagtgatttagaattagctcttcagattgcaatcgttcttttaaaggttcgtctattttctgacgcagcgtcaacaagcaaatttgaaagctctgatagtgaaattgcggatgctagattccagattaataaatggcaaactgcagttaaatctcgtaaacttaaaactggcatcacagttgaattagcgcaagatttagaagcaaatggattcgatgctcctaatttcttggaagatttgcttgcaactgaaatggcagatgaaatcaataaagacattctgcagtctttgattacagtgtcaaaacgctataaagttacaggaattactgatagtggattcatcgatttgagttatgcatctgctcctgaagctggtcgttcattataccgaatggtatgtgaaatggtttcgcatatccaaaaagaatcaacttatacagcaacgttctgtgttgcttcagctcgtgccgctgcgattcttgctgcatcaggctggttaaaacataaaccagaagatgacaaatatctttcacaaaatgcctacgggttcttagctaatggtttaccgctttattgcgatactaacagcccattagattatgtaatcgttggcgtagtagaaaatatcggtgaaaaagaaattgttggatcaattttctatgctccgtatacagaaggtctcgacttagatgaccctgaacatgtaggtgcatttaaagttgttgttgatccagaaagcttacaaccatctatcggtttattagttagatatgctttatcagcaaatccttatactgtagcaaaagatgaaaaagaagcaagaataattgacggtggagacatggataaaatggcaggtcgttcagatttgtctgttttattaggtgttaagctaccaaaaattatcattgatgaataaaacaaagggacctttcggtccctttttatttaacttaccaactcaatccaagctggacgaagtacatcttgtaccattttaactaattcctttttaatcaaagaaggattatctgcttgagttagagtaataccttcacgagaagtttcttccaaaatatcttgaacagttagccccatcacctttccaaaatcctttggaccaatttcgccaattttagaaataacgttatttacgcggttcagtgtaacgtaacaagctaaaattcccaccaatttgttatcagcttctgatagctcaactttagctttaataggcttatcagactttttcttttcactaaatttagagttcttgcatttaatcgctacacgatttccattacgaagccaagaaggataacaaggtttcaatacatatccttcagcagtaaatacttcgccttttgcttcggcattccaaacgcatttatttgcatcaactaatccagcatggtctactgtaaaattataatcttggacgacagaatctaaatcatttggcaatttaataagctcttcaaatttaccgcgacctaaaagtggagccattttaaatttaaatgtattacagaatgattccatcatataatcatctacataagtcacatcaccgctttctgtagtaacaataatgtcaaatacataaaaatctttatcacaataatcaacattcttctgaatgccaggtccagcgaattcgccaaagacttgataagatacaaccgctgaggtttccataatatcttgtacagctttaatggaatcagcataattcttcaaaataatttcatacccaaagaaatcttcagcaggaagaatcggtccagtgcgtttagcgcaagtcactttatcacgctcaataatcaatgagaaatttgtgccgtgaatcttttcacgagctacccactccccaccagtcaatcccaagctataaagtttttcaataaatttagagttgtaatgattttcaagactgctatactttttaaacataattaatcctcaaaatgtaatttctaaccaatcaccatcacgctgatcactattgactttaaagctgaatccttcttttctcagccaatcaccaatttcttctgtaatcaatttatcacgagcaatacaataataattaaaatgtgttttaccttgttcagctgctttattagcaagttctgaaaaatctttaataaaacactctagcttaaactgtttactttttaatgctttttcgcgtaattgattagcaaaagattcattttcataaagatcatactgttccatttttcacctttttattgatatgtctttttctatagacaactttttctcgagcccataatacagccacttcttttgcctgtaagtttaattcacgagcaatttcaatgaatgactttccagactcatgaagagtaaacaccacaacctcagttctcataatcaatctcatgttatcgagttggtgccattatatacatcattttctgattgtgttttgtgtgctttcaaaatgaagaaaggggccgaagccccttatgattatggataggtatagatgataccagtttctaaagcagttttatgaatgatgtatccattacgcgattcttggacatcaacttctggatagtctttcatcatcttctggagagtgtaacgatgcaggtaatatttactatctgggtcgtcagttttccaatctttaccttcttcggtcattttttggatttcatccataacccaccaaccgcaccagatgtaagctgagctacggtgtggaagaggatgaacataaggtaattcaccttctggttctggagcaactttagccgtgactgttacattaccagttttggtaacggttacagggttataatctgcagcagtaacagttgcagtaacttcaatagtttgacttccaacagatgaggtatcgacagtatatacgttagttgacccttctacaggagaagaatctttcttccatgagtaagtaatttgtgcttcttctggagcacccgtaacattagccgtaaatgtagccgaagcatcttgctgaacattaatagaaggaggagtcaatgtaacctgtggattcattgtcttcttattaaccgttaatgatacttcattagaagtaacgcttagtgcatcataatctgtcgcggttacttgggctacgcatttaattctttttactccacttgtagttggagtatagctaaatgtagagttagtttctccaccaacttgtgaatcatctacataccactgatacgtagcagatgctccatcaggttgagaagctaaggcagcagtaaattgaactggggttccaatcactccagccgcaggactagcaggagttacggctaaggtagtcgtctgtgtcttatttttaactgtgatagttgttgtcgcttcagccgtttccgggcctccttcagaaagtgtatttgttgcaactactttaatagtcttttgaccggcaggtccttttagtacataactaaaagttgcttcagctccatcttgtggaacattatctacgctccaagcatatgtaatagttccgcctccagtttgaccactgggtgtagcagtaaactgcttagtttcatcaataacccctgtaggtgttttaggagttatatcaactgtaaaagtcataagttatccttattttaatgttacgaaagaagagttgcgtgtttcacgaattaaaactgatccatcgcgattaatgtaataaattaagctaaataaagtttggtgtgctgacgcatgttcaaaactagttgggtgagatttccaatcaggagtttcagcaatccattgataaatccaccaaggaacagtacagaatcctagattttttccaatcagttgaagattcggactaaagttttccggaagagtaaatacagacggcttttcagattcaataatctcagctacagcctgttcgaatttttcttcaacaaaaggagtatcttcaatcaaaacatcggtattttcaggaattttatccgtttctactacttcaattttaatgtcagatttaatcggagaatcaatcagaagtgctgcttctggattgacttcttcatcgtcatattttaatccctctgcggcatcagcagcatcaattaagtctttaatagataacccatcagtctctggcataggttcactagcgagcttctggagggcttcttcaatatcaacaacgatattatcaaaagatttattctttttgacctttataccaaactgttcagcatattcagctaatttagctttagcttctttgttatcatcaagagccttcagctctgcaatataatctttatctatcataatatttcctcagtataaatatagatatatttattactcggaaaatagtatgtaccactttgtatatgaaacaacaaatctaataaatggtaaaaagtatataggaaagcactctactgatgacttgaatgatggttaccttggttccggtaaggcaattcagcaggctataaagaaatatggtgaaaacaatttctctagaacaatactaaaagagtttaaaacttccgaagaagcgtacatgtatgaagaagaaattataactcctgaactaataaaaagcaaaaattattataatatgaaacctggtggaattggtggaattgttatgactacagatgttatagcaaagatgaaagaatcttccgctaaaagatttgaaaactcaccgggcacggtattaggtaaaacttgttatactaatggaactaaaaatatttttattaaacctggagaacttgttccagaaggatttgtaaaagggatggttcatcctaatagaaagtccagaaaaggatgtaaagtcaaaccgactaccacaggaactttttgggtcaataatggcgcaataaataaattaatacaaccagacggtattattcccgacggatttattaaaggtcgtctcatgaaaagagattctaaaggcaaatttagtaaggcataattatggatattaaagtacattttcacgacttcagtcatgtacgcatcgattgtgaagagagcacgttccacgaattaagagatttcttttcgtttgaggccgatggatatagatttaatcctcgcttcagatatggcaactgggatggacgaatccgtcttttagattataatcgtcttcttccattcggcttagtcgggcaaattaaaaaattctgtgataattttggctataaagcctggattgacccacaaattaacgaaaaagaagaattatcaagaaaagattttgatgaatggctttctaaattagaaatctattcaggaaataaaagaattgaaccgcactggtatcaaaaagatgcagtgttcgaaggattagttaatcgtcgtagaattcttaatcttccaacatctgcaggtaaatctttaattcaagctttgcttgcgcgatattatttggaaaattatgaaggtaaaattcttatcattgttccaacaactgctctgacaactcagatggctgatgacttcgtcgactatcgtttattcagccatgcaatgataaagaaaattggtggcggagcatcaaaagatgataaatataaaaatgatgcaccagtcgttgttggtacatggcaaactgtagtaaaacaaccgaaagaatggttctcacagtttggaatgatgatgaatgatgaatgccatcttgctacaggaaaaagtatttcatctatcatatcaggtttaaataactgcatgttcaaattcggtttgtctggttcattacgtgatggcaaagccaatatcatgcagtatgttggaatgtttggtgaaatatttaaaccagtaacgacttctaaattaatggaagatggacaagtaactgagctaaaaattaatagtatttttcttcgctatcccgatgagttcactactaaattaaagggaaaaacttaccaagaagaaataaaaattattactgggcttagtaaaagaaataaatggatcgctaaattagctattaagcttgcgcaaaaagatgaaaacgcttttgtcatgtttaaacatgtatcgcatggtaaagctattttcgatttaattaaaaatgaatacgataaagtttattacgtatcaggggaagttgataccgaaacccgcaatataatgaaaaccttagctgaaaatggtaaaggaataattatagtagctagttatggtgtattttctactggtatttcagttaaaaatctgcatcacgttgttttagcgcacggtgttaaatcaaaaatcattgtattacaaacaatcggtcgtgtattacgtaagcatggttctaagacaatagcaacagtctgggacctcatagatagcgcaggcgtcaagccaaaatctgctaatacgaaaaagaaatatgttcatttgaactatcttttaaaacacggcattgatcgtattcagcgctacgcagatgaaaaatttaattacgtaatgaaaacagttaatttaataagcttcggccctttggagaaaaagatgttactagaatttaaacaatttctttatgaagcttctattgatgaatttatgggtaaaattgcctcttgtcaaacattagaaggtttagaagaacttgaagcttattataagaaaagagtcaaagaaactgaattaaaagatactgatgacatctctgtgagagatgctttggcaggaaaaagagctgaattagaagattcagacgatgaagtagaagaaagcttttaaattaaaaaaggcccaaccaaaaaggaagggccaaaactatagactaaaggtcacactatagcaaaagttgtgtttcatttaattgttcttccgaactttctgaaactggtagttctttaatgtaattatagcaaggcccaggatgtacaggacctttgtctgtttcaacaaccaatgcagaatcgattggagttttacagacaacacaaatcttatctgacatgattgtctcctctgaattatatctatttatacaactctcatatgcatatcaatgcccatatctttagaataaaaatattcatcaagatatccggcaaattttcctttaatataaaggacatcttcaccacacgggtggtcggccaggatacgaatatcctgacgcttaagattatgctttttcattaagaattgaatttccgtttcaaattcttcttcataattaaaagcatcatcaatgctatatctcattattttccagcctcaaatgctcgcatgtcttgaatatgcttaatagcaaatccacgtgatttaatagcatcaagagctccgctacagaaatctaataaaatcccccaatactgcaacgaggtatcaacctttaaaacatccttatccgctgatagaactgtcttcatttctgatttctcgtaacgatccatactaaattcatcaccatctcctcgtcccgagtagtagtctaatctagctttaagagcaacttttttctgtgcttcaattctaagcatttcctttttaatacttgaatgcttattaagccatttactatataacatcacattattagctgcttcatactgtaatttagtcgaatctataaacacatctttcttcaattcttcttgaagatcttctaatctcatattgttctctattcaattgttattggttgttattggatggacttagattcattataccacgttttaacgtgaagcattatactctattactggaagccagctgcagttttatctgctcaatatcatcaggattatcgatgaccgaaaagcgtatttctactatcagagtataatcgtcataaacgggtatcacattaactgctaatttatcaatacgtggctcatagtttcttactgcgctttcgatattgcgttcaaccgtgtcagcagtaagaggagtcatattttcaaaaagctggtctgataaatcacatccaaattcagggtcaaacggtcttgaaccttttcttgttgtaataattcccaaaagactgtttttaattgaccttaatccaagcgatctggaaacgtctttgttccaatccattttcatttccgggtcaatatcagaataaagcttattaatatttgccattatagtaactcaaagaactctttgaggcctcttattacgtgagcatgggtttttccacactctggacacttaattggaacagccaaataaacggtaggctttaaaagcatatcttttatagctacaatatctgactctgtgatgatagaatataaatcttctagttccttttcatttaagtcttcaactggaatgctttccccgttagcatgaatcgtttctatacatgatactatcatgtgggctatatttttatcatcaaaaattttagggtatcggaatttaattttaatgtcacctagtgtataccagaggtcttctggtgcatctatttgtgtatgtaatagatttatatgggttggtatttcagttccacaggtgcacttccaggagttttcgtgattaacttcaccgagagaatgtgcccataaatgaatcaacaatagttctgattcttggcggtttaaatcttttgcatttgtgcagtctttgattagctttttaacaattacttctacggaaccattatttttggcagtaataagttctagatattctttaagcgtgaatgcgcgacaattgattattttagaaccaactctcacatcaaatttgtattcatacatatttagctcctttatttatcatatttataaatagaataaaaggagcatctatggcaaacattattcgttgtaaattaccagatggtgttcatcgttttaaaccatttacggtagaagattatcgagattttttgttagttcgaaacgatatagaacatcggtcaccacaagaacaaaagcaaataattactgatttaattgatgattattttggagactatccgaagacttggcaaccatttatatttttgcaggtatttgtagggtcaataggtaaaactaaaagtacggtcacatttatatgtccaaaatgtaaaaaagaaaagacagttccatttgaaatatatcaaaaagaattaaaggaccttgtttttgatgtagctaatgttaaaattaaattaaagtttccttctgagttttatgaaaataaagcaaagatgattactgaaaatattcattctgttcaagtagatgaaatatggtatgattggaaggaaattagcgagtccagtcaaatagaactagttgacgccatcgagatagaaacattagaaaaaattctcgatgcaatgaatcctattaatttaactctacacatgtcatgctgtaataagtacattaaaaaatacactgatatagtagacgtgtttaagctattagttaacccagatgagatatttactttttatcaaattaatcacacactcgtaaaaagtaattatagcttaaattcaataagtaaaatgattcctgccgagcgcggattcgtattaaaactgattgagaaggataaacaataatgagtatgttgcaacgccccggatatccaaatctcagcgttaaattatttgatagctacgacgcttggagtaataatagatttgttgaattagctgctactattaccacattaactatgcgggattctctttatggccgaaatgaaggaatgctgcagttttatgattctaaaaacatccatacaaaaatggatggaaatgaaataattcagatttctgtagctaatgcaaatgatattaataatgttaaaacacgaatttatggatgtaagcatttttccgtgtcagtagattcaaaaggtgataacatcattgctattgaattgggaactattcattctatagaaaatcttaaatttggtagaccatttttccctgatgcaggtgaatctataaaagaaatgcttggtgtcatttatcaggatcgcacattattaactccagcaataaatgctataaatgcttatgttcctgatattccatggactagcacatttgaaaactatttgtcatatgtaagagaagttgctctagctgtaggaagcgacaaatttgtatttgtatggcaagacatcatgggcgttaacatgatggactatgatatgatgataaatcaagaaccatatccaatgattgtcggtgagccatctttaataggtcaattcatccaagaattaaaatatccattagcatatgatttcgtttggttgactaaatcgaatcctcacaaacgtgacccaatgaaaaacgctactatctatgcgcattcatttttagattcttcaataccaatgattactacaggaaagggtgaaaactctattgtggtgtcaaggtcaggtgcttattctgaaatgacttataggaatggatatgaagaagctattcgtcttcaaactatggcacaatatgacggctatgctaaatgttctactatcggtaattttaacttgactcctggtgttaaaattatttttaatgatagtaaaaaccaatttaaaacagaattttacgttgatgaagttatccatgaattatccaataataattcagtaactcatctatatatgttcactaatgcaacgaaactggaaacaatagacccagttaaggttaaaaatgaatttaaatctgatactaccactgaagaaagtagttcttccaataagcaataaagaagtttctattcctaaaatgggtcttaaacattataacattttaaaagatgttaaaggtcctgatgaaaatttaaaacttctcattgattctatttgtccgaatttatcaccggcagaagttgatttcgtttctattcatttattggaatttaatggaaagattaaatctcgtaaagaaatagatggttatacttatgacattaatgatgtttatgtatgccaaagattggaatttcaataccaaggaaatacattttattttagacctcctggaaaatttgaacaatttttaacggtgagcgatatgttatctaaatgcttacttagggtcaacgatgaagttaaagaaattaattttcttgagatgccagcattcgttttaaaatgggcaaatgatatttttacaactttagcaattcctggccctaatggtccaataactggaattggcaatattattggattatttgaatgaaaaagccacaagaaatgcaaacgatgcgtagaaaagttatttcagataataaaccaacacaggaagcggctaaatccgcttctaatactttatctgggcttaatgacatatctacgaaattggatgatgctcaagctgcttctgaattaatagctcaaactgtcgaagaaaaatcgaatgaaataattggagcaattgacaatgtagaaagcgcagtgagtgatacatctgccggttctgagttaattgctgaaactgtcgaaattggcaacaatattaataaagaaatcggtgaatcgctcggaagcaaattagataaattaacaagtttactagagcaaaaaatccagacagctggaattcaacagactggaactagtttagctacggttgaaagcgctattcctgttaaagtcgttgaggatgatactgctgaatctgtgggtcctttattaccagctcctgaagcagttaataatgatcctgacgctgattttttccctacccctcagccagttgagccaaagcaagaatcaccagaagaaaaacagaaaaaagaagcatttaacttaaaattatctcaagctttagataaattaacgaagactgttgattttggatttaaaaaatccatttcaattactgataaaatatcaagcatgctatttaagtacaccgtcagtgctgctattgaagctgctaaaatgactgcaatgatattggctgttgttgttggaatagaccttttgatgattcactttaaatactggtcagataaattttcaaaagcctgggatttgtttagtacagactttaccaaattctctagcgaaaccggaacttggggtcctttattacagagcatctttgattctattgataaaattaaacaactttgggaagcgggagattggggtggattgacagtagctattgttgaagggcttggaaaggttctttttaatttaggtgaacttattcaattaggtatggctaaattatctgcagcaattcttcgagtcattcctggtatgaaggatactgctgatgaagtagaaggaagagcattagaaaatttccaaaattctactggagcatctctcaataaagaagaccaagaaaaagtagcaaattatcaagataaacgaatgaatggagaccttggcccaatagcagaaggactagacaaaatctctaactggaaaactcgtgcatctaactggattcgtggtgtagataataaagaagcgctgactaccgacgaagagcgtgcggcagaagaagaaaaattaaagcaactttcaccggaagaaagaaaaaatgctttaatgaaggctaatgaagctcgtgctgcgatgattcgttttgaaaaatatgccgattcagctgatatgagtaaagactcaacggttaaatcagttgaagctgcctatgaagaccttaaaaaacggatggatgacccggatttaaataattcaccggcagttaaaaaagaacttgctgctagattttctaaaattgatgctacttatcaagagctcaagaaaaatcagcctaatgccaaacctgaaacttctgctaaatcaccagaagcgaaacaagtccaggtgattgaaaagaacaaagcacagcaagctcctgttcaacaagcatctccttcgatcaataatactaataatgttattaagaaaaatactgtcgttcataatatgacacctgtaacgagcacgactgctcctggtgtatttgatgcgactggagttaattaaggaataatatggcaattgttaaagaaataactgctgatttaattaaaaagtccggtgagaaaatttcagccggacagagtactaaatcagaagtaggaactaaaacatacacagcccagtttccaactgggcgtgctagtggtaatgacactacagaggacttccaggtaacagatctatataagaatggattattatttactgcatacaatatgtcatctagggattctggaagtcttagatcgatgagatctaactactcttcttcatcttcgagtattttacgtacagctagaaacactattagtagtacagtatcaaaactatcaaatggattaatatcaaataataattcaggaacaataagtaaatctcctatcgcaaacattcttttaccgagatctaaatctgatgttgatacatcatcacatagatttaatgatgttcaagaaagccttatcagtagaggcggaggtactgctactggtgtgctaagtaatattgcttcaaccgcagtatttggggcactggaaagtataacacaaggtataatggctgataataatgaacagatttatacgacagccagaagtatgtatggtggtgctgaaaatagaactaaagtgtttacatgggatttgactccacgttcaacagaagatttaatggctattattaatatctatcaatattttaactatttttcttatggtgaaacgggtaaatctcaatatgctgctgaaataaaggggtatttagatgattggtatcgttctacgttaattgaacctttatctccggaagacgcagctaaaaataaaacactatttgagaaaatgacatcgagtttaactaacgttctagtagtttcaaacccgacagtttggatggtgaaaaactttggcgcaacatctaagtttgatggaaaaacggaaatatttggtccatgtcaaatacagagcattagatttgataaaacacctaatggtaactttaacggattagctattgctccaaacctccctagtacatttactctcgagattactatgagagaaattatcacgttaaaccgtgcttctttatatgcggggactttttaatgtattctttagaggaatttaataatcaagcaataaacgcagatttccaacgtaataatatgtttagctgcgtttttgcgacaactccatcaactaaaagctcttcgttgataagttcaattagcaacttttcttataataacttgggcctaaattcagattggttaggattaactcaaggtgatattaatcagggaattaccacgctaattacagctggcacacaaaaactgataagaaaatcaggagtcagtaaatatcttattggtgccatgagtcaacgtacagttcaaagtttattaggctcatttacagttggtacatatttaattgacttctttaacatggcatataactcatctggattgatgatatactctgtaaaaatgccagagaatagattatcctatgaaactgactggaactataattctcctaatattcgtataaccggaagagaattagaccctttggttatttcatttagaatggattcagaagcttgtaactatcgtgcaatgcaagactgggttaactccgttcaagacccagtaactggactgcgtgctttgccacaagatgtcgaggcagatattcaggttaatcttcattctcgcaatggattacctcatactgcggtgatgttcacgatgcattcaatatcagtgagcgctcctgagttatcatatgatggagataaccaaataactacatttgatgttacttttgcgtacagagtgatgcaggctggagcagttgataggcaacgtgcgcttgaatggcttgaatctgctgctataaatggtattcaaagcgttctcggaaatagtggaggtgttactggactatctaattcgctttcacgacttagtagattagggggaactgcaggaagcatttcaaacattaatactatgacaggaattgtcaattcgcagagtaaaatattaggagcaatataacaatggggaccgaaaggtccatatttttatttacggaatgaaatgaaagcagcaactgaagcaactaaactgtcttcaatataaacttcaatttttacaggagcttctgactcaaatttacctgttactacaccctgaaaaatactttcagtctgttctggctttgaaaaattttcagaaggaaaaattccgaactttttatctgttccaaaaattttgataaattcatcgtaaaccgcttcgttaaaagcattatcagcaggaataacgccttcaactacaagttcttgacctaaaaagcgtaaagaagatttcattttgtgttcctcatgttatgttagtaagactactataacacaacacgagggacttgtaaactacattttgaactttttagtacgcgtaataggcatgcgtcgattttatactgtttcattgtttgaagagcagtatcaaaaacagcattaatgacaccaattggatttccacccaagtttttaagcttaactaatgatagtttctcattaacacctatcattacgatatgcatcattttatcgcctggtttaacgttttatttgtatcaccgccagaggtgtatgtacaaaatctaaatccaggaagtacactttcttcaccggcttgaatagcaaaaatttgtggtattttatgcttaggatttaatgtaacaaccggcgccgaagcgccgtcaaataattctgtaataagttccatgatttatccttgaacgaacttgtaaggcatgtttgcaatatctatgcaagacgcaataattccaagagatgattctactttctgtttaacatttgatgaaacaaatgatgcaaagtcaactctatcttctatatcacctgtcattgtaaccaattcaccagtttccattaaatggtctccgtcatagattaccgattcggtgagttcttctgtggtcataacttcagcttgaataagcttgttgttagttttaactgttccatcattgtaagatgcatcggttattttattaactttgaccattaatccacgtggcaaaatgacttccatttcatttgaaggcgctaaacttccaccgggtaaaacaacattgaccttatcagccccagtaataacccatccaattccaactaaattatcgctagaatttacaagtccttcatcagttttatcaatagaaacgcttaaacgcttttcgtctggtaaaacacctatagatgaatcagtcatccaagtaccaaaaatatttggatataatgatgttgacacaaagtttctaaaataaaaaactcgattttttaccattgcttcgtatattgaaggtaacattcgttgtgaacgatacaaagtaataccttttggtaatcgttcaccatttttaaaggctgaatctaaattatcaatagctttttctatgtcagatgctgtcaaaatacttgtacgctcatctggattatataatcccaaaagagcattatttatgtctacatatcctgaacctacgtattcacgaattccgcgtttttgcgctggtgtgtatttagatgaatctttattttcgactataggatgtaatgaccatccagcggtaagagcatatccacgtaattctcttcgaataatctttgtttttattgcattccaagaattttgtactaactcatttgcagcatcttggtttaaatgcgaatgtttgttaatatttctttcaaaccaagcacccttatatttttctaaagtatcatcgacgatagaagcaatagtttctaaggtttttattgaagtaatagattcttttcgtaatctttctaaagctttattttttatttctgcgttaataatggattctttatcttcaggaattatagaagcttctctgaatgccatcccagctgtaacactgctaagagcattctctagtgcaaatcctgaagtagaaattaccgttaattcattagaatcagaaattaaaggcgcgtccgctggtttatttaattcggccgctgaagcctcaaatttttgaaacattggtgtttcaaatctagaagattccaatgactgactttgcgcaattgctctacgggaaattttaactttaacgattacagcttggtcagaacgtttatcattttcttgcgcaatagatgctgcaattgcctcatttttagttacttgagccccagtatctttattgatataaacatcaccgaccttcgattcaactttagtaaagagctcggtactaatttccggaactcctggaatgtcttctagtgatacatttttgcgatgtataagaatatatgcatactttttatcgtaatcccagagttccttaagaaggacgtatctaccacctgaacgactacggataagtctatcagcaataacttgaatttgtcgagcttggccagcagttttagacttaagaatacggagcatacaggcatcaattttatactggcgcattgtttgcattgcaacagtaaaaactgaattgatataattaattgggcttggaccaagaccttttaatttagcaattgaacctttagcagttaatgtaaaaggaacaatatgcatcattttatcgcccatctttaaatcacgattagtatcacctccagatgtataggtacataaacgaaagcctggttgttcaattgcatcatcaacatgaactgaaaaaatttgcggtattttcttctttggatataagtttgtaattggaagagtagtatcttcgtcaaataattctgtaataagttccatcatatcctctctagtgtttattctattctatttataaaattaaaggcccgaaggcctttaataatctattggtaagagagtacgatatatttcaaactttggacctttttcataagcatcaaatgtttctgtgaatttattataagcatatgcatctataaattcaatcatgatttgtgatacagaagtagaaacatctccaccttctttttgagccacgacaattgtttctaagtaagctttcatagaccagttacctcatgaaaatcaccaaatacatcttcgaatgtattagctttagttttatcttcacgtaaacgaatcgcaatcggaagaaataatttaacgtaatcagtgcggccatcagattttaaccaaccgttgcattcgcactctagaatttttccaatataataattttggttttccataatgcgagtacggtcaagttcatgcgattttacaccggctttatcttttaagcctgaaccagcatttaccttaatttttccacactctgactcaagaataaatccacccgctttagtagggtctttacggtgaggataaattcctacaatttttaaatcaacatcaattacttctttaaatttataaagattttttgaacgagcattttcccataatccatcgatatttttgagaataataccttcaagaccttggtcaatatactttttataaattaccttagcttcatctaggttatttactacctggttttcaattaaaattactttatcatatccagatgtcatttgttctagtttagaaaaacgtacatcatatttcaaacgaaatgcaggaagactgtatatttctaccaacgggacataatcccagacctgaaacttcatgcattgtgcttctttttcagaaatggttccctttaaagatttattggcgattccattagaagcagtacgtgattcagctacttcggcgaattctttagctttactgttttcaggataagcatcaaaaagaaaatctaggccttctggctcctttttaacttgctcatggtataccaattcgccatcaatcaacacaccttctggatgaatctggcgggcttcagcggtcattttaattaactcttccttaagaagatctaatcctagatattcattaccagctcgtgataaaagacgaacatcatctaattcatcacctctaacttcagcaaaacaccgagctccatcagcttttaactgagcaaaggctggaaatttgatattcttattaatgcctttttcatcataagaacttgcgagcatttgaggttgttcaggaattaaacctggccaaactttgtttgcaatagatactgaagcaccacattcaaggtctcgcatcatcactcgacgcaaaacttcaacatcatcttttttaccatcggtgatatatccagttaattcctcaattgctgcatttccagtcaatttccgagtagctaatgtgaattcaatgaagtcaagcatatcggtaagagtcaacattccaaaactctgggtagcaataccaggtttaggccatttcttgatataatactgtaacccacgagaataagtcagacgatatactcgtttaagcaattcattatctttattcttttcaagaattgcttgcttctgtttagttgaaccaatagatgctatttcgttcagaattttaagaatcattgttcatcctttagagtttggtttacagctctattataaatcaattcatcattaagctcagtcaaagacctgtggtacgtggttctaactttatttccttgcatccagtgcttgatataaatgaaaccttgctctacacattttttaaaaattcgttcgtctttttgagctcggaattctggattgcatctaaaaaattgatttacgtgaccgtaatcacgtgtagtattaccttcattttcataaatagtgtgaacaacaaacattagaatgctccttggaaaatattatcaccacaagtaggtctattatacaaatactctataccgccgggcttaatatagttccatgtctgaaacggatgcgtctgatatggatgatatggattataaggattaaatccaggagttctccaggtagtgttccaaggaaaagaatcgtttttaatcatcttttcaattacatcttttatagctttttcactatcaaaactttctttattttcctttggtgaaaaaagcttattctctacatcgttccatgtataaactcgctgagcagtttttggaatattgtcacgctctcctcgagccatccaatacacaggaacacgtaatatttcactcgcgtgatcgcagtggtgagcgagatcgtcaatataacaaattacgttatatttctcttttgcttttttgaacaactcttcttttgaagaatcatgactacacatcagtacttctgagaaagcaccaggaaaaagagcattcaaattaaattgacgatttaatagagcgtcaatagaatcacccaatgctgtaacagcaacaaaattataatcttcttttaatttgttaattacacacagagcatctttatatggagacaagtaacgaataaaatccgaacgattgtatttttcaattaacttgacgccaagttcttcatcacagttaaagagtttaccaggagaaataaatttctcatcttggatcatttttaaaatatgttctaacggaagattatatttctgagcaaaataaggaaggcctgattgccagcttaaacatactccatcaatatcagttaaaatagtaggcttcatagagagtctcttaataggtttaacacatcaataaattcagcttcggttagtattgtatcatcttttgttagaccactagcaatgctgtgcttcaaaacttttcctttcgaggcttgtaatgcatcacgaaagcccttgttttgaatcgctgcttcaaaatatgcatttgtgtataattctttccacgccggggagtatcttgaaaacggaactccaagccaaaagagggtcccacggtcctgagctctagcataagaccttccagcttgttgggcggcaagcccggataacccaaatatacgacgttgttgttcaacatttttcaccttacacccttggaggaatccttcaagacctccaaattgaataccatccataacgaaaggccattgggcgaaattacttaatgcacatgatggccacctaaaattgcttctaatctctaactcagacattttcaatgcttataatttcaacatcagcccaatgaccatagcaaggaagacgaaattcaactggccagttagggtccctttctaatataatagactcaacttgtggttcttcgtgttcatcagtatacggatttttatctgttactttatatgtgaccttaatgtactgaattccaaaaatcttattaattatattcatactaattcctttaatccgtagataggagataattcatcacccatacgaaggtcttcatttccatctacccaggaaacaatataagcctcttttatttgaataccactccatttaaatggaggtagaaccttagaaattaatccaggtataccaactccctttaattcaacagtttgacctaaaaagaatttcattagaacctcatctgaaaaccgtgcgatttaacattaccgccgccaatatcagaaatgttaatttcacgcgcaattgaagggtcaatgtcaatttcacgattgagtttagtgatagctaaagtatcacgcccatttacagtacggaactctaaaggacaaaccacatcaacgtagtttttaatcgattcgctagtaagttgcaaatgtgcaggaaggtctttaggtgcctttgagaaaaccacttcacagaaatttttgcgggtatcaaaataagtagtcataaacataatattttcctcagtaaggggctgaagcccctcattttattttaaatatcaaattcattaagaactacatcaaagattgcttcaagatgctcaggtttagctctgttactcagaatatgacgaatccaagttttaactaagagtttacgattagcaccattccagcaaggatgagtccctaaatcgcgttggcggaaatcatcatccagagcgattttgaagttggaacctttcatcgtgattgaaaccgtgataccgttttcaaatcgcatataaacgtagttaggagtcatatactgttcaatttcgcatactgatccattttgatgtttccagaggcaaatagtatcaatagaacctgcaataccattagaaacatatttacgttcaaagttaatgtagttcatttttattctccgagatgtttaattgcggtacaggtatataatatcatatcctgtaccaaagtaaacaattattttactactttccaatgctgcatgtcaagtttaccaacttttttcatcttctcaattaagcgttctgcacgttggcgagctgtaacataatgccattcgcctaattcattttgttcaatttttccaacgattactgtattcaattcataaatccaaccagtaaagaaattatgaacttgaattgtaaaggtgaaatctgttcccataccttctgttgtttctacttcaataatatcaccttcaactgccattaagaaccacatagtttcatcatatttaccattgaagcatttagttttaactgcagcgttcagattaatcgttttcattttattctcctttgtttgtgtaagataatactatcacaaaggaactatactgtaaacaactttgtgcaatctttggaaaataaaaaaggactcccgaaggagtcctcaacttatgctttctgcttaccaaaacgagaagcatcatctcgaagaaccgcacgtgctcggcgcatgatcttctcaacagtttgattgatacgagagttcgacccacgcttgtagccagcgcgtttagaatcaccaactttcttttcaactgctttctttgctttagcttgttttgccattataaattctcttttaaatgaaaatgcaggacttattggcattgcctgcgcaagccctcaaggggaacataggtttttggatatttaacgaccaggataaccataaacccgtcatcattcacattcaagaggtacaccgtaaaactgtcggggtcttaaaactataatgattcgcaaatcattaatcagacagttcgacggctcctcgatttagctcacactaaggcagtgaatctccaataaattacttcagtgttaccacaaagtgacgaactgcttttcgtgcagcagaagccagaggcttagcatatttaagttcatctttttcctgaagctcagcagctaatgcagtttgagcaggattcagatgtttgaaataacgcaggatttcaagagcttcggcttcaacatcaatagatgcgccatagttttcgtgaccattattccatgcgtttcgttgcagttcaagagcgtgttgtaattgtttaatcatttaaaaattctcgttagagattaaaactcggtagtcacgttcttctgaatttcatcttctttcgacagatctctcagttgtagactaccacatagaattgttcggttaacttattattccgacacccaattcatattattatttatatcacttataaagacacggaatagctttatagtgacaggtaacgaatttttgtttaatttcttttggctgcttaagacccagagctacaaaaggatgcggaacatttcgaatttgaccaactggaagagaagtcaaatcaccaacttcgcagaaaccttcaggaacatcaggaccgacagagtgaactacacacagttcaggaacttcaccttgaacacgtttaccgataataagtcctgattctgtaacttcttcatcaccggcttgtgcaggttcagaaactaaaataacatattcaccgacagcacgaattggtagctgttgtacttcagacatcgtttttcctttttgttaacagatgaattaataataacaaatagttcttaaagcatttatttaccaataaattgaagcaaatgctcaactttcataccattaacggaaatcaatttgtcaatagaaaaacctcgccacgcaccaagctcaacatcaaatactggaatcatgtcagtagattctttccgagtagattcagtcaatttgccagtttgcatggttggcataaagtctgcatcacgagtacctttcatagtacgaatagtaccatcagacttttcaaaaactacgtttgaaacacccatggacaatttagttttcaaaatttcacgaattgctactttctgctcagttgtcagtttcatttatttacctattacagttttaatatgagttgttccacgttctttaagggtggaaagtaatttttggcatttttctaaatcagatttccaactatatggtctatcaatacaaacccaatttgtcttataatactgtttccatttagagaaaaaatatttcttatactctacagcaaatgagatgttctcgttagaataagaactaattgctgtgagttttaccaaacgaaatttcattattcaccacagaattcgttgatattttcccagtttaacttattcaagtttttcttaggaacattaaacacttcaatacctgcatttcgcagaatatcatcccaaccgggtttatttttgtcgtatgtttcacaataaaccagctttttaataccagattgagctatcgcttttgcgcaatctggacaaggagaaagtgttacatacatagtagcaccttcaatagaagaaccatttcgtgcagcaaacaaaattgcatttagttcagcatgaatttcatttttagatgaccattccgagtgagcactacgatgttctttcgccaaaacaaaacgatcagttgaaccaaatgatacgcattcaggcttatgaccttgaatgatagcatgtttaggcttattcaacaaccatccttgctcagcagcataatcacaacagttcacaccccctgcgggtgaaccattatacccagtagaaataatacgtccattcttttcaattactgctcctaccttccaggagcaacattttgattcctgcgatactaaatatgcaatttgaagtactgtactcgctttcatttcataatcaccagataagcagatttagcagtttcaacacgataaatttcgtgacgaagtttagttatacttttaataacagaactaattatattctgcccatctttaaagcggtttttcttatcaataaaaactgcgccagtcatctttttgtgaagctcaactggatacttcgtcacaataatagcatcatacacagaaggatgaatactattcaccagagtatcattcattaaagttattctaatgaactgtgctgtttcagaatcaagcgctctatgatcgccagtatcattttcaagacaattatcaattatatcagttaaattcatcatagtacgccatacaccctttgtgcttcaactaatccatcaaaatccagtttaagatgcgatatttgatcgccatcacctggattcacaattactaatactgaacgaggagtttcggtaataacacgaaccgatgtttcaggaaatcgttcagaaaccttatttactaattcctgcgcaaatagtttaactttttcttggaattcctttaacagtaatcggtttttcacttagcaacattttgttttcctcatttgttttggtagagctataatatcacaactctaccgtaaagtaaaccattaaatcgctttgaattccgcagtttgagattcaaagcgaatatcgcctttgataacaagctcagcatcaagaccaaatacgacaatgatatgcgcggaacctggatacagtgtaatggcaatagaatccacctggtctggaagcaaagtgttcaatacatgagtcacttgagcatggattcgaagctcagctgcgttatcaagtttttcaaacatattattagcgataatttggctaaacactacttctacgattttagagtaagtcggaaacatatttacctcacataattttcttcaagccaatcaataacatccaacgcattatcaaaagttgaaccatctactctgtcttctgtttcataatcaagaacatctaggcctactcttccgtcaacaataggccatagacaaaatagatatttcttttctttttcaattttatcacaaagacgataaatcttttctaggttattcataagttttccatggtaaaggcagtttagttttctttactactagttcaacatcgggattcttttctcttaatttaagacattcctcccatgctctattttcactagtaaatacacaaaattgcccattactagtaccaactaaaccgctatttacaataacaatagcccaagtttcatggtgccaagccattaaaaatctcccgaagcgacttgccagcattcaacaccgatacgacgccacatttcaactacttgagttcggtcatcaatagctaatttcacgtcaaaatgcggtgcaatgtgtttccagaaaatttcttctttaactacatcgtctttacgggtatcgccttgttcgcgctgacattgcataactaatggaacgccagcaatgtcctcaacccatttacgggtcatacgataatatttcgttgggtcttctttagttccactttcacgacctgaaacgactacgatttgataacccataagagcatacatcttagacagttcaacaaccataggattgataacatcggtatcgcatttttcaaggtcataaggaccacgaccattcattttagctagtgtaccatcaacatcaaaaataactgcttttggtttaccaggagtcccattatatactggaagaccgagatactctcgcatgcttttatacattgaacgtaaaacatcaattggtactgctttagttccgcgttttgagttacgtttaaccaattcagtccaaggaacatcaaacactttatgttcaactttccagccgtattctttggcaaaagtttcccatgctaggcgacgttcaggattcaggttagtatctgaaatgattactcccttaacagaatcgccaccgtacagaatacttttagctgtatcaaactgcataccagttacgataccttctttctttttggtatacttgtactcatcgcgttcttcatgcgccataatagattggcgatagtcatcacgattgatattataaaacccgggattcttagcaataaattcacgagcccaagtactcttaccagaaccaggacagccaatagtcaaaataatctttttcatttattttttctcaactaatgattgaatataatcatgtaggtctttagatgctttaccccacttattttgatattcatttttgagattagcacgtgattgagctaataaaacatcattagttggaggtaaagattctaaccgctgaatctggcgtccataaatcattgcagccatctcggattcataaatcaatcctttgagatgttcaaattgatgccatgaaatcatttacatttatcctcttttagctcttgacgataataacatrtcatagttttctggtcatgtacatatcgttttacatcattaagccaaatacgaaattcctgagaatcttcaaatggcataccgacccaggctttaccatcaataactttaacttgccaagatagtttagcttcatcatatgactttatttgcacaggccaattaggatgaactgtttctttctttacttctagaggctttgtcgaacaaccaactagaagaccaatagataatattactgctgatagtttaatcatttagaaaggtcctggatgtcttctgcgaacttgttgaaggagttgttgatttgtttttcaaccaatcctggcttatgagccaccacatccgccttctttgcatctttgcgcagtttttcattttcacgctcaatagcagcaattgcctcacgatttttattattcatcgcatcaatataattatactgaattcgcaaattatttaatgctaaggcgttttcattggccgtttttgtaatttcagtaactgatgtttctaatctttctaccttatgttttaaaataatagatgttccgccaaatgctattacaagtaatagcaatccagctgtaaaattacttacatgcataaagttttaataacctctacaatatcgtcttgagaaagaccgttaattaaaatatgatgttcagctggagatttagaaattttaaagcatgcctcaacatcttctgccatatccgatgcactacgatttggattactaataccaaggcgatgttttcccgtcaaaggattaacgatgatatagcatttgcagctattgatatgaacattgggttgagtctgattaatgaacacttcacaatcatacttagcaagttgattttctaaaaagactttcatctcttcaaccgcatcaggaagcatatcacgggcttgctcaagacgacgatttcgatattctttaatggtcgttttccgcttgacttgcttagctaaatctttcttaagaccagtgatatatccaactcgacgatttcctttgaatacagaaatcccatctgtagtatcaccgtatgcttcaacgaccatttcagtagtaataagttgtaaatccatcataaagtcctcatgttatgtcagtaagactactataacacaacacgagggacttgtaaacaacttagtatccttctgggataaattttttataatttttcaaaaaattctgttcgatttcacacatgaccttttcttgactatcgtacccctggtataagctcatgatgataccgaacaggtgatccattccagcacctttagcaacaccttgtgcttccattgcataagtctttctatccttaccgcaatgcttattatgacagtcaagaactaaaaacagagctcggtctaagtacttcagataagtcgtttcaaatgcttcaatttttctgtatgaatattcatcgtcagcatacattgctttaagatcatctgatgcaccatcaataatagtcttaaacaatttttctggattatctaatgaactttttgtactatgaagagacacgtaccagtcagacttaattttaaaatgagaaccatctttcatcacagcaacatagccttcgatgttttctgcatttttagcttcttctatccatttagggctatcgatttcgtatcgttcaactagatacggacgaagagtagcatctttataaatatcatcgtatgaaatgtattcacccgtttcgttttcacgaacattcagtaaaataattttcatctcttgataagcaagaacgattctattcgtcggggcaacgaattcgaagttagcagtaaatccatcttcagctaattctttaagtctatcacgcaaccgatggtgattaatattcatcaaaattccattagccattaaagcctgctcagatttgattgaaccctttgatttgaacagaatttcatcaccgtctaaataagttgataccaaagacccgtcttcttttgttagaatataatcaacatcgtttaaatcgatattcatcgtgaacggattttcattcaagttaaaaaacttttccataggacgagaagcaattcttactggtttttctccatccatttcaaacataattccacgacattctagtgcatctggaagtaaccaatcagaataagatgcataattatatgagaaaattctgtaagttcttccagatgcacttacatcatctgagtaaaaaaacttacgctgcgaatccttacatagttccattaaattgttaaaaagttcttgcattgtgtatcctcttttgtgttttgaatatagtaccacactccatgtggaagcatcattttttcttgtgttgaatattccaaggcgggttaaacagtttaatgaatagaggctcctctaagtcaatcgttgcgattgtcattgtacctaactcatttgtcatagaaagattaaaacattggcgggcgtaaaattcaactttgcttccttcctttagcgcagaatgaattaatgcagatttagtagaatcagacgttttgtctttgcggttaatagcagttctataatagtttattcttttacgtaaatttttagtttttccaatataaacaagctcatcatttatagcaatagcataaattacgttatacttgtttggaatagataattgttttatacttccattgtcgtctaattctagctcagtatatttaataaatgaatattctgttgcaatttctttcataataaaatgggccttgcggcccactccttaaaagtattttttaaaactcatcataactttatcatcaacatcattatcaatctgtgcaacaaggtaagatgacagttctacttcttgcggcgcggattgaacattatcagaattaagatattcacgaatccaaggatatggatgtttaaccggagcatcggtaattgggcatggaagaccgcactgtttcatacgagatacagttaagtaatcaataaagctccacatgctatttgtatttaatccaggaacatcaccatctttaaataaatgaactgcccagtctttttcctggcggttaacttccatgaaaatatcaactgcttcttgttcacactcttgggcaattttaacccattcatcaccatcagtaccggattgaagttgacgaataatatattgggtgcctttaaggtgaagctgttcatcacgtgcaatgaacttcataatcttggcattaccttccatgatttccatgttcttatggaagttaaaggtaccgtatgttcgattgagctcgctaattctcaacccgttctcttatgaactgctgcatgttaccatgcagtccagactatatcacaatcccggagggattctccccatttcgagtcgcttgaccctacgtccgaagactagtcgttgaaccttgttttgcaattgtcgccgtgccatcgtttatatgttgatggtgataaatctttcttatcacaatatggacaattcaattgaattctatttggatttaatttacatctgtcattatgttttagataaaatccaggacctttaccaatgtgaccacagaaatcacatgtgatttctttttgtgcaggatgggtcccgttcttaaccatttctaatgtttttgctgatgttcttttcttatgttcttccatcagaatctttaggtataggtccgttagcatctatccaaatttttctatagttcaattgtgcatcctcttaaaagtataatcatatttatattatactaattaaaggtgcaagcaaaaccttggctgctagttttccataaaggactttccagcaattaaaggagttttcgataaacgttaccgtttaaaggcgcattttacgcaaaagatacataaaaacgaatagcttctaaggcattaataacgtgcaaacagaggtaaagagatttcatcagatctcgtttagctctttgctcaacgtctttatcagctagaattagaccttggtctttataatattcaaccatgtctttagcattttcccattcacgggttttaaccaagacatcatcgtaatatcgcccaatggactcagcacgtttcataatagcttcatctaatacaatctcatcaaacaccttcgatggatcagtataaagatttcgcatgatatgagtatatgaacgactgtgaatagtttcactaaaagtccatgtagcaacccatgtatcaaggcttgggtctgaaattaatgacataagtacagcagatggcgcacgaccctgaatgctatccaaaagtgattgatacttcaggttgttagtaaaaatattttgctgatactgaggaagcttattaaattgcgcagcatccatcattaagtttacttcttcaggacgccaaaaaaaactgatctgccgctcaatgagttcttcaaatactcgatgtcgttgaatatcatatcgagctaaacctaatccagaaccaaagaacatcggttcattcaaaacatcaactggatttgtattaaaaactgtactcatttagaatccttaaatttacatttatcataatgccatcttaaagcattgcctttattaactttcttaccacagtgcgggcaaggtggatactctgctccttgtttagttctcaaagaaatcaaatcacgggtttctttagtatgaggaacatctcttgtcggagaaattgtaccatacatcggattaagcacgccgactttagcagcagctatattctttttagcttcttcagttttaggctttctcatctttttcttaggtttcttcgcttttagcccttcctttggttgccgccgatattttgtgtttggtttcttctgttaaacgcagacctgttttcgctttttgacattttcagtctcgtttcttcagaaatgaccgtacctttgcgaaattgcgaatttaacttctttgcatgggcatatattttagaatgaactttataatgacgtttcttagttcctttcatattgcacatcatgaaaaatgcgaatataacagattttaccggataaatctttgataaaatagcatgcgctataaaatgctctctagctgttaattcaactaaattttctttatcatcagaacctcccatgcatctagggattatatgatgtgtctctttatattcggataaaggttcccgagcctgagctcgggaaattaggtcgttatagattttttgataattcattacaatttacacgctgcacaatcatcggctttaggagtttctatttcataatcatcagtaccagaaccatcacgggtattatgataatagaaatttttaatgccataataccatccgtatagcatgtcatcaatcattattgacattggaacctttccttttggaaaaatctgcgggtcataatatgtattcgctgaagctgattgacatacccatttcagcataatagctacctgcgtaagataaggtttattacctttcttagctaatttccatgtataatcatagaggtctatgttatgctcaatattgggcacgacttgattaaaggaaccctcttttgattctttaacacttaccggtccacgtggaggctcgtagccgtttgtactgttagaaacttgggaagatgactcacatggcataagtgctgataatgtgctattacggatgccaaatagcttaaggtcttcccgcagcgccgaccagtcacaaacgtattttggagctgcgatttggtcaatctttttattgtaccagtcgataggtaattcgcctcgagaccaacgagtgtctgaataatattccgaaggtcctttttctttggcgagcttaatggatgctttaatgagtccatactgtaatctctcaaatagttcatgtgttaaatcgttagcatcttcataagaagcaaagttacttgccagccaagctgcatagttggtaacacctacaccgaggttacgacgctttttagctttttctgcttcaggaaccggatatccttggtaatccaacagattatcaagagcacgaacttgaacttctgccaattcattaattttatcttggtcttgccagtcaaaattatctaatacgaatgcagagagagtacacaatccaatttcagcatcagggctattcacatcatttgttggaatagcaatttcacagcacaagttactctgacgaataggtgccttttcacgaataaacggagtatagttattcgtattatcaatgaactgcacataaatccttgctgttcctgaacgttcagtcatgagcaattcaaatagttcacgggctttaatacgctttttacgaatattagggtctttttctgctgcttcgtataattcacggaaacggtcttggtctttaaaataagaataataaagctcgccacccatttcatgcggactgaacaaagtaatgtaatcgttttttccaaaacgttccatcatcaaatcattcagttgaacaccataatccatatgacgaatgcggttttcttctacgcctttgttatttttcaaaacgagaagattttcaacttccaaatgccaaataggataataagcagtagcagcgccgccacgaattccaccctgtgaacatgatttaactgcagtctgaaaatgtttccaaaaaggaataacaccagtatggcgtacttcacccatgccaatcttagaaccttcggcacgaatcataccaacgttaataccaattccagcgcgtttagagatatattcaacaattgaagcagaagccttattgatagacttcaatgaatctcctgcctcaataacaacacatgaactaaactgtcgagtcggagtacgacaaccagccataataggagttggcaatgaaatctgtcgagttgatactgcttcataaaaacggataacatgttttaatctatcaacaggttcatcttgatgcagtgccattccaatagtcataaatgcaaactgtggagtttcataaatttgaccagtggttttatctttaactagatatttttcttttaattgcattgccccggaataagtaaattccatatcccgttcgtgcttaatttttgattctaaaaatgtaatttcttctgctgaatattttgacaataattcagggtcgtatttacctgcatttacacaataagaaatatggtcaataaatgaacgtggttcatactgcccataaacatgcttacgaagagcaaacattaaacagcgtgcagctacatattgataatcaggttcttcaaccgaaatagaattcgcagcagccttaatgacaatagtctgaatatcatcagttgtcattccatcacggagataagatttaatattttcatataattcataaggatctactgatgttccttcagctgcccaagataaaactttaataattttttgtgggtcaaagctctgagaaacaccactacttttgataacattaattaattgcataagtcctcaacttgaaaatcgtctttaaacaatcggttaactatatgagctattatatcaccatgacacggctttggtttacatgtgcatcctagcctcattccacgtaaaggctctaaatgtgctttagttatttctccggatttaattcgacgtataaaatcttttttgaataattcaatggcagcctcccggctgccagcatctttaccgacgtaatttccccaaaatgtaccgcggtgaatattaacatcaaagtcggatttatatttattcactacccggcatagacggcccacgctggaataattcgtcatattgtttttccgttaaaacagtaatatcgtagtaacagtcagaagaagttttaactgtggaaattttattatcaaaatactcacgagtcattttatgagtatagtattttttaccataaatggtaataggctgttctggtcctggaacttctaactcgcttgggttaggaagtgtaaaaagaactacaccagaagtatctttaaatcgtaaaatcatatatcctcgcaataataaaattacaccgccatctttcctttaataggagggtgtgatacatagttgttaagaacgaaatctttaggcctaagtttaagaacatattttaattgttctttagtagaaagatatcggaatttataaggtagaccacttattaccagctcacaaagctctttaggttcacgcctcaaaatttctttacattgttctacgtgattcatatagatatgagtattaccaccagaaaatatcaaatcccctggaataagattacacatcttagctacaatatgaactaacgtagcatatgacgcaatattaaacggtagcattatgttcagataaggtcgttaatcttaccccggaattatatccagctgcatgtcaccatgcagagcagactatatctccaacttgttaaagcaagttgtctatcgtttcgagtcacttgaccctactccccaaagggatagtcgttaggcatttatgtagaaccaattccatttatcagattttacacgataagtaactaatccagacgaaattttaaaatgtctagctgcatctgctgcacaatcaaaaataaccccatcacatgaaatctttttaatattactaggctttttacctttcatcttttctgatattttagatttagttatgtctgaatgcttatgattaaagaatgaattattttcacctgaacgatttctgcatttactacaagtataagcagaagtttgtatgcgaacaccgcacttacaaaacttatgggtttctggattccaacgcccgtttttacttccgggtttactgtaaagagctttccgaccatcaggtccaagtttaagcatcttagctttaacagtttcagaacgtttcttaataatttcttcttttaatggatgcgtagaacatgtatcaccaaacgttgcatcagcaatattgtatccattaattttagaattaagctctttaatccaaaaattttctcgttcaataatcaaatctttctcatatggaatttcttccaaaatagaacattcaaacacattaccatgtttgttaaaagacctctgaagttttatagaagaatggcatcctttttctaaatctttaaaatgcctcttccatctcttttcaaaatctttagcacttcctacatatactttattgtttaaagtatttttaatctgataaattccgcttttcataaatacctctttaaatatagaagtatttattaaagggcaagtcctacaatttagcacgggattgtctactagagaggttccccgtttagatagattacaagtataagtcaccttatactcaggcctcaattaacccaagaaaacatctactgagcgttgataccactgcaaatccaaatagccattacgcacattaaactgatagaacatatgacaaggcggtaatgccatatatttaagttcagctggattccatgcagaaacaatttgacgcctatcatttggcagttttttaatacgatcaataacttctataatttggtctacaccaccaaaatcacgccactgttttccataaattggaccaagttcaccgctatggtatcctaaatcttttgcttgattttcgtaattttcatcccagactgttttgccttggattaacgaatcgtgttgaattaatcgtaaatcattgacatttgtgcttcctgataaaaaccatattagctcagcaatgcaagctttccaggcgagcttcttagttgttaccgcaggaaaacctttagttaaatcccagcgtaatttagatccgaacagagcaattgttcctgtgcctgtacgatcatcggtttcataaccattttcaaaaatgtctttaattaaatcttggtattgtttcatttatatactgattccgtaagggttgttacttcatctattttataccaatgcgtttcaaccatttcacgcttgcttatatcatcaagaaaacttgcgtctaattgaactgttgaattaacacgatgccttttaacgatgcgagaaacaactacttcatctgcataaggtaatgcagcatataacagagcaggcccgccaattacacttactttagaattctgatcaagcatagtttcgaatggtgcattagggcttgacacttgaatttcgccgccagaaatgtaagttatatattgctcccaagtaatatagaaatgtgctaaatcgccgtctttagttacaggataatcacgcgcaaggtcacacaccacaatatggctacgaccaggaagtaatgtaggcaatgactggaacgttttagcacccataatcataattgtgccttcagtacgagctttaaaattctggaggtcctttttaactcgtccccatggtaaaccatcacctaaaccgaatgctaattcattaaagccgtcgaccgttttagttggagaataacggaataccaatttaatcattacgtaaatcctattttaattgaaaacgaatgcttacttggataatttcaatgacatacataatattttcctcaaacagactttttcacaattttccaatcagctttaaactgctcgacgtcagaatggtaaatccaaaatcctgcgctttctccgtcttcataaagaggacatccatcgcattcatcttcccatcccatatcacgtaaaagatgttcagctttttcaacaagttcagaatctttaccgatgatattaaaataccatttacctctaacttctgaatctttgatgctctggcgttgtaatctcattttattctccttagcaagctttaatcaaaagatataaacagaccaacataactgctgccataatataaggtgcgaacattttcttttctccattagttttgatagggtaatagtatcacactactaccctgatgtaaactactttttgaaagtttttcgcaaaagttcaatgatttcatctacattgttttcgtcaacaatgcagtgaatttttgttacgccagaaaccttgtctttgacttcatcttcttcagaagtcggttctttatattcgcggaaacaataaaactcttcttcactaagttcaaaataatcatcacccataccatcatcattatagatttcaccattagcacaaatgatttcggttacataatcaaagccatctaaacttgatattgatttaacttcaaaccaaccgccattttcttgaatgatgccgaccatactagcatttgatgaactaatatcaatgaaagatttaatacgatgtggatttaactcgtattttttgccgatttccattttgattttcctcattttaataggggcttgatagccccttgataattattgttcaatcagtcccatgtaaaattctgcgtcttcagaatccatgccatcacaatattcattagccataaagcgggtgaggtcttcaagaggaccttcaataacgatttgaatactccaaaacttagaatcttgcacgcttgtgatactaagttcaggataacgattacgaataatctcttcaatatattcaaaatcaacgatgtcaatatcaactttagccatattattttcctctttaattattagcagtattgccgatagttgtatagtaccataaagctttatgcttgtaaaccgttttgtgaaaaaatttttaaaataaaaaaggggacctctagggtccccaattaattagtaatataatctattaaaggtcattcaaaaggtcatccaggtccgtgtcatcagcactagatgaactaccagagcttgagctcataaaatcatcttcagtttttgtattgaagtcatcaacattgaatgcatccaaatcatcagccactttatcagctttcttagcagcagttgcagcagcaccgcccatcacagcagttcccataacttgaccgaatttagtattaagttcttcaaacgatttgaatttatctttagaagtcatttcagaaaggtcaaccatttgttcgaacagttctttctggaaagattcatcgtcaatgtttggaatcgcagattgattcaggaatttagattcatcgtagttactaaatccagaaacttgtttaactttcagtacaaagttagcaccttcccacggacaagttacatcaactggagtttcacccatttcaacatcaaccgcaatcattgcattgattttatcccagattttcttaccaaagcggtatttaaatactttaccttcgttttctggagcagctgggtcttttactacaagaatgttagcccagtaagaagttttacgtttaacaagactgtactctttattgtcagtgttgtatagatcatttttactgatgtattgacatactgggcaagaatcgtaatcaccatgggtagatgaacatgtttcaatataccatttaccatttttcttgaaaccgtgatttacaagaattgcgaatggtgcttgttcatcatttttagacggaagaaaacgaattactgcttgaccgttacccgcattatcgagtttcagtttccactcgcctttatcttcagaagaaaaacctttattgccattcagtttagccatttgtgcagcgagttcagcagtagatttacgtttaaacatttttatttcctttttaatttaatttaattaacagttggtgctatgacactttacctcatagctggcataattcgcaatactctgggtcttcgagaggtatccaacctgagttgaaatactttaccatcgatttagcagttgtatcagttatatttatattacctttaactcttcgccatccaggagttttaccgtacagattagaggataataataacacataattctcgtaagcaatatgagataatttccaagactctatattagctcgtgatgttttccaaggtctaaaatcgtcacggttcatataattagccaatctcatatgctctctaacttccgggtctttggctggatgagtttcaccactcacaccaaatccaccaccagcatataccagattaaaatagtctggattatctctggcatttacttcaagttggtattttcgttctgcttcaataacatccaagtcatcatcaatttgaattattttaacgctcggtttttgaagcattagcgcattcaaaaatcttttttgtttacatgagctccagtattcctttccggaagagtcatatattattccgttctcaaatgagcaatttaatttactaccgatatagtagtatggcggagtcttatttttgacacggtcttcaaatgtaaaccaatatactatattcatatcaatacttgcaagatttcacagtttcaatgaaaacatttttagctttctgtgaatcaatatttaaaatttttctataagcctttaactttatagaataattattccagactaaattatcagtctgttcatcatgtttatcaattatatttaaaaacgaatcaagcaagataaacgtctcaaacgaaattatgttcgattgcagaagtttaaaaatataacttgattgaacttttggattatactcaaaaatttctttaaaagcagaaacttcaacttttttactaaaataataaatgttgcgaatatcttcttcaaacttaaatttaatttgctttaagcgtccgatatattcacgataaaacacaagtgcatcagcgtcagagatgtcaccaatccaagcatcttggttagcaaccaaattgcttataaagattaaagcaagttcctttaatttatatttttctgataacttctggaaaaaatacttatcccttcgcttttgataagcggcatcagacacccgcatgcaccaattatacttaattacatcatactttccattcatatgttgttttatcattaagtataatttataaactgatttaccatcaatgtatctttcaccaccagcaggcatgcggagtttaatcatagtagaaaatctaatgtattagttttttcacaacgaacaacagaaggacgtaaaagattttcgtcaatagcttctgactgaattttttcaattatacccgaaggaataaatttagcaaattgagtttcaggaatagaattttcttctaagaatgctgttgtagcttcaagataactcattccaaactcttctaccattttttcaataataaatccattttcttggcggtcaagaagctttgctatttcatccttttctttcttaattgaaagttctttttctgaaagaccggtctcatcgaccggacgaatatcatttagagaaaactgtgtcataaagttcaactacctcttcagtttcagcttcaaacacatcacggttatctttatgatacaaagctaatagacgattaaacatcttaccatcaacgccaagttcatctttagcacgaattcgaatatctttaatcagttcattataaccggaaattttcagtttatgatcagatgcttctttaataaatttagccaagtcttcgccatggatagcttcatcaaattcaaccatttcttttttagccattattcacctcaaaattcattaatgctattagttaatttagaaagacccgcttttacaaaatatgaataaattttgccacgcggtggtaatttatatgaattatagtaattcacaatgtttgaagcaatattatcaggaatataatcaaaatcaattagaactaaattttctttataacgattatattcagattcagtgagaagcaccttagcttgctcacggtcattagcaatagcttcaacgattgaagttttcattgaaggagttcgttcaccttcaactctggtaaaccaaaagtcagatcgtactttaactgaagcaacgttatcctttttgtcgcctttaaggattttagtcatacagtcaatttcagcagaaccgcttttaattttaacccatttcttatgcatcggagaccattgcttaacatttggatatttgtgaagctgagtaaagtcaccatctgacgaaatgattaaaatcttatgtccttctaaagagaactttttaacaagaacagcaatgtggtcatctgcttcatacttatcaatatccataacaatgtatggcatataagctttcaattcatctataactttatggctggattcaaaataaccttcccagtcccaagtagattcttctcgtgcttttccacggtttttcttataataataagcgaaatcacgacgccaatatccagatttcgcgttatcaatacacagtacaattttagtgtatccaagcgtttttgcttttttgacattaaacttaattgagttcaatatcaaatgacgaaccattgataaattaattttttctttatctgggaagtttaccaaagcagttgaaagcgcaatttgactaaagtcaattaagcagattccttctttgtaatcttcatccaacatcatttctaaatccatatgaacctcgttcaattagtgagatttctattatataccatccaaatcttaaagtaaacaagtataaatacttattattgaaaacacaataggagcccgggagaatggccgagattaaaagagaattcagagcagaagatggtctggacgcaggtggtgataaaataatcaacgtagctttagctgatcgtaccgtaggaactgacggtgttaacgttgattacttaattcaagaaaacacagttcaacagtatgatccaactcgtggatatttaaaagattttgtaatcatttatgataaccgcttttgggctgctataaatgatattccaaaaccagcaggagcttttaatagcggacgctggagagcattacgtaccgatgctaactggattacggtttcatctggttcatatcaattaaaatctggtgaagcaatttcggttaacaccgcagctggaaatgacatcacgtttactttaccatcttctccaattgatggtgatactatcgttctccaagatattggaggaaaacctggagttaaccaagttttaattgtagctccagtacaaagtattgtaaactttagaggtgaacaggtacgttcagtactaatgactcatccaaagtcacagctagttttaatttttagtaatcgtctgtggcaaatgtatgttgctgattatagtagagaagctatagttgtaacaccagcgaatacttatcaagcgcaatccaacgattttatcgtacgtagatttacttctgctgcaccaattaatgtcaaacttccaagatttgctaatcatggcgatattattaatttcgtcgatttagataaactaaatccgctttatcatacaattgttactacatacgatgaaacgacttcagtacaagaagttggaactcattccattgaaggccgtacatcgattgacggtttcttgatgtttgatgataatgagaaattatggagactgtttgacggggatagtaaagcgcgtttacgtatcataacgactaattcaaacattcgtccaaatgaagaagttatggtatttggtgcgaataacggaacaactcaaacaattgagcttaagcttccaactaatatttctgttggtgatactgttaaaatttccatgaattacatgagaaaaggacaaacagttaaaatcaaagctgctgatgaagataaaattgcttcttcagttcaattgctgcaattcccaaaacgctcagaatatccacctgaagctgaatgggttacagttcaagaattagtttttaacgatgaaactaattatgttccagttttggagcttgcttacatagaagattctgatggaaaatattgggttgtacagcaaaacgttccaactgtagaaagagtagattctttaaatgattctactagagcaagattaggcgtaattgctttagctacacaagctcaagctaatgtcgatttagaaaattctccacaaaaagaattagcaattactccagaaacgttagctaatcgtactgctacagaaactcgcagaggtattgcaagaatagcaactactgctcaagtgaatcagaacaccacattctcttttgctgatgatattatcatcactcctaaaaagctgaatgaaagaactgctacagaaactcgtagaggtgtcgcagaaattgctacgcagcaagaaactaatgcaggaaccgatgatactacaatcatcactcctaaaaagcttcaagctcgtcaaggttctgaatcattatctggtattgtaacctttgtatctactgcaggtgctactccagcttctagccgtgaattaaatggtacgaatgtttataataaaaacactgataatttagttgtttcacctaaagctttggatcagtataaagctactccaacacagcaaggtgcagtaattttagcagttgaaagtgaagtaattgctggacaaagtcagcaaggatgggcaaatgctgttgtaacgccagaaacgttacataaaaagacatcaactgatggaagaattggtttaattgaaattgctacgcaaagtgaagttaatacaggaactgattatactcgtgcagtcactcctaaaactttaaatgaccgtagagcaactgaaagtttaagtggtatagctgaaattgctacacaagttgaattcgacgcaggcgtcgacgatactcgtatctctacaccattaaaaattaaaaccagatttaatagtactgatcgtacttctgttgttgctctatctggattagttgaatcaggaactctctgggaccattatacacttaatattcttgaagcaaatgagacacaacgtggtacacttcgtgtagctacgcaggtcgaagctgctgcgggaacattagataatgttttaataactcctaaaaagcttttaggtactaaatctactgaagcgcaagagggtgttattaaagttgcaactcagtctgaaactgtgactggaacgtcagcaaatactgctgtatctccaaaaaatttaaaatggattgcgcagagtgaacctacttgggcagctactactgcaataagaggttttgttaaaacttcatctggttcaattacattcgttggtaatgatacagtcggttctacccaagatttagaactgtatgagaaaaatagctatgcggtatcaccatatgaattaaaccgtgtattagcaaattatttgccactaaaagcaaaagctgctgatacaaatttattggatggtctagattcatctcagttcattcgtagggatattgcacagacggttaatggttcactaaccttaacccaacaaacgaatctgagtgcccctcttgtatcatctagtactggtgaatttggtggttcattggccgctaatagaacatttaccatccgtaatacaggagccccgactagtatcgttttcgaaaaaggtcctgcatccggggcaaatcctgcacagtcaatgagtattcgtgtatggggtaaccaatttggcggcggtagtgatacgacccgttcgacagtgtttgaagttggcgatgacacatctcatcacttttattctcaacgtaataaagacggtaatatagcgtttaacattaatggtactgtaatgccaataaacattaatgcttccggtttgatgaatgtgaatggcactgcaacattcggtcgttcagttacagccaatggtgaattcatcagcaagtctgcaaatgcttttagagcaataaacggtgattacggattctttattcgtaatgatgcctctaatacctattttttgctcactgcagccggtgatcagactggtggttttaatggattacgcccattattaattaataatcaatccggtcagattacaattggtgaaggcttaatcattgccaaaggtgttactataaattcaggcggtttaactgttaactcgagaattcgttctcagggtactaaaacatctgatttatatacccgtgcgccaacatctgatactgtaggattctggtcaatcgatattaatgattcagccacttataaccagttcccgggttattttaaaatggttgaaaaaactaatgaagtgactgggcttccatacttagaacgtggcgaagaagttaaatctcctggtacactgactcagtttggtaacacacttgattcgctttaccaagattggattacttatccaacgacgccagaagcgcgtaccactcgctggacacgtacatggcagaaaaccaaaaactcttggtcaagttttgttcaggtatttgacggaggtaaccctcctcaaccatctgatatcggtgctttaccatctgataatgctacaatggggaatcttactattcgtgatttcttgcgaattggtaatgttcgcattgttcctgacccagtgaataaaacggttaaatttgaatgggttgaataagaggtattatggaaaaatttatggccgagtttggacaaggatatgtccaaacgccatttttatcggaaagtaattcagtaagatataaaataagtatagcgggttcttgcccgctttctacagcaggaccatcatatgttaaatttcaggataatcctgtaggaagtcaaacatttagcgcaggcctccatttaagagtttttgacccttccaccggagcattagttgatagtaagtcatatgccttttcgacttcaaatgatactacatcagctgcttttgttagtttcatgaattctttgacgaataatcgaattgttgctatattaactagtggaaaggttaattttcctcctgaagtagtatcttggttaagaaccgccggaacgtctgcctttccatctgattctatattgtcaagatttgacgtatcatatgctgctttttatacttcttctaaaagagctatcgcattagagcatgttaaactgagtaatagaaaaagcacagatgattatcaaactattttagatgttgtatttgacagtttagaagatgtaggggctaccgggtttccaagaggaacgtatgaaagtgttgagcaattcatgtcggcagttggtggaactaatgacgaaattgcgagattgccaacttcagctgctataagtaaattatctgattataatttaattcctggagatgttctttatcttaaagctcagttatatgctgatgctgatttacttgctcttggaactacaaatatatctatccgtttttataatgcatctaacggatatatttcttcaacacaagctgaatttactgggcaagctgggtcatgggaattaaaggaagattatgtagttgttccagaaaacgcagtaggatttacgatatacgcacagagaactgcacaagctggccaaggtggcatgagaaatttaagcttttctgaagtatcaagaaatggcggcatttcgaaacctgctgaatttggcgtcaatggtattcgtgttaattatatctgcgaatccgcttcacccccggatataatggtacttcctacgcaagcatcgtctaaaactggtaaagtgtttgggcaagaatttagagaagtttaaattgagggacccttcgggttccctttttctttataaatactattcaaataaaggggcatacaatggctgatttaaaagtaggttcaacaactggaggctctgtcatttggcatcaaggaaattttccattgaatccagccggtgacgatgtactctataaatcatttaaaatatattcagaatataacaaaccacaagctgctgataacgatttcgtttctaaagctaatggtggtacttatgcatcaaaggtaacatttaacgctggcattcaagtcccatatgctccaaacatcatgagcccatgcgggatttatgggggtaacggtgatggtgctacttttgataaagcaaatatcgatattgtttcatggtatggcgtaggatttaaatcgtcatttggttcaacaggccgaactgttgtaattaatacacgcaatggtgatattaacacaaaaggtgttgtgtcggcagctggtcaagtaagaagtggtgcggctgctcctatagcagcgaatgaccttactagaaaggactatgttgatggagcaataaatactgttactgcaaatgcaaactctagggtgctacggtctggtgacaccatgacaggtaatttaacagcgccaaactttttctcgcagaatcctgcatctcaaccctcacacgttccacgatttgaccaaatcgtaattaaggattctgttcaagatttcggctattattaagaggacttatggctactttaaaacaaatacaatttaaaagaagcaaaatcgcaggaacacgtcctgctgcttcagtattagccgaaggtgaattggctataaacttaaaagatagaacaatttttactaaagatgattcaggaaatatcatcgatctaggttttgctaaaggcgggcaagttgatggcaacgttactattaacggacttttgagattaaatggcgattatgtacaaacaggtggaatgactgtaaacggacccattggttctactgatggcgtcactggaaaaattttcagatctacacagggttcattttatgcaagagcaacaaacgatacttcaaatgcccatttatggtttgaaaatgccgatggcactgaacgtggcgttatatatgctcgccctcaaactacaactgacggtgaaatacgccttagggttagacaaggaacaggaagcactgccaacagtgaattctatttccgctctataaatggaggcgaatttcaggctaaccgtattttagcatcagattcgttagtaacaaaacgcattgcggttgataccgttattcatgatgccaaagcatttggacaatatgattctcactctttggttaattatgtttatcctggaaccggtgaaacaaatggtgtaaactatcttcgtaaagttcgcgctaagtccggtggtacaatttatcatgaaattgttactgcacaaacaggcctggctgatgaagtttcttggtggtctggtgatacaccagtatttaaactatacggtattcgtgacgatggcagaatgattatccgtaatagccttgcattaggtacattcactacaaatttcccgtctagtgattatggcaacgtcggtgtaatgggcgataagtatcttgttctcggcgacactgtaactggcttgtcatacaaaaaaactggtgtatttgatctagttggcggtggatattctgttgcttctattactcctgacagtttccgtagtactcgtaaaggtatatttggtcgttctgaggaccaaggcgcaacttggataatgcctggtacaaatgctgctctcttgtctgttcaaacacaagctgataataacaatgctggagacggacaaacccatatcgggtacaatgctggcggtaaaatgaaccactatttccgtggtacaggtcagatgaatatcaatacccaacaaggtatggaaattaacccgggtattttgaaattggtaactggctctaataatgtacaattttacgctgacggaactatttcttccattcaacctattaaattagataacgagatatttttaactaaatctaataatactgcgggtcttaaatttggagctcctagccaagttgatggcacaaggactatccaatggaacggtggtactcgcgaaggacagaataaaaactatgtgattattaaagcatggggtaactcatttaatgccactggtgatagatctcgcgaaacggttttccaagtatcagatagtcaaggatattatttttatgctcatcgtaaagctccaaccggcgacgaaactattggacgtattgaagctcaatttgctggggatgtttatgctaaaggtattattgccaacggaaattttagagttgttgggtcaagcgctttagccggcaatgttactatgtctaacggtttgtttgtccaaggtggttcttctattactggacaagttaaaattggcggaacagcaaacgcactgagaatttggaacgctgaatatggtgctattttccgtcgttcggaaagtaacttttatattattccaaccaatcaaaatgaaggagaaagtggagacattcacagctctttgagacctgtgagaataggattaaacgatggcatggttgggttaggaagagattcttttatagtagatcaaaataatgctttaactacgataaacagtaactctcgcattaatgccaactttagaatgcaattggggcagtcggcatacattgatgcagaatgtactgatgctgttcgcccggcgggtgcaggttcatttgcttcccagaataatgaagacgtccgtgcgccgttctatatgaatattgatagaactgatgctagtgcatatgttcctattttgaaacaacgttatgttcaaggcaatggctgctattcattagggactttaattaataatggtaatttccgagttcattaccatggcggcggagataacggttctacaggtccacagactgctgattttggatgggaatttattaaaaacggtgattttatttcacctcgcgatttaatagcaggcaaagtcagatttgatagaactggtaatatcactggtggttctggtaattttgctaacttaaacagtacaattgaatcacttaaaactgatatcatgtcgagttacccaattggtgctccgattccttggccgagtgattcagttcctgctggatttgctttgatggaaggtcagacctttgataagtccgcatatccaaagttagctgttgcatatcctagcggtgttattccagatatgcgcgggcaaactatcaagggtaaaccaagtggtcgtgctgttttgagcgctgaggcagatggtgttaaggctcatagccatagtgcatcggcttcaagtactgacttaggtactaaaaccacatcaagctttgactatggtacgaagggaactaacagtacgggtggacacactcactctggtagtggttctactagcacaaatggtgagcacagccactacatcgaggcatggaatggtactggtgtaggtggtaataagatgtcatcatatgccatatcatacagggcgggtgggagtaacactaatgcagcagggaaccacagtcacactttctcttttgggactagcagtgctggcgaccattcccactctgtaggtattggtgctcatacccacacggtagcaattggatcacatggtcatactatcactgtaaatagtacaggtaatacagaaaacacggttaaaaacattgcttttaactatatcgttcgtttagcataaggagaggggcttcggcccttctaaatatgaaaatatatcattattattttgacactaaagaattttacaaagaagaaaattacaaaccggttaaaggcctcggtcttcctgctcattcaacaattaaaaaacctttagaacctaaagaaggatacgcggttgtatttgatgaacgtactcaggattggatttatgaagaagaccatcgcggaaaacgcgcatggacttttaataaagaagaaatttttataagtgacattggaagcccggttggtataactttcgatgagcccggcgaatttgatatatggactgatgacggttggaaagaagacgaaacatataagcgagttttaattcgtaatagaaaaattgaagaattatataaagagttccaagttttaaataatatgattgaagcttctgtcgccaataaaaaggaaaaattctattataaaaaccttaagcggttctttgctcttttagaaaagcatgagcatttaggtggtgaattcccttcatggcctgaaaaagaacagaagccttggtataagcgtttattcaagcattacgtataaatatcttaaaaggagggtctatggcagcacctagaatatcattttcgccctctgatattctatttggtgttctagatcgcttgttcaaagataacgctaccgggaaggttcttgcttcccgggtagctgtcgtaattcttttgtttataatggcgattgtttggtataggggagatagtttctttgagtactataagcaatcaaagtatgaaacatacagtgaaattattgaaaaggaaagaactgcacgctttgaatctgtcgccctggaacaactccagatagttcatatatcatctgaggcagactttagtgcggtgtattctttccgccctaaaaacttaaactattttgttgatattatagcatacgaaggaaaattaccttcaacaataagtgaaaaatcacttggaggatatcctgttgataaaactatggatgaatatacagttcatttaaatggacgtcattattattccaactcaaaatttgcttttttaccaactaaaaagcctactcccgaaataaactacatgtacagttgtccatattttaatttggataatatctatgctggaacgataaccatgtactggtatagaaatgatcatataagtaatgaccgccttgaatcaatatgtgctcaggcggccagaatattaggaagggctaaataattatttgttcgtatacatctctagatatcgatatacaccctcaaaaccctcgttgaattcgtcgatgagggttttcttatcttcttgagttaattcagaaacaattttacggaatgaattttgatttaactttctaccttcatgcgttactccaatctcatttagaaatgcaataaaattagcacgattctcaacaatatcttctctggaaaatttaatcaaaatagatgcaacagtaataatttcacgaactgtatcaatgtttttattcattaactataccactcaattagttgactttgttataatatcatcagacgcttgatttgtaaactggtctgtgtaattttcttcaaaaattttttctacgaattccttgaacgattcacgttcctgagctacattatgctcgattaccttttcaagattatgactcattcgaaataatcttcaatttcataatcatggacataaatcattatagtttttaatacatcatcaatattttttcctggagctggaattacgtaaaaataccctgcttttgagaggtctttataagttccaatcaagaaatcattattctcaagatgtaactcttcaactaattcattgacaattgaatggtataggtttggcagaaacttatatagcttttctagaatatcaattttgaatgtatattgaaccacggactgagaatcaataatcatagaccttccccttatgtttctgtttgcgattagattctttaaacgctttcttcttatccttatgaacagaagctttattaaaattatgctttgcgactaaattgttcatagtgctgaattacctctcttaaacatttgcatgtgaatgaaaactttttagctacaccacattcaaatatatgttctcttaaatcgcgtgtatcggtatatcccatctcaacaataaaatgccgtattagatttttatctttatcgtttagagaattaaaataatcagattttgaattaatttccctggccaaattgaatcaccttcagttgacgttttaactcttttatcatctcttcgttcatcgcaatataaagatcgcgtagagcaggttttagcattccatttactggagaactaaatggacatacataatcttttcctacgagctttttagtgaattccatatcacagaactgaaatcccggctcattggtataaattccccaattagttgacatcattttattggcatattccagtgcctggatttgattcataattccatcaatttgaaactttttaatattcattagtaaaggtcctcagagtaaagttctttttcactaccacgttcaatacttacttgtccagcgtaagttgcaataatcattgcttcttcacgtgtccaataattactatattggtcaataaacccttggtcatcatcacaaacttgctgagtaactaattgaggtttaactacatctaaaacttctgccatatctttagaataatgacgagcacctggaataataagagttcgtccatcttttaatttaaaacggttggctgcgcaaacaattcgtcgttgatacttttggttttcatcccagtacgcagtctgccaacagatttcaggaacttctttcagaatatcttcttctgtgcatttataaccatgcgctttaaatttttcaacaagactttctggagtttcacgagataaaggaacattcagcatttttaaacgattgataaatgggttcatttaaaccatcctttaatacgctgccacaaagttttctgttgagctttgttgacgccaattgagcgaataaccggttgagattcctggaattctttataatcagcaaggtaaatttcgtaagctgcatccgtaaatgaacttatcgctgccataaaattattgcgaatacctactggagcatctttactttcacgaatgatcatgtatttaccagtcttaatctttacgatagttccaagataagctccatggtaccaaatatcccaaccctcttgagtaggttctgcgcaacgacgaagttcattgacaatttctaacttgtttattatttattcctcacagttcagatgctacagtgattacagcttcaatgttttctgccgagcgtttaatgtcaagatacacattaccgtttttagcgattttacatgacattccgatgtcagtaaatttctgaatatgatgttccatcattttgtatccaaaaattcgcatatttccattgttattaatttcaaaattacgaattccgttagtgcgtttttctaaaatagcaagataattactacgataaatttcaacctttttaagaacaaatccatttttatctaaaagttttaacatgaggtctttatcttcttccatatcggaagtaatctcgcgagctttacgagttgctcgttttttcagcagttccggagcattttcctgtgcatataaagttgctgcatttgaaataatatcctgagcttcaccagtaatgattaatccatcaccagatttctccaccaggccttttttaatcaataccccaatattactattaactactgcgttacctaaatctggatgcacctcacgaacttctgcagctgtaatgaaatctttcttagcaatggtaattaaaatcgtagcagttttttcattcagaacatcgttagaagctttgatgatgtaagttactttagacattttctaatctccgtaattctgtatcagtagttgatagttgtatagtaccacagtatgctttggttgtaaaccgttttgtgaaaaaatttttaaaataaaaaagggagagcctcggctctccctaaaattactgcatgactgtgataactgtcatgataacacgttgaattccgaacgcaagaagacctcctgctacggctggaacaacgcctaaacccgccagtaaaatgctaccagatactaatgcagcgcttgtaataccaatgaatggactcatttgatttcctctaaatctttggtgtattcagtaactacatcagtagttttccaatattcgttttcttcttttttagctttagcttcttcagcaagtttctttgcttcatcggaagtcatatgaaaaatattcattccaactagtttatcaacataagaagaatacatatcgattttcgaaagttcttcggtcagttctttacgagttttaccctgtacaacaatttcacctgaaattactttcttaatgaaatgtgctttggcaaaggctaaacgaaaagctgactcagtttctttaattttgttatcaattcgtttttggacataagttttacgaacttcaacaaagtctttaattaaatcaactacgttatcgtaaacttgcagctttcctttctcattaataaccgtaatattctgggaacgacgctcaatcagtccgaagtctttcataatttttgcatggcgttcttcttcgttatcgctcaaagaatattctttgcggaatttaactttgaagccaaaaccatgctcaccacaagcatcatcccatgtaatgaagcctttattttcaagtgggtctaagattttactcacataagtttcacgatcatacttatacggaatctcagtgatatgcatttgagttcgtgaagtaaacttatatgttccacgaatttcatattgcccatcaatttcaacgacttcaccacgaaattctgggaattctactttcggtttagttactttctttccttgaagagcttgcagtacagctttcttgacagaagaaacactatgaggaagaatgtaagttgcataaccagttgcaataccggaaacgccattaagaagaacagtaggaataataggcaaatagaaagcaggcggaatgtgttctttatcttgatgtaccggagcatattcagtatctttatatacgttatagaaatttttacttacacgagcaaaaatataacgacttgccgctgccttttggacagtacgagaaccaaagtttccttgaccatctaacagaggaaagttattattccaagtattagccatcaaagcacctgcgtcttgtgcagagttttcaccatgatgatatccaaggtccgctacaccacctgcaatagaagcgagtttgtgaaacttatctttatttcctcgtgccaaatcaagagctcgagcaataacaaatcgttgaactggcttaaatccatcaatcatatttgggatagcacgattttcaaccgtgtacatagcataagccaatgcttcattatcaatgatactttttaaatcgcgattattcagttgcataaatttaccatactagtgaatgtagtgccataataacatcagaaatgaaaagcacgacttgaattaatccgaacattactccgtaatatagtgctaccaataaagcagcaagggctaatgaatagcccaagattttcttaatcattagataacaacacaaatgttaaatatgcacacataccctgggctaaagcttgtgaaaacacactgctagcatcgatacagatagttaaaacacatgctactatccaacaaataaatgaaataactcctaataattttgcaatattcatattttcctcactggcgtccgaagacgcctttagttttaagattgttacgatagaactgcatcacgtgttcgttatggaaattactcattaatatgcctgtaaaacaaatttaaagttatcagccaacatacggttcatttcttcgagtgtttgatactcagaatgatgattacgagtaaacgccaaagctagctgaccttttccaaatcccgtcgttagaggtttcatcttagaagcaggcagataaaacactgtgtatggaacattgttatttgcaatagtacgcgcaagttgagaccggcgttgacgaatatgacttaaaaccgcactgaatccttgcttagaacgctgattacctacataaaatcgtgcagatacgcatggattactaaatggaccaccgagtttactcactaaaaagtaaaatccaggtttagataaaatatctttatgcggagttcctaaaaaccattcaccacccttgattgtaccaataacagtagcgcctgcatcattcagatcagtaacagtcatatatttcatattaatttcctctaaattattttctactccaaggccgcatgaatacacacggccattaaattactcgtcgcagtcgacgctcaattcccaaaactcttctacagtataagtttcagtatcattttcaatacagaaacgttcattactattatttgctaaagtagcattaactgtcattttttcgctagtgctcttaagaggtgaaatacgaattaactgatcaccgttatctaaacaaaaaatttcaccaacttttacatctttaaaacttttcataattcacctcaaggagtataaaatccaaatgcagttgttgaccatcccatccaatatggaaaatttacaccaatgtaaaacataagaatataaaaccaaccgctcagcaaattcatcattttacaccattccaaattgtttcaaccacggattttaaaccattttgatgaatatccattcctactaccgccatcaaataaattccaactacaactgaacctaaggcaaaaatcagcatgaaaatgaataaagccggaaaaatattatcgaaaaaccattcaataaatgtaaaagcactgcgtttacgttcatattttcctcacataaatccaaagtaaacgtttaatacatcaatcattaaaacgattgggaatatactcaaaactattagtattataactacattccatatagctttaataatctttttcattttctgttcctccatagttgatagggtaatagtaccacggaagaacagtcttgtaaacaacttttttaaaaatattcgtaataaatgtgaataccaactactaccgctgaaacctgtgcaacccaccacgcacaagcaataagtacagaattcaaaattttcataataacctcattacaaaagtaaatgttaaacaaattactggaatactaattaaccaaacaaaacaccaccataatgaactcatagttcaatctcagcgattttcattttattctccaaatccgtatcagtagttgatagttgtatagtaccacggtccttgtggtatgtaaactgttttgtgaaattttttaaatggaaagataccatccgttgtagttgctttttcttacaactttacgaaggtcttctctgtcaccgatgaacttcggagtgtactggatgacacctggatgaatttctttagtgttgaatataattatacagtcagcgacttgatgatttagaatgggccctagatttattccagaaccatatggatactctccgctgcatcccgttgttaccgaaatccaacgtgagtcagtttgatgtgtcttaacttctacacgaagcccgcagtattttggatgagccaatacatcccatgcatatgtgtacggatcatcgacatcctcttggcctttattgacatatccactcaaccaatctgccacaaaaaactctgcgtacacagcgatacggcatctttcgataacttctgccttatcttgatttgggttttgttttaaagagtatcttgcagtatcagcaattttgaccttcatttcacaggtcaagtcactgttcgatagggtaaatgtcggaatctgaaatagtctctgtaacccaggattcgttttctgcatttaaactttcctttatgtcggaatcaccgatattcatataaatcataatttctcttaaaacaaaaggccgaagccctttattttacttgaattgtgcaattcttttctctagacattcagcataagatttcattgagatgaactgcgaaagtagcagttcttgctcaactgcgctaactgttagaaactttgcgctttctaaaaatttgctcagtgcattaattttgagcattaattgatcgtattcttcttttactcgtgcttgataagctaacataattttccttagttaagggccgaagccttatttaaattgttcagtaacgtcttcaactacttcatattggcaggtacgcattttagcatcgttgtaatcaatcggaattgatactacatcgcgaggatgaactttaacttttacaactcggctggttgaactaccaaagtgacgaatataagatttagaacacacatgcaaaccacgagaacaagtttgtgtatcatcgtcattcacacgagtacgtggcattttaactactttacccggactgttatcaaaggtgtttgagtgacagtcaaagtaattgctgcgaactactttccaagcatagaagtaaccatcttctgtaatttcaatatcgtttgctaccaagaaatcaaagagtcgagataccgctttttggcttgggttttccaacagattttccaagaacggaaaataaaattcaaagttttcgcctttttccatcgagtcaagaatacgatcaaccaaaccagaccgcaattcaatattttgatagaacaagcttccaccttcaattcgaacatcgccggaaatatatttttcaacagcacgacgaacattaattttttgtgccgcttcttccaacttatccgctacaagcagattaagaatttcctggaagtttgaatgagtattaggagttgcgttataagttacgccatcaacagtaattgaaatgaattttttagatgcattccaaataatgtcagatttagcaactggagcaataactgcatcgctattaactttaactgtaatatcaccgctaatagtaactttagggcgtttagcttcttcagcatttttcaaaacacgacggattgtgtcaaccgatacaccttgccaatcagccaattcctgttgggtgtaattaccacttgaatacagtttaacaatttcagcttgttcgtttttggtcaggcatttaatattgtacat

SEQ ID NO: 6 - DNA, 32 bp, synthetic construct
  source 1..32: mol_type = other DNA
cgaatgaccaggcatttaccgaccagcccatc

SEQ ID NO: 7 - DNA, 32 bp, synthetic construct
  source 1..32: mol_type = other DNA
agtgggaagggttgcaggacaccgtctttgcc

SEQ ID NO: 8 - DNA, 32 bp, synthetic construct
  source 1..32: mol_type = other DNA
ccgatgttaccttcctgaatcaaatccgcctg

SEQ ID NO: 9 - DNA, 32 bp, synthetic construct
  source 1..32: mol_type = other DNA
tgattgacggctacggtaaaccggcaacgttc

SEQ ID NO: 10 - DNA, 32 bp, synthetic construct
  source 1..32: mol_type = other DNA
gctgttaacgtacgtaccgcgccgcatccggc